(19) United States
(12) Patent Application Publication
(10) Pub. No.: US 2026/0327214 A1
(43) Pub. Date: Oct. 22, 2026

(54) 1,3,4-OXADIAZOLE DERIVATIVE COMPOUNDS AS HISTONE DEACETYLASE 6 INHIBITOR, AND USES THEREOF

(71) Applicant: CHONG KUN DANG PHARMACEUTICAL CORP., Seoul (KR)

(72) Inventors: Jae Kwang LEE, Gyeonggi-do (KR); Jaeki MIN, Gyeonggi-do (KR); Jinkyung IN, Gyeonggi-do (KR); Yi Hyun KIM, Gyeonggi-do (KR); Bomi JEON, Gyeonggi-do (KR); Hyunjin Michael KIM, Gyeongii-do (KR)

(73) Assignee: CHONG KUN DANG PHARMACEUTICAL CORP., Seoul (KR)

(21) Appl. No.: 18/996,396

(22) PCT Filed: Sep. 7, 2023
(86) PCT No.: PCT/KR2023/013453

§ 371 Date: Jan. 17, 2025

(30) Foreign Application Priority Data
Sep. 8, 2022 (KR) 10-2022-0114318

(51) Int. Cl. C07D 413/14; A61K 31/4439; A61K 31/444; A61K 31/4545; A61K 31/4709; A61K 31/4725; A61K 31/496; A61K 31/497; A61K 31/5377; A61K 31/5395; C07D 417/14; C07D 471/04; C07D 495/04

(52) U.S. Cl. CPC C07D 413/14; A61K 31/4439; A61K 31/444; A61K 31/4545; A61K 31/4709; A61K 31/4725; A61K 31/496; A61K 31/497; A61K 31/5377; A61K 31/5395; C07D 417/14; C07D 471/04; C07D 495/04

(57) ABSTRACT

The present invention relates to a novel compound having histone deacetylase 6 (HDAC6) inhibitory activity, a method for preparing the same, and uses thereof. The novel compound according to the present invention, a stereoisomer thereof or a pharmaceutically acceptable salt thereof has HDAC6 inhibitory activity, and is effective for preventing or treating HDAC6-related diseases including cancer, inflammatory diseases, autoimmune diseases, neurological or neurodegenerative diseases.

### TECHNICAL FIELD

The present invention relates to a novel structural 1,3,4-oxadiazole derivative compound having a histone deacetylase 6 (HDAC 6) inhibitory activity, a method for preparing the same, and uses thereof.

### BACKGROUND ART

Post-translational modifications such as acetylation in cells are very important regulatory modules at the center of biological processes and are strictly controlled by a number of enzymes. Histones are core proteins constituting chromatin, and help DNA condensation by acting as an axis around which DNA is wound. In addition, the balance between acetylation and deacetylation of histones plays a critical role in gene expression.

Histone deacetylases (HDACs) are enzymes that remove acetyl groups of lysine residues of histone proteins constituting chromatin, and are known to be related to gene silencing and to induce cell cycle arrest, angiogenesis inhibition, immunoregulation, cell death, and the like (Hassig et al., Curr. Opin. Chem. Biol. 1997, 1, 300-308). Further, inhibition of HDAC enzyme function has been reported to reduce the activity of cancer cell survival-related factors in vivo and activate cancer cell death-related factors, thereby inducing self-death of cancer cells (Warrell et al, J. Natl. Cancer Inst. 1998, 90, 1621-1625).

In humans, 18 HDACs are known, and are classified into four classes depending on homology with yeast HDACs. Here, the 11 HDACs using zinc as a cofactor may be divided into 3 groups of Class I (HDAC1, 2, 3 and 8), Class II (IIa: HDAC4, 5, 7 and 9; IIb: HDAC6 and 10) and Class IV (HDAC 11). Additionally, 7 HDACs of Class III (SIRT 1-7) use NAD+ as a cofactor instead of zinc (Bolden et al., Nat. Rev. Drug Discov. 2006, 5(9), 769-784).

Even though various HDAC inhibitors are in the preclinical or clinical development stage, up to date, only non-selective HDAC inhibitors are known as anticancer agents, wherein vorinostat (SAHA) and romidepsin (FK228) have been approved for the treatment of cutaneous T-cell lymphoma, and panobinostat (LBH-589) has been approved for the treatment of multiple myeloma. However, non-selective HDACs inhibitors are generally known to cause side effects such as fatigue, nausea, and the like, at high doses (Piekarz et al., Pharmaceuticals 2010, 3, 2751-2767). These side effects have been reported to be caused by inhibition of class I HDACs, and due to these side effects, non-selective HDACs inhibitors have been limited in drug development in areas other than anticancer drugs (Witt et al., Cancer Letters 277, (2009), 8-21).

Meanwhile, it has been reported that selective class II HDAC inhibition may not exhibit the toxicity seen in class I HDAC inhibition, and the development of selective HDAC inhibitors could solve side effects such as toxicity caused by non-selective HDAC inhibition. Thus, the selective HDAC inhibitors have the potential to be developed as effective treatments for various diseases (Matthias et al., Mol. Cell. Biol. 2008, 28, 1688-1701).

HDAC6, one of the class IIb HDACs, is known to be mainly present in cytoplasma and be involved in the deacetylation of a number of non-histone substrates (HSP90, coractin, etc.) including a tubulin protein (Yao et al., Mol. Cell 2005, 18, 601-607). HDAC6 may have two catalytic domains, and a zinc finger domain of C-terminal may bind to ubiquitinated proteins. HDAC6 is known to play an important role in various diseases such as cancer, inflammatory diseases, autoimmune diseases, neurological diseases, and neurodegenerative disorders, and the like, since it has a number of nonhistone proteins as substrates (Santo et al., Blood 2012 119, 2579-2589; Vishwakarma et al., International Immunopharmacology 2013, 16, 72-78; Hu et al., J. Neurol. Sci. 2011, 304, 1-8).

Common structural features of various HDAC inhibitors are composed of a cap group, a linker group, and a zinc binding group (ZBG), as shown in the structure of vorinostat below. A number of researchers have conducted studies on the inhibitory activity and selectivity to the enzyme through structural modifications of the cap group and linker group. The zinc-binding group is known to play a more important role in enzyme inhibitory activity and selectivity (Wiest et al., J. Org. Chem. 2013 78: 5051-5065; Methot et al., Bioorg. Med. Chem. Lett. 2008, 18, 973-978).

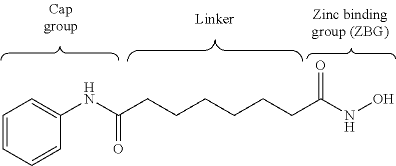 [SMILES: O=C(CCCCCCC(=O)Nc1ccccc1)NO]

Most of the zinc-binding groups are hydroxamic acid or benzamide, wherein the hydroxamic acid derivative show strong HDAC inhibitory effects, but have problems such as low bioavailability and serious off-target activity. The benzamide contains aniline, and thus also has a problem of generating toxic metabolites in vivo (Woster et al., Med. Chem. Commun. 2015, online publication).

Therefore, for the treatment of cancer, inflammatory diseases, autoimmune diseases, neurological diseases, and neurodegenerative disorders, and the like, there is a need to develop a selective HDAC6 inhibitor having a zinc-binding group with no side effects and improved bioavailability, unlike nonselective inhibitors with side effects.

### PRIOR ART DOCUMENT

WO 2011/091213: ACY-1215
WO 2011/011186: Tubastatin
WO 2013/052110: Sloan-K
WO 2013/041407: Cellzome
WO 2013/134467: Kozi
WO 2013/=08162: Novartis
WO 2013/080120: Novartis
WO 2013/066835: Tempero
WO 2013/066838: Tempero
WO 2013/066833: Tempero
WO 2013/066839: Tempero

### DISCLOSURE OF INVENTION

### Technical Problem

An object of the present invention is to provide a compound having selective histone deacetylase 6 (HDAC6) inhibitory activity, a stereoisomer thereof or a pharmaceutically acceptable salt thereof.

Another object of the present invention is to provide a pharmaceutical composition comprising a compound having selective HDAC6 inhibitory activity, a stereoisomer thereof or a pharmaceutically acceptable salt thereof.

Still another object of the present invention is to provide a method for preparing the same.

Still another object of the present invention is to provide a pharmaceutical composition for preventing or treating HDAC6 activity-related diseases.

Still another object of the present invention is to provide uses of the same for the preparation of a medicament for preventing or treating HDAC6 activity-related diseases.

Still another object of the present invention is to provide a method for preventing or treating HDAC6 activity-related diseases comprising: administering a therapeutically effective amount of the compounds as described above.

Still another object of the present invention is to provide uses for preventing or treating HDAC6 activity-related diseases.

### Solution to Problem

The present inventors discovered a oxadiazole derivative compound having histone deacetylase 6 (HDAC6) inhibitory activity and used the oxadiazole derivative compound to inhibit or treat HDAC6 activity-related diseases, thereby completing the present invention.

Hereinafter, it will be described in detail. Within the scope of the present invention are all combinations of the various elements disclosed herein. Furthermore, the scope of the present invention is not limited by the following specific description.

### 1,3,4-Oxadiazole Derivative Compound

In one general aspect, the present invention provides a 1,3,4-oxadiazole derivative compound represented by the following Chemical Formula I, a stereoisomer thereof or a pharmaceutically acceptable salt thereof:

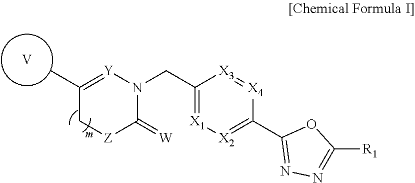

Ring V is aryl, heteroaryl or hydroheteroaryl {wherein at least one H of the aryl, heteroaryl or hydroheteroaryl ring may be substituted with —C₁₋₄alkyl, —C₁₋₄aminoalkyl, —C₁₋₄hydroxyalkyl, —C₁₋₄haloalkyl, -halo, —(CH₂)n-cycloalkyl, —(CH₂)n-heterocycloalkyl or —(CH₂)n-heteroaryl [wherein at least one H of the —(CH₂)n-cycloalkyl, —(CH₂)n-heterocycloalkyl or —(CH₂)n-heteroaryl ring may be substituted with —C₁₋₄alkyl, —C₁₋₄ haloalkyl, -halo, cycloalkyl or heterocycloalkyl (wherein at least one H of the cycloalkyl or heterocycloalkyl ring may be substituted with —C₁₋₄alkyl)]}; and
n is 0, 1 or 2.

According to an embodiment of the present invention, the compound represented by Chemical Formula I may be in the following ranges:
R₁ is —C₁₋₄haloalkyl;
X₁ is N;
X₂ to X₄ are each independently CR_{X};
R_{X} is —H or -halo;
Y is CR_{Y} or N;
R_{Y} is —H
Z is NR_{Z}, O or S;
R_{Z} is —C₁₋₄alkyl;
W is O or S;
m is 0 or 1;
Ring V is aryl, heteroaryl or hydroheteroaryl {wherein, at least one H of the aryl, heteroaryl or hydroheteroayl ring may be substituted with —C₁₋₄alkyl, —C₁₋₄aminoalkyl, -halo, —(CH₂)n-cycloalkyl, —(CH₂)n-heterocycloalkyl or —(CH₂)n-heteroayl [wherein, at least one H of the —(CH₂)n-cycloalkyl, —(CH₂)n-heterocycloalkyl or —(CH₂)n-heteroaryl ring may be substituted with —C₁₋₄alkyl, -halo, cycloalkyl or heterocycloalkyl (wherein, at least one H of the cycloalkyl or heterocycloalkyl ring may be substituted with —C₁₋₄ alkyl)]}; and
n is 0 or 1.

In addition, according to an embodiment of the present invention, the compound represented by Chemical Formula I may be in the following ranges:
R₁ is —CF₂H or —CF₃.

In addition, according to an embodiment of the present invention, the compound represented by Chemical Formula I may be in the following ranges:
X₁ is N; and
X₂ to X₄ are each independently CH or CF.

In addition, according to an embodiment of the present invention, the compound represented by Chemical Formula I may be in the following ranges:
Y is CH or N;
Z is N—C₁₋₄alkyl, O or S;
W is O or S; and
m is 0 or 1.

In addition, according to an embodiment of the present invention, the compound represented by Chemical Formula I may be in the following ranges:

Ring V is phenyl, 5-10 membered heteroaryl or 9-10 membered hydroheteroaryl {wherein, at least one H of the phenyl, 5-10 membered heteroaryl or 9-10 membered hydroheteroaryl ring may be substituted with —C₁₋₄alkyl, —C₁₋₄aminoalkyl, -halo, —(CH₂)n-cycloalkyl, —(CH₂)n-heterocycloalkyl or —(CH₂)n-heteroaryl [wherein, at least one H of the —(CH₂)n-cycloalkyl, —(CH₂)n-heterocycloalkyl or —(CH₂)n-heteroaryl ring may be substituted with —C₁₋₄alkyl, -halo, 4-6 membered cycloalkyl or 4-6 membered heterocycloalkyl (wherein, at least one H of the 4-6 membered cycloalkyl or 4-6 membered heterocycloalkyl ring may be substituted with —C₁₋₄alkyl)]}; and
n is 0 or 1.

In addition, according to an embodiment of the present invention, specific compounds represented by Chemical Formula I of the present invention are shown in Table 1 below:

TABLE 1

| Example | Compound | |
|---|---|---|
| No. | No. | Structural Formula |
| 1 | 1 | 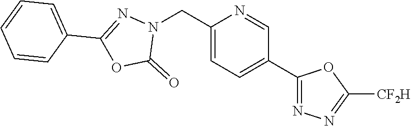 [SMILES: O=c1oc(-c2ccccc2)nn1Cc1ccc(-c2nnc(C(F)F)o2)cn1] |
| 2 | 2 | 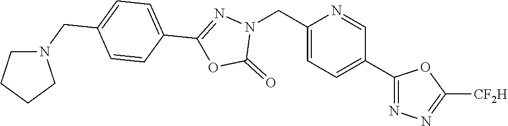 [SMILES: O=c1oc(-c2ccc(CN3CCCC3)cc2)nn1Cc1ccc(-c2nnc(C(F)F)o2)cn1] |
| 3 | 3 | 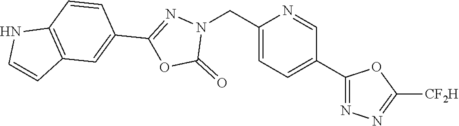 [SMILES: O=c1oc(-c2ccc3[nH]ccc3c2)nn1Cc1ccc(-c2nnc(C(F)F)o2)cn1] |
| 4 | 4 | 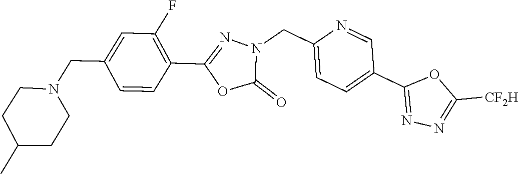 [SMILES: CC1CCN(Cc2ccc(-c3nn(Cc4ccc(-c5nnc(C(F)F)o5)cn4)c(=O)o3)c(F)c2)CC1] |
| 5 | 5 | 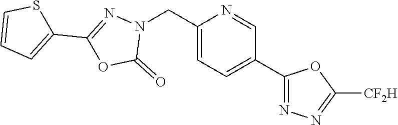 [SMILES: O=c1oc(-c2cccs2)nn1Cc1ccc(-c2nnc(C(F)F)o2)cn1] |
| 6 | 6 | 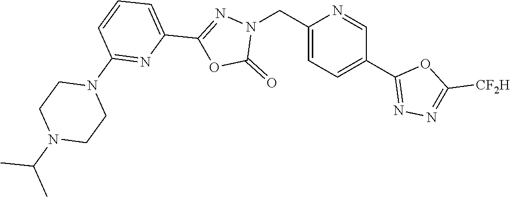 [SMILES: CC(C)N1CCN(c2cccc(-c3nn(Cc4ccc(-c5nnc(C(F)F)o5)cn4)c(=O)o3)n2)CC1] |
| 7 | 7 | 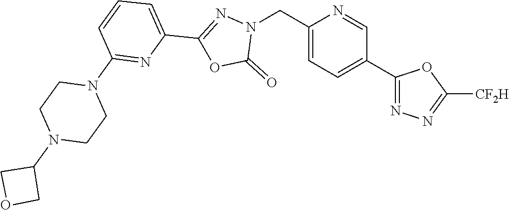 [SMILES: O=c1oc(-c2cccc(N3CCN(C4COC4)CC3)n2)nn1Cc1ccc(-c2nnc(C(F)F)o2)cn1] |
| 8 | 8 | 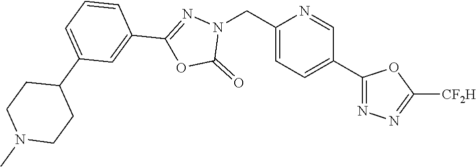 [SMILES: CN1CCC(c2cccc(-c3nn(Cc4ccc(-c5nnc(C(F)F)o5)cn4)c(=O)o3)c2)CC1] |
| 9 | 9 | 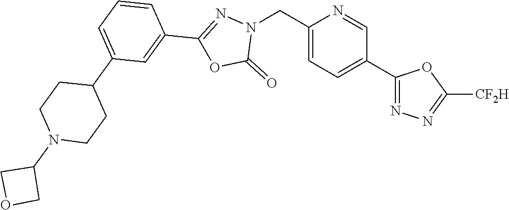 [SMILES: O=c1oc(-c2cccc(C3CCN(C4COC4)CC3)c2)nn1Cc1ccc(-c2nnc(C(F)F)o2)cn1] |
| 10 | 10 | 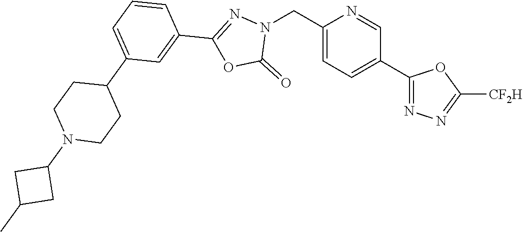 [SMILES: CC1CC(N2CCC(c3cccc(-c4nn(Cc5ccc(-c6nnc(C(F)F)o6)cn5)c(=O)o4)c3)CC2)C1] |
| 11 | 11 | 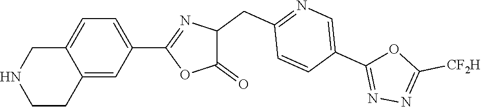 [SMILES: O=C1OC(c2ccc3c(c2)CCNC3)=NC1Cc1ccc(-c2nnc(C(F)F)o2)cn1] |
| 12 | 12 | 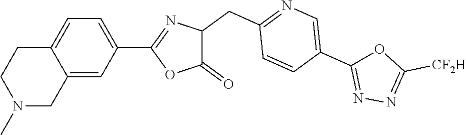 [SMILES: CN1CCc2ccc(C3=NC(Cc4ccc(-c5nnc(C(F)F)o5)cn4)C(=O)O3)cc2C1] |
| 13 | 13 | 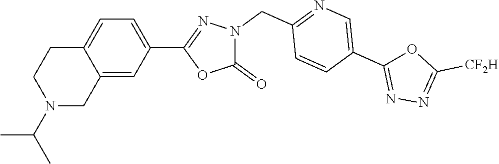 [SMILES: CC(C)N1CCc2ccc(-c3nn(Cc4ccc(-c5nnc(C(F)F)o5)cn4)c(=O)o3)cc2C1] |
| 14 | 14 | 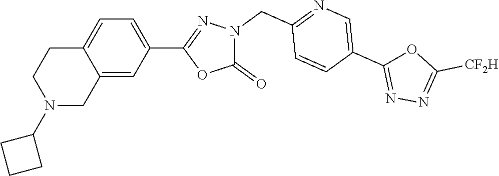 [SMILES: O=c1oc(-c2ccc3c(c2)CN(C2CCC2)CC3)nn1Cc1ccc(-c2nnc(C(F)F)o2)cn1] |
| 15 | 15 | 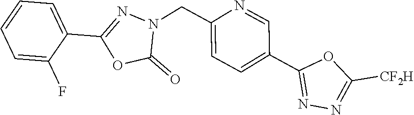 [SMILES: O=c1oc(-c2ccccc2F)nn1Cc1ccc(-c2nnc(C(F)F)o2)cn1] |
| 16 | 16 | 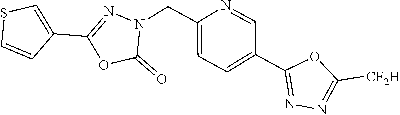 [SMILES: O=c1oc(-c2ccsc2)nn1Cc1ccc(-c2nnc(C(F)F)o2)cn1] |
| 17 | 17 | 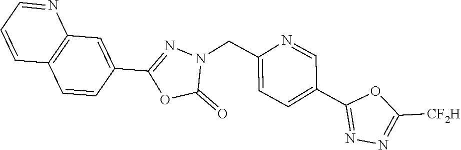 [SMILES: O=c1oc(-c2ccc3cccnc3c2)nn1Cc1ccc(-c2nnc(C(F)F)o2)cn1] |
| 18 | 18 | 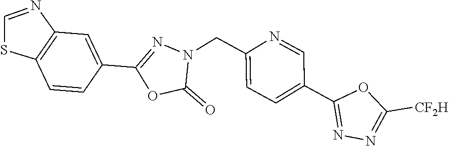 [SMILES: O=c1oc(-c2ccc3scnc3c2)nn1Cc1ccc(-c2nnc(C(F)F)o2)cn1] |
| 19 | 19 | 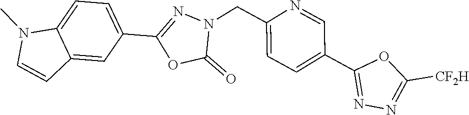 [SMILES: Cn1ccc2cc(-c3nn(Cc4ccc(-c5nnc(C(F)F)o5)cn4)c(=O)o3)ccc21] |
| 20 | 20 | 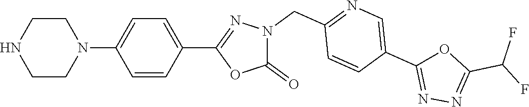 [SMILES: O=c1oc(-c2ccc(N3CCNCC3)cc2)nn1Cc1ccc(-c2nnc(C(F)F)o2)cn1] |
| 21 | 21 | 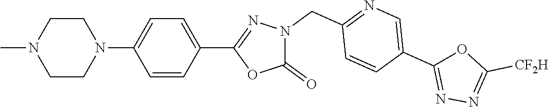 [SMILES: CN1CCN(c2ccc(-c3nn(Cc4ccc(-c5nnc(C(F)F)o5)cn4)c(=O)o3)cc2)CC1] |
| 22 | 22 | 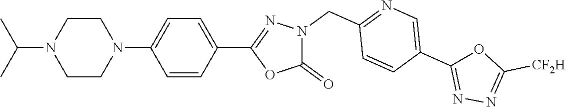 [SMILES: CC(C)N1CCN(c2ccc(-c3nn(Cc4ccc(-c5nnc(C(F)F)o5)cn4)c(=O)o3)cc2)CC1] |
| 23 | 23 | 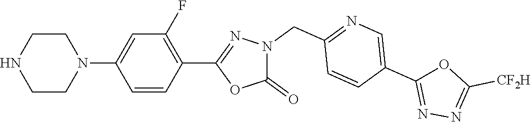 [SMILES: O=c1oc(-c2ccc(N3CCNCC3)cc2F)nn1Cc1ccc(-c2nnc(C(F)F)o2)cn1] |
| 24 | 24 | 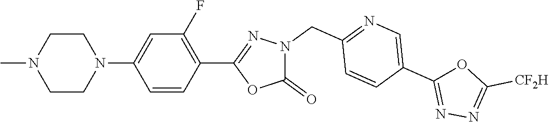 [SMILES: CN1CCN(c2ccc(-c3nn(Cc4ccc(-c5nnc(C(F)F)o5)cn4)c(=O)o3)c(F)c2)CC1] |
| 25 | 25 | 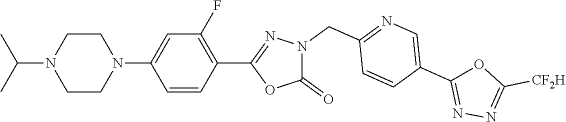 [SMILES: CC(C)N1CCN(c2ccc(-c3nn(Cc4ccc(-c5nnc(C(F)F)o5)cn4)c(=O)o3)c(F)c2)CC1] |
| 26 | 26 | 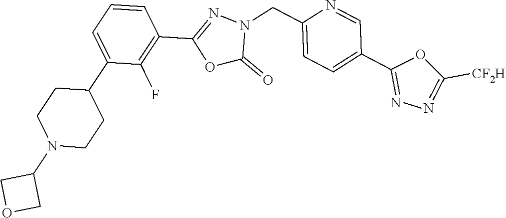 [SMILES: O=c1oc(-c2cccc(C3CCN(C4COC4)CC3)c2F)nn1Cc1ccc(-c2nnc(C(F)F)o2)cn1] |
| 27 | 27 | 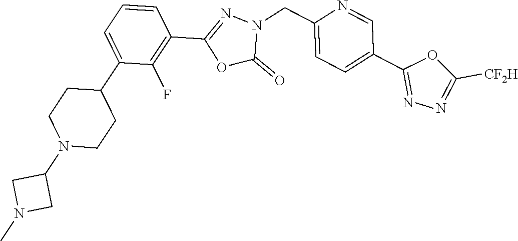 [SMILES: CN1CC(N2CCC(c3cccc(-c4nn(Cc5ccc(-c6nnc(C(F)F)o6)cn5)c(=O)o4)c3F)CC2)C1] |
| 28 | 28 | 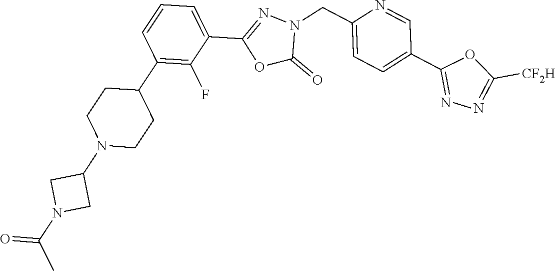 [SMILES: CC(=O)N1CC(N2CCC(c3cccc(-c4nn(Cc5ccc(-c6nnc(C(F)F)o6)cn5)c(=O)o4)c3F)CC2)C1] |
| 29 | 29 | 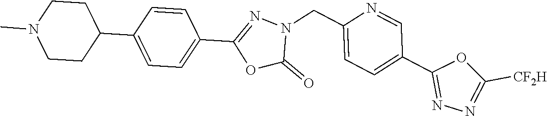 [SMILES: CN1CCC(c2ccc(-c3nn(Cc4ccc(-c5nnc(C(F)F)o5)cn4)c(=O)o3)cc2)CC1] |
| 30 | 30 | 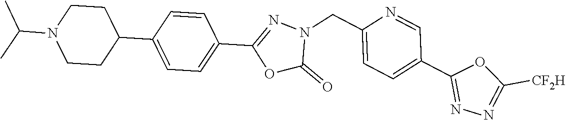 [SMILES: CC(C)N1CCC(c2ccc(-c3nn(Cc4ccc(-c5nnc(C(F)F)o5)cn4)c(=O)o3)cc2)CC1] |
| 31 | 31 | 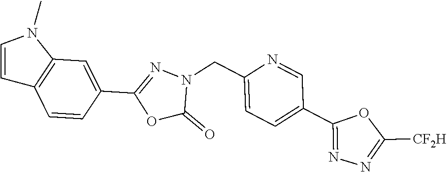 [SMILES: Cn1ccc2ccc(-c3nn(Cc4ccc(-c5nnc(C(F)F)o5)cn4)c(=O)o3)cc21] |
| 32 | 32 | 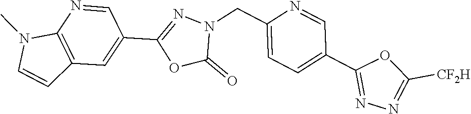 [SMILES: Cn1ccc2cc(-c3nn(Cc4ccc(-c5nnc(C(F)F)o5)cn4)c(=O)o3)cnc21] |
| 33 | 33 | 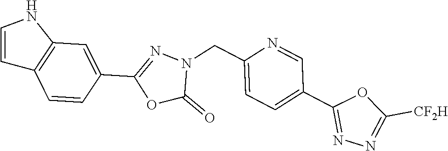 [SMILES: O=c1oc(-c2ccc3cc[nH]c3c2)nn1Cc1ccc(-c2nnc(C(F)F)o2)cn1] |
| 34 | 34 | 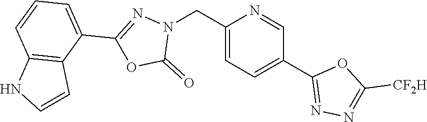 [SMILES: O=c1oc(-c2cccc3[nH]ccc23)nn1Cc1ccc(-c2nnc(C(F)F)o2)cn1] |
| 35 | 35 | 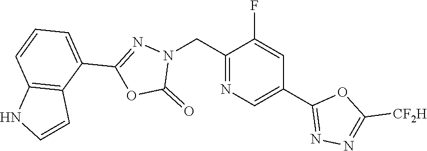 [SMILES: O=c1oc(-c2cccc3[nH]ccc23)nn1Cc1ncc(-c2nnc(C(F)F)o2)cc1F] |
| 36 | 36 | 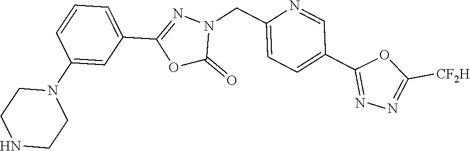 [SMILES: O=c1oc(-c2cccc(N3CCNCC3)c2)nn1Cc1ccc(-c2nnc(C(F)F)o2)cn1] |
| 37 | 37 | 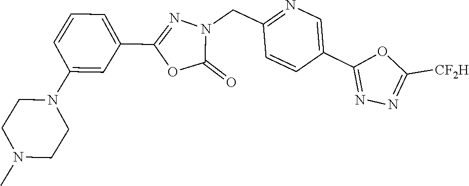 [SMILES: CN1CCN(c2cccc(-c3nn(Cc4ccc(-c5nnc(C(F)F)o5)cn4)c(=O)o3)c2)CC1] |
| 38 | 38 | 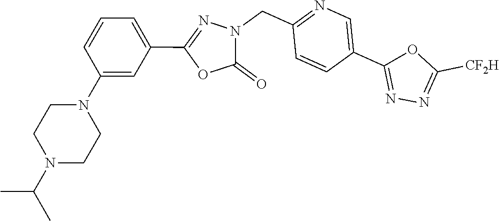 [SMILES: CC(C)N1CCN(c2cccc(-c3nn(Cc4ccc(-c5nnc(C(F)F)o5)cn4)c(=O)o3)c2)CC1] |
| 39 | 39 | 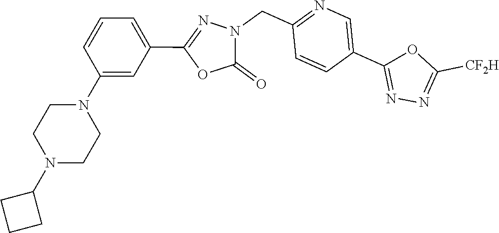 [SMILES: O=c1oc(-c2cccc(N3CCN(C4CCC4)CC3)c2)nn1Cc1ccc(-c2nnc(C(F)F)o2)cn1] |
| 40 | 40 | 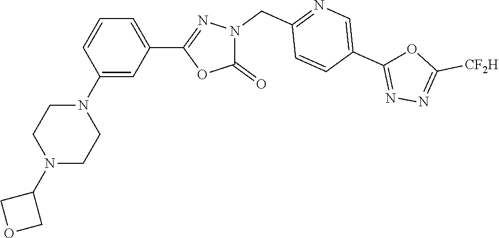 [SMILES: O=c1oc(-c2cccc(N3CCN(C4COC4)CC3)c2)nn1Cc1ccc(-c2nnc(C(F)F)o2)cn1] |
| 41 | 41 | 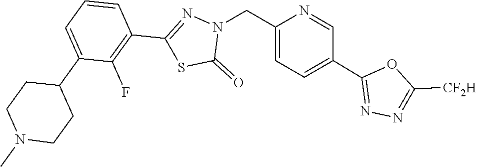 [SMILES: CN1CCC(c2cccc(-c3nn(Cc4ccc(-c5nnc(C(F)F)o5)cn4)c(=O)s3)c2F)CC1] |
| 42 | 42 | 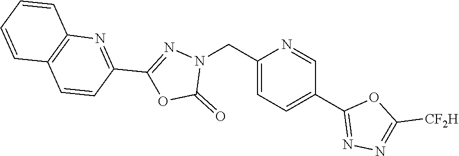 [SMILES: O=c1oc(-c2ccc3ccccc3n2)nn1Cc1ccc(-c2nnc(C(F)F)o2)cn1] |
| 43 | 43 | 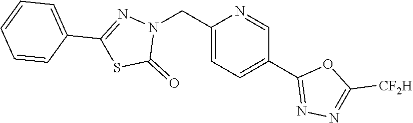 [SMILES: O=c1sc(-c2ccccc2)nn1Cc1ccc(-c2nnc(C(F)F)o2)cn1] |
| 44 | 44 | 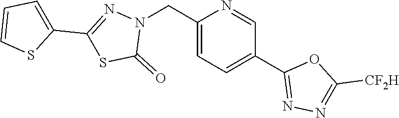 [SMILES: O=c1sc(-c2cccs2)nn1Cc1ccc(-c2nnc(C(F)F)o2)cn1] |
| 45 | 45 | 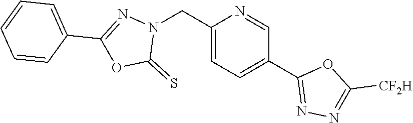 [SMILES: FC(F)c1nnc(-c2ccc(Cn3nc(-c4ccccc4)oc3=S)nc2)o1] |
| 46 | 46 | 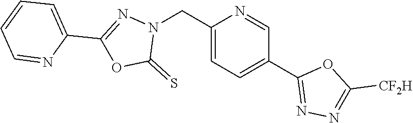 [SMILES: FC(F)c1nnc(-c2ccc(Cn3nc(-c4ccccn4)oc3=S)nc2)o1] |
| 47 | 47 | 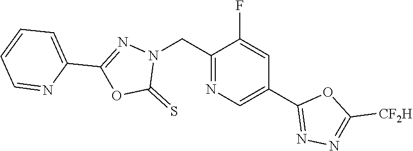 [SMILES: Fc1cc(-c2nnc(C(F)F)o2)cnc1Cn1nc(-c2ccccn2)oc1=S] |
| 48 | 48 | 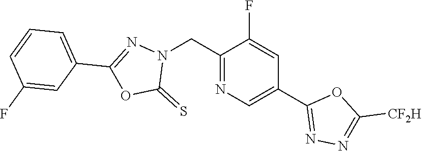 [SMILES: Fc1cccc(-c2nn(Cc3ncc(-c4nnc(C(F)F)o4)cc3F)c(=S)o2)c1] |
| 49 | 49 | 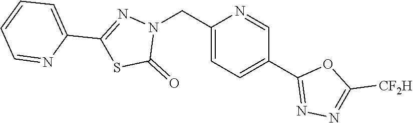 [SMILES: O=c1sc(-c2ccccn2)nn1Cc1ccc(-c2nnc(C(F)F)o2)cn1] |
| 50 | 50 | 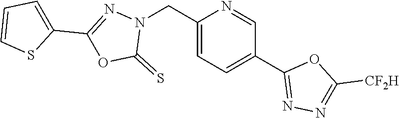 [SMILES: FC(F)c1nnc(-c2ccc(Cn3nc(-c4cccs4)oc3=S)nc2)o1] |
| 51 | 51 | 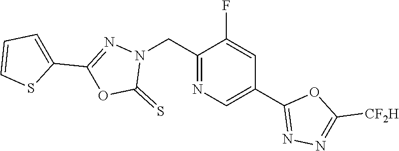 [SMILES: Fc1cc(-c2nnc(C(F)F)o2)cnc1Cn1nc(-c2cccs2)oc1=S] |
| 52 | 52 | 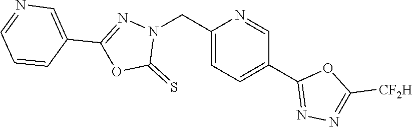 [SMILES: FC(F)c1nnc(-c2ccc(Cn3nc(-c4cccnc4)oc3=S)nc2)o1] |
| 53 | 53 | 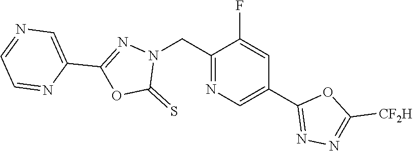 [SMILES: Fc1cc(-c2nnc(C(F)F)o2)cnc1Cn1nc(-c2cnccn2)oc1=S] |
| 54 | 54 | 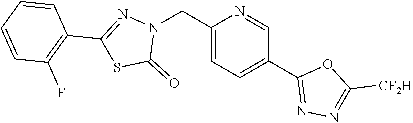 [SMILES: O=c1sc(-c2ccccc2F)nn1Cc1ccc(-c2nnc(C(F)F)o2)cn1] |
| 55 | 55 | 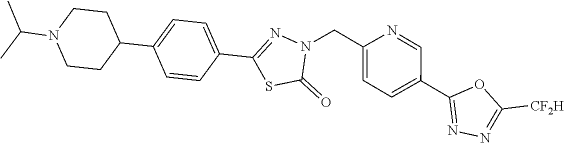 [SMILES: CC(C)N1CCC(c2ccc(-c3nn(Cc4ccc(-c5nnc(C(F)F)o5)cn4)c(=O)s3)cc2)CC1] |
| 56 | 56 | 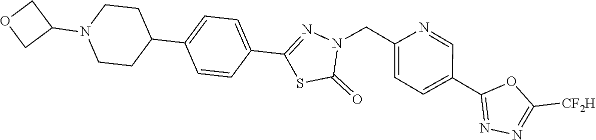 [SMILES: O=c1sc(-c2ccc(C3CCN(C4COC4)CC3)cc2)nn1Cc1ccc(-c2nnc(C(F)F)o2)cn1] |
| 57 | 57 | 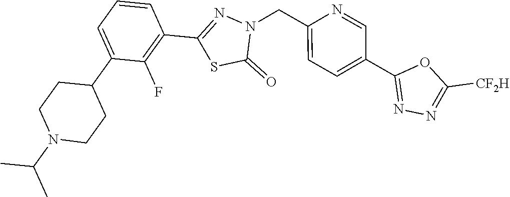 [SMILES: CC(C)N1CCC(c2cccc(-c3nn(Cc4ccc(-c5nnc(C(F)F)o5)cn4)c(=O)s3)c2F)CC1] |
| 58 | 58 | 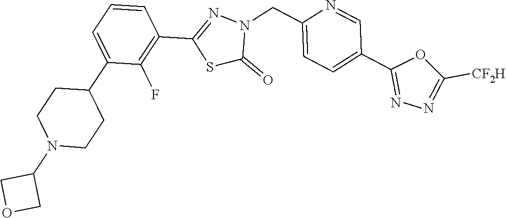 [SMILES: O=c1sc(-c2cccc(C3CCN(C4COC4)CC3)c2F)nn1Cc1ccc(-c2nnc(C(F)F)o2)cn1] |
| 59 | 59 | 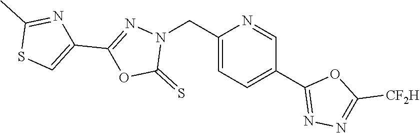 [SMILES: Cc1nc(-c2nn(Cc3ccc(-c4nnc(C(F)F)o4)cn3)c(=S)o2)cs1] |
| 60 | 60 | 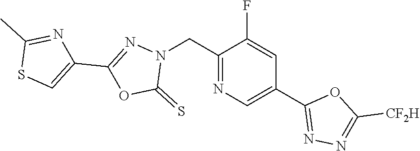 [SMILES: Cc1nc(-c2nn(Cc3ncc(-c4nnc(C(F)F)o4)cc3F)c(=S)o2)cs1] |
| 61 | 61 | 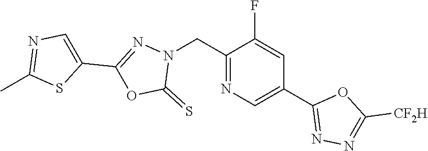 [SMILES: Cc1ncc(-c2nn(Cc3ncc(-c4nnc(C(F)F)o4)cc3F)c(=S)o2)s1] |
| 62 | 62 | 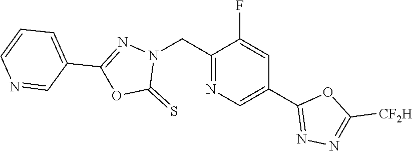 [SMILES: Fc1cc(-c2nnc(C(F)F)o2)cnc1Cn1nc(-c2cccnc2)oc1=S] |
| 63 | 63 | 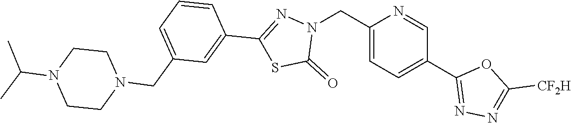 [SMILES: CC(C)N1CCN(Cc2cccc(-c3nn(Cc4ccc(-c5nnc(C(F)F)o5)cn4)c(=O)s3)c2)CC1] |
| 64 | 64 | 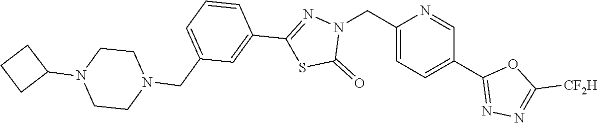 [SMILES: O=c1sc(-c2cccc(CN3CCN(C4CCC4)CC3)c2)nn1Cc1ccc(-c2nnc(C(F)F)o2)cn1] |
| 65 | 65 | 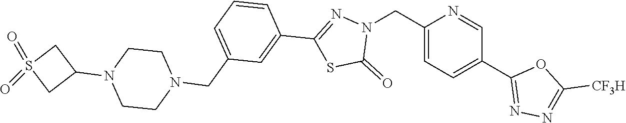 |
| 66 | 66 | 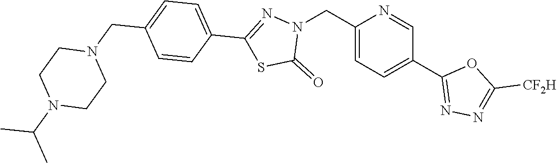 [SMILES: CC(C)N1CCN(Cc2ccc(-c3nn(Cc4ccc(-c5nnc(C(F)F)o5)cn4)c(=O)s3)cc2)CC1] |
| 67 | 67 | 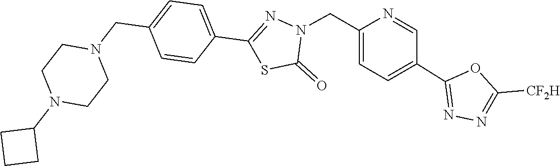 [SMILES: O=c1sc(-c2ccc(CN3CCN(C4CCC4)CC3)cc2)nn1Cc1ccc(-c2nnc(C(F)F)o2)cn1] |
| 68 | 68 | 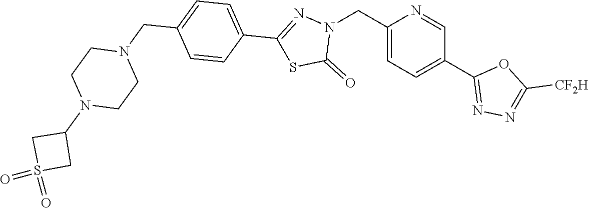 [SMILES: O=c1sc(-c2ccc(CN3CCN(C4CS(=O)(=O)C4)CC3)cc2)nn1Cc1ccc(-c2nnc(C(F)F)o2)cn1] |
| 69 | 69 | 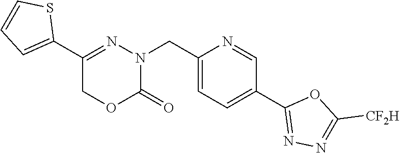 [SMILES: O=C1OCC(c2cccs2)=NN1Cc1ccc(-c2nnc(C(F)F)o2)cn1] |
| 70 | 70 | 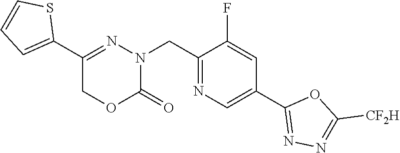 [SMILES: O=C1OCC(c2cccs2)=NN1Cc1ncc(-c2nnc(C(F)F)o2)cc1F] |
| 71 | 71 | 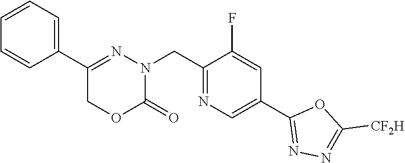 [SMILES: O=C1OCC(c2ccccc2)=NN1Cc1ncc(-c2nnc(C(F)F)o2)cc1F] |
| 72 | 72 | 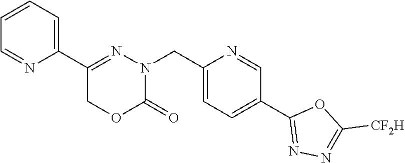 [SMILES: O=C1OCC(c2ccccn2)=NN1Cc1ccc(-c2nnc(C(F)F)o2)cn1] |
| 73 | 73 | 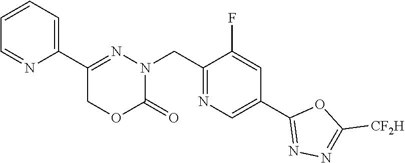 [SMILES: O=C1OCC(c2ccccn2)=NN1Cc1ncc(-c2nnc(C(F)F)o2)cc1F] |
| 74 | 74 | 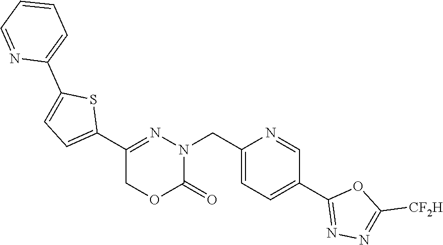 [SMILES: O=C1OCC(c2ccc(-c3ccccn3)s2)=NN1Cc1ccc(-c2nnc(C(F)F)o2)cn1] |
| 75 | 75 | 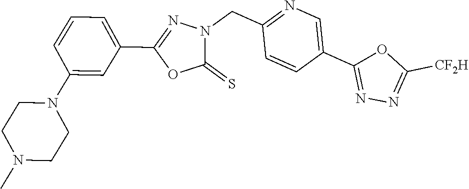 [SMILES: CN1CCN(c2cccc(-c3nn(Cc4ccc(-c5nnc(C(F)F)o5)cn4)c(=S)o3)c2)CC1] |
| 76 | 76 | 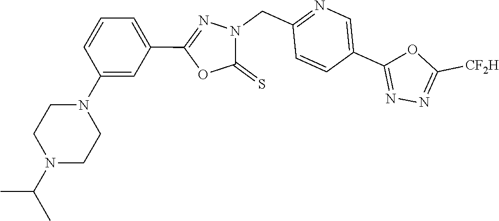 [SMILES: CC(C)N1CCN(c2cccc(-c3nn(Cc4ccc(-c5nnc(C(F)F)o5)cn4)c(=S)o3)c2)CC1] |
| 77 | 77 | 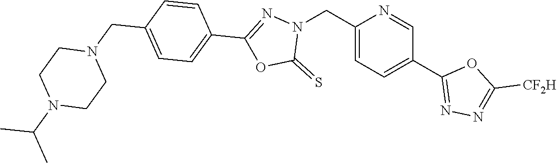 [SMILES: CC(C)N1CCN(Cc2ccc(-c3nn(Cc4ccc(-c5nnc(C(F)F)o5)cn4)c(=S)o3)cc2)CC1] |
| 78 | 78 | 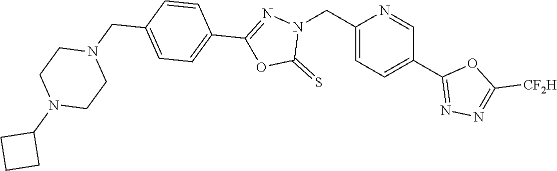 [SMILES: FC(F)c1nnc(-c2ccc(Cn3nc(-c4ccc(CN5CCN(C6CCC6)CC5)cc4)oc3=S)nc2)o1] |
| 79 | 79 | 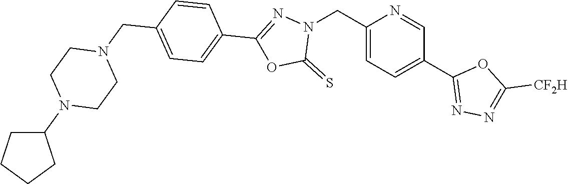 [SMILES: FC(F)c1nnc(-c2ccc(Cn3nc(-c4ccc(CN5CCN(C6CCCC6)CC5)cc4)oc3=S)nc2)o1] |
| 80 | 80 | 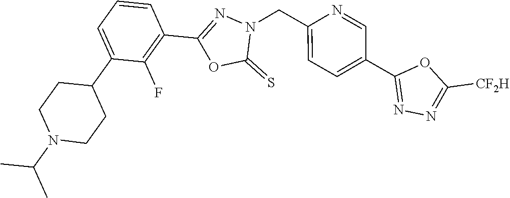 [SMILES: CC(C)N1CCC(c2cccc(-c3nn(Cc4ccc(-c5nnc(C(F)F)o5)cn4)c(=S)o3)c2F)CC1] |
| 81 | 81 | 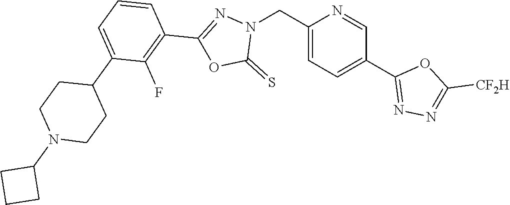 [SMILES: Fc1c(-c2nn(Cc3ccc(-c4nnc(C(F)F)o4)cn3)c(=S)o2)cccc1C1CCN(C2CCC2)CC1] |
| 82 | 82 | 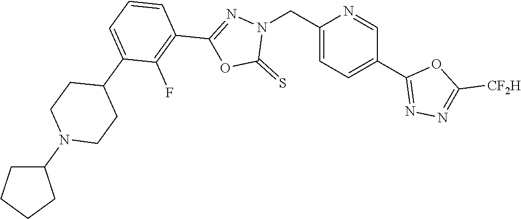 [SMILES: Fc1c(-c2nn(Cc3ccc(-c4nnc(C(F)F)o4)cn3)c(=S)o2)cccc1C1CCN(C2CCCC2)CC1] |
| 83 | 83 | 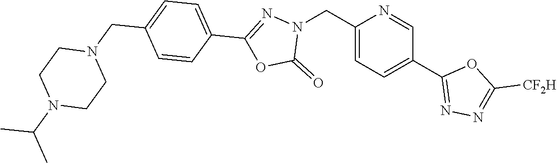 [SMILES: CC(C)N1CCN(Cc2ccc(-c3nn(Cc4ccc(-c5nnc(C(F)F)o5)cn4)c(=O)o3)cc2)CC1] |
| 84 | 84 | 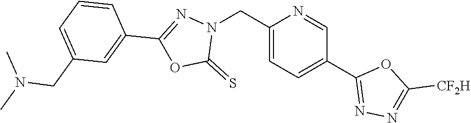 [SMILES: CN(C)Cc1cccc(-c2nn(Cc3ccc(-c4nnc(C(F)F)o4)cn3)c(=S)o2)c1] |
| 85 | 85 | 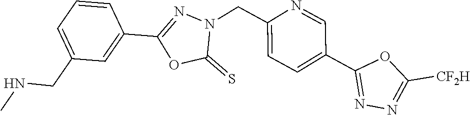 [SMILES: CNCc1cccc(-c2nn(Cc3ccc(-c4nnc(C(F)F)o4)cn3)c(=S)o2)c1] |
| 86 | 86 | 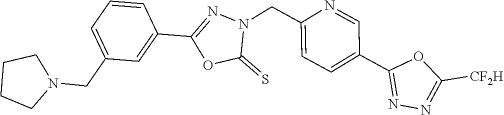 [SMILES: FC(F)c1nnc(-c2ccc(Cn3nc(-c4cccc(CN5CCCC5)c4)oc3=S)nc2)o1] |
| 87 | 87 | 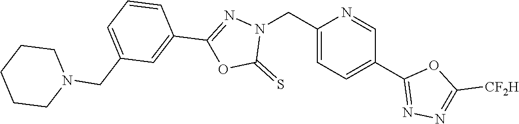 [SMILES: FC(F)c1nnc(-c2ccc(Cn3nc(-c4cccc(CN5CCCCC5)c4)oc3=S)nc2)o1] |
| 88 | 88 | 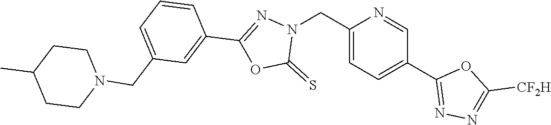 [SMILES: CC1CCN(Cc2cccc(-c3nn(Cc4ccc(-c5nnc(C(F)F)o5)cn4)c(=S)o3)c2)CC1] |
| 89 | 89 | 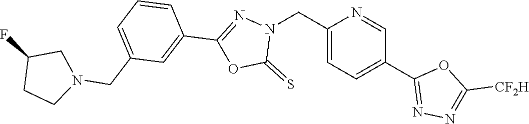 [SMILES: FC(F)c1nnc(-c2ccc(Cn3nc(-c4cccc(CN5CC[C@@H](F)C5)c4)oc3=S)nc2)o1] |
| 90 | 90 | 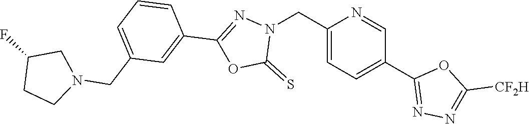 [SMILES: FC(F)c1nnc(-c2ccc(Cn3nc(-c4cccc(CN5CC[C@H](F)C5)c4)oc3=S)nc2)o1] |
| 91 | 91 | 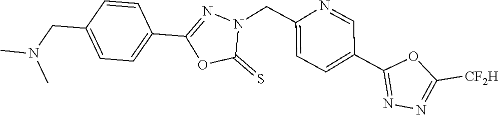 [SMILES: CN(C)Cc1ccc(-c2nn(Cc3ccc(-c4nnc(C(F)F)o4)cn3)c(=S)o2)cc1] |
| 92 | 92 | 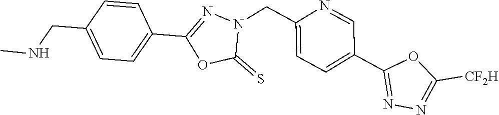 [SMILES: CNCc1ccc(-c2nn(Cc3ccc(-c4nnc(C(F)F)o4)cn3)c(=S)o2)cc1] |
| 93 | 93 | 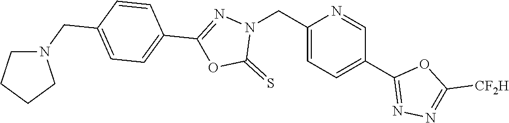 [SMILES: FC(F)c1nnc(-c2ccc(Cn3nc(-c4ccc(CN5CCCC5)cc4)oc3=S)nc2)o1] |
| 94 | 94 | 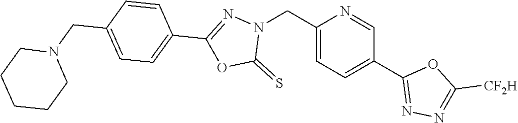 [SMILES: FC(F)c1nnc(-c2ccc(Cn3nc(-c4ccc(CN5CCCCC5)cc4)oc3=S)nc2)o1] |
| 95 | 95 | 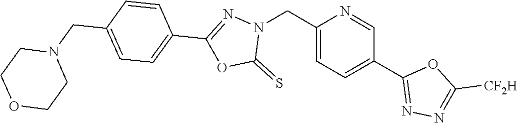 [SMILES: FC(F)c1nnc(-c2ccc(Cn3nc(-c4ccc(CN5CCOCC5)cc4)oc3=S)nc2)o1] |
| 96 | 96 | 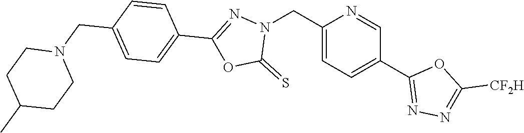 [SMILES: CC1CCN(Cc2ccc(-c3nn(Cc4ccc(-c5nnc(C(F)F)o5)cn4)c(=S)o3)cc2)CC1] |
| 97 | 97 | 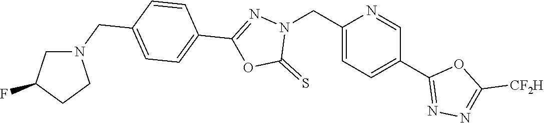 [SMILES: FC(F)c1nnc(-c2ccc(Cn3nc(-c4ccc(CN5CC[C@@H](F)C5)cc4)oc3=S)nc2)o1] |
| 98 | 98 | 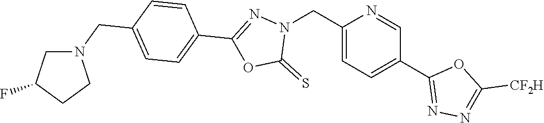 [SMILES: FC(F)c1nnc(-c2ccc(Cn3nc(-c4ccc(CN5CC[C@H](F)C5)cc4)oc3=S)nc2)o1] |
| 99 | 99 | 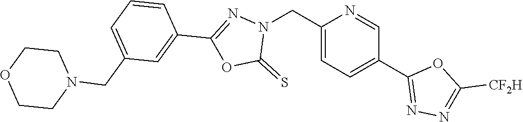 [SMILES: FC(F)c1nnc(-c2ccc(Cn3nc(-c4cccc(CN5CCOCC5)c4)oc3=S)nc2)o1] |
| 100 | 100 | 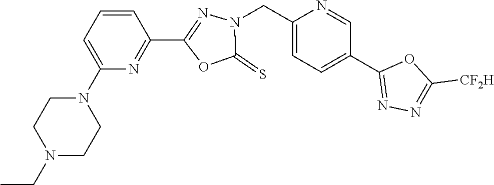 [SMILES: CCN1CCN(c2cccc(-c3nn(Cc4ccc(-c5nnc(C(F)F)o5)cn4)c(=S)o3)n2)CC1] |
| 101 | 101 | 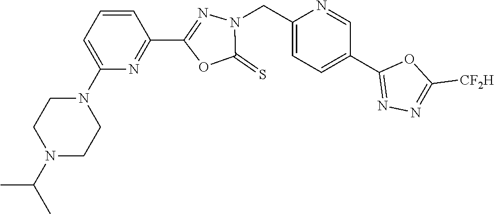 [SMILES: CC(C)N1CCN(c2cccc(-c3nn(Cc4ccc(-c5nnc(C(F)F)o5)cn4)c(=S)o3)n2)CC1] |
| 102 | 102 | 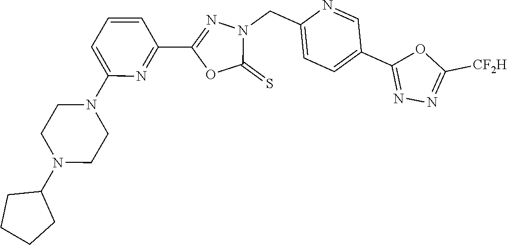 [SMILES: FC(F)c1nnc(-c2ccc(Cn3nc(-c4cccc(N5CCN(C6CCCC6)CC5)n4)oc3=S)nc2)o1] |
| 103 | 103 | 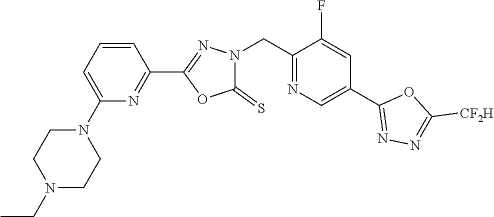 [SMILES: CCN1CCN(c2cccc(-c3nn(Cc4ncc(-c5nnc(C(F)F)o5)cc4F)c(=S)o3)n2)CC1] |
| 104 | 104 | 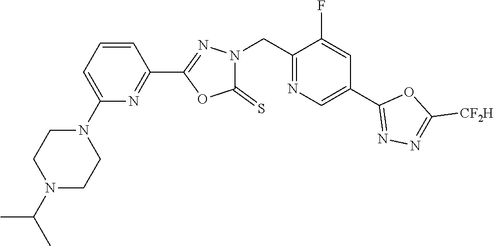 [SMILES: CC(C)N1CCN(c2cccc(-c3nn(Cc4ncc(-c5nnc(C(F)F)o5)cc4F)c(=S)o3)n2)CC1] |
| 105 | 105 | 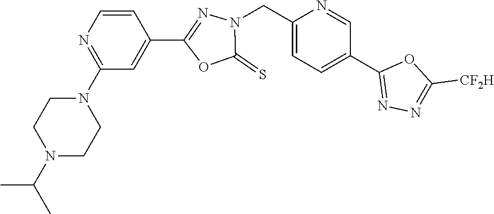 [SMILES: CC(C)N1CCN(c2cc(-c3nn(Cc4ccc(-c5nnc(C(F)F)o5)cn4)c(=S)o3)ccn2)CC1] |
| 106 | 106 | 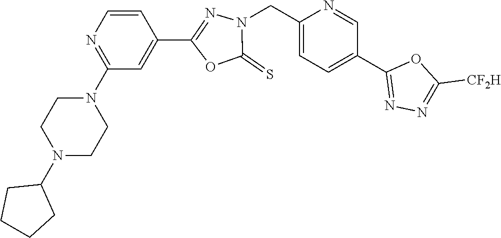 [SMILES: FC(F)c1nnc(-c2ccc(Cn3nc(-c4ccnc(N5CCN(C6CCCC6)CC5)c4)oc3=S)nc2)o1] |
| 107 | 107 | 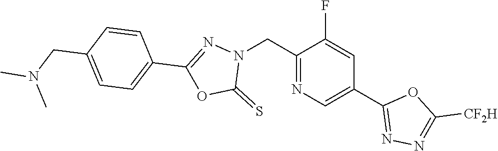 [SMILES: CN(C)Cc1ccc(-c2nn(Cc3ncc(-c4nnc(C(F)F)o4)cc3F)c(=S)o2)cc1] |
| 108 | 108 | 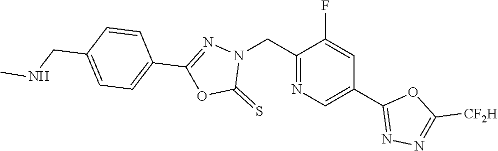 [SMILES: CNCc1ccc(-c2nn(Cc3ncc(-c4nnc(C(F)F)o4)cc3F)c(=S)o2)cc1] |
| 109 | 109 | 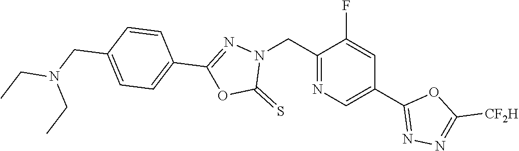 [SMILES: CCN(CC)Cc1ccc(-c2nn(Cc3ncc(-c4nnc(C(F)F)o4)cc3F)c(=S)o2)cc1] |
| 110 | 110 | 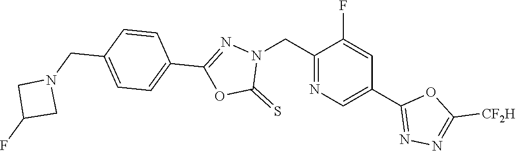 [SMILES: Fc1cc(-c2nnc(C(F)F)o2)cnc1Cn1nc(-c2ccc(CN3CC(F)C3)cc2)oc1=S] |
| 111 | 111 | 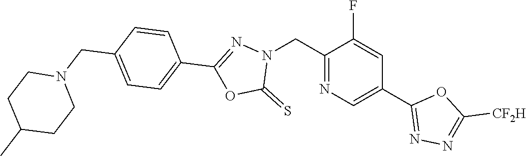 [SMILES: CC1CCN(Cc2ccc(-c3nn(Cc4ncc(-c5nnc(C(F)F)o5)cc4F)c(=S)o3)cc2)CC1] |
| 112 | 112 | 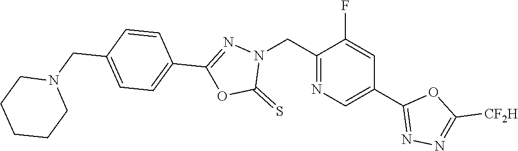 [SMILES: Fc1cc(-c2nnc(C(F)F)o2)cnc1Cn1nc(-c2ccc(CN3CCCCC3)cc2)oc1=S] |
| 113 | 113 | 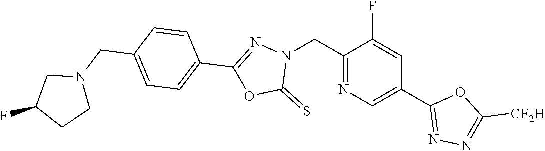 [SMILES: Fc1cc(-c2nnc(C(F)F)o2)cnc1Cn1nc(-c2ccc(CN3CC[C@@H](F)C3)cc2)oc1=S] |
| 114 | 114 | 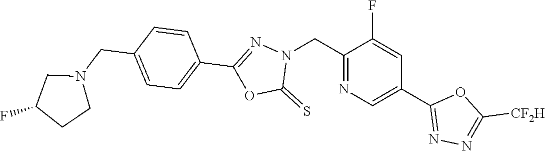 [SMILES: Fc1cc(-c2nnc(C(F)F)o2)cnc1Cn1nc(-c2ccc(CN3CC[C@H](F)C3)cc2)oc1=S] |
| 115 | 115 | 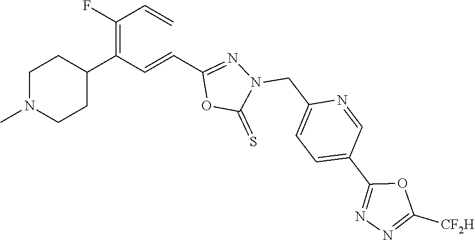 [SMILES: C=C/C(F)=C(\C=C\c1nn(Cc2ccc(-c3nnc(C(F)F)o3)cn2)c(=S)o1)C1CCN(C)CC1] |
| 116 | 116 | 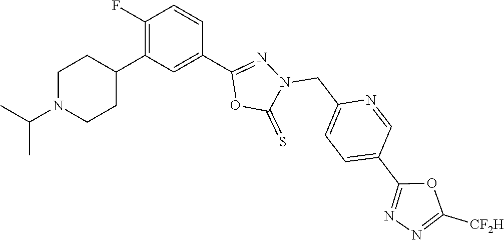 [SMILES: CC(C)N1CCC(c2cc(-c3nn(Cc4ccc(-c5nnc(C(F)F)o5)cn4)c(=S)o3)ccc2F)CC1] |
| 117 | 117 | 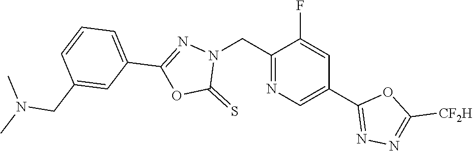 [SMILES: CN(C)Cc1cccc(-c2nn(Cc3ncc(-c4nnc(C(F)F)o4)cc3F)c(=S)o2)c1] |
| 118 | 118 | 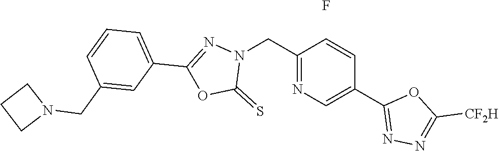 |
| 119 | 119 | 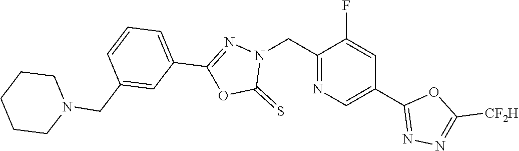 [SMILES: Fc1cc(-c2nnc(C(F)F)o2)cnc1Cn1nc(-c2cccc(CN3CCCCC3)c2)oc1=S] |
| 120 | 120 | 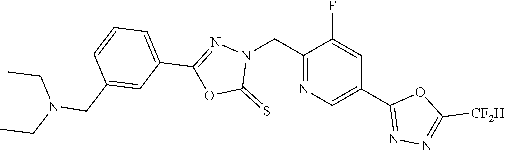 [SMILES: CCN(CC)Cc1cccc(-c2nn(Cc3ncc(-c4nnc(C(F)F)o4)cc3F)c(=S)o2)c1] |
| 121 | 121 | 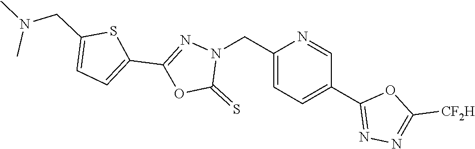 [SMILES: CN(C)Cc1ccc(-c2nn(Cc3ccc(-c4nnc(C(F)F)o4)cn3)c(=S)o2)s1] |
| 122 | 122 | 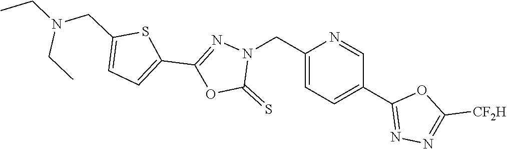 [SMILES: CCN(CC)Cc1ccc(-c2nn(Cc3ccc(-c4nnc(C(F)F)o4)cn3)c(=S)o2)s1] |
| 123 | 123 | 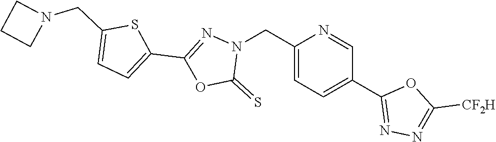 [SMILES: FC(F)c1nnc(-c2ccc(Cn3nc(-c4ccc(CN5CCC5)s4)oc3=S)nc2)o1] |
| 124 | 124 | 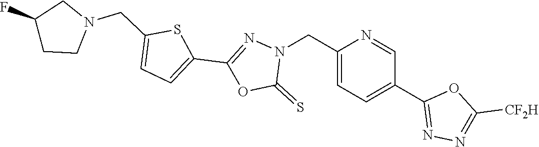 [SMILES: FC(F)c1nnc(-c2ccc(Cn3nc(-c4ccc(CN5CC[C@@H](F)C5)s4)oc3=S)nc2)o1] |
| 125 | 125 | 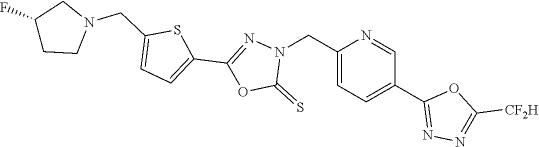 [SMILES: FC(F)c1nnc(-c2ccc(Cn3nc(-c4ccc(CN5CC[C@H](F)C5)s4)oc3=S)nc2)o1] |
| 126 | 126 | 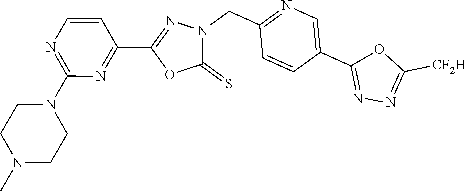 [SMILES: CN1CCN(c2nccc(-c3nn(Cc4ccc(-c5nnc(C(F)F)o5)cn4)c(=S)o3)n2)CC1] |
| 127 | 127 | 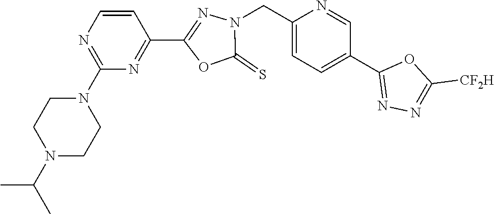 [SMILES: CC(C)N1CCN(c2nccc(-c3nn(Cc4ccc(-c5nnc(C(F)F)o5)cn4)c(=S)o3)n2)CC1] |
| 128 | 128 | 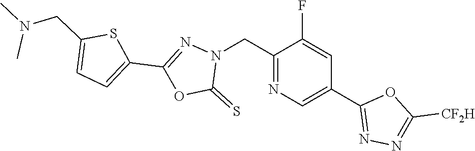 [SMILES: CN(C)Cc1ccc(-c2nn(Cc3ncc(-c4nnc(C(F)F)o4)cc3F)c(=S)o2)s1] |
| 129 | 129 | 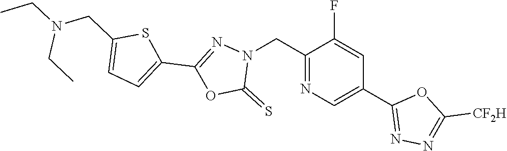 [SMILES: CCN(CC)Cc1ccc(-c2nn(Cc3ncc(-c4nnc(C(F)F)o4)cc3F)c(=S)o2)s1] |
| 130 | 130 | 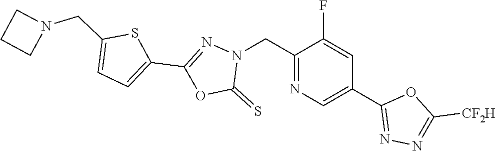 [SMILES: Fc1cc(-c2nnc(C(F)F)o2)cnc1Cn1nc(-c2ccc(CN3CCC3)s2)oc1=S] |
| 131 | 131 | 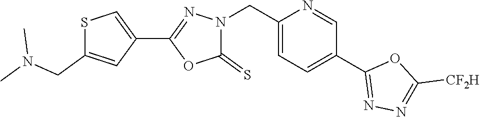 [SMILES: CN(C)Cc1cc(-c2nn(Cc3ccc(-c4nnc(C(F)F)o4)cn3)c(=S)o2)cs1] |
| 132 | 132 | 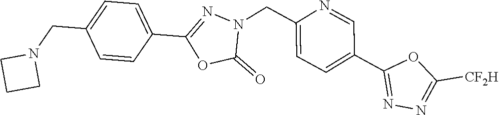 [SMILES: O=c1oc(-c2ccc(CN3CCC3)cc2)nn1Cc1ccc(-c2nnc(C(F)F)o2)cn1] |
| 133 | 133 | 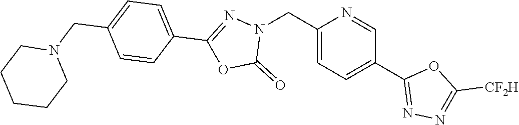 [SMILES: O=c1oc(-c2ccc(CN3CCCCC3)cc2)nn1Cc1ccc(-c2nnc(C(F)F)o2)cn1] |
| 134 | 134 | 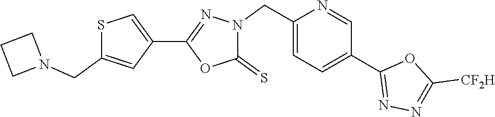 [SMILES: FC(F)c1nnc(-c2ccc(Cn3nc(-c4csc(CN5CCC5)c4)oc3=S)nc2)o1] |
| 135 | 135 | 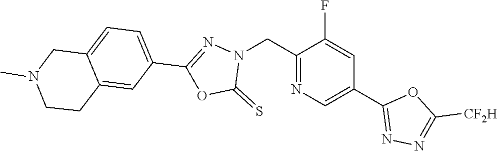 [SMILES: CN1CCc2cc(-c3nn(Cc4ncc(-c5nnc(C(F)F)o5)cc4F)c(=S)o3)ccc2C1] |
| 136 | 136 | 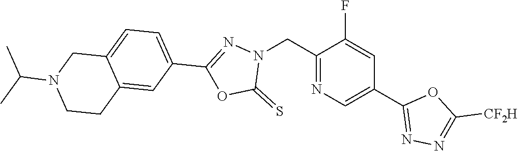 [SMILES: CC(C)N1CCc2cc(-c3nn(Cc4ncc(-c5nnc(C(F)F)o5)cc4F)c(=S)o3)ccc2C1] |
| 137 | 137 | 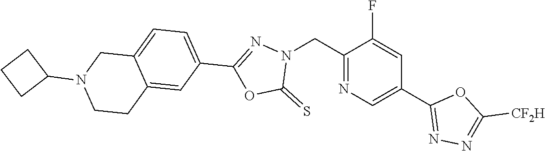 [SMILES: Fc1cc(-c2nnc(C(F)F)o2)cnc1Cn1nc(-c2ccc3c(c2)CCN(C2CCC2)C3)oc1=S] |
| 138 | 138 | 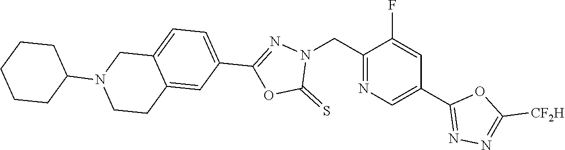 [SMILES: Fc1cc(-c2nnc(C(F)F)o2)cnc1Cn1nc(-c2ccc3c(c2)CCN(C2CCCCC2)C3)oc1=S] |
| 139 | 139 | 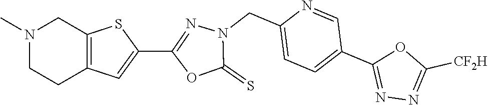 [SMILES: CN1CCc2cc(-c3nn(Cc4ccc(-c5nnc(C(F)F)o5)cn4)c(=S)o3)sc2C1] |
| 140 | 140 | 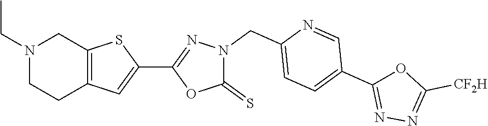 [SMILES: CCN1CCc2cc(-c3nn(Cc4ccc(-c5nnc(C(F)F)o5)cn4)c(=S)o3)sc2C1] |
| 141 | 141 | 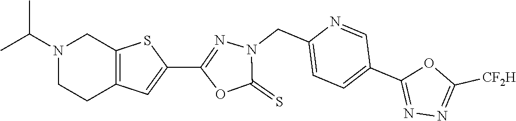 [SMILES: CC(C)N1CCc2cc(-c3nn(Cc4ccc(-c5nnc(C(F)F)o5)cn4)c(=S)o3)sc2C1] |
| 142 | 142 | 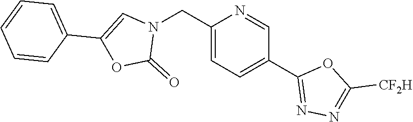 [SMILES: O=c1oc(-c2ccccc2)cn1Cc1ccc(-c2nnc(C(F)F)o2)cn1] |
| 143 | 143 | 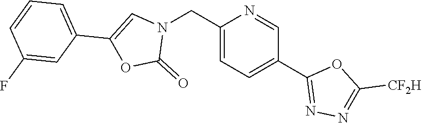 [SMILES: O=c1oc(-c2cccc(F)c2)cn1Cc1ccc(-c2nnc(C(F)F)o2)cn1] |
| 144 | 144 | 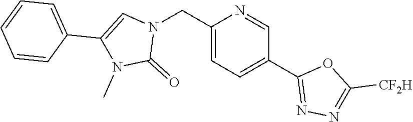 [SMILES: Cn1c(-c2ccccc2)cn(Cc2ccc(-c3nnc(C(F)F)o3)cn2)c1=O] |

In the present invention, the term “alkyl” as used herein may refer to a straight-chain or branched-chain acyclic, cyclic or saturated hydrocarbon in which the carbon atoms are connected, unless otherwise specified. For example, “C₁₋₄alkyl” may mean an alkyl containing 1 to 4 carbon atoms. The acyclic alkyl may include, for example, methyl, ethyl, n-propyl, n-butyl, isopropyl, sec-butyl, isobutyl, tert-butyl, and the like, but is not limited to thereto. The cyclic alkyl may be used interchangeably with “cycloalkyl” in the present specification, and may include, as an example, cyclopropyl, cyclobutyl, cyclopentyl, cyclohexyl, cycloheptyl, cyclooctyl, and the like, but is not limited thereto.

In the present invention, “alkoxy” may refer to —(O-alkyl) as an alkyl ether group, wherein the alkyl is the same as defined above. For example, “C₁₋₄alkoxy” may mean an alkoxy containing C₁₋₄alkyl, i.e. —(O—C₁₋₄alkyl); and examples of the alkoxy may include, but are not limited to, methoxy, ethoxy, n-propoxy, isopropoxy, n-butyoxy, isobutoxy, sec-butoxy, tert-butoxy, and the like.

In the present invention, “halo” may be F, Cl, Br or I.

In the present invention, the term “haloalkyl” may mean a straight or branched chain alkyl (hydrocarbon) having carbon atoms substituted with at least one halo as defined herein. Examples of the haloalkyl include, but are no limited to, methyl, ethyl, propyl, isopropyl, isobutyl or n-butyl independently substituted with at least one halogen such as F, Cl, Br, or I.

In the present invention, the term “hydroxyalkyl” may refer to a straight or branched chain alkyl (hydrocarbon) having carbon atoms substituted with —OH. Examples of the hydroxyalkyl include, but are not limited to, methyl, ethyl, propyl, isopropyl, isobutyl or n-butyl independently substituted with at least one hydroxy.

In the present invention, the term “aminoalkyl” may refer to a straight or branched chain alkyl (hydrocarbon) having carbon atoms substituted with amino-(NR′R″). Herein, R′ and R″ may each independently be selected from the group consisting of hydrogen and C₁₋₄alkyl, and the selected R′ and R″ may each independently be substituted or unsubstituted.

In the present invention, the term “heterocycloalkyl” may mean a ring containing 1 to 5 heteroatoms selected from N, O and S as atoms forming the ring, and may be saturated or partially unsaturated. Herein, when unsaturated, the heterocycloalkyl may be referred to as a heterocycloalkene. Unless otherwise stated, the heterocycloalkyl may be a single ring or multiple rings such as spiro rings, bridged rings or fused rings. Further, “3 to 12-membered heterocycloalkyl” may mean a heterocycloalkyl containing 3 to 12 atoms forming a ring. Examples of the heterocycloalkyl may include, but are not limited to, pyrrolidine, piperidin, imidazolidine, pyrazolidine, butyrolactam, valerolactam, imidazolidinone, hydantoin, dioxolane, phthalimide, piperidine, pyrimidine-24(1H,3H)-dione, 1,4-dioxane, morpholine, thiomorpholine, thiomorpholine-S-oxide, thiomorpholine-S,S-oxide, piperazine, pyran, pyridone, 3-pyrroline, thiopyran, pyrone, tetrahydrofuran, tetrahydrothiophene, quinuclidine, tropane, 2-azaspiro[3.3]heptane, (1R,5S)-3-azabicyclo[3.2.1]octane, (1S,4S)-2azabicyclo[2.2.2]octane, or (1R,4R)-2-oxa-5-azabicyclo[2.2.2]octane, and the like.

In the present invention, “arene” may mean an aromatic hydrocarbon ring. The arene may be monocyclic arene or polycyclic arene. The number of ring carbon atoms in the arene may be 5 or more and 30 or less, 5 or more and 20 or less, or 5 or more and 15 or less. Examples of arene may include, but are not limited to, benzene, naphthalene, fluorene, anthracene, phenanthrene, bibenzene, terbenzene, quaterbenzene, quinquebenzene, sexibenzene, triphenylene, pyrene, benzofluoranthene, chrysene, and the like. In the present specification, a moiety obtained by removing one hydrogen atom from the above “arene” is referred to as “aryl”.

In the present invention, “heteroarene” may be a ring containing one or more of O, N, P, Si, and S as heterogeneous elements. The number of ring carbon atoms of the heteroarene may be 2 or more and 30 or less, or 2 or more and 20 or less. The heteroarene may be a monocyclic heteroarene or a polycyclic heteroarene. The polycyclic heteroarene may have, for example, a bicyclic or tricyclic structure. Examples of the heteroarene include, but not limited to, thiophene, purine, pyrrole, pyrazole, imidazole, thiazole, oxazole, isothiazole, oxadiazole, triazole, pyridine, bipyridyl, triazine, acridyl, pyridazine, pyrazine, quinoline, quinazoline, quinoxaline, phenoxazine, phthalazine, pyrimidine, pyridopyrimidine, pyridopyrazine, pyrazinopyrazine, isoquinoline, indole, carbazole, imidazopyridazine, imidazopyridine, imidazopyrimidine, pyrazolopyrimidine, imidazopyrazine or pyrazolopyridine, N-arylcarbazole, N-heteroarylcarbazole, N-alkylcarbazole, benzoxazole, benzoimidazole, benzothiazole, benzocarbazole, benzothiophene, dibenzothiophene, thienothiophene, benzofuran, phenanthroline, isoxazole, oxadiazole, thiadiazole, benzothiazole, tetrazole, phenothiazine, dibenzosilole, dibenzofuran, and the like. In an embodiment of the present invention, the heteroarene may also include a bicyclic heterocyclo-arene including an arene ring fused to a heterocycloalkyl ring or a heteroarene fused to a cycloalkyl ring. In the present specification, a moiety obtained by removing one hydrogen atom from the above “heteroarene” is referred to as “heteroaryl”.

The compound represented by Chemical Formula I of the present invention may contain at least one asymmetric carbon and thus may be present as a racemate, a racemic mixture, a single enantiomer, a diastereomeric mixture and each diastereomer. These stereoisomers may be separated by conventional techniques, and for example, the compound represented by Chemical Formula I may be separated by column chromatography, HPLC, or the like. Otherwise, each stereoisomer of the compound represented by Chemical Formula I may be stereospecifically synthesized using optically pure starting materials and/or reagents with known configurations.

In the present invention, the term “enantiomer” as used herein means a compound having the same chemical formula or molecular formula but different in spatial arrangement, or a salt thereof. Each of these enantiomers and mixtures thereof are also included within the scope of the present invention. Unless otherwise specified, a solid bond (—) connected to an asymmetric carbon atom may include a wedged solid bond ( [symbol] ) or wedge dashed bond ( [symbol] ) representing the absolute arrangement of stereocenters.

The compound represented by Chemical Formula I of the present invention may be present in the form of a “pharmaceutically acceptable salt”. As the salt, an acid addition salt formed by a pharmaceutically acceptable free acid is useful. The term “pharmaceutically acceptable salt” as used herein refers to a concentration having a relatively non-toxic and harmless effective effect on patients, which includes any organic acid or inorganic acid addition salt of the compound represented by Chemical Formula I in which side effects caused by these salts do not reduce the beneficial efficacy of the compound.

Acid addition salts are prepared by conventional methods, for example, by dissolving a compound in an excess aqueous acid solution and precipitating the salt using a water-miscible organic solvent, such as methanol, ethanol, acetone or acetonitrile. An equimolar amounts of the compound and an acid or alcohol in water may be heated, and then the mixture may be evaporated to dryness, or the precipitated salt may be suction filtered.

In this case, as the free acid, organic acids and inorganic acids may be used, wherein the inorganic acid may include hydrochloric acid, phosphoric acid, sulfuric acid, nitric acid, and the like, and the organic acid may include methanesulfonic acid, p-toluenesulfonic acid, acetic acid, trifluoroacetic acid, maleic acid, succinic acid, oxalic acid, benzoic acid, tartaric acid, fumaric acid, mandelic acid, propionic acid, citric acid, lactic acid, glycolic acid, gluconic acid, galacturonic acid, glutamic acid, glutaric acid, glucuronic acid, aspartic acid, ascorbic acid, carbonic acid, vanillic acid, hydroiodic acid, and the like. However, the organic acid and inorganic acid are not limited thereto.

Further, a pharmaceutically acceptable metal salt may be prepared using a base. The alkali metal salt or alkaline earth metal salt is obtained, for example, by dissolving the compound in an excess alkali metal hydroxide or alkaline earth metal hydroxide solution, and filtering an insoluble compound salt, followed by evaporating and drying the filtrate. Here, as the metal salt, sodium, potassium, or calcium salt is particularly suitable in a pharmaceutical aspect, but the metal salt is not limited thereto. In addition, the corresponding silver salt may be obtained by reacting an alkali metal or alkaline earth metal salt with a suitable silver salt (for example, silver nitrate).

The pharmaceutically acceptable salt of the present invention, unless otherwise indicated, includes salts of acidic or basic groups that may be present in the compounds represented by Chemical Formula I above. For example, the pharmaceutically acceptable salt may include sodium, calcium, and potassium salts of a hydroxy group, and other pharmaceutically acceptable salts of an amino group may include hydrobromide, sulfate, hydrogen sulfate, phosphate, hydrogen phosphate, dihydrogen phosphate, acetate, succinate, citrate, tartrate, lactate, mandelate, methanesulfonate (mesylate), and p-toluenesulfonate (tosylate) salts, and the like, which may be prepared through a salt preparation method known in the art.

### Method for Preparing 1,3,4-oxadiazole Derivative Compound

The present invention provides a method for preparing a 1,3,4-oxadiazole derivative compound represented by the following Chemical Formula I, a stereoisomer thereof, or a pharmaceutically acceptable salt thereof:

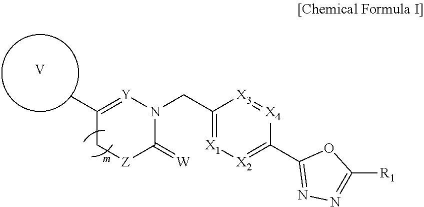

The Chemical Formula I is as defined above.

In the present invention, preferred methods for preparing the oxadiazole derivative compound represented by Chemical Formula I, a stereoisomer thereof or a pharmaceutically acceptable salt thereof are shown in the following [Reaction Scheme 1] to [Reaction Scheme 8], and preparation methods thereof that are modified to a level obvious to those skilled in the art are also included therein.

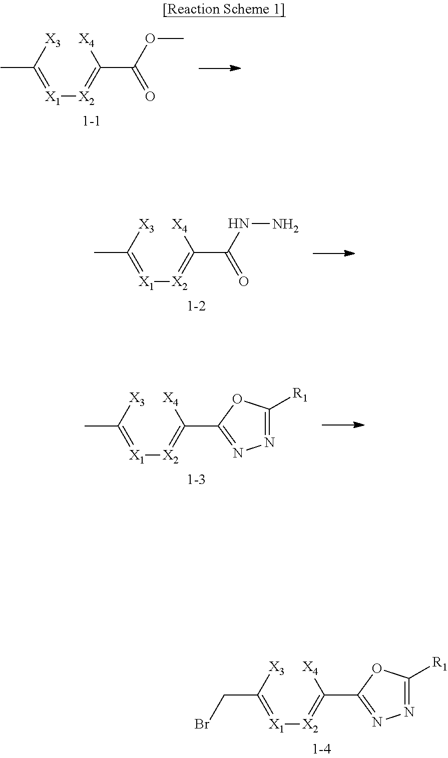

According to Reaction Scheme 1 above, hydrazine is added to Compound 1-1 to prepare Compound 1-2, followed by reaction with trifluoroacetic anhydride or difluoroacetic anhydride to prepare Compound 1-3, and then a bromination reaction is performed to synthesize Compound 1-4. Compound 1-4 is Zn-Binder moiety of HDAC 6 inhibitor and is used as an intermediate in the synthesis of all compounds.

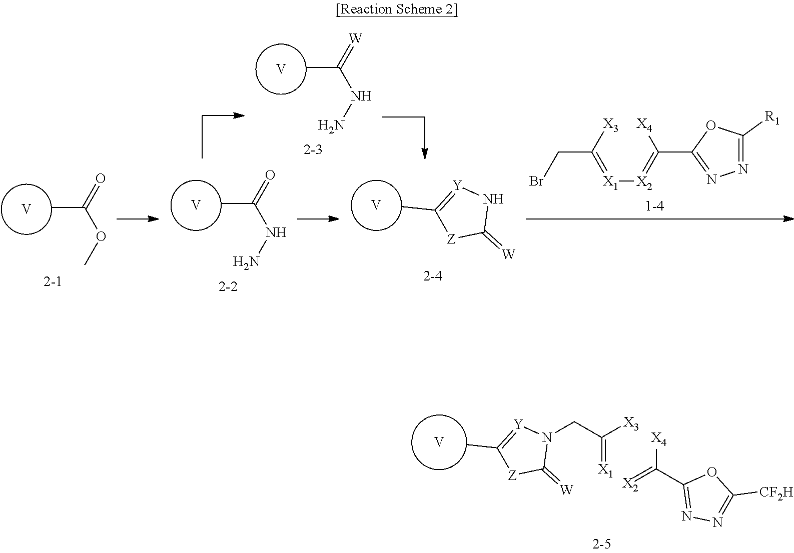

According to Reaction Scheme 2 above, Compound 2-2 is prepared from Compound 2-1 using hydrazine, and then 1,3,4-oxadiazol-2(3H)-thione Compound 2-4 is prepared by employing CDI and using 1,3,4-oxadiazol-2(3H)-one and potassium ethylxanthate. In addition, Compound 2-3 may be prepared by adding Lawesson's reagent to Compound 2-2, and then 1,3,4-thiadiazol-2(3H)-one Compound 2-4 may be prepared by using CDI. Compound 2-5 is prepared through a substitution reaction with Compound 2-4 and Compound 1-4. Examples of Compound 2-5 to be prepared by Reaction Scheme 2 above may include Compound Nos. 1, 5, 15, 16, 17, 18, 42, 43, 44, 45, 46, 47, 48, 49, 50, 51, 52, 53, 54, 59, 60, 61, 62, and the like.

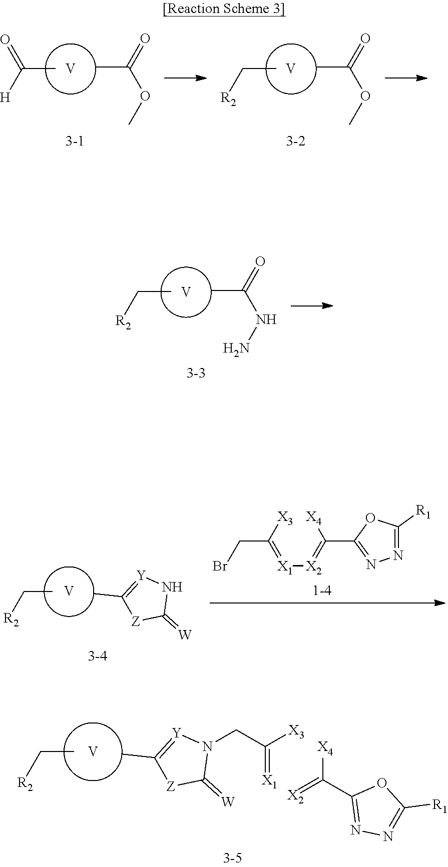

In Reaction Scheme 3, Compound 3-1 having an aldehyde structure is subjected to reductive amination to prepare Compound 3-2, then Compound 3-3 is prepared using hydrazine, and then 1,3,4-oxadiazol-2(3H)-one Compound 3-4 is prepared using CDI. Then, Compound 3-5 may be prepared through a substitution reaction with Compound 1-4. Examples of Compound 3-5 to be prepared by Reaction Scheme 3 above may include Compound Nos. 2 and 4, and the like.

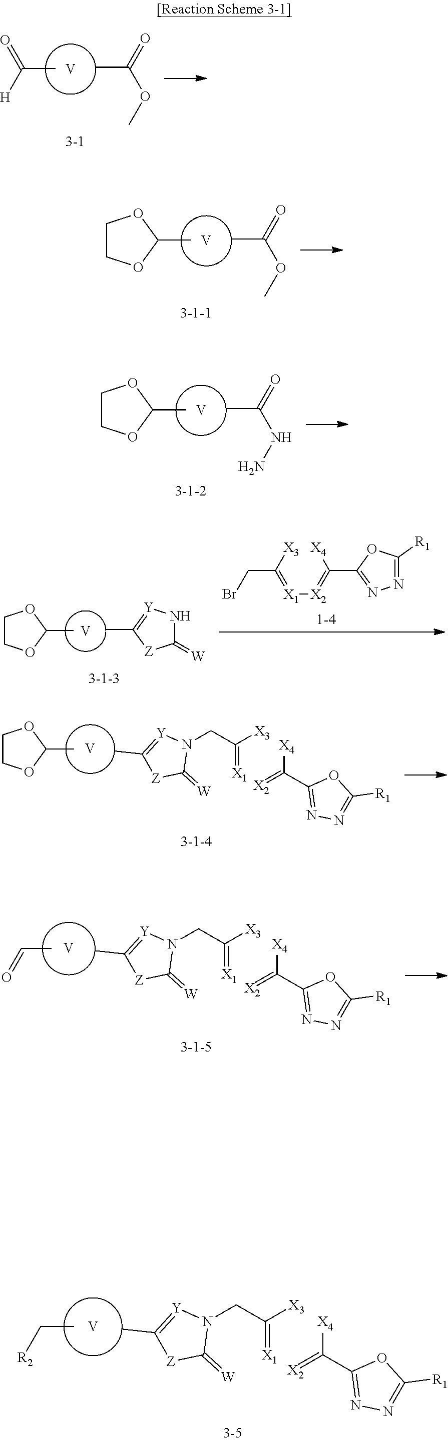

In Reaction Scheme 3-1, dioxolane compound 3-1-1 protecting the aldehyde structure is prepared using ethylene glycol from Compound 3-1, and then Compound 3-1-2 is prepared using hydrazine. Then, 1,3,4-oxadiazol-2(3H)-thione Compound 3-1-3 is prepared by employing CDI and using 1,3,4-oxadiazol-2(3H)-one and potassium ethylxanthate. Then, Compound 3-1-4 is prepared through a substitution reaction with Compound 1-4, and Compound 3-1-5 is prepared by removing dioxolane, an aldehyde protecting group, using iron (III) chloride hexahydrate. Then, Compound 3-5 may be prepared through reductive amination. Examples of Compound 3-5 to be prepared by Reaction Scheme 3-1 may include Compound Nos. 84, 85, 86, 87, 88, 89, 90, 91, 92, 93, 94, 95, 96, 97, 98, 99, 107, 108, 109, 110, 111, 112, 113, 114, 117, 118, 119, 120, 121, 122, 123, 124, 125, 128, 129, 130, 131, 132, 133, 134, and the like.

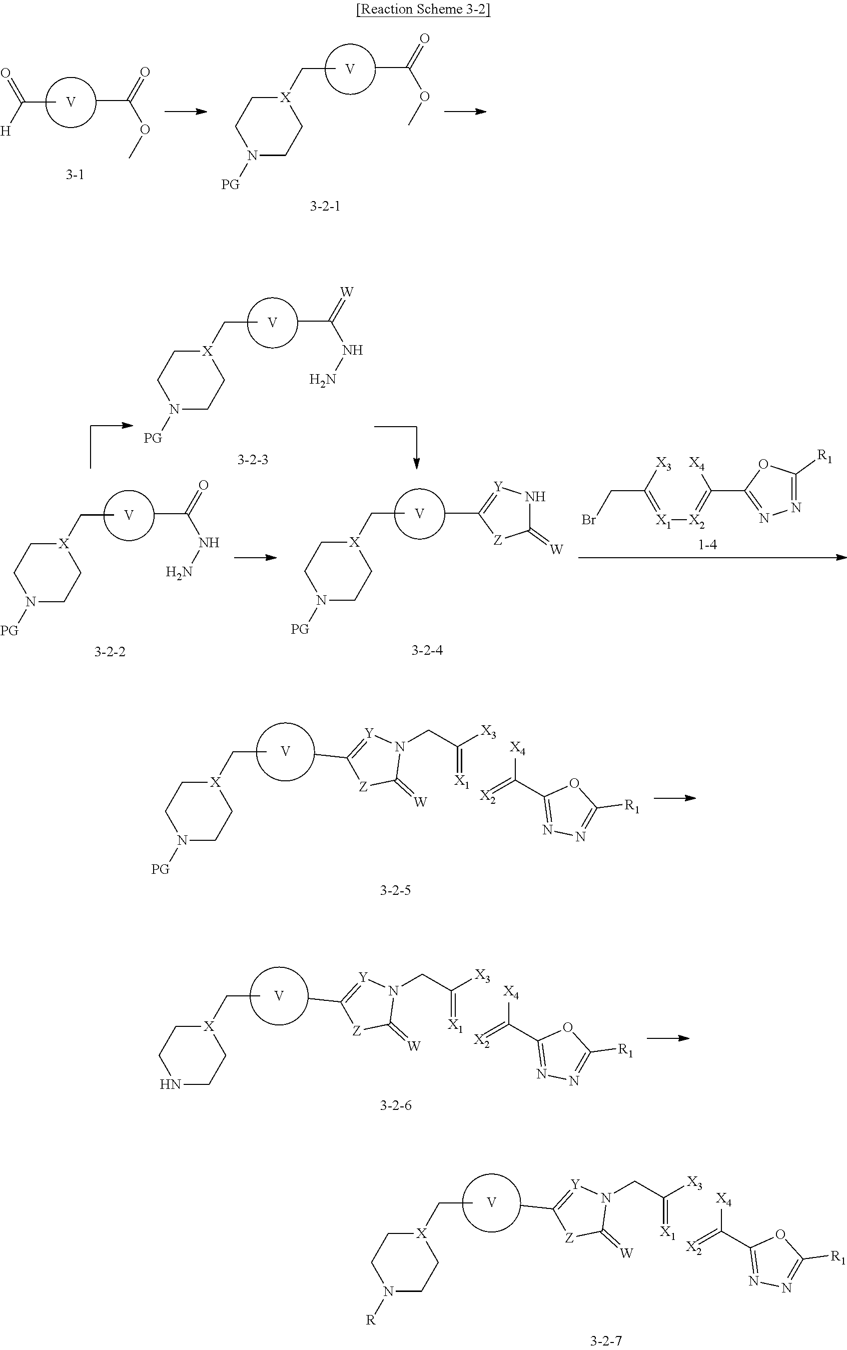

In Reaction Scheme 3-2, Compound 3-1 having an aldehyde structure is subjected to reductive amination to prepare Compound 3-2-1, then Compound 3-2-2 is prepared using hydrazine, and then 1,3,4-oxadiazol-2(3H)-thione Compound 3-2-4 is prepared by employing CDI and using 1,3,4-oxadiazol-2(3H)-one and potassium ethylxanthate. In addition, Compound 3-2-3 is prepared by adding Lawesson's reagent to Compound 3-2-2, and then 1,3,4-thiadiazol-2(3H)-one Compound 3-2-4 is prepared by using CDI. Then, Compound 3-2-5 is prepared using a substitution reaction with Compound 3-2-4 and Compound 1-4, and Compound 3-2-6 is prepared by removing the amine protecting group, followed by reductive amination, to prepare Compound 3-2-7. Examples of Compound 3-2-7 to be prepared by Reaction Scheme 3-2 above may include Compound Nos. 63, 64, 65, 66, 67, 68, 77, 78, 79, 83, and the like.

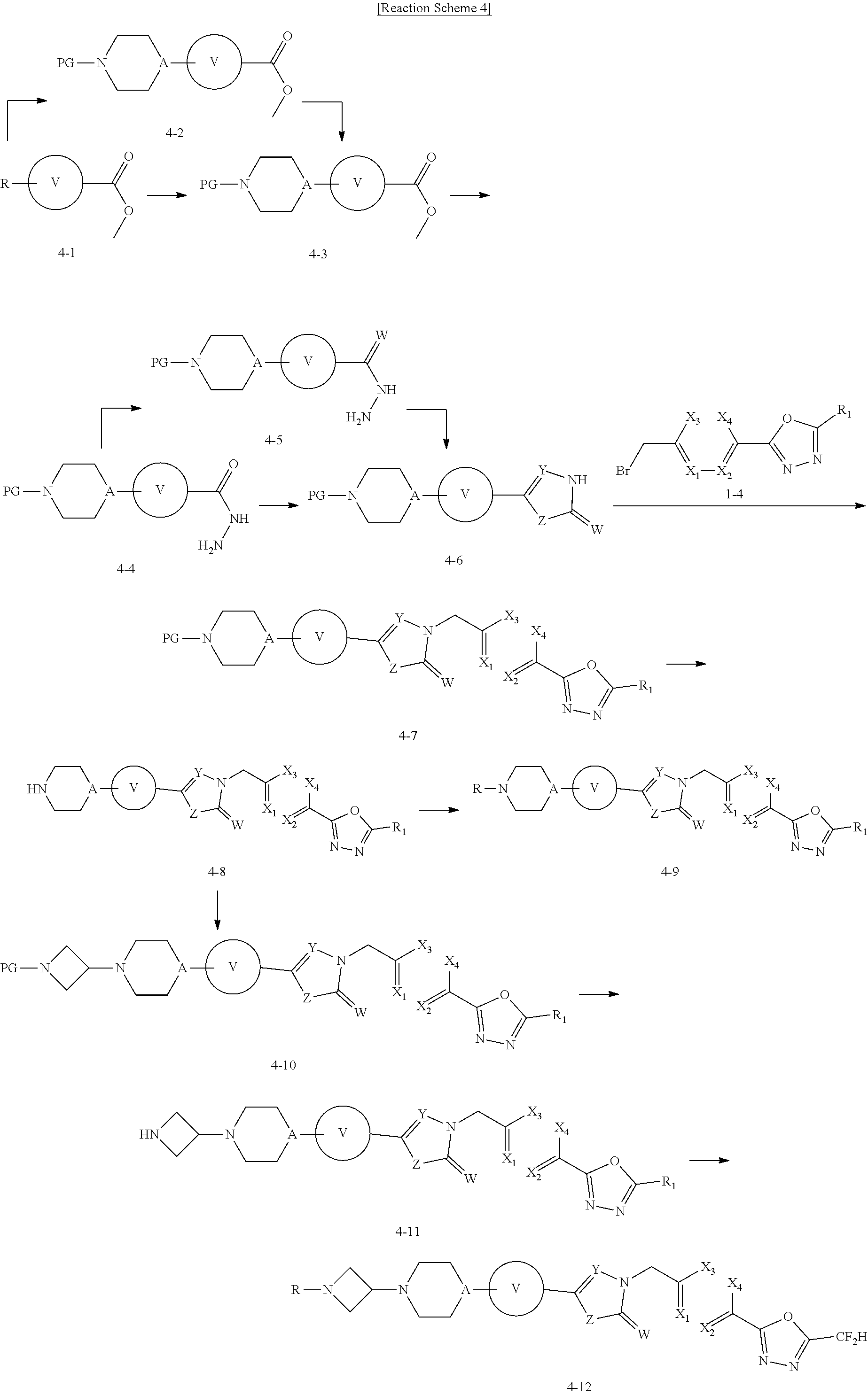

In Reaction Scheme 4, ester compound 4-1 having a halogen element is subjected to C—C coupling (Suzuki-coupling) reaction to prepare Compound 4-2, followed by a reduction reaction to prepare Compound 4-3. In addition, Compound 4-3 is prepared through C—N coupling (Buchwald-reaction) and substitution reaction from Compound 4-1. Then, Compound 4-4 is prepared using hydrazine, and then 1,3,4-oxadiazol-2 (3H)-thione Compound 4-6 is prepared by employing CDI and using 1,3,4-oxadiazol-2 (3H)-one and potassium ethylxanthate. In addition, Compound 4-5 is prepared by adding Lawesson's reagent to Compound 4-4, and then 1,3,4-thiadiazol-2 (3H)-one Compound 4-6 is prepared by using CDI. Next, Compound 4-7 is prepared through a substitution reaction with Compound 1-4, and then Compound 4-8 is prepared by removing an amine protecting group. Examples of Compound 4-8 may include Compound Nos. 20, 23, and 36, and the like. Then, Compound 4-9 may be prepared through reductive amination. Examples of Compound 4-9 to be prepared by Reaction Scheme 4 above may include Compound Nos. 8, 9, 10, 21, 22, 24, 25, 26, 29, 30, 37, 38, 39, 40, 41, 55, 56, 57, 58, 75, 76, 80, 81, 82, 100, 101, 102, 103, 104, 105, 106, 115, 116, 126, 127, and the like.

In addition, Compound 4-10 may be prepared through reductive amination from Compound 4-8 prepared according to Reaction Scheme 4, and Compound 4-11 may be prepared by removing the amine protecting group, followed by reductive amination to prepare Compound 4-12. Examples of Compound 4-12 may include Compound Nos. 27, 28, and the like.

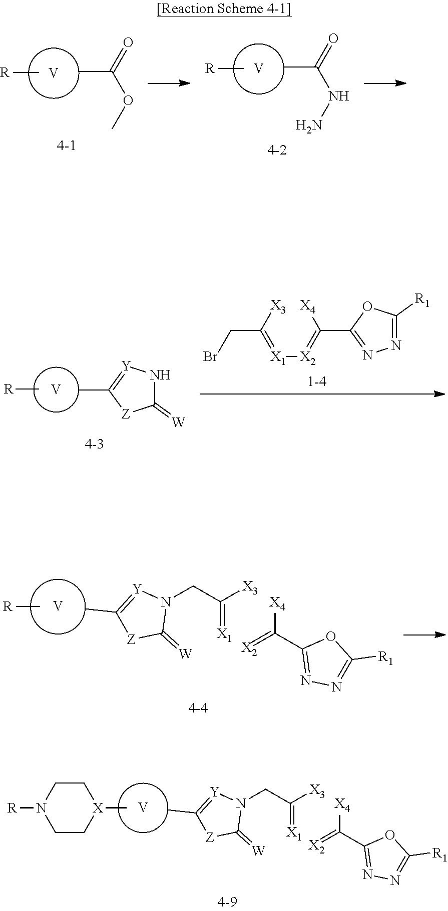

In Reaction Scheme 4-1, Compound 4-2 is prepared using hydrazine from ester compound 4-1 having a halogen element, and 1,3,4-oxadiazol-2(3H)-one Compound 4-3 is prepared using CDI. Then, Compound 4-4 is prepared through a substitution reaction with Compound 1-4, followed by an amine substitution reaction to prepare Compound 4-9. Examples of compound to be prepared by Reaction Scheme 4-1 above may include Compound Nos. 6, 7, and the like.

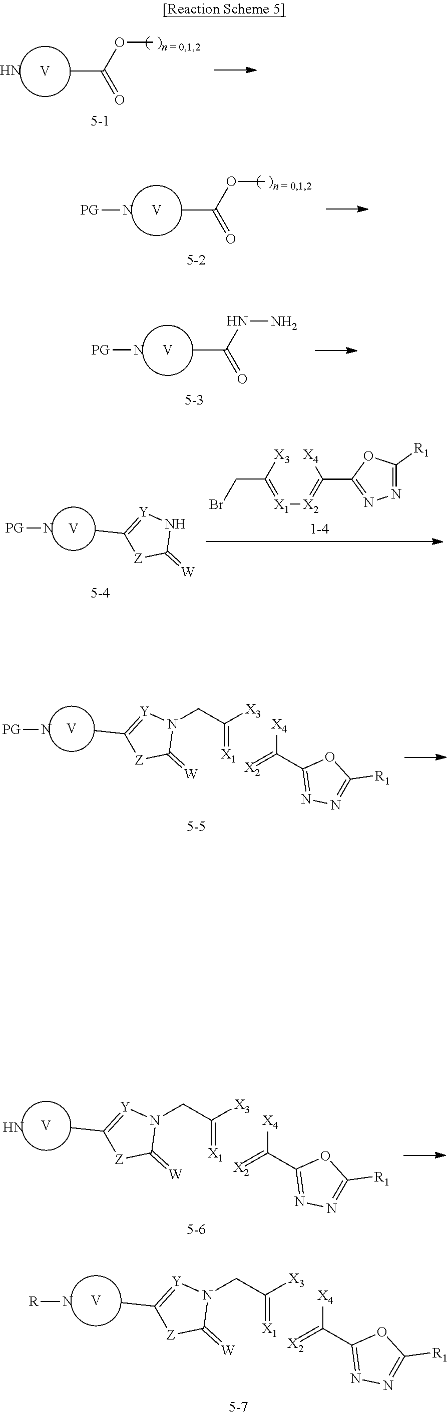

In Reaction Scheme 5, Compound 5-1 containing an amine is subjected to an amine protecting group and an alkylation reaction to prepare Compound 5-2, then Compound 5-3 is prepared using hydrazine, and then 1,3,4-oxadiazol-2(3H)-thione Compound 5-4 is prepared by employing CDI and using 1,3,4-oxadiazol-2(3H)-one and potassium ethylxanthate. Then, Compound 5-5 is prepared through a substitution reaction with Compound 1-4. Examples of Compound 5-5 to be prepared by Reaction Scheme 5 may include Compound Nos. 19, 31, 32, and the like.

In addition, Compound 5-6 may be prepared by removing the amine protecting group of Compound 5-5 prepared according to Reaction Scheme 5, and examples of Compound 5-6 may include Compound Nos. 3, 11, 33, 34, 35, and the like.

Then, Compound 5-7 is prepared through reductive amination. Examples of Compound 5-7 to be prepared by Reaction Scheme 5 above may include Compound Nos. 12, 13, 14, 135, 136, 137, 138, 139, 140, 141, and the like.

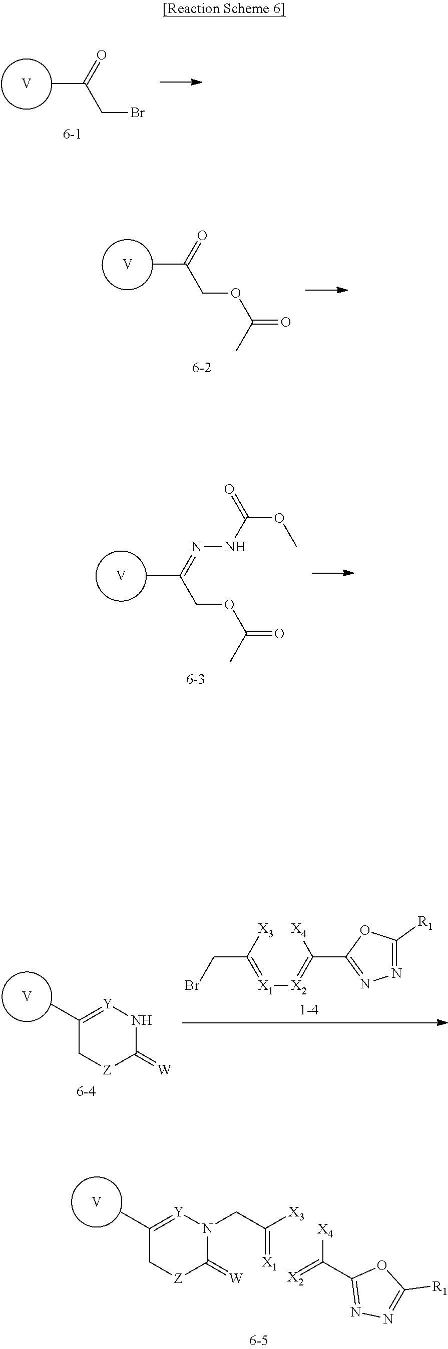

In Reaction Scheme 6, acetate compound 6-2 is prepared through a substitution reaction from bromo compound 6-1, followed by a substitution reaction to prepare hydrazine carboxylate compound 6-3, and then 1,3,4-oxadiazin-2-one Compound 6-4 is prepared by using sodium ethylate. Then, Compound 6-5 is prepared through a substitution reaction with Compound 1-4. Examples of Compound 6-5 to be prepared by Reaction Scheme 6 above may include Compound Nos. 69, 70, 71, 72, 73, 74, and the like.

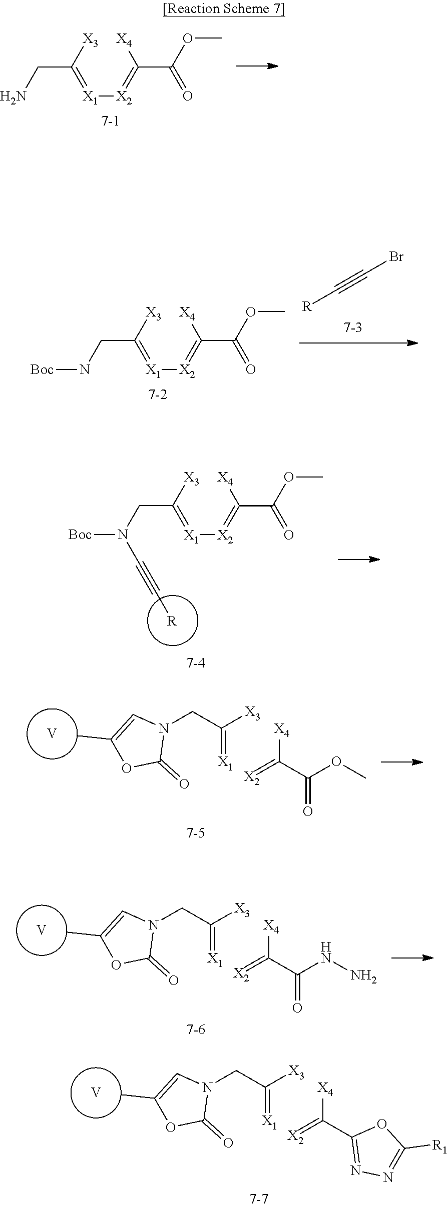

In Reaction Scheme 7, Compound 7-2 protected by an amine protecting group of Compound 7-1 is prepared, then a triple bond compound 7-4 is prepared through a substitution reaction with Compound 7-3, and then oxazol-2(3H)-one compound 7-5 is prepared using silver bis(trifluoromethanesulfonyl)imide. Next, Compound 7-6 may be prepared using hydrazine, followed by reaction with trifluoroacetic anhydride or difluoroacetic anhydride, to prepare Compound 7-7. Examples of compound to be prepared by Reaction Scheme 7 may include Compound Nos. 142, 143, and the like.

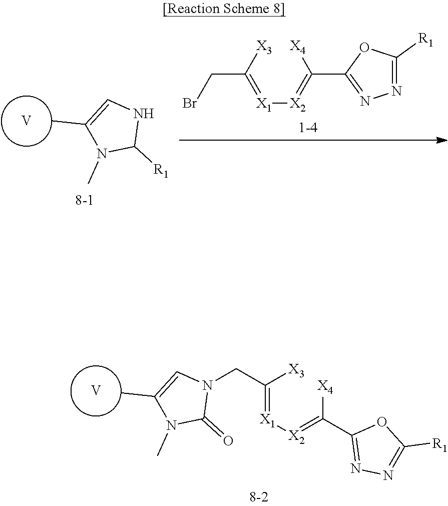

In Reaction Scheme 8, Compound 8-2 may be prepared through a substitution reaction with Compound 1-4 from imidazol-2-one compound 8-1. Examples of compound to be prepared by Reaction Scheme 8 may include Compound Nos. 144, and the like.

Uses of 1,3,4-oxadiazole Derivative Compound

The present invention provides uses of a compound represented by the following Chemical Formula I, a stereoisomer thereof or a pharmaceutically acceptable salt thereof:

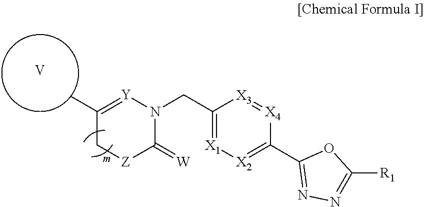

The Chemical Formula I is as defined above.

According to an embodiment of the present invention, the present invention provides a pharmaceutical composition comprising the compound represented by Chemical Formula I, a stereoisomer thereof or a pharmaceutically acceptable salt thereof as an active ingredient.

Further, according to an embodiment of the present invention, the present invention provides a pharmaceutical composition for preventing or treating histone deacetylase 6 activity-related diseases, comprising the compound represented by Chemical Formula I as described above, a stereoisomer thereof, or a pharmaceutically acceptable salt thereof as an active ingredient. The pharmaceutical composition of the present invention selectively inhibits histone deacetylase 6, thereby exhibiting remarkable effects in preventing or treating histone deacetylase 6 activity-related diseases.

The histone deacetylase 6 (HDAC6) activity-related diseases include cancer, inflammatory diseases, autoimmune diseases, neurological or neurodegenerative diseases, and specifically, lung cancer, colon cancer, breast cancer, prostate cancer, liver cancer, brain cancer, ovarian cancer, stomach cancer, skin cancer, pancreatic cancer, glioma, neuroblastoma, leukemia, lymphoma, multiple myeloma, solid cancer, Wilson's disease, spinocerebellar ataxia, prion disease, Parkinson's disease, Huntington's disease, amyotrophic lateral sclerosis, amyloidosis, Alzheimer's disease, alcoholic liver disease, spinal muscular atrophy, rheumatoid arthritis or osteoarthritis, in addition to symptoms or diseases related with abnormal function of histone deacetylases.

According to an embodiment of the present invention, examples of the histone deacetylase-mediated disease may include infectious diseases; neoplasm; endocrine, nutritional and metabolic diseases; mental and behavioral disorders; neurological diseases; eye and ocular adnexal diseases; circulatory diseases; respiratory diseases; digestive diseases; skin and subcutaneous tissue diseases; musculoskeletal and connective tissue diseases; or congenital malformations, alterations, and chromosomal abnormalities. Further, the histone deacetylase-mediated disease includes symptoms or diseases related with abnormal function of histone deacetylase 6.

In the present invention, the infectious disease may be prion disease. In addition, the neoplasm may be a benign tumor (for example, myelodysplasia syndrome) or a malignant tumor (for example, multiple myeloma, lymphoma, leukemia, lung cancer, colorectal cancer, colon cancer, prostate cancer, urothelial cell carcinoma, breast cancer, melanoma, skin cancer, liver cancer, brain cancer, stomach cancer, ovarian cancer, pancreatic cancer, head and neck cancer, oral cancer, or glioma). In addition, the endocrine, nutritional and metabolic disease may be Wilson's disease, amyloidosis or diabetes. Further, the mental and behavioral disorder may be depression or Rett syndrome. In addition, the neurological diseases may include central nervous system atrophy (for example, Huntington's disease, spinal muscular atrophy (SMA), spinocerebellar ataxia (SCA)), neurodegenerative disease (for example, Alzheimer's disease), movement disorder (for example, Parkinson's disease), neuropathy (for example, hereditary neuropathy (Charcot-Marie-Tooth disease), sporadic neuropathy, inflammatory neuropathy, drug-induced neuropathy), motor neuron disease (for example, amyotrophic lateral sclerosis (ALS)), or central nervous system demyelinating disease (for example, multiple sclerosis (MS)). In addition, the eye and ocular adnexal disease may be uveitis. Further, the circulatory disease may be atrial fibrillation or stroke. In addition, the respiratory disease may be asthma. Further, the digestive disease may be alcoholic liver disease, inflammatory bowel disease, Crohn's disease, or ulcerative bowel disease. In addition, the skin and subcutaneous tissue disease may be psoriasis. Further, the musculoskeletal and connective tissue disease may be rheumatoid arthritis, osteoarthritis or systemic lupus erythematosus (SLE). In addition, the congenital malformation, alterations and chromosomal abnormality may be autosomal dominant polycystic kidney disease.

The stereoisomer and pharmaceutically acceptable salt are as described above in the stereoisomer and pharmaceutically acceptable salt of the compound represented by Chemical Formula I of the present invention.

For administration, the pharmaceutical composition of the present invention may further comprise at least one or more pharmaceutically acceptable carrier in addition to the compound represented by Chemical Formula I, a stereoisomer or a pharmaceutically acceptable salt thereof. The pharmaceutically acceptable carrier may be saline, sterile water, Ringer's solution, buffered saline, dextrose solution, maltodextrin solution, glycerol, ethanol, or a mixture of one or more of these ingredients, and, if necessary, may contain other conventional additives such as antioxidants, buffers, bacteriostatic agents, and the like. Further, diluents, dispersants, surfactants, binders and lubricants may be additionally added and may be formulated into injectable formulations such as aqueous solutions, suspensions, emulsions, and the like, pills, capsules, granules or tablets. Accordingly, the pharmaceutical composition of the present invention may be a patch, liquid, pill, capsule, granule, tablet, suppository, or the like. These formulations may be prepared by conventional methods used for formulation in the art or a method disclosed in the document [see, Remington's Pharmaceutical Science (latest edition), Mack Publishing Company, Easton PA], and may be formulated into various formulations depending on each disease or component.

The composition of the present invention may be administered orally or parenterally (for example, intravenous, subcutaneous, intraperitoneal or topical application) according to the desired method, and the dosage varies depending on the patient's weight, age, sex, health condition, diet, administration time, administration method, excretion rate, and severity of the disease. The daily dosage of the compound represented by Chemical Formula I of the present invention is about 1 to 1000 mg/kg, preferably 5 to 100 mg/kg, and may be divided and administered once or several times a day.

The pharmaceutical composition of the present invention may further comprise at least one active ingredient exhibiting the same or similar efficacy in addition to the compound represented by Chemical Formula I, a stereoisomer or a pharmaceutically acceptable salt thereof.

According to an embodiment of the present invention, the present invention provides a method for preventing or treating histone deacetylase 6 activity-related diseases, comprising: administering a therapeutically effective amount of the compound represented by Chemical Formula I, a stereoisomer thereof, or a pharmaceutically acceptable salt thereof to a subject in need thereof. The subject may be a mammal including a human.

The method for preventing or treating histone deacetylase 6 activity-related diseases comprises not only dealing with the disease itself prior to the onset of symptoms, but also inhibiting or avoiding symptoms thereof by administering the compound represented by Chemical Formula I, a stereoisomer or a pharmaceutically acceptable salt thereof. In addition, the method for preventing or treating histone deacetylase 6 activity-related diseases of the present invention may further administering a therapeutically effective amount of an additional active agent that is helpful in treating the diseases together with the compound represented by Chemical Formula I, wherein the additional active agent may exhibit synergistic or adjuvant effects together with the compound represented by Chemical Formula I above.

The term “therapeutically effective amount” used herein refers to an amount of the compound represented by Chemical Formula I effective for the treatment or prevention of histone deacetylase 6 activity-related diseases. Specifically, the “pharmaceutically effective amount” means an amount sufficient to treat a disease at a reasonable benefit/risk ratio applicable to medical treatment, and the effective dose level may be determined depending on factors including the subject type and severity, age, sex, type of diseases, the activity of the drug, the sensitivity to the drug, the time of administration, the route of administration, the rate of excretion, the duration of treatment, drugs used concurrently, and other factors well known in the medical field. The pharmaceutical composition of the present invention may be administered as an individual therapeutic agent or in combination with other therapeutic agents, and may be administered sequentially or simultaneously with a commercially available therapeutic agent. In addition, the pharmaceutical composition of the present invention may be administered singly or in multiple doses. In consideration of all of the above factors, it is important to administer an amount capable of obtaining the maximum effect with the minimum amount without side effects, which may be easily determined by those skilled in the art. The administration dose of the pharmaceutical composition of the present invention may be determined by specialists according to various factors such as the patient's condition, age, sex, complications, and the like. Since the active ingredient of the pharmaceutical composition of the present invention has excellent safety, the active ingredient may be used even above the predetermined dose.

According to an embodiment of the present invention, the present invention provides a method for selectively inhibiting histone deacetylase 6 (HDAC6) by administering the compound represented by Chemical Formula I, a stereoisomer thereof, or a pharmaceutically acceptable salt thereof to mammals, including humans.

According to an embodiment of the present invention, the present invention provides uses of the compound represented by Chemical Formula I, the stereoisomer thereof, or the pharmaceutically acceptable salt thereof.

According to an embodiment of the present invention, the present invention provides uses of a compound represented by Chemical Formula I, a stereoisomer thereof, or a pharmaceutically acceptable salt thereof for the manufacture of a medicament for the treatment of histone deacetylase 6 activity-related diseases. The compound represented by Chemical Formula I for the manufacture of medicaments may be mixed with acceptable adjuvants, diluents, carriers, and the like, and may be prepared as a combined preparation with other active agents to have a synergistic effect of the active ingredients.

According to an embodiment of the present invention, the present invention provides uses of a compound represented by Chemical Formula I, a stereoisomer thereof or a pharmaceutically acceptable salt thereof for preventing or treating the histone deacetylase 6-mediated diseases. The compound represented by Chemical Formula I for preventing or treating the histone deacetylase 6-mediated diseases may be mixed with acceptable adjuvants, diluents, carriers, and the like, and may be prepared as a combined preparation with other active agents to have a synergistic effect of the active ingredients.

Matters mentioned in the uses, compositions and treatment methods of the present invention are applied equally as long as they do not contradict each other.

Exemplary embodiments of the present invention may be modified in various other forms, and the scope of the present invention is not limited to the exemplary embodiments described below. In addition, the exemplary embodiments of the present invention are provided to more completely explain the present invention to an ordinary person skilled in the art. Further, “including” a component throughout the specification does not mean excluding other components, but rather it means that other components may be further included, unless otherwise stated.

### Advantageous Effects of Invention

The compound represented by Chemical Formula I, a stereoisomer thereof, or a pharmaceutically acceptable salt thereof according to the present invention may selectively inhibit HDAC6 to thereby have a remarkably excellent preventive or therapeutic effect on histone deacetylase 6 activity-related diseases.

### BEST MODE FOR CARRYING OUT THE INVENTION

Hereinafter, the present invention will be described in more detail through Examples and Experimental Examples. However, these Examples and the like are only presented as examples of the present invention, and the scope of the present invention is not limited only to these Examples.

### Preparation of 1,3,4-oxadiazole Derivative Compound

A specific method for preparing a compound represented by Chemical Formula I is described as follows.

### Example 5: Synthesis of Compound 5, 3-[[5-[5-(difluoromethyl)-1,3,4-oxadiazol-2-yl]-2-pyridyl]methyl]-5-(2-thienyl)-1,3,4-oxadiazol-2-one

### [Step 1] Synthesis of 6-methylnicotinohydrazide

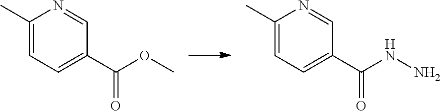

To a solution in which methyl 6-methylnicotinate (10.000 g, 66.151 mmol) was dissolved in ethanol (200 mL) at room temperature, hydrazine monohydrate (32.151 mL, 661.507 mmol) was added, and the mixture was stirred at the same temperature for 18 hours. The solvent was removed from the reaction mixture under reduced pressure to obtain a concentrate. Into the concentrate, water was poured, followed by extraction with dichloromethane. The organic layer was washed with a saturated aqueous solution of sodium hydrogen carbonate, dried over anhydrous magnesium sulfate, filtered, and concentrated under reduced pressure. The obtained product was used without further purification (8.248 g, 82.5%, white solid).

### [Step 2] Synthesis of 2-(difluoromethyl)-5-(6-methylpyridin-3-yl)-1,3,4-oxadiazole

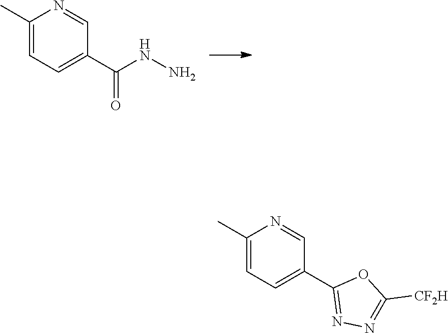

To a solution in which 6-methylnicotinohydrazide (8.248 g, 54.561 mmol) synthesized in Step 1 was dissolved in tetrahydrofuran (200 mL) at 0° C., triethylamine (38.024 mL, 272.805 mmol) was added and stirred at the same temperature for 10 minutes. To the reaction mixture, 2,2-difluoroacetic anhydride (20.349 mL, 163.683 mmol) was added and further stirred at 80° C. for 3 hours, and then the reaction was terminated by lowering the temperature to room temperature. A saturated aqueous solution of sodium hydrogen carbonate was poured into the reaction mixture, followed by extraction with dichloromethane. The organic layer was washed with a saturated aqueous sodium chloride solution, dried over anhydrous magnesium sulfate, filtered, and concentrated under reduced pressure. The concentrate was purified and concentrated by column chromatography (SiO₂, 80 g cartridge; ethyl acetate/hexane=0% to 50%) to obtain the title compound (8.019 g, 69.6%) in the form of a yellow solid.

### [Step 3] Synthesis of 2-(6-(bromomethyl)-pyridin-3-yl)-5-(difluoromethyl)-1,3,4-oxadiazole

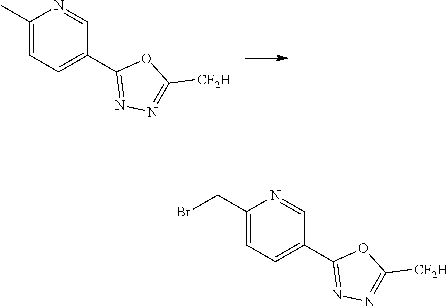 [SMILES: Cc1ccc(-c2nnc(C(F)F)o2)cn1 ; FC(F)c1nnc(-c2ccc(CBr)nc2)o1]

A solution in which 2-(difluoromethyl)-5-(6-methylpyridin-3-yl)-1,3,4-oxadiazole (8.019 g, 37.974 mmol) synthesized in Step 2, 1-bromopyrrolidin-2,5-one (NBS, 8.110 g, 45.569 mmol) and azobisisobutyronitrile (AIBN, 0.624 g, 3.797 mmol) were dissolved in 1,2-dichloroethane (120 mL) at room temperature was stirred for 6 hours at the same temperature. Water was poured into the reaction mixture, and the mixture was extracted with dichloromethane. The organic layer was washed with a saturated aqueous ammonium chloride solution, dried over anhydrous magnesium sulfate, filtered, and concentrated under reduced pressure. The concentrate was purified and concentrated by column chromatography (SiO₂, 80 g cartridge; ethyl acetate/hexane=0% to 40%) to obtain the title compound (3.960 g, 36.0%) in the form of a brown solid.

### [Step 4] Synthesis of thiophene-2-carbohydrazide

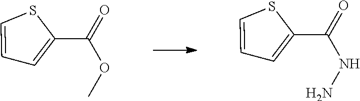

To a solution in which methylthiophene-2-carboxylate (100.00% solution, 0.163 mL, 1.410 mmol) was dissolved in ethanol (10 mL) at room temperature, hydrazine monohydrate (100.00% solution, 1.367 mL, 28.100 mmol) was added and stirred at 60° C. for 5 hours, and then the reaction was terminated by lowering the temperature to room temperature. The solvent was removed from the reaction mixture under reduced pressure to obtain a concentrate. Into the concentrate, a saturated aqueous solution of sodium hydrogen carbonate was poured, and the reaction mixture was extracted with dichloromethane. The organic layer was washed with a saturated aqueous sodium chloride solution, dried over anhydrous magnesium sulfate, filtered, and concentrated under reduced pressure. The obtained product was used without further purification (0.2 g, 99.8%, pale yellow solid).

### [Step 5] Synthesis of 5-(2-thienyl)-3H-1,3,4-oxadiazol-2-one

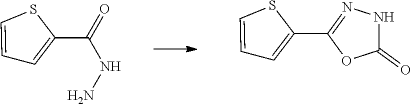 [SMILES: NNC(=O)c1cccs1 ; O=c1[nH]nc(-c2cccs2)o1]

To a solution in which thiophene-2-carbohydrazide (100.00%. 0.200 g. 1.407 mmol) synthesized in Step 4 and 1,1′-carbonylbis-1H-imidazole (100.00%, 0.274 g, 1.690 mmol) were dissolved in tetrahydrofuran (10 mL) at room temperature, triethylamine (100.00% solution, 0.273 mL, 2.000 mmol) was added and stirred overnight at the same temperature. Water was poured into the reaction mixture, and the mixture was extracted with dichloromethane. The organic layer was washed with a saturated aqueous sodium chloride solution, dried over anhydrous magnesium sulfate, filtered, and concentrated under reduced pressure. The concentrate was purified and concentrated by column chromatography (SiO₂, 4 g cartridge; methanol/dichloromethane=0% to 10%) to obtain the title compound (0.143 g, 60.450%) as a pale yellow solid.

### [Step 6] Synthesis of Compound 5

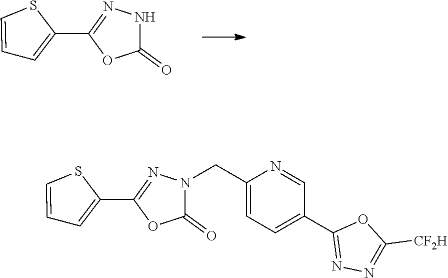 [SMILES: O=c1[nH]nc(-c2cccs2)o1 ; O=c1oc(-c2cccs2)nn1Cc1ccc(-c2nnc(C(F)F)o2)cn1]

To a solution in which 5-(2-thienyl)-3H-1,3,4-oxadiazol-2-one (100.00%, 0.063 g, 0.350 mmol) synthesized in Step 5 and potassium carbonate (100.00%, 0.049 g, 0.494 mmol) were dissolved in N,N-dimethylformamide (2 mL) at room temperature, 2-[6-(bromomethyl)-3-pyridyl]-5-(difluoromethyl)-1,3,4-oxadiazole (100.00%, 0.122 g, 0.421 mmol) synthesized in Step 3 was added and stirred overnight at the same temperature. Water was poured into the reaction mixture, and the mixture was extracted with ethyl acetate. The organic layer was washed with a saturated aqueous sodium chloride solution, dried over anhydrous magnesium sulfate, filtered, and concentrated under reduced pressure. The concentrate was purified and concentrated by column chromatography (SiO₂, 4 g cartridge; methanol/dichloromethane=0% to 10%), and then the obtained product was purified and concentrated again by chromatography (SiO₂, 4 g cartridge; methanol/dichloromethane=0% to 10%) to obtain the title compound (0.036 g, 26.44%, yellow solid).

¹H NMR (400 MHz, CDCl₃) δ 9.34-9.33 (m, 1H), 8.44 (dd, J=8.2, 2.2 Hz, 1H), 7.65 (dd, J=4.0, 1.2 Hz, 1H), 7.55-7.52 (m, 2H), 7.17-7.15 (m, 1H), 6.96 (t, J=51.6 Hz, 1H), 5.22 (s, 2H); LRMS (ES) m/z 378.7 (M⁺+1).

Compounds in Table 2 below were synthesized according to substantially the same method as in Example 5.

TABLE 2

| Ex- | Com- | |
|---|---|---|
| am- | pound | |
| ple | No. | Compound Name, ¹H-NMR, MS (ESI) |
| 1 | 1 | 3-((5-(5-(difluoromethyl)-1, 3, 4-oxadiazol-2- |
| | | yl)pyridin-2-yl)methyl)-5-phenyl-1, 3, 4- |
| | | oxadiazol-2 (3H)-one |
| | | ¹H NMR (400 MHz, CDCl₃) δ 9.34 (d, J = 1.6 |
| | | Hz, 1H), 8.44 (dd, J = 8.0, 2.0 Hz, 1H), 7.88 (d, |
| | | J =7.2 Hz, 1H), 7.55-7.48 (m, 4H), 6.96 (t, J = |
| | | 51.6 Hz, 1H), 5.25 (s, 2H) ; LRMS (ES) m/z |
| | | 372.7 (M⁺ + 1) . |
| 15 | 15 | 3-[[5-[5-(difluoromethyl)-1, 3, 4-oxadiazol-2- |
| | | yl]-2-pyridyl]methyl]-5-(2-fluorophenyl)-1, 3, |
| | | 4-oxadiazol-2-one |
| | | ¹H NMR (400 MHz, CDCl₃) δ 9.33 (d, J = 1.6 |
| | | Hz, 1H), 8.44 (dd, J = 8.2, 2.2 Hz, 1H), 7.87- |
| | | 7.83 (m, 1H), 7.57-7.51 (m, 2H), 7.32-7.21 |
| | | (m, 2H), 6.96 (t, J = 51.8 Hz, 1H), 5.28 (s, 2H); |
| | | LRMS (ES) m/z 390.7 (M⁺ + 1). |
| 16 | 16 | 3-[[5-[5-(difluoromethyl)-1, 3, 4-oxadiazol-2- |
| | | yl]-2-pyridyl]methyl]-5-(3-thienyl)-1, 3, 4- |
| | | oxadiazol-2-one |
| | | ¹H NMR (400 MHz, CDCl₃) δ 9.33 (d, J = 1.6 |
| | | Hz, 1H), 8.44 (dd, J = 8.2, 2.2 Hz, 1H), 7.93 |
| | | (dd, J = 2.8, 1.2 Hz, 1H), 7.53 (d, J = 8.4 Hz, |
| | | 1H), 7.49 (dd, J = 5.2, 1.2 Hz, 1H) , 7.45-7.43 |
| | | (m, 1H), 6.96 (t, J = 51.6 Hz, 1H), 5.22 |
| | | (s, 2H); LRMS (ES) m/z 378.6 (M⁺ + 1) . |
| 17 | 17 | 3-[[5-[5-(difluoromethyl)-1, 3, 4-oxadiazol-2- |
| | | yl]-2-pyridyl]methyl]-5-(7-quinolyl)-1, 3, 4- |
| | | oxadiazol-2-one |
| | | ¹H NMR (400 MHz, CD₃OD) δ 9.28 (d, J = 1.6 |
| | | Hz, 1H), 8.96 (dd, J = 4.4, 1.6 Hz, 1H), 8.55- |
| | | 8.52 (m, 2H), 8.07 (d, J = 1.6 Hz, 2H), 7.74 (d, |
| | | J = 8.4 Hz, 1H), 7.64 (dd, J = 8.4, 4.4 Hz, 1H), |
| | | 7.20 (t, J = 51.6 Hz, 1H), 5.32 (s, 2H); |
| | | LRMS (ES) m/z 423.7 (M⁺ + 1). |
| 18 | 18 | 5-(1, 3-benzothiazol-5-yl)-3-[[5-[5- |
| | | (difluoromethyl)-1, 3, 4-oxadiazol-2-yl]-2- |
| | | pyridyl]methyl]-1, 3, 4-oxadiazol-2-one |
| | | ¹H NMR (400 MHz, CD₃OD) δ 9.37 (s, 1H), |
| | | 9.27 (d, J = 1.6 Hz, 1H), 8.61 (d, J = 1.2 Hz, |
| | | 1H), 8.53 (dd, J = 8.2, 2.2 Hz, 1H), 8.20 |
| | | (d, J = 8.4 Hz, 1H), 8.06 (dd, J = 8.4, 1.6 Hz, |
| | | 1H), 7.73 (d, J = |
| | | 8.0 Hz, 1H), 7.22 (t, J = 51.6 Hz, 1H), 5.29 |
| | | (s, 2H); LRMS (ES) m/z 429.7 (M⁺ + 1) . |
| 42 | 42 | 3-[[5-[5-(difluoromethyl)-1, 3, 4-oxadiazol-2- |
| | | yl]-2-pyridyl]methyl]-5-(2-quinolyl)-1, 3, 4- |
| | | oxadiazol-2-one |
| | | ¹H NMR (400 MHz, DMSO-d₆) δ 9.22 (d, J = |
| | | 1.6 Hz, 1H), 8.58 (d, J = 8.4 Hz, 1H), 8.49 |
| | | (dd, J = 8.2, 2.2 Hz, 1H), 8.15 (d, J = 8.4 Hz, |
| | | 1H), 8.10 (d, J = 8.4 Hz, 1H), 8.02 (d, J = 8.4 |
| | | Hz, 1H), 7.92-7.87 (m, 1H), 7.81 (d, J = 8.4 Hz, |
| | | 1H), 7.77-7.72 (m, 1H), 7.58 (t, J = 51.2 Hz, |
| | | 1H), 5.35 (s, 2H); LRMS (ES) m/z |
| | | 423.9 (M⁺ + 1) . |

### Example 45: Synthesis of Compound 45, 3-((5-(5-(difluoromethyl)-1,3,4-oxadiazol-2-yl)pyridin-2-yl)methyl)-5-phenyl-1,3,4-oxadiazol-2(3H)-thione

### [Step 1] Synthesis of benzohydrazide

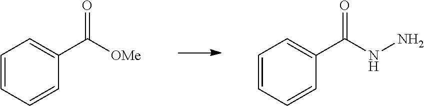 [SMILES: COC(=O)c1ccccc1 ; NNC(=O)c1ccccc1]

A solution in which methyl benzoate (0.500 g, 2.203 mmol) and hydrazine monohydrate (0.920 mL, 7.308 mmol) were dissolved in ethanol (12 mL) at room temperature was heated to reflux for 18 hours, and then the reaction was terminated by lowering the temperature to room temperature. After removing the solvent from the reaction mixture under reduced pressure, the obtained product was used without further purification (0.500 g, 100.0%, white solid).

### [Step 2] Synthesis of 5-phenyl-1,4-oxadiazol-2(3H)-thione

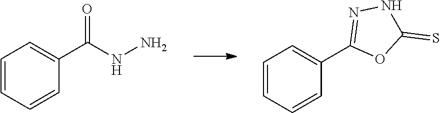 [SMILES: NNC(=O)c1ccccc1 ; S=c1[nH]nc(-c2ccccc2)o1]

To a solution in which the benzohydrazide (100.00%, 0.500 g, 3.672 mmol) synthesized in Step 1 was dissolved in ethanol (12 mL) at room temperature, potassium ethylxanthate (100.00%, 0.589 g, 3.674 mmol) was added and the mixture was stirred at the same temperature. After removing the solvent from the reaction mixture under reduced pressure, the precipitated solid was filtered and washed with water, followed by drying to obtain the title compound (0.45 g, 68.759%) as a pale yellow solid.

### [Step 3] Synthesis of Compound 45

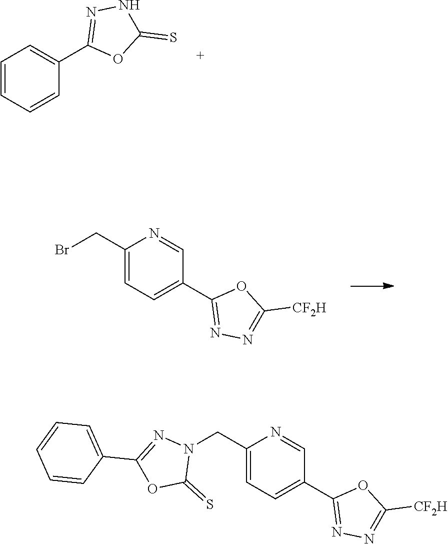 [SMILES: S=c1[nH]nc(-c2ccccc2)o1 ; FC(F)c1nnc(-c2ccc(CBr)nc2)o1 ; FC(F)c1nnc(-c2ccc(Cn3nc(-c4ccccc4)oc3=S)nc2)o1]

To a solution in which 5-phenyl-1,3,4-oxadiazol-2(3H)-thione (100.00%, 50.000 mg, 0.281 mmol) synthesized in Step 1 was dissolved in N,N-dimethylformamide (2 mL) at room temperature, potassium carbonate (100.00%, 40.000 mg, 0.404 mmol) was added and stirred at the same temperature for 0.3 hours. To the reaction mixture, 2-[6-(bromomethyl)-3-pyridyl]-5-(difluoromethyl)-1,3,4-oxadiazole (100.00%, 100.000 mg, 0.345 mmol) was added and further stirred at 35° C. for 18 hours. Water was poured into the reaction mixture, and the mixture was extracted with ethyl acetate. The organic layer was washed with a saturated aqueous sodium chloride solution, dried over anhydrous magnesium sulfate, filtered, and concentrated under reduced pressure. The concentrate was purified and concentrated by column chromatography (SiO₂, 4 g cartridge; hexane/ethyl acetate=100% to 50%) to obtain the title compound (58 mg, 53.36%) in the form of a white solid.

¹H NMR (400 MHz, CDCl₃) δ 9.32 (d, J=1.6 Hz, 1H), 8.40 (dd, J=8.2, 2.2 Hz, 1H), 8.01 (dd, J=8.2, 1.4 Hz, 2H), 7.83 (d, J=8.4 Hz, 1H), 7.56-7.51 (m, 3H), 6.96 (t, J=51.6 Hz, 1H), 4.75 (s, 2H); LRMS (ES) m/z 388.8 (M⁺+1).

Compounds in Table 3 below were synthesized according to substantially the same method as in Example 45.

TABLE 3

| Ex- | Com- | |
|---|---|---|
| am- | pound | |
| ple | No. | Compound Name, ¹H-NMR, MS (ESI) |
| 46 | 46 | 3-[[5-[5-(difluoromethyl)-1, 3, 4-oxadiazol-2- |
| | | yl]-2-pyridyl]methyl]-5-(2-pyridyl)-1, 3, 4- |
| | | oxadiazol-2-thione |
| | | ¹H NMR (400 MHz, CDCl₃) δ 9.39 (d, J = |
| | | 1.6 Hz, 1H), 8.78 (d, J = 1.6 Hz, 1H), 8.40 |
| | | (dd, J = 8.2, 2.2 Hz, 1H), 8.21 (td, J = 4.5, 2.7 |
| | | Hz, 1H), 7.90 (td, J = 7.8, 1.6 Hz, 1H), 7.83 |
| | | (d, J = 8.4 Hz, 1H), 6.96 (t, J = 51.6 Hz, 1H), |
| | | 4.78 (s, 2H);LRMS (ES) m/z 389.8 (M⁺ + 1). |
| 47 | 47 | 3-[[5-[5-(difluoromethyl)-1, 3, 4-oxadiazol-2- |
| | | yl]-3-fluoro-2-pyridyl]methyl]-5-(2-pyridyl)- |
| | | 1, 3, 4-oxadiazol-2-thione |
| | | ¹H NMR (400 MHz, CDCl₃) δ 9.08 (d, J = |
| | | 1.6 Hz, 1H), 8.79 (dd, J = 1.6, 0.8 Hz, 1H), |
| | | 8.23 (dt, J = 8.0, 1.0 Hz, 1H), 8.16 (dd, J = |
| | | 9.2, 1.6 Hz, 1H), 7.91 (td, J = 7.8, 1.6 Hz, 1H), |
| | | 7.50-7.02 (m, 1H), 6.96 (t, J = 51.6 Hz, 1H), |
| | | 4.87 (d, J = 2.0 Hz, 2H); LRMS (ES) m/z |
| | | 407.7 (M⁺ + 1). |
| 48 | 48 | 3-[[5-[5-(difluoromethyl)-1, 3, 4-oxadiazol-2- |
| | | yl]-3-fluoro-2-pyridyl]methyl]-5-(3- |
| | | fluorophenyl)-1, 3, 4-oxadiazol-2-thione |
| | | ¹H NMR (400 MHz, CDCl₃) δ 9.18 (s, 1H), |
| | | 8.17 (dd, J = 9.0, 1.8 Hz, 1H), 7.84 (dd, |
| | | J = 4.4, 3.6 Hz, 1H), 7.82-7.77 (m, 1H), 7.54- |
| | | 7.48 (m, 1H), 7.27-7.24 (m, 1H), 6.96 (t, J = |
| | | 51.6 Hz, 1H), 4.83 (d,J = 2.0 Hz, 2H); LRMS |
| | | (ES) m/z 424.7 (M⁺ + 1). |
| 50 | 50 | 3-[ [5-[5- (difluoromethyl) -1, 3, 4-oxadiazol- |
| | | 2-yl] -2-pyridyl ]methyl] -5-(2-thienyl)- |
| | | 1, 3, 4-oxadiazol-2-thione |
| | | ¹H NMR (400 MHz, CDCl₃) δ 9.32 (dd, J = |
| | | 2.0, 0.8 Hz, 1H), 8.39 (dd, J = 8.2, 2.2 Hz, |
| | | 1H), 7.82 (d, J = 7.6 Hz, 1H), 7.72 (dd, J = |
| | | 3.6, 1.2 Hz, 1H), 7.56 (dd, J = 4.8, 1.2 Hz, |
| | | 1H), 7.18 (dd, J = 5.2, 3.6 Hz, 1H), 6.96 |
| | | (t, J = 51.8 Hz, 1H), 4.73 (s, 2H); |
| | | LRMS (ES) m/z 394.3 (M⁺ + 1). |
| 51 | 51 | 3-[[5-[5-(difluoromethyl)-1, 3, 4-oxadiazol-2- |
| | | yl]-3-fluoro-2-pyridyl]methyl]-5-(2-thienyl)- |
| | | 1, 3, 4-oxadiazol-2-thione |
| | | ¹H NMR (400 MHz, CDCl₃) δ 7.90-7.80 (m, |
| | | 2H), 7.71 (dd, J = 3.6, 1.2 Hz, 1H), 7.57 (dd, |
| | | J = 4.8, 1.2 Hz, 1H), 7.18 (dd, J = 5.2, 3.6 Hz, |
| | | 1H), 6.93 (t, J = 51.6 Hz, 1H), 4.59 |
| | | (s, 2H); LRMS (ES) m/z 411.7 (M⁺ + 1). |
| 52 | 52 | 3-[[5-[5-(difluoromethyl)-1, 3, 4-oxadiazol-2- |
| | | yl]-2-pyridyl]methyl]-5-(3-pyridyl)-1, 3, 4- |
| | | oxadiazol-2-thione |
| | | ¹H NMR (400 MHz, DMSO-d₆) δ 9.14 (d, J = |
| | | 1.6 Hz, 1H), 9.08 (d, J = 1.6 Hz, 1H), 8.80 |
| | | (dd, J = 4.8, 1.6 Hz, 1H), 8.46 (dd, J = 8.2, 2.2 |
| | | Hz, 1H), 8.34 (dt, J = 8.0, 2.0 Hz, 1H), 7.86 |
| | | (d, J = 8.4 Hz, 1H), 7.66-7.58 (m, 1H), 7.58 |
| | | (t, J = 51.2 Hz, 1H), 4.87 (s, 2H); LRMS (ES) |
| | | m/z 389.5 (M⁺ + 1). |
| 53 | 53 | 3-[[5-[5-(difluoromethyl)-1, 3, 4-oxadiazol-2- |
| | | yl]-3-fluoro-2-pyridyl]methyl]-5-pyrazine- |
| | | 2-yl-1, 3, 4-oxadiazol-2-thione |
| | | ¹H NMR (400 MHz, DMSO-d₆) δ 9.33 (d, J = |
| | | 1.6 Hz, 1H), 9.04 (d, J = 1.2 Hz, 1H), |
| | | 8.89-8.86 (m, 2H), 8.46 (dd, J = 9.6, 1.6 Hz, |
| | | 1H), 7.59 (t, J = 51.2 Hz, 1H), 4.95 (d, J = 1.6 |
| | | Hz, 2H); LRMS (ES) m/z 408.7 (M⁺ + 1). |
| 59 | 59 | 3-[[5-[5-(difluoromethyl)-1, 3, 4-oxadiazol-2- |
| | | yl]-2-pyridyl]methyl]-5-(2-methylthiazol-4- |
| | | yl)-1, 3, 4-oxadiazol-2-thione |
| | | ¹H NMR (400 MHz, DMSO-d₆) δ 9.17 (s, 1H), |
| | | 8.46 (d, J = 6.4 Hz, 1H), 8.39 (s, 1H), 7.84 |
| | | (d, J = 10.0 Hz, 1H), 7.58 (t, J = 50.0 Hz, 1H), |
| | | 4.83 (s, 2H), 2.74 (s, 3H); LRMS (ES) |
| | | m/z 409.7 (M⁺ + 1). |
| 60 | 60 | 3-[[5-[5-(difluoromethyl)-1, 3, 4-oxadiazol- |
| | | 2-yl]-3-fluoro-2-pyridyl]methyl]-5- |
| | | (2-methylthiazol-4-yl)-1, 3, 4-oxadiazol- |
| | | 2-thione |
| | | ¹H NMR (400 MHz, DMSO-d₆) δ 9.03 (s, 1H), |
| | | 8.45 (d, J = 8.4 Hz, 1H), 8.39 (s, 1H), 7.59 |
| | | (t, J = 51.0 Hz, 1H) , 4. 87 (s, 2H), 2.74 |
| | | (s, 3H); LRMS (ES) m/z 427.7 (M⁺ + 1). |
| 61 | 61 | 3-[[5-[5-(difluoromethyl)-1, 3, 4-oxadiazol-2- |
| | | yl]-3-fluoro-2-pyridyl]methyl]-5-(2- |
| | | methylthiazol- |
| | | 5-yl)-1, 3, 4-oxadiazol-2-thione |
| | | ¹H NMR (400 MHz, DMSO-d₆) δ 9.03 (s, 1H), |
| | | 8.45 (d, J = 10.0 Hz, 1H), 8.31 (s, 1H), 7.59 |
| | | (t, J = 51.0 Hz, 1H), 4.87 (s, 2H), 2.73 (s, 3H); |
| | | LRMS (ES) m/z 427.6 (M⁺ + 1). |
| 62 | 62 | 3-[[5-[5-(difluoromethyl)-1, 3, 4-oxadiazol-2- |
| | | yl]-3-fluoro-2-pyridyl]methyl]-5-(3-pyridyl)- |
| | | 1, 3, 4-oxadiazol-2-thione |
| | | ¹H NMR (400 MHz, DMSO-d₆) δ 9.14 (s, 1H), |
| | | 9.04 (s, 1H), 8.81 (s, 1H), 8.45 (d, J = 9.2 Hz, |
| | | 1H), 8.34 (d, J = 7.6 Hz, 1H), 7.65 (t, J = 3.8 |
| | | Hz, 1H), 7.59 (t, J = 50.6 Hz, 1H), 4.92 |
| | | (s, 2H); LRMS (ES) m/z 407.5 (M⁺ + 1). |

### Example 43: Synthesis of Compound 43, 3-[[5-[5-(difluoromethyl)-1,3,4-oxadiazol-2-yl]-2-pyridyl]methyl]-5-phenyl-1,3,4-thiadiazol-2-one

### [Step 1] Synthesis of Benzohydrazide

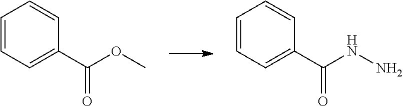

A solution in which methyl benzoate (100.00%, 1.000 g, 7.345 mmol) and hydrazine monohydrate (100.00%, 3.677 g, 73.452 mmol) were dissolved in ethanol (80 mL) at 90° C. was stirred overnight at the same temperature, and then the reaction was terminated by lowering the temperature to room temperature. The solvent was removed from the reaction mixture under reduced pressure to obtain a concentrate. Into the concentrate, water was poured, followed by extraction with dichloromethane. The organic layer was washed with a saturated aqueous solution of sodium hydrogen carbonate, dried over anhydrous magnesium sulfate, filtered, and concentrated under reduced pressure. The obtained product was used without further purification (0.88 g, 88.000%, white solid).

### [Step 2] Synthesis of benzenecarbothiohydrazide

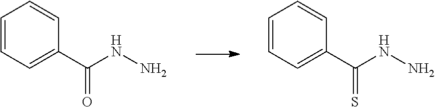 [SMILES: NNC(=O)c1ccccc1 ; NNC(=S)c1ccccc1]

A solution in which benzohydrazide (100.00%, 0.500 g, 3.672 mmol) synthesized in Step 1 and Lawesson's reagent (100.00%, 1.782 g, 4.406 mmol) were dissolved in toluene (20 mL) at 100° C. was stirred overnight at the same temperature, and then the reaction was terminated by lowering the temperature to room temperature. Water was poured into the reaction mixture, and the mixture was extracted with ethyl acetate. The organic layer was washed with a saturated aqueous sodium chloride solution, dried over anhydrous magnesium sulfate, filtered, and concentrated under reduced pressure. The concentrate was purified and concentrated by column chromatography (SiO₂, 12 g cartridge; ethyl acetate/hexane=0% to 30%) to obtain the title compound (0.31 g, 55.455%, white solid).

### [Step 3] Synthesis of 5-phenyl-3H-1,3,4-thiadiazol-2-one

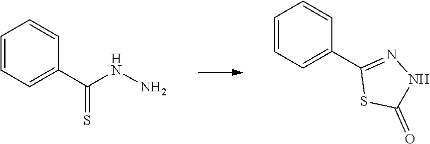 [SMILES: NNC(=S)c1ccccc1 ; O=c1[nH]nc(-c2ccccc2)s1]

A solution in which benzenecarbothiohydrazide (100.00%, 0.280 g, 1.839 mmol) synthesized in Step 2 and 1,1′-carbonylbis-1H-imidazole (100.00%, 0.358 g, 2.208 mmol) were dissolved in di-chloromethane (20 mL) at 50° C. was stirred overnight at the same temperature. Then, the reaction was terminated by lowering the temperature to room temperature. Water was poured into the reaction mixture, and the mixture was extracted with dichloromethane. The organic layer was washed with a saturated aqueous sodium chloride solution, dried over anhydrous magnesium sulfate, filtered, and concentrated under reduced pressure. The concentrate was purified and concentrated by column chromatography (SiO₂, 24 g cartridge; ethyl acetate/hexane=0% to 30%) to obtain the title compound (0.25 g, 76.264%, white solid).

### [Step 4] Synthesis of Compound 43

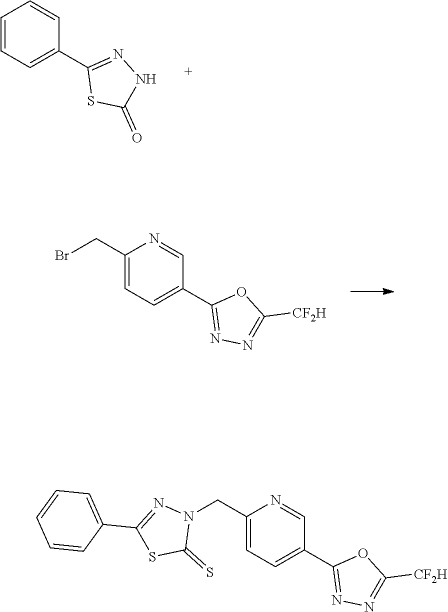 [SMILES: O=c1[nH]nc(-c2ccccc2)s1 ; FC(F)c1nnc(-c2ccc(CBr)nc2)o1 ; FC(F)c1nnc(-c2ccc(Cn3nc(-c4ccccc4)sc3=S)nc2)o1]

A solution in which 5-phenyl-3H-1,3,4-thiadiazol-2-one (100.00%, 0.050 g, 0.281 mmol) synthesized in Step 3 and potassium carbonate (100.00%, 0.058 g, 0.420 mmol) were dissolved in N,N-dimethylformamide (5 mL) was stirred at room temperature for 30 minutes. Then, 2-[6-(bromomethyl)-3-pyridyl]-5-(difluoromethyl)-1,3,4-oxadiazole (100.00%, 0.085 g, 0.293 mmol) and potassium iodide (100.00%, 0.023 g, 0.139 mmol) were added and further stirred overnight at the same temperature. Water was poured into the reaction mixture, and the mixture was extracted with ethyl acetate. The organic layer was washed with a saturated aqueous ammonium chloride solution, dried over anhydrous magnesium sulfate, filtered, and concentrated under reduced pressure. The concentrate was purified and concentrated by column chromatography (SiO₂, 4 g cartridge; ethyl acetate/hexane=0% to 50%) to obtain the title compound (0.054 g, 49.68%, white solid).

¹H NMR (400 MHz, CDCl₃) δ 9.33 (d, J=1.6 Hz, 1H), 8.42 (dd, J=8.2, 2.2 Hz, 1H), 7.71-7.69 (m, 2H), 7.48-7.43 (m, 4H), 7.08 (s, 0.2H), 6.95 (s, 0.5H), 6.82 (s, 0.2H), 5.43 (s, 2H); LRMS (ES) m/z 388.0 (M⁺+1).

Compounds in Table 4 below were synthesized according to substantially the same method as in Example 43.

TABLE 4

| | Com- | |
|---|---|---|
| Ex- | pound | Compound Name, ¹H-NMR, MS (ESI) |
| ample | No. | |
| 44 | 44 | 3-[[5-[5-(difluoromethyl)-1, 3, 4-oxadiazol-2- |
| | | yl]2-pyridyl]methyl]-5-(2-thienyl)-1, 3, 4- |
| | | thiadiazol-2-one |
| | | ¹H NMR (400 MHz, CDCl₃) δ 9.33 (d, J = 1.7 |
| | | Hz, 1H), 8.42 (dd, J = 8.2, 2.2 Hz, 1H), |
| | | 7.47-7.45 (m, 2H), 7.36-7.35 (m, 1H), |
| | | 7.11-7.09 (m, 1H), 7.08 (s, 0.2H), 6.95 (s, 0.5H), |
| | | 6.82 (s, 0.2H), 5.39 (s, 2H) ; LRMS (ES) m/z |
| | | 394.6 (M⁺ + 1). |
| 49 | 49 | 3-[[5-[5-(difluoromethyl)-1, 3, 4-oxadiazol-2- |
| | | yl]-2-pyridyl]methyl]-5-(2-pyridyl)-1, 3, 4- |
| | | thiadiazol-2-one |
| | | ¹H NMR (400 MHz, CDCl₃) δ 9.33 (d, J = 1.7 |
| | | Hz, 1H), 8.63-8.62 (m, 1H), 8.42 (dd, J = 8.2, |
| | | 2.2 Hz, 1H), 7.98-7.96 (m, 1H), 7.81-7.77 |
| | | (m, 1H), 7.49 (d, J = 8.2 Hz, 1H), 7.39-7.36 |
| | | (m, 1H), 7.08 (s, 0.2H), 6.95 (s, 0.5H), 6.82 |
| | | (s, 0.2H), 5.45 (s, 2H); LRMS (ES) m/z |
| | | 389.76 (M⁺ + 1). |
| 54 | 54 | 3-[[5-[5-(difluoromethyl)-1, 3, 4-oxadiazol-2- |
| | | yl]-2-pyridyl]methyl]-5-(2-fluorophenyl)- |
| | | 1, 3, 4-thiadiazol-2-one |
| | | ¹H NMR (400 MHz, CDCl₃) δ 9.34 (d, J = 1.6 |
| | | Hz, 1H), 8.42-8.40 (m, 1H), 7.94-7.90 (m, 1H), |
| | | 7.49-7.43 (m, 2H), 7.26-7.17 (m, 2H), 7.08 |
| | | (s, 0.2H), 6.95 (s, 0.5H), 6.82 (s, 0.2H), 5.45 |
| | | (s, 2H);LRMS (ES) m/z 406.67 (M⁺ + 1). |

### Example 4: Synthesis of Compound 4, 3-[[5-[5-(difluoromethyl)-1,3,4-oxadiazol-2-yl]-2-pyridyl]methyl]-5-[2-fluoro-4-[(4-methyl-1-piperidyl)methyl]phenyl]-1,3,4-oxadiazol-2-one

### [Step 1] Synthesis of methyl 2-fluoro-4-[(4-methyl-1-piperidyl)methyl]benzoate

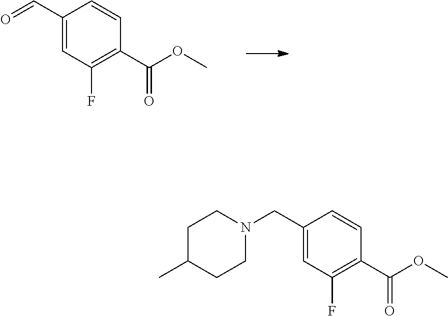

A solution in which methyl 2-fluoro-4-formyl-benzoate (100.00%, 0.300 g, 1.647 mmol), 4-methylpiperidine (100.00%, 2.000 equiv., 3.294 mmol) and sodium triacetoxyborohydride (100.00%, 2.000 equiv., 3.294 mmol) were dissolved in dichloromethane (10 mL) at room temperature was stirred overnight at the same temperature. Water was poured into the reaction mixture, and the mixture was extracted with dichloromethane. The organic layer was washed with a saturated aqueous sodium chloride solution, dried over anhydrous magnesium sulfate, filtered, and concentrated under reduced pressure. The concentrate was purified and concentrated by column chromatography (SiO₂, 4 g cartridge; ethyl acetate/hexane=0% to 100%) to obtain the title compound (0.43 g, 98.41%, yellow oil).

### [Step 2] Synthesis of 2-fluoro-4-[(4-methyl-1-piperidyl)methyl]benzohydrazide

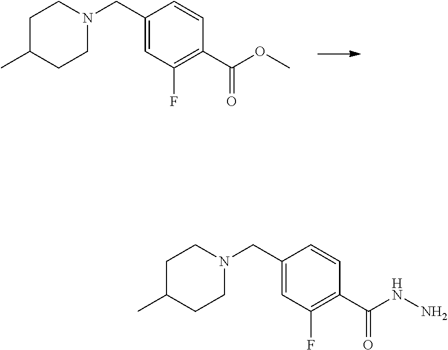

A solution in which methyl 2-fluoro-4-[(4-methyl-1-piperidyl)methyl]benzoate (100.00%, 0.420 g, 1.583 mmol) synthesized in Step 1 and hydrazine monohydrate (100.00%, 10.000 equiv., 15.830 mmol) were dissolved in ethanol (10 mL) at 80° C. was stirred overnight at the same temperature, and then the reaction was terminated by lowering the temperature to room temperature. After removing the solvent from the reaction mixture under reduced pressure, the obtained product was used without further purification (0.42 g, 100.0%, white solid).

### [Step 3] Synthesis of 5-[2-fluoro-4-[(4-methyl-1-piperidyl)methyl]phenyl]-3H-1,3,4-oxadiazol-2-one

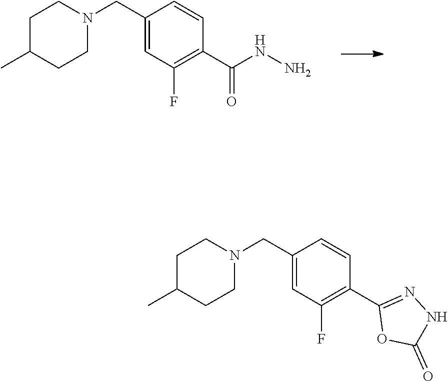 [SMILES: CC1CCN(Cc2ccc(C(=O)NN)c(F)c2)CC1 ; CC1CCN(Cc2ccc(-c3n[nH]c(=O)o3)c(F)c2)CC1]

A solution in which 2-fluoro-4-[(4-methyl-1-piperidyl)methyl]benzohydrazide G (100.00%, 0.420 g, 1.583 mmol) synthesized in Step 2, triphosgene (100.00%, 0.400 equiv., 0.633 mmol) and N,N-diisopropylethylamine (100.00%, 2.000 equiv., 3.166 mmol) were dissolved in dichloromethane (10 mL) at room temperature was stirred at the same temperature. Water was poured into the reaction mixture, and the mixture was extracted with dichloromethane. The organic layer was washed with a saturated aqueous sodium chloride solution, dried over anhydrous magnesium sulfate, filtered, and concentrated under reduced pressure. The concentrate was purified and concentrated by column chromatography (SiO₂, 12 g cartridge; ethyl acetate/hexane=0% to 100%) to obtain the title compound (0.275 g, 59.63%, white solid).

### [Step 4] Synthesis of Compound 4

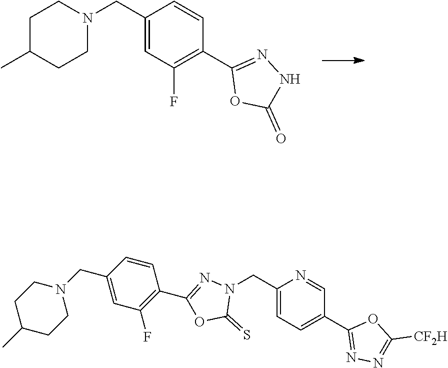 [SMILES: CC1CCN(Cc2ccc(-c3n[nH]c(=O)o3)c(F)c2)CC1 ; CC1CCN(Cc2ccc(-c3nn(Cc4ccc(-c5nnc(C(F)F)o5)cn4)c(=S)o3)c(F)c2)CC1]

A solution in which 5-[2-fluoro-4-[(4-methyl-1-piperidyl)methyl]phenyl]-3H-1,3,4-oxadiazol-2-one (100.00%, 0.090 g, 0.309 mmol) synthesized in Step 3, 2-[6-(bromomethyl)-3-pyridyl]-5-(difluoromethyl)-1,3,4-oxadiazole (100.00%, 1.200 equiv., 0.371 mmol), potassium carbonate (100.00%, 2.000 equiv., 0.618 mmol) and potassium iodide (100.00%, 1.100 equiv., 0.340 mmol) were dissolved in N,N-dimethylformamide (5 mL) at room temperature was stirred overnight at the same temperature. Water was poured into the reaction mixture, and the mixture was extracted with dichloromethane. The organic layer was washed with a saturated aqueous sodium chloride solution, dried over anhydrous magnesium sulfate, filtered, and concentrated under reduced pressure. The concentrate was purified and concentrated by column chromatography (SiO₂, 4 g cartridge; ethyl acetate/hexane=0% to 100%) to obtain the title compound (0.07 g, 45.27%, white solid).

¹H NMR (400 MHz, CDCl₃) δ 9.25 (s, 1H), 8.37 (d, J=10.0 Hz, 1H), 7.69 (t, J=7.6 Hz, 1H), 7.49 (d, J=8.0 Hz, 1H), 7.21-7.18 (m, 2H), 6.93 (t, J=51.6 Hz, 1H), 5.22 (s, 2H), 3.47 (s, 2H), 2.76 (d, J=11.2 Hz, 2H), 1.95 (t, J=10.8 Hz, 2H), 1.57 (d, J=12.0 Hz, 2H), 1.34-1.20 (m, 3H), 0.89 (d, J=6.0 Hz, 3H); LRMS (ES) m/z 501.4 (M⁺+1).

Compounds in Table 5 below were synthesized according to substantially the same method as in Example 4.

TABLE 5

| | Com- | |
|---|---|---|
| Exam- | pound | |
| ple | No. | Compound Name, ¹H-NMR, MS (ESI) |
| 2 | 2 | 3-((5-(5-(difluoromethyl)-1,3,4-oxadiazol-2- |
| | | yl)pyridin-2-yl)methyl)-5-(4-(pyrrolidin-1- |
| | | ylmethyl)phenyl)-1,3,4-oxadiazol-2(3H)-one |
| | | ¹H NMR (400 MHz, CDCl₃) δ 9.25 (d, J = 2.0 Hz, |
| | | 1H), 8.40 (dd, J = 8.0, 2.4 Hz, 1H), 7.78 (d, J = |
| | | 8.0 Hz, 2H), 7.51 (d, J = 8.0 Hz, 1H), 7.43- |
| | | 7.28 (m, 2H), 6.94 (t, J = 51.6 Hz, 1H), 5.18 (s, |
| | | 2H), 3.64 (s, 2H), 2.58-2.51 (m, 4H), 1.92-1.78 |
| | | (m, 4H); LRMS (ES) m/z 455.4 (M⁺ + 1). |

### Example 84: Synthesis of Compound 84, 3-((5-(5-(difluoromethyl)-1,3,4-oxadiazol-2-yl)pyridin-2-yl)methyl)-5-(3-((dimethylamino)methyl)phenyl)-1,3,4-oxadiazol-2(3H)-thione

### [Step 1] Synthesis of methyl 3-(1,3-dioxolan-2-yl)benzoate

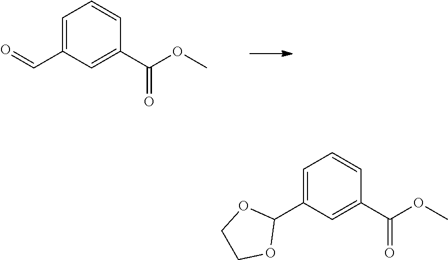

Methyl 3-formylbenzoate (100.00%, 2.000 g, 12.183 mmol), ethylene glycol (100.00%, 3.781 g, 60.920 mmol) and 4-methylbenzenesulfonic acid hydrate (100.00%, 0.232 g, 1.220 mmol) were dissolved in toluene (120 mL) at room temperature, and the resulting mixture was heated to reflux overnight. The mixture was cooled to room temperature, and water was poured into the reaction mixture, followed by extraction with ethyl acetate. The organic layer was washed with a saturated aqueous ammonium chloride solution, dried over anhydrous magnesium sulfate, filtered, and concentrated under reduced pressure. The concentrate was purified and concentrated by column chromatography (SiO₂, 80 g cartridge; ethyl acetate/hexane=0% to 10%) to obtain the title compound (1.8 g, 70.959%, white solid).

### [Step 2] Synthesis of 3-(1,3-dioxolan-2-yl)benzohydrazide

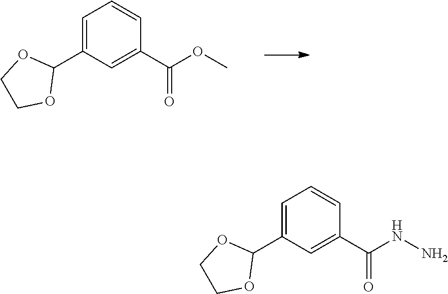

A solution in which methyl 3-(1,3-dioxolan-2-yl)benzoate (100.00%, 1.130 g, 5.427 mmol) synthesized in Step 1 and hydrazine monohydrate (100.00%, 2.717 g, 54.275 mmol) were dissolved in ethanol (80 mL) at 80° C. was stirred overnight at the same temperature, and then the reaction was terminated by lowering the temperature to room temperature. Water was poured into the reaction mixture, and the mixture was extracted with dichloromethane. The organic layer was washed with a saturated aqueous solution of sodium hydrogen carbonate, dried over anhydrous magnesium sulfate, filtered, and concentrated under reduced pressure. The concentrate was purified and concentrated by column chromatography (SiO₂, 24 g cartridge; methanol/dichloromethane=0% to 5%) to obtain the title compound (1.1 g, 97.345%, white solid).

### [Step 3] Synthesis of 5-[3-(1,3-dioxolan-2-yl)phenyl]-3H-1,3,4-oxadiazol-2-thione

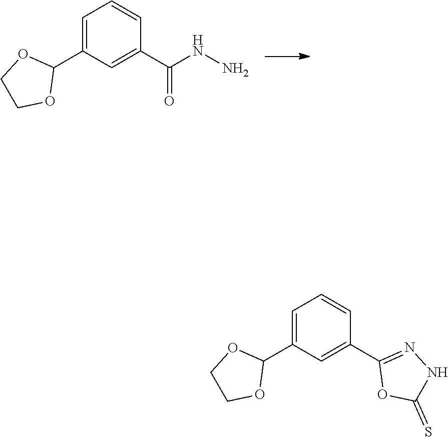 [SMILES: NNC(=O)c1cccc(C2OCCO2)c1 ; S=c1[nH]nc(-c2cccc(C3OCCO3)c2)o1]

A solution in which 3-(1,3-dioxolan-2-yl)benzohydrazide (100.00%, 0.900 g, 4.323 mmol) synthesized in Step 2 and potassium ethylxanthate (100.00%, 0.762 g, 4.754 mmol) were dissolved in ethanol (50 mL) at 80° C. was stirred overnight at the same temperature, and then the reaction was terminated by lowering the temperature to room temperature. Water was poured into the reaction mixture, and the mixture was extracted with dichloromethane. The organic layer was washed with a saturated aqueous sodium chloride solution, dried over anhydrous magnesium sulfate, filtered, and concentrated under reduced pressure. The concentrate was purified and concentrated by column chromatography (SiO₂, 24 g cartridge; ethyl acetate/hexane=0% to 30%) to obtain the title compound (0.7 g, 64.70%, white solid).

### [Step 4] Synthesis of 3-[[5-[5-(difluoromethyl)-1,3,4-oxadiazol-2-yl]-2-pyridyl]methyl]-5-[3-(1,3-dioxolan-2-yl)phenyl]-1,3,4-oxadiazol-2-thione

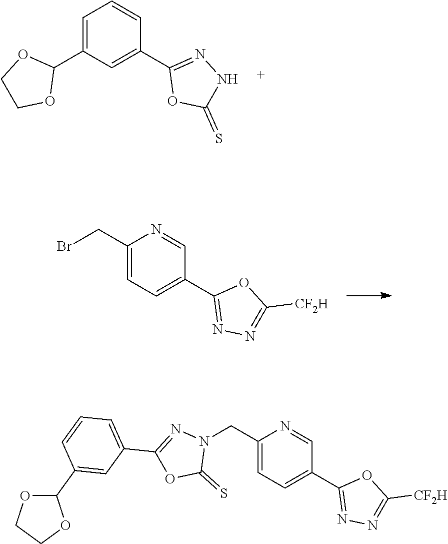 [SMILES: S=c1[nH]nc(-c2cccc(C3OCCO3)c2)o1 ; FC(F)c1nnc(-c2ccc(CBr)nc2)o1 ; FC(F)c1nnc(-c2ccc(Cn3nc(-c4cccc(C5OCCO5)c4)oc3=S)nc2)o1]

A solution in which 5-[3-(1,3-dioxolan-2-yl)phenyl]-3H-1,3,4-oxadiazol-2-thione (100.00%, 0.350 g, 1.398 mmol) synthesized in Step 3, 2-[6-(bromomethyl)-3-pyridyl]-5-(difluoromethyl)-1,3,4-oxadiazole (100.00%, 0.426 g, 1.469 mmol), potassium carbonate (100.00%, 0.208 g, 2.099 mmol), and potassium iodide (100.00%, 0.116 g, 0.699 mmol) were dissolved in N,N-dimethylformamide (30 mL) was stirred at room temperature for 30 minutes, and then further stirred overnight at the same temperature. Water was poured into the reaction mixture, and the mixture was extracted with ethyl acetate. The organic layer was washed with a saturated aqueous ammonium chloride solution, dried over anhydrous magnesium sulfate, filtered, and concentrated under reduced pressure. The concentrate was purified and concentrated by column chromatography (SiO₂, 12 g cartridge; ethyl acetate/hexane=0% to 50%) to obtain the title compound (0.51 g, 79.39%, white solid).

### [Step 5] Synthesis of 3-[4-[[5-[5-(difluoromethyl)-1,3,4-oxadiazol-2-yl]-2-pyridyl]methyl]-5-thioxo-1,3,4-oxadiazol-2-yl]benzaldehyde

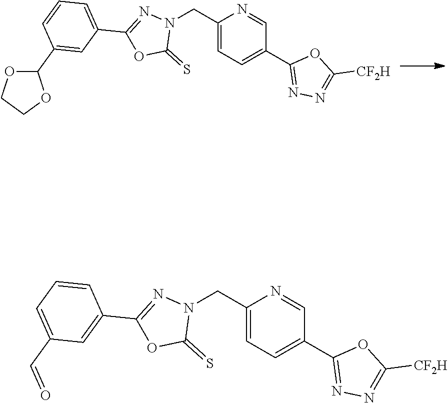 [SMILES: FC(F)c1nnc(-c2ccc(Cn3nc(-c4cccc(C5OCCO5)c4)oc3=S)nc2)o1 ; O=Cc1cccc(-c2nn(Cc3ccc(-c4nnc(C(F)F)o4)cn3)c(=S)o2)c1]

A solution in which 3-[[5-[5-(difluoromethyl)-1,3,4-oxadiazol-2-yl]-2-pyridyl]methyl]-5-[3-(1,3-dioxolan-2-yl)phenyl]-1,3,4-oxadiazol-2-thione (100.00%, 0.500 g, 1.088 mmol) synthesized in Step 4 and ferric chloride hexahydrate (100.00%, 1.030 g, 3.811 mmol) were dissolved in dichloromethane (30 mL) was stirred overnight at the same temperature. Water was poured into the reaction mixture, and the mixture was extracted with dichloromethane. The organic layer was washed with a saturated aqueous sodium chloride solution, dried over anhydrous magnesium sulfate, filtered, and concentrated under reduced pressure. The concentrate was purified and concentrated by column chromatography (SiO₂, 12 g cartridge; ethyl acetate/hexane=0% to 40%) to obtain the title compound (0.350 g, 77.41%) in the form of a white solid.

### [Step 6] Synthesis of Compound 84

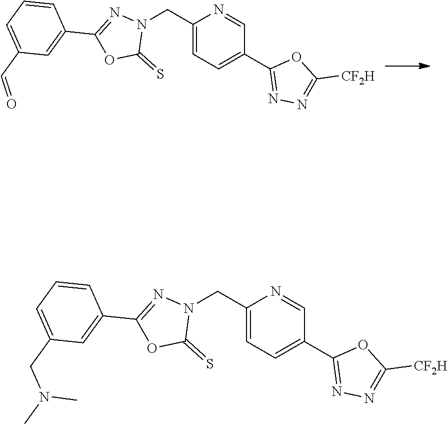 [SMILES: O=Cc1cccc(-c2nn(Cc3ccc(-c4nnc(C(F)F)o4)cn3)c(=S)o2)c1 ; CN(C)Cc1cccc(-c2nn(Cc3ccc(-c4nnc(C(F)F)o4)cn3)c(=S)o2)c1]

To a solution in which 4-[4-[[5-[5-(difluoromethyl)-1,3,4-oxadiazol-2-yl]-2-pyridyl]methyl]-5-thioxo-1,3,4-oxadiazol-2-yl]benzaldehyde (100.00%, 0.030 g, 0.072 mmol) synthesized in Step 5 was dissolved in dichloromethane (5 mL) at room temperature, N-methylmethanamine (100.00%, 0.007 g, 0.155 mmol) was added and stirred at the same temperature for 30 minutes. Water was poured into the reaction mixture, and the mixture was extracted with dichloromethane. The organic layer was washed with a saturated aqueous solution of sodium hydrogen carbonate, dried over anhydrous magnesium sulfate, filtered, and concentrated under reduced pressure. The concentrate was purified and concentrated by column chromatography (SiO₂, 4 g cartridge; methanol/dichloromethane=0% to 5%) to obtain the title compound (0.011 g, 34.27%, white solid).

¹H NMR (400 MHz, CDCl₃) δ 9.32 (d, J=1.7 Hz, 1H), 8.39 (dd, J=8.2, 2.2 Hz, 1H), 7.94-7.90 (m, 2H), 7.83 (d, J=8.2 Hz, 1H), 7.52-7.45 (m, 2H), 7.08 (s, 0.2H), 6.95 (s, 0.5H), 6.82 (s, 0.2H), 4.74 (s, 2H), 3.50 (s, 2H), 2.28 (s, 6H); LRMS (ES) m/z 445.23 (M⁺+1).

Compounds in Table 6 below were synthesized according to substantially the same method as in Example 84.

TABLE 6

| | Compound | |
|---|---|---|
| Example | No. | Compound Name, ¹H-NMR, MS (ESI) |
| 85 | 85 | 3-[[5-[5-(difluoromethyl)-1,3,4-oxadiazol-2-yl]- |
| | | 2-pyridyl]methyl]-5-[3- |
| | | (methylaminomethyl)phenyl]-1,3,4-oxadiazol-2- |
| | | thione |
| | | ¹H NMR (400 MHz, CDCl₃) δ 9.32 (d, J = 2.0 Hz, |
| | | 1H), 8.39 (dd, J = 8.2, 2.2 Hz, 1H), 7.99 (s, |
| | | 1H), 7.92 (d, J = 7.7 Hz, 1H), 7.83 (d, J = 8.2 |
| | | Hz, 1H), 7.57-7.55 (m, 1H), 7.50-7.46 (m, 1H), |
| | | 7.08 (s, 0.2H), 6.95 (s, 0.5H), 6.82 (s, 0.2H), |
| | | 4.74 (s, 2H), 3.88 (s, 2H), 2.50 (s, 3H); LRMS |
| | | (ES) m/z 431.81 (M⁺ + 1). |
| 86 | 86 | 3-[[5-[5-(difluoromethyl)-1,3,4-oxadiazol-2-yl]- |
| | | 2-pyridyl]methyl]-5-[3-(pyrrolidin-1- |
| | | ylmethyl)phenyl]-1,3,4-oxadiazol-2-thione |
| | | ¹H NMR (400 MHz, CDCl₃) δ 9.32 (d, J = 2.0 Hz, |
| | | 1H), 8.39 (dd, J = 8.2, 2.2 Hz, 1H), 7.97 (s, |
| | | 1H), 7.90 (d, J = 8.0 Hz, 1H), 7.83 (d, J = 8.0 |
| | | Hz, 1H), 7.55 (d, J = 8.0 Hz, 1H), 7.48-7.44 (m, |
| | | 1H), 7.08 (s, 0.2H), 6.95 (s, 0.5H), 6.82 (s, |
| | | 0.2H), 4.74 (s, 2H), 3.72 (s, 2H), 2.58 (s, 4H), |
| | | 1.84 (s, 4H); LRMS (ES) m/z 471.79 (M⁺ + 1). |
| 87 | 87 | 3-[[5-[5-(difluoromethyl)-1,3,4-oxadiazol-2-yl]- |
| | | 2-pyridyl]methyl]-5-[3-(1- |
| | | piperidylmethyl)phenyl]-1,3,4-oxadiazol-2-thione |
| | | ¹H NMR (400 MHz, CDCl₃) δ 9.32 (d, J = 1.6 Hz, |
| | | 1H), 8.39 (dd, J = 8.2, 2.2 Hz, 1H), 7.95 (s, |
| | | 1H), 7.89 (d, J = 7.6 Hz, 1H), 7.83 (d, J = 8.0 |
| | | Hz, 1H), 7.53-7.51 (m, 1H), 7.47-7.43 (m, 1H), |
| | | 7.08 (s, 0.2H), 6.95 (s, 0.5H), 6.82 (s, 0.2H), |
| | | 4.74 (s, 2H), 3.54 (s, 2H), 2.41 (brs, 4H), 1.63- |
| | | 1.57 (m, 4H), 1.47-1.32 (m, 2H); LRMS (ES) m/z |
| | | 485.3 (M⁺ + 1). |
| 88 | 88 | 3-[[5-[5-(difluoromethyl)-1,3,4-oxadiazol-2-yl]- |
| | | 2-pyridyl]methyl]-5-[3-[(4-methyl-1- |
| | | piperidyl)methyl]phenyl]-1,3,4-oxadiazol-2- |
| | | thione |
| | | ¹H NMR (400 MHz, CDCl₃) δ 9.32 (d, J = 1.6 Hz, |
| | | 1H), 8.39 (dd, J = 8.2, 2.2 Hz, 1H), 7.95 (s, |
| | | 1H), 7.89 (d, J = 8.0 Hz, 1H), 7.83 (d, J = 8.4 |
| | | Hz, 1H), 7.53-7.51 (m, 1H), 7.47-7.43 (m, 1H), |
| | | 7.08 (s, 0.2H), 6.95 (s, 0.5H), 6.82 (s, 0.2H), |
| | | 4.74 (s, 2H), 3.55 (s, 2H), 2.87-2.84 (m, 2H), |
| | | 2.02-1.96 (m, 2H), 1.67-1.44 (m, 5H), 0.97-0.85 |
| | | (m, 3H); LRMS (ES) m/z 499.9 (M⁺ + 1). |
| 89 | 89 | 3-[[5-[5-(difluoromethyl)-1,3,4-oxadiazol-2-yl]- |
| | | 2-pyridyl]methyl]-5-[3-[[(3R)-3- |
| | | fluoropyrrolidin-1-yl]methyl]phenyl]-1,3,4- |
| | | oxadiazol-2-thione |
| | | ¹H NMR (400 MHz, CDCl₃) δ 9.32 (d, J = 2.0 Hz, |
| | | 1H), 8.39 (dd, J = 8.2, 2.2 Hz, 1H), 7.96 (s, |
| | | 1H), 7.91 (d, J = 8.0 Hz, 1H), 7.83 (d, J = 8.4 |
| | | Hz, 1H), 7.55-7.53 (m, 1H), 7.48-7.44 (m, 1H), |
| | | 7.08 (s, 0.2H), 6.95 (s, 0.5H), 6.82 (s, 0.2H), |
| | | 5.27-5.25 (m, 0.5H), 5.13-5.11 (m, 0.5H), 4.74 |
| | | (s, 2H), 3.73 (s, 2H), 2.92-2.71 (m, 3H), 2.52- |
| | | 2.46 (m, 1H), 2.23-2.04 (m, 2H); LRMS (ES) m/z |
| | | 489.75 (M⁺ + 1). |
| 90 | 90 | 3-[[5-[5-(difluoromethyl)-1,3,4-oxadiazol-2-yl]- |
| | | 2-pyridyl]methyl]-5-[3-[[(3S)-3- |
| | | fluoropyrrolidin-1-yl]methyl]phenyl]-1,3,4- |
| | | oxadiazol-2-thione |
| | | ¹H NMR (400 MHz, CDCl₃) δ 9.32 (d, J = 1.6 Hz, |
| | | 1H), 8.39 (dd, J = 8.2, 2.2 Hz, 1H), 7.96 (s, |
| | | 1H), 7.91 (d, J = 8.0 Hz, 1H), 7.83 (d, J = 8.0 |
| | | Hz, 1H), 7.55-7.53 (m, 1H), 7.48-7.44 (m, 1H), |
| | | 7.08 (s, 0.2H), 6.95 (s, 0.5H), 6.82 (s, 0.2H), |
| | | 5.28-5.25 (m, 0.5H), 5.14-5.11 (m, 0.5H), 4.74 |
| | | (s, 2H), 3.73 (s, 2H), 2.92-2.72 (m, 3H), 2.52- |
| | | 2.46 (m, 1H), 2.23-2.04 (m, 2H); LRMS (ES) m/z |
| | | 489.84 (M⁺ + 1). |
| 91 | 91 | 3-[[5-[5-(difluoromethyl)-1,3,4-oxadiazol-2-yl]- |
| | | 2-pyridyl]methyl]-5-[4- |
| | | [(dimethylamino)methyl]phenyl]-1,3,4-oxadiazol- |
| | | 2-thione |
| | | ¹H NMR (400 MHz, CDCl₃) δ 9.32 (d, J = 1.8 Hz, |
| | | 1H), 8.39 (dd, J = 8.2, 2.0 Hz, 1H), 7.96 (d, J = |
| | | 8.0 Hz, 2H), 7.83 (d, J = 8.2 Hz, 1H), 7.46 (d, |
| | | J = 8.1 Hz, 2H), 7.08 (s, 0.2H), 6.95 (s, 0.5H), |
| | | 6.82 (s, 0.2H), 4.74 (s, 2H), 3.49 (s, 2H), 2.27 |
| | | (s, 6H); LRMS (ES) m/z 445.7 (M⁺ + 1). |
| 92 | 92 | 3-[[5-[5-(difluoromethyl)-1,3,4-oxadiazol-2-yl]- |
| | | 2-pyridyl]methyl]-5-[4- |
| | | (methylaminomethyl)phenyl]-1,3,4-oxadiazol-2- |
| | | thione |
| | | ¹H NMR (400 MHz, CDCl₃) δ 9.32 (d, J = 1.9 Hz, |
| | | 1H), 8.39 (dd, J = 8.2, 2.0 Hz, 1H), 7.97 (d, J = |
| | | 8.2 Hz, 2H), 7.83 (d, J = 8.2 Hz, 1H), 7.47 (d, |
| | | J = 8.1 Hz, 2H), 7.08 (s, 0.2H), 6.95 (s, 0.5H), |
| | | 6.82 (s, 0.2H), 4.74 (s, 2H), 3.84 (s, 2H), 2.49 |
| | | (s, 3H); LRMS (ES) m/z 431.81 (M⁺ + 1). |
| 93 | 93 | 3-[[5-[5-(difluoromethyl)-1,3,4-oxadiazol-2-yl]- |
| | | 2-pyridyl]methyl]-5-[4-(pyrrolidin-1- |
| | | ylmethyl)phenyl]-1,3,4-oxadiazol-2-thione |
| | | ¹H NMR (400 MHz, CDCl₃) δ 9.32 (d, J = 1.6 Hz, |
| | | 1H), 8.39 (dd, J = 8.2, 2.0 Hz, 1H), 7.95 (d, J = |
| | | 8.3 Hz, 2H), 7.83 (d, J = 8.2 Hz, 1H), 7.50 (d, |
| | | J = 8.2 Hz, 2H), 7.08 (s, 0.2H), 6.95 (s, 0.5H), |
| | | 6.82 (s, 0.2H), 4.74 (s, 2H), 3.71 (s, 2H), 2.57 |
| | | (brs, 4H), 1.85-1.81 (m, 4H); LRMS (ES) m/z 471.79 |
| | | (M⁺ + 1). |
| 94 | 94 | 3-[[5-[5-(difluoromethyl)-1,3,4-oxadiazol-2-yl]- |
| | | 2-pyridyl]methyl]-5-[4-(1- |
| | | piperidylmethyl)phenyl]-1,3,4-oxadiazol-2-thione |
| | | ¹H NMR (400 MHz, CDCl₃) δ 9.32 (d, J = 1.8 Hz, |
| | | 1H), 8.39 (dd, J = 8.2, 2.2 Hz, 1H), 7.94 (d, J = |
| | | 8.2 Hz, 2H), 7.83 (d, J = 8.2 Hz, 1H), 7.48 (d, |
| | | J = 8.2 Hz, 2H), 7.08 (s, 0.2H), 6.95 (s, 0.5H), |
| | | 6.82 (s, 0.2H), 4.74 (s, 2H), 3.56 (s, 2H), 2.42 |
| | | (brs, 4H), 1.64-1.60 (m, 4H), 1.48-1.46 (m, 2H); |
| | | LRMS (ES) m/z 485.78 (M⁺ + 1). |
| 95 | 95 | 3-[[5-[5-(difluoromethyl)-1,3,4-oxadiazol-2-yl]- |
| | | 2-pyridyl]methyl]-5-[4- |
| | | (morpholinomethyl)phenyl]-1,3,4-oxadiazol-2- |
| | | thione |
| | | ¹H NMR (400 MHz, CDCl₃) δ 9.31 (d, J = 2.0 Hz, |
| | | 1H), 8.39 (dd, J = 8.2, 2.2 Hz, 1H), 7.95 (d, J = |
| | | 8.2 Hz, 2H), 7.82 (d, J = 8.2 Hz, 1H), 7.49 (d, |
| | | J = 8.1 Hz, 2H), 7.08 (s, 0.2H), 6.95 (s, 0.5H), |
| | | 6.82 (s, 0.2H), 4.73 (s, 2H), 3.75-3.72 (m, 4H), |
| | | 3.57 (s, 2H), 2.48-2.46 (m, 4H); LRMS (ES) m/z |
| | | 487.76 (M⁺ + 1). |
| 96 | 96 | 3-[[5-[5-(difluoromethyl)-1,3,4-oxadiazol-2-yl]- |
| | | 2-pyridyl]methyl]-5-[4-[(4-methyl-1- |
| | | piperidyl)methyl]phenyl]-1,3,4-oxadiazol-2- |
| | | thione |
| | | ¹H NMR (400 MHz, CDCl₃) δ 9.32 (d, J = 1.9 Hz, |
| | | 1H), 8.39 (dd, J = 8.2, 2.2 Hz, 1H), 7.94 (d, J = |
| | | 8.2 Hz, 2H), 7.83 (d, J = 8.2 Hz, 1H), 7.47 (d, |
| | | J = 8.1 Hz, 2H), 7.08 (s, 0.2H), 6.95 (s, 0.5H), |
| | | 6.82 (s, 0.2H), 4.73 (s, 2H), 3.55 (s, 2H), 2.86- |
| | | 2.84 (m, 2H), 2.02-1.97 (m, 2H), 1.63-1.60 (m, |
| | | 2H), 1.39-1.28 (m, 3H), 0.98-0.91 (m, 3H); LRMS |
| | | (ES) m/z 499.86 (M⁺ + 1). |
| 97 | 97 | 3-[[5-[5-(difluoromethyl)-1,3,4-oxadiazol-2-yl]- |
| | | 2-pyridyl]methyl]-5-[4-[[(3R)-3- |
| | | fluoropyrrolidin-1-yl]methyl]phenyl]-1,3,4- |
| | | oxadiazol-2-thione |
| | | ¹H NMR (400 MHz, CDCl₃) δ 9.32 (d, J = 1.9 Hz, |
| | | 1H), 8.39 (dd, J = 8.2, 2.2 Hz, 1H), 7.96 (d, J = |
| | | 8.2 Hz, 2H), 7.83 (d, J = 8.2 Hz, 1H), 7.49 (d, |
| | | J = 8.2 Hz, 2H), 7.08 (s, 0.2H), 6.95 (s, 0.5H), |
| | | 6.82 (s, 0.2H), 5.28-5.25 (0.5H), 5.14-5.11 (m, |
| | | 0.5H), 4.74 (s, 2H), 3.70 (s, 2H), 2.93-2.81 (m, |
| | | 2H), 2.52-2.46 (m, 1H), 2.25-2.03 (m, 3H); LRMS |
| | | (ES) m/z 489.84 (M⁺ + 1). |
| 98 | 98 | 3-[[5-[5-(difluoromethyl)-1,3,4-oxadiazol-2-yl]- |
| | | 2-pyridyl]methyl]-5-[4-[[(3S)-3- |
| | | fluoropyrrolidin-1-yl]methyl]phenyl]-1,3,4- |
| | | oxadiazol-2-thione |
| | | ¹H NMR (400 MHz, CDCl₃) δ 9.32 (d, J = 2.0 Hz, |
| | | 1H), 8.39 (dd, J = 8.2, 2.2 Hz, 1H), 7.96 (d, J = |
| | | 8.2 Hz, 2H), 7.83 (d, J = 8.2 Hz, 1H), 7.49 (d, |
| | | J = 8.2 Hz, 2H), 7.08 (s, 0.2H), 6.95 (s, 0.5H), |
| | | 6.82 (s, 0.2H), 5.28-5.25 (0.5H), 5.14-5.11 (m, |
| | | 0.5H), 4.74 (s, 2H), 3.70 (s, 2H), 2.93-2.81 (m, |
| | | 2H), 2.52-2.48 (m, 1H), 2.25-2.05 (m, 3H); LRMS |
| | | (ES) m/z 489.84 (M⁺ + 1). |
| 99 | 99 | 3-[[5-[5-(difluoromethyl)-1,3,4-oxadiazol-2-yl]- |
| | | 2-pyridyl]methyl]-5-[3- |
| | | (morpholinomethyl)phenyl]-1,3,4-oxadiazol-2- |
| | | thione |
| | | ¹H NMR (400 MHz, CDCl₃) δ 9.32 (d, J = 1.6 Hz, |
| | | 1H), 8.39 (dd, J = 8.2, 2.2 Hz, 1H), 7.98 (s, |
| | | 1H), 7.90 (d, J = 7.6 Hz, 1H), 7.83 (d, J = 8.2 |
| | | Hz, 1H), 7.53-7.44 (m, 2H), 7.08 (s, 0.2H), 6.95 |
| | | (s, 0.5H), 6.82 (s, 0.2H), 4.75 (s, 2H), 3.74- |
| | | 3.72 (m, 4H), 3.57 (s, 2H), 2.48-2.46 (m, 4H); |
| | | LRMS (ES) m/z 487.76 (M⁺ + 1). |
| 107 | 107 | 3-[[5-[5-(difluoromethyl)-1,3,4-oxadiazol-2-yl]- |
| | | 3-fluoro-2-pyridyl]methyl]-5-[4- |
| | | [(dimethylamino)methyl]phenyl]-1,3,4-oxadiazol- |
| | | 2-thione |
| | | ¹H NMR (400 MHz, CDCl₃) δ 9.12 (s, 1H), 8.16 (dd, |
| | | J = 9.0, 1.7 Hz, 1H), 7.98 (d, J = 8.2 Hz, 2H), |
| | | 7.48 (d, J = 8.2 Hz, 2H), 7.09 (s, 0.2H), 6.96 |
| | | (s, 0.5H), 6.83 (s, 0.2H), 4.81 (s, 2H), 3.51 (s, |
| | | 2H), 2.28 (s, 6H); LRMS (ES) m/z 463.9 (M⁺ + 1). |
| 108 | 108 | 3-[[5-[5-(difluoromethyl)-1,3,4-oxadiazol-2-yl]- |
| | | 3-fluoro-2-pyridyl]methyl]-5-[4- |
| | | (methylaminomethyl)phenyl]-1,3,4-oxadiazol-2- |
| | | thione |
| | | ¹H NMR (400 MHz, CDCl₃) δ 9.11 (s, 1H), 8.16 (dd, |
| | | J = 9.0, 1.5 Hz, 1H), 7.99 (d, J = 8.2 Hz, 2H), |
| | | 7.49 (d, J = 8.2 Hz, 2H), 7.09 (s, 0.2H), 6.96 |
| | | (s, 0.5H), 6.83 (s, 0.2H), 4.81 (s, 2H), 3.85 (s, |
| | | 2H), 2.50 (s, 3H); LRMS (ES) m/z 449.8 (M⁺ + 1). |
| 109 | 109 | 5-[4-(diethylaminomethyl)phenyl]-3-[[5-[5- |
| | | (difluoromethyl)-1,3,4-oxadiazol-2-yl]-3-fluoro- |
| | | 2-pyridyl]methyl]-1,3,4-oxadiazol-2-thione |
| | | ¹H NMR (400 MHz, CDCl₃) δ 9.12 (s, 1H), 8.16 (dd, |
| | | J = 9.0, 1.6 Hz, 1H), 7.96 (d, J = 8.2 Hz, 2H), |
| | | 7.51 (d, J = 8.2 Hz, 2H), 7.09 (s, 0.2H), 6.96 |
| | | (s, 0.5H), 6.83 (s, 0.2H), 4.80 (s, 2H), 3.68 (s, |
| | | 2H), 2.59-2.54 (m, 4H), 1.09-1.05 (m, 6H); LRMS |
| | | (ES) m/z 491.9 (M⁺ + 1). |
| 110 | 110 | 3-[[5-[5-(difluoromethyl)-1,3,4-oxadiazol-2-yl]- |
| | | 3-fluoro-2-pyridyl]methyl]-5-[4-[(3- |
| | | fluoroazetidine-1-yl)methyl]phenyl]-1,3,4- |
| | | oxadiazol-2-thione |
| | | ¹H NMR (400 MHz, CDCl₃) δ 9.11 (s, 1H), 8.16 (dd, |
| | | J = 9.0, 1.5 Hz, 1H), 7.98 (d, J = 8.2 Hz, 2H), |
| | | 7.43 (d, J = 8.2 Hz, 2H), 7.09 (s, 0.2H), 6.96 |
| | | (s, 0.5H), 6.83 (s, 0.2H), 5.25-5.23 (m, 0.5H), |
| | | 5.11-5.08 (m, 0.5H), 4.81 (s, 2H), 3.74 (s, 2H), |
| | | 3.72-3.66 (m, 2H), 3.28-3.25 (m, 1H), 3.22-3.19 |
| | | (m, 1H); LRMS (ES) m/z 493.81 (M⁺ + 1). |
| 111 | 111 | 3-[[5-[5-(difluoromethyl)-1,3,4-oxadiazol-2-yl]- |
| | | 3-fluoro-2-pyridyl]methyl]-5-[4-[(4-methyl-1- |
| | | piperidyl)methyl]phenyl]-1,3,4-oxadiazol-2- |
| | | thione |
| | | ¹H NMR (400 MHz, CDCl₃) δ 9.11 (s, 1H), 8.16 (dd, |
| | | J = 9.0, 1.6 Hz, 1H), 7.96 (d, J = 8.2 Hz, 2H), |
| | | 7.48 (d, J = 8.2 Hz, 2H), 7.09 (s, 0.2H), 6.96 |
| | | (s, 0.5H), 6.83 (s, 0.2H), 4.80 (s, 2H), 3.55 (s, |
| | | 2H), 2.86-2.84 (m, 2H), 2.02-1.97 (m, 2H), 1.81- |
| | | 1.60 (m, 2H), 1.39-1.29 (m, 3H), 0.97-0.90 (m, |
| | | 3H); LRMS (ES) m/z 517.82 (M⁺ + 1). |
| 112 | 112 | 3-[[5-[5-(difluoromethyl)-1,3,4-oxadiazol-2-yl]- |
| | | 3-fluoro-2-pyridyl]methyl]-5-[4-(1- |
| | | piperidylmethyl)phenyl]-1,3,4-oxadiazol-2-thione |
| | | ¹H NMR (400 MHz, CDCl₃) δ 9.11 (s, 1H), 8.16 (dd, |
| | | J = 9.0, 1.7 Hz, 1H), 7.97 (d, J = 8.2 Hz, 2H), |
| | | 7.49 (d, J = 8.2 Hz, 2H), 7.09 (s, 0.2H), 6.96 |
| | | (s, 0.5H), 6.83 (s, 0.2H), 4.80 (s, 2H), 3.58 (s, |
| | | 2H), 2.45 (brs, 4H), 1.65-1.59 (m, 4H), 1.48-1.47 |
| | | (m, 2H); LRMS (ES) m/z 503.83 (M⁺ + 1). |
| 113 | 113 | 3-[[5-[5-(difluoromethyl)-1,3,4-oxadiazol-2-yl]- |
| | | 3-fluoro-2-pyridyl]methyl]-5-[4-[[(3R)-3- |
| | | fluoropyrrolidin-1-yl]methyl]phenyl]-1,3,4- |
| | | oxadiazol-2-thione |
| | | ¹H NMR (400 MHz, CDCl₃) δ 9.12 (s, 1H), 8.16 (dd, |
| | | J = 9.0, 1.6 Hz, 1H), 7.98 (d, J = 8.2 Hz, 2H), |
| | | 7.50 (d, J = 8.1 Hz, 2H), 7.09 (s, 0.2H), 6.96 |
| | | (s, 0.5H), 6.83 (s, 0.2H), 5.28-5.25 (m, 0.5H), |
| | | 5.14-5.12 (m, 0.5H), 4.81 (s, 2H), 3.71 (s, 2H), |
| | | 2.94-2.72 (m, 3H), 2.52-2.47 (m, 1H), 2.23-2.02 |
| | | (m, 2H); LRMS (ES) m/z 507.80 (M⁺ + 1). |
| 114 | 114 | 3-[[5-[5-(difluoromethyl)-1,3,4-oxadiazol-2-yl]- |
| | | 3-fluoro-2-pyridyl]methyl]-5-[4-[[(3S)-3- |
| | | fluoropyrrolidin-1-yl]methyl]phenyl]-1,3,4- |
| | | oxadiazol-2-thione |
| | | ¹H NMR (400 MHz, CDCl₃) δ 9.11 (s, 1H), 8.16 (dd, |
| | | J = 9.0, 1.5 Hz, 1H), 7.98 (d, J = 8.2 Hz, 2H), |
| | | 7.50 (d, J = 8.2 Hz, 2H), 7.09 (s, 0.2H), 6.96 |
| | | (s, 0.5H), 6.83 (s, 0.2H), 5.32-5.25 (m, 0.5H), |
| | | 5.14-5.12 (m, 0.5H), 4.81 (s, 2H), 3.78 (s, 2H), |
| | | 2.94-2.72 (m, 3H), 2.53-2.47 (m, 1H), 2.25-2.01 |
| | | (m, 2H); LRMS (ES) m/z 507.80 (M⁺ + 1). |
| 117 | 117 | 3-[[5-[5-(difluoromethyl)-1,3,4-oxadiazol-2-yl]- |
| | | 3-fluoro-2-pyridyl]methyl]-5-[3- |
| | | [(dimethylamino)methyl]phenyl]-1,3,4-oxadiazol- |
| | | 2-thione |
| | | ¹H NMR (400 MHz, CDCl₃) δ 9.13 (s, 1H), 8.16 (dd, |
| | | J = 8.8, 1.6 Hz, 1H), 7.97-7.93 (m, 2H), 7.53- |
| | | 7.46 (m, 2H), 6.96 (t, J = 51.4 Hz, 1H), 4.81 (d, |
| | | J = 1.6 Hz, 2H), 3.51 (s, 2H), 2.29 (s, 6H); LRMS |
| | | (ES) m/z 461.8 (M⁻ − 1). |
| 118 | 118 | 5-[3-(azetidine-1-ylmethyl)phenyl]-3-[[5-[5- |
| | | (difluoromethyl)-1,3,4-oxadiazol-2-yl]-3-fluoro- |
| | | 2-pyridyl]methyl]-1,3,4-oxadiazol-2-thione |
| | | ¹H NMR (400 MHz, CDCl₃) δ 8.16 (dd, J = 9.0, 1.8 |
| | | Hz, 1H), 7.94-7.90 (m, 2H), 7.50-7.44 (m, 2H), |
| | | 6.96 (t, J = 51.4 Hz, 1H), 4.81 (d, J = 2.0 Hz, |
| | | 2H), 3.66 (s, 2H), 3.28 (t, J = 7.0 Hz, 4H), 2.17- |
| | | 2.10 (m, 2H); LRMS (ES) m/z 473.7 (M⁻ − 1). |
| 119 | 119 | 3-[[5-[5-(difluoromethyl)-1,3,4-oxadiazol-2-yl]- |
| | | 3-fluoro-2-pyridyl]methyl]-5-[3-(1- |
| | | piperidylmethyl)phenyl]-1,3,4-oxadiazol-2-thione |
| | | ¹H NMR (400 MHz, CDCl₃) δ 9.13 (s, 1H), 8.16 (dd, |
| | | J = 9.0, 1.8 Hz, 1H), 7.98 (s, 1H), 7.91 (d, J = |
| | | 7.6 Hz, 1H), 7.54-7.53 (m, 1H), 7.48-7.44 (m, |
| | | 1H), 6.96 (t, J = 51.6 Hz, 1H), 4.82 (d, J = 2.0 |
| | | Hz, 2H), 3.55 (s, 2H), 2.42 (s, 4H), 1.63-1.58 |
| | | (m, 4H), 1.47-1.46 (m, 2H); LRMS (ES) m/z 503.9 |
| | | (M⁺ + 1). |
| 120 | 120 | 5-[3-(diethylaminomethyl)phenyl]-3-[[5-[5- |
| | | (difluoromethyl)-1,3,4-oxadiazol-2-yl]-3-fluoro- |
| | | 2-pyridyl]methyl]-1,3,4-oxadiazol-2-thione |
| | | ¹H NMR (400 MHz, CDCl₃) δ 9.13 (d, J = 0.8 Hz, |
| | | 2H), 8.16 (dd, J = 9.0, 1.8 Hz, 1H), 8.00 (s, |
| | | 1H), 7.90 (d, J = 7.6 Hz, 1H), 7.56-7.54 (m, 1H), |
| | | 7.48-7.44 (m, 1H), 6.96 (t, J = 51.6 Hz, 1H), |
| | | 4.81 (d, J = 2.0 Hz, 2H), 3.64 (s, 2H), 2.56 (q, |
| | | J = 7.2 Hz, 4H), 1.07 (t, J = 7.2 Hz, 6H); LRMS |
| | | (ES) m/z 491.9 (M⁺ + 1). |
| 121 | 121 | 3-[[5-[5-(difluoromethyl)-1,3,4-oxadiazol-2-yl]- |
| | | 2-pyridyl]methyl]-5-[5-[(dimethylamino)methyl]- |
| | | 2-thienyl]-1,3,4-oxadiazol-2-thione |
| | | ¹H NMR (400 MHz, CDCl₃) δ 9.32 (d, J = 1.6 Hz, |
| | | 1H), 8.39 (dd, J = 8.2, 2.2 Hz, 1H), 7.81 (d, J = |
| | | 8.0 Hz, 1H), 7.56 (d, J = 3.6 Hz, 1H), 6.97 (s, |
| | | 1H), 6.96 (t, J = 51.6 Hz, 1H), 4.72 (s, 2H), |
| | | 3.69 (s, 2H), 2.32 (s, 6H); LRMS (ES) m/z 451.7 |
| | | (M⁺ + 1). |
| 122 | 122 | 5-[5-(diethylaminomethyl)-2-thienyl]-3-[[5-[5- |
| | | (difluoromethyl)-1,3,4-oxadiazol-2-yl]-2- |
| | | pyridyl]methyl]-1,3,4-oxadiazol-2-thione |
| | | ¹H NMR (400 MHz, CDCl₃) δ 9.32 (d, J = 1.6 Hz, |
| | | 1H), 8.39 (dd, J = 8.2, 2.2 Hz, 1H), 7.81 (d, J = |
| | | 8.4 Hz, 1H), 7.55 (d, J = 4.0 Hz, 1H), 6.96 (t, |
| | | J = 51.6 Hz, 1H), 6.94 (d, J = 3.6 Hz, 1H), 4.71 |
| | | (s, 2H), 3.83 (s, 2H), 2.59 (q, J = 7.1 Hz, 4H), |
| | | 1.09 (t, J = 7.0 Hz, 6H); LRMS (ES) m/z 479.4 |
| | | (M⁺ + 1). |
| 123 | 123 | 5-[5-(azetidine-1-ylmethyl)-2-thienyl]-3-[[5-[5- |
| | | (difluoromethyl)-1,3,4-oxadiazol-2-yl]-2- |
| | | pyridyl]methyl]-1,3,4-oxadiazol-2-thione |
| | | ¹H NMR (400 MHz, CDCl₃) δ 9.32 (d, J = 1.6 Hz, |
| | | 1H), 8.38 (dd, J = 8.2, 2.2 Hz, 1H), 7.81 (d, J = |
| | | 8.0 Hz, 1H), 7.55 (d, J = 3.6 Hz, 1H), 6.96 (t, |
| | | J = 51.6 Hz, 1H), 6.94 (d, J = 4.0 Hz, 1H), 4.71 |
| | | (s, 2H), 3.79 (s, 2H), 3.31 (t, J = 7.0 Hz, 4H), |
| | | 2.17-2.10 (m, 2H); LRMS (ES) m/z 463.7 (M⁺ + 1). |
| 124 | 124 | 3-[[5-[5-(difluoromethyl)-1,3,4-oxadiazol-2-yl]- |
| | | 2-pyridyl]methyl]-5-[5-[[(3R)-3- |
| | | fluoropyrrolidin-1-yl]methyl]-2-thienyl]-1,3,4- |
| | | oxadiazol-2-thione |
| | | ¹H NMR (400 MHz, CDCl₃) δ 9.32-9.31 (m, 1H), 8.39 |
| | | (dd, J = 8.2, 2.2 Hz, 1H), 7.81 (d, J = 8.0 Hz, |
| | | 1H), 7.55 (d, J = 3.6 Hz, 1H), 6.98 (d, J = 4.0 |
| | | Hz, 1H), 6.96 (t, J = 51.6 Hz, 1H), 5.29-5.11 (m, |
| | | 1H), 4.72 (s, 2H), 3.92 (s, 2H), 2.95-2.87 (m, |
| | | 3H), 2.65-2.60 (m, 1H), 2.26-2.03 (m, 2H); LRMS |
| | | (ES) m/z 495.7 (M⁺ + 1). |
| 125 | 125 | 3-[[5-[5-(difluoromethyl)-1,3,4-oxadiazol-2-yl]- |
| | | 2-pyridyl]methyl]-5-[5-[[(3S)-3- |
| | | fluoropyrrolidin-1-yl]methyl]-2-thienyl]-1,3,4- |
| | | oxadiazol-2-thione |
| | | ¹H NMR (400 MHz, CDCl₃) δ 9.32 (d, J = 2.0 Hz, |
| | | 1H), 8.39 (dd, J = 8.2, 2.2 Hz, 1H), 7.81 (d, J = |
| | | 8.4 Hz, 1H), 7.55 (d, J = 3.6 Hz, 1H), 6.98 (d, |
| | | J = 3.6 Hz, 1H), 6.96 (t, J = 51.6 Hz, 1H), 5.28- |
| | | 5.12 (m, 1H), 4.72 (s, 2H), 3.93 (s, 2H), 2.95- |
| | | 2.87 (m, 3H), 2.65-2.60 (m, 1H), 2.26-2.03 (m, |
| | | 2H); LRMS (ES) m/z 495.3 (M⁺ + 1). |
| 128 | 128 | 3-[[5-[5-(difluoromethyl)-1,3,4-oxadiazol-2-yl]- |
| | | 3-fluoro-2-pyridyl]methyl]-5-[5- |
| | | [(dimethylamino)methyl]-2-thienyl]-1,3,4- |
| | | oxadiazol-2-thione |
| | | ¹H NMR (400 MHz, CDCl₃) δ 9.12 (s, 1H), 8.15 (dd, |
| | | J = 8.9, 1.6 Hz, 1H), 7.59 (d, J = 3.7 Hz, 1H), |
| | | 7.09 (s, 0.2H), 7.01-6.99 (m, 1H), 6.96 (s, 0.5H), |
| | | 6.83 (s, 0.2H), 4.78 (s, 2H), 3.72 (s, 2H), 2.34 |
| | | (s, 6H); LRMS (ES) m/z 469.71 (M⁺ + 1). |
| 129 | 129 | 5-[5-(diethylaminomethyl)-2-thienyl]-3-[[5-[5- |
| | | (difluoromethyl)-1,3,4-oxadiazol-2-yl]-3-fluoro- |
| | | 2-pyridyl]methyl]-1,3,4-oxadiazol-2-thione |
| | | ¹H NMR (400 MHz, CDCl₃) δ 9.11 (s, 1H), 8.15 (dd, |
| | | J = 9.0, 1.6 Hz, 1H), 7.59 (d, J = 3.7 Hz, 1H), |
| | | 7.09 (s, 0.2H), 7.01-6.99 (m, 1H), 6.96 (s, 0.5H), |
| | | 6.83 (s, 0.2H), 4.77 (s, 2H), 3.89 (s, 2H), 2.65- |
| | | 2.63 (m, 4H), 1.14-1.11 (m, 6H); LRMS (ES) m/z |
| | | 497.69 (M⁺ + 1). |
| 130 | 130 | 5-[5-(azetidine-1-ylmethyl)-2-thienyl]-3-[[5-[5- |
| | | (difluoromethyl)-1,3,4-oxadiazol-2-yl]-3-fluoro- |
| | | 2-pyridyl]methyl]-1,3,4-oxadiazol-2-thione |
| | | ¹H NMR (400 MHz, CDCl₃) δ 9.11 (s, 1H), 8.15 (dd, |
| | | J = 9.0, 1.7 Hz, 1H), 7.62-7.57 (m, 1H), 7.09 (s, |
| | | 0.3H), 7.01-6.97 (m, 1H), 6.96 (s, 0.5H), 6.83 |
| | | (s, 0.2H), 4.77 (s, 2H), 3.82 (s, 2H), 3.36-3.33 |
| | | (m, 4H), 2.19-2.12 (m, 2H); LRMS (ES) m/z 481.71 |
| | | (M⁺ + 1). |
| 131 | 131 | 3-[[5-[5-(difluoromethyl)-1,3,4-oxadiazol-2-yl]- |
| | | 2-pyridyl]methyl]-5-[5-[(dimethylamino)methyl]- |
| | | 3-thienyl]-1,3,4-oxadiazol-2-thione |
| | | ¹H NMR (400 MHz, CDCl₃) δ 9.32 (d, J = 1.6 Hz, |
| | | 1H), 8.39 (dd, J = 8.2, 2.2 Hz, 1H), 7.89 (d, J = |
| | | 1.2 Hz, 1H), 7.81 (d, J = 8.0 Hz, 1H), 7.41 (s, |
| | | 1H), 6.96 (t, J = 51.6 Hz, 1H), 4.73 (s, 2H), |
| | | 3.67 (s, 2H), 2.31 (s, 6H); LRMS (ES) m/z 451.6 |
| | | (M⁺ + 1). |
| 134 | 134 | 5-[5-(azetidine-1-ylmethyl)-3-thienyl]-3-[[5-[5- |
| | | (difluoromethyl)-1,3,4-oxadiazol-2-yl]-2- |
| | | pyridyl]methyl]-1,3,4-oxadiazol-2-thione |
| | | ¹H NMR (400 MHz, CDCl₃) δ 9.31 (d, J = 2.0 Hz, |
| | | 1H), 8.39 (dd, J = 8.0, 2.0 Hz, 1H), 7.86 (s, |
| | | 1H), 7.81 (d, J = 8.0 Hz, 1H), 7.39 (s, 1H), 6.96 |
| | | (t, J = 51.6 Hz, 1H), 4.72 (s, 2H), 3.77 (s, 2H), |
| | | 3.29 (t, J = 7.0 Hz, 4H), 2.17-2.10 (m, 2H); LRMS |
| | | (ES) m/z 463.7 (M⁺ + 1). |

### Example 132: Synthesis of Compound 132, 5-[4-(azetidin-1-ylmethyl)phenyl]-3-[[5-[5-(difluoromethyl)-1,3,4-oxadiazol-2-yl]-2-pyridyl]methyl]-1,3,4-oxadiazol-2-one

### [Step 1] Synthesis of methyl 4-(1,3-dioxolan-1-yl)benzoate

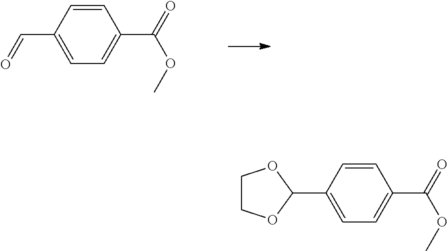

Methyl 4-formylbenzoate (100.00%, 3,000 g, 18.275 mmol), ethylene glycol (100.00%, 5.672 g, 91.380 mmol) and 4-methylbenzenesulfonic acid hydrate (100.00%, 0.348 g, 1.830 mmol) were dissolved in toluene (150 mL) at room temperature, and the resulting mixture was heated to reflux overnight. The mixture was cooled to room temperature, and water was poured into the reaction mixture, followed by extraction with ethyl acetate. The organic layer was washed with a saturated aqueous sodium chloride solution, dried over anhydrous magnesium sulfate, filtered, and concentrated under reduced pressure. The concentrate was purified and concentrated by column chromatography (SiO₂, 80 g cartridge; ethyl acetate/hexane=0% to 10%) to obtain the title compound (3.5 g, 91.984%, white solid).

### [Step 2] Synthesis of 4-(1,3-dioxolan-2-yl)benzohydrazide

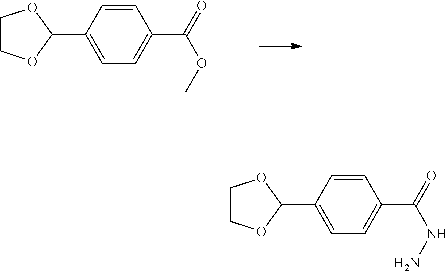

Methyl 4-(1,3-dioxolan-2-yl)benzoate (100.00%, 2.000 g, 9.606 mmol) synthesized in Step 1 and hydrazine monohydrate (100.00%, 4.809 g, 96.065 mmol) were dissolved in ethanol (100 mL) at room temperature, and the resulting mixture was heated to reflux overnight. The mixture was cooled to room temperature, and water was poured into the reaction mixture, followed by extraction with dichloromethane. The organic layer was washed with a saturated aqueous solution of sodium hydrogen carbonate, dried over anhydrous magnesium sulfate, filtered, and concentrated under reduced pressure. The concentrate was purified and concentrated by column chromatography (SiO₂, 40 g cartridge; methanol/dichloromethane=0% to 5%) to obtain the title compound (1.8 g, 90,000%, white solid).

### [Step 3] Synthesis of 5-[4-(1,3-dioxolan-2-yl)phenyl]-3H-1,3,4-oxadiazol-2-one

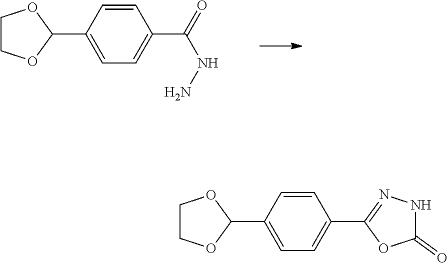 [SMILES: NNC(=O)c1ccc(C2OCCO2)cc1 ; O=c1[nH]nc(-c2ccc(C3OCCO3)cc2)o1]

4-(1,3-Dioxolan-2-yl)benzohydrazide (100.00%, 0.650 g, 3.122 mmol) synthesized in Step 2 and 1,1′-carbonylbis-1H-imidazole (100.00%, 0.607 g, 3.744 mmol) were dissolved in dichloromethane (50 mL) at room temperature, and the resulting mixture was heated to reflux overnight. The mixture was cooled to room temperature, and water was poured into the reaction mixture, followed by extraction with dichloromethane. The organic layer was washed with a saturated aqueous sodium chloride solution, dried over anhydrous magnesium sulfate, filtered, and concentrated under reduced pressure. The concentrate was purified and concentrated by column chromatography (SiO₂, 12 g cartridge; ethyl acetate/hexane=0% to 50%) to obtain the title compound (0.45 g, 61.545%, white solid).

### [Step 4] Synthesis of 3-[[5-[5-(difluoromethyl)-1,3,4-oxadiazol-2-yl]-2-pyridyl]methyl]-5-[4-(1,3-dioxolan-2-yl)phenyl]-1,3,4-oxadiazol-2-one

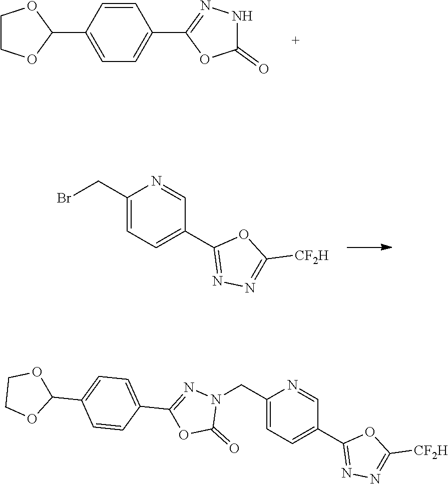 [SMILES: O=c1[nH]nc(-c2ccc(C3OCCO3)cc2)o1 ; FC(F)c1nnc(-c2ccc(CBr)nc2)o1 ; O=c1oc(-c2ccc(C3OCCO3)cc2)nn1Cc1ccc(-c2nnc(C(F)F)o2)cn1]

To a solution in which 5-[4-(1,3-dioxolan-2-yl)phenyl]-3H-1,3,4-oxadiazol-2-one (100.00%, 0.500 g, 2.135 mmol) synthesized in Step 3 and potassium carbonate (100.00%, 0.443 g, 3.205 mmol) were dissolved in N,N-dimethylformamide (30 mL) at room temperature, 2-[6-(bromomethyl)-3-pyridyl]-5-(difluoromethyl)-1,3,4-oxadiazole (100.00%, 0.650 g, 2.241 mmol) and potassium iodide (100.00%, 0.177 g, 1.066 mmol) were added and stirred at the same temperature for 30 minutes. Water was poured into the reaction mixture, and the mixture was extracted with ethyl acetate. The organic layer was washed with a saturated aqueous ammonium chloride solution, dried over anhydrous magnesium sulfate, filtered, and concentrated under reduced pressure. To the concentrate, ethyl acetate (5 mL) was added and stirred to precipitate a solid, and the precipitated solid was filtered, washed with hexane, and dried to obtain the title compound (0.74 g, 78.18%, white solid).

### [Step 5] Synthesis of 4-[4-[[5-[5-(difluoromethyl)-1,3,4-oxadiazol-2-yl]-2-pyridyl]methyl]-5-oxo-1,3,4-oxadiazol-2-yl]benzaldehyde

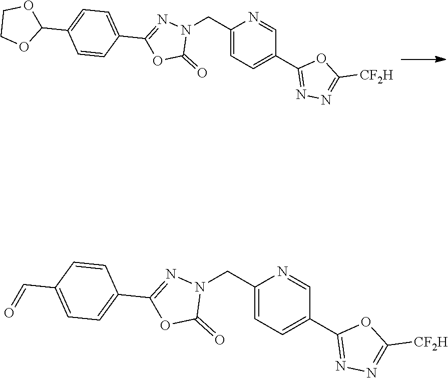 [SMILES: O=c1oc(-c2ccc(C3OCCO3)cc2)nn1Cc1ccc(-c2nnc(C(F)F)o2)cn1 ; O=Cc1ccc(-c2nn(Cc3ccc(-c4nnc(C(F)F)o4)cn3)c(=O)o2)cc1]

A solution in which 3-[[5-[5-(difluoromethyl)-1,3,4-oxadiazol-2-yl]-2-pyridyl]methyl]-5-[4-(1,3-dioxolan-2-yl)phenyl]-1,3,4-oxadiazol-2-one (100.00%, 0.280 g, 0.632 mmol) synthesized in Step 4 and ferric chloride hexahydrate (100.00%, 0.512 g, 1.894 mmol) were dissolved in dichloromethane (30 mL) was stirred overnight at the same temperature. Water was poured into the reaction mixture, and the mixture was extracted with dichloromethane. The organic layer was washed with a saturated aqueous sodium chloride solution, dried over anhydrous magnesium sulfate, filtered, and concentrated under reduced pressure. The concentrate was purified and concentrated by column chromatography (SiO₂, 12 g cartridge; ethyl acetate/hexane=0% to 50%) to obtain the title compound (0.18 g, 71.39%, white solid).

### [Step 6] Synthesis of Compound 132

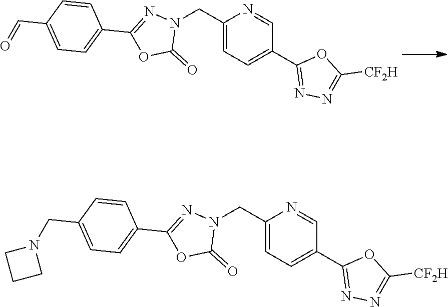 [SMILES: O=Cc1ccc(-c2nn(Cc3ccc(-c4nnc(C(F)F)o4)cn3)c(=O)o2)cc1 ; O=c1oc(-c2ccc(CN3CCC3)cc2)nn1Cc1ccc(-c2nnc(C(F)F)o2)cn1]

A solution in which 4-[4-[[5-[5-(difluoromethyl)-1,3,4-oxadiazol-2-yl]-2-pyridyl]methyl]-5-oxo-1,3,4-oxadiazol-2-yl]benzaldehyde (100.00%, 0.030 g, 0.075 mmol) synthesized in Step 5 and azetidine hydrochloride (100.00%, 0.014 g, 0.150 mmol) were dissolved in dichloromethane (5 mL) was stirred at room temperature for 30 minutes. Then, sodium triacetoxyborohydride (100.00%, 0.048 g, 0.227 mmol) was added and further stirred overnight at the same temperature. Water was poured into the reaction mixture, and the mixture was extracted with dichloromethane. The organic layer was washed with a saturated aqueous solution of sodium hydrogen carbonate, dried over anhydrous magnesium sulfate, filtered, and concentrated under reduced pressure. The concentrate was purified and concentrated by column chromatography (SiO₂, 4 g cartridge; methanol/dichloromethane=0% to 5%) to obtain the title compound (0.019 g, 57.42%, white solid).

¹H NMR (400 MHz, CDCl₃) δ 9.33 (d, J=1.5 Hz, 1H), 8.43 (dd, J=8.2, 2.2 Hz, 1H), 7.82 (d, J=8.3 Hz, 2H), 7.55-7.52 (m, 1H), 7.43 (d, J=8.1 Hz, 2H), 7.08 (s, 0.2H), 6.95 (s, 0.5H), 6.82 (s, 0.2H), 5.24 (s, 2H), 3.67 (s, 2H), 3.32-3.28 (m, 4H), 2.19-2.12 (in, 2H); LRMS (ES) m/z 441.3 (M⁺+1).

Compounds in Table 7 below were synthesized according to substantially the same method as in Example 132.

TABLE 7

| | Com- | |
|---|---|---|
| Exam- | pound | |
| ple | No. | Compound Name, ¹H-NMR, MS (ESI) |
| 133 | 133 | 3-[[5-[5-(difluoromethyl)-1,3,4-oxadiazol-2-yl]- |
| | | 2-pyridyl]methyl]-5-[4-(1- |
| | | piperidylmethyl)phenyl]-1,3,4-oxadiazol-2-one |
| | | ¹H NMR (400 MHz, CDCl₃) δ 9.32 (s, 1H), 8.43 (dd, |
| | | J = 8.2, 2.0 Hz, 1H), 7.82 (d, J = 8.1 Hz, 2H), |
| | | 7.53-7.48 (m, 3H), 7.08 (s, 0.2H), 6.95 (s, 0.5H), |
| | | 6.82 (s, 0.2H), 5.23 (s, 2H), 3.58 (s, 2H), 2.44 |
| | | (brs, 4H), 1.63 (brs, 4H), 1.47 (brs, 2H); LRMS |
| | | (ES) m/z 469.3 (M⁺ + 1). |

### Example 83: Synthesis of Compound 83, 3-[[5-[5-(difluoromethyl)-1,3,4-oxadiazol-2-yl]-2-pyridyl]methyl]-5-[4-[(4-isopropylpiperazin-1-yl)methyl]phenyl]-1,3,4-oxadiazol-2-one

### [Step 1] Synthesis of tert-butyl 4-[(4-methoxycarbonylphenyl)methyl]piperazin-1-carboxylate

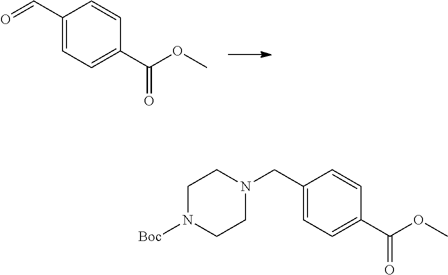 [SMILES: COC(=O)c1ccc(C=O)cc1 ; COC(=O)c1ccc(CN2CCN(C(=O)OC(C)(C)C)CC2)cc1]

A solution in which methyl 4-formylbenzoate (100.00%, 0.500 g, 3.046 mmol), tert-butyl piperazin-1l-carboxylate (100.00%, 0.681 g, 3.656 mmol) and sodium triacetoxyborohydride (100.00%, 1.291 g, 6.091 mmol) were dissolved in dichloromethane (20 mL) at room temperature was stirred overnight at the same temperature. Water was poured into the reaction mixture, and the mixture was extracted with dichloromethane. The organic layer was washed with a saturated aqueous solution of sodium hydrogen carbonate, dried over anhydrous magnesium sulfate, filtered, and concentrated under reduced pressure. The concentrate was purified and concentrated by column chromatography (SiO₂, 24 g cartridge; ethyl acetate/hexane=0% to 30%) to obtain the title compound (0.89 g, 87.38%, white solid).

### [Step 2] Synthesis of tert-butyl 4-[[4-(hydrazinecarbonyl)phenyl]methyl]piperazin-1-carboxylate

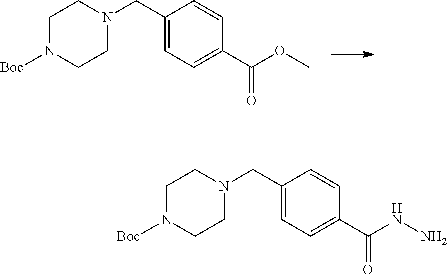 [SMILES: COC(=O)c1ccc(CN2CCN(C(=O)OC(C)(C)C)CC2)cc1 ; CC(C)(C)OC(=O)N1CCN(Cc2ccc(C(=O)NN)cc2)CC1]

A solution in which tert-butyl 4-[(4-methoxycarbonylphenyl)methyl]piperazin-1-carboxylate (100.00%, 0.300 g, 0.897 mmol) synthesized in Step 1 and hydrazine monohydrate (100.00%, 0.449 g, 8.969 mmol) were dissolved in ethanol (20 mL) at 90° C. was stirred overnight at the same temperature, and then the reaction was terminated by lowering the temperature to room temperature. Water was poured into the reaction mixture, and the mixture was extracted with dichloromethane. The organic layer was washed with a saturated aqueous solution of sodium hydrogen carbonate, dried over anhydrous magnesium sulfate, filtered, and concentrated under reduced pressure. The obtained product was used without further purification (0.29 g, 96.67%, white solid).

### [Step 3] Synthesis of tert-butyl 4-[[4-(2-oxo-3H-1,3,4-oxadiazol-5-yl)phenyl]methyl]piperazin-1-carboxylate

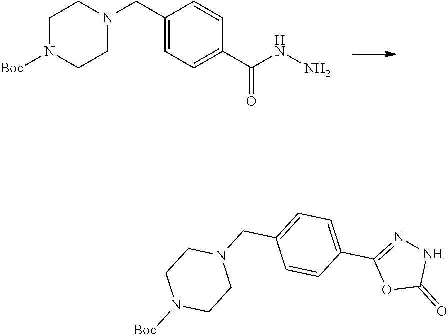 [SMILES: CC(C)(C)OC(=O)N1CCN(Cc2ccc(C(=O)NN)cc2)CC1 ; CC(C)(C)OC(=O)N1CCN(Cc2ccc(-c3n[nH]c(=O)o3)cc2)CC1]

To a solution in which tert-butyl 4-[[4-(hydrazinecarbonyl)phenyl]methyl]piperazin-1-carboxylate (100.00%, 0.500 g, 1.495 mmol) synthesized in Step 2 and 1,1′-carbonylbis-1H-imidazole (100.00%, 0.291 g, 1.795 mmol) were dissolved in tetrahydrofuran (10 mL) at room temperature, triethylamine (100.00% solution, 0.29 mL, 2.100 mmol) was added and stirred overnight at the same temperature. Water was poured into the reaction mixture, and the mixture was extracted with dichloromethane. The organic layer was washed with a saturated aqueous sodium chloride solution, dried over anhydrous magnesium sulfate, filtered, and concentrated under reduced pressure. The concentrate was purified and concentrated by column chromatography (SiO₂, 24 g cartridge; methanol/dichloromethane=0% to 10%) to obtain the title compound (0.468 g, 86.85%, white solid).

### [Step 4] Synthesis of tert-butyl 4-[[4-[4-[[[5-(difluoromethyl)-1,3,4-oxadiazol-2-yl]-2-pyridyl]methyl]-5-oxo-1,3, 4-oxadiazol-2-yl]phenyl]methyl]piperazin-1-carboxylate

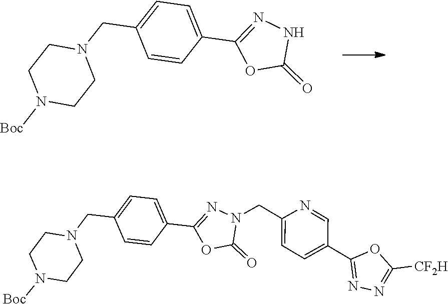 [SMILES: CC(C)(C)OC(=O)N1CCN(Cc2ccc(-c3n[nH]c(=O)o3)cc2)CC1 ; CC(C)(C)OC(=O)N1CCN(Cc2ccc(-c3nn(Cc4ccc(-c5nnc(C(F)F)o5)cn4)c(=O)o3)cc2)CC1]

To a solution in which tert-butyl 4-[[4-(2-oxo-3H-1,3,4-oxadiazol-5-yl)phenyl]methyl]piperazin-1-carboxylate (100.00%, 0.236 g, 0.655 mmol) synthesized in Step 3 and potassium carbonate (100.00%, 0.091 g, 0.918 mmol) were dissolved in N,N-dimethylformamide (3 mL) at room temperature, 2-[6-(bromomethyl)-3-pyridyl]-5-(difluoromethyl)-1,3,4-oxadiazole (100.00%, 0.228 g, 0.786 mmol) prepared in Step 3 of Example 5 was added and stirred overnight at the same temperature. Water was poured into the reaction mixture, and the mixture was extracted with ethyl acetate. The organic layer was washed with a saturated aqueous sodium chloride solution, dried over anhydrous magnesium sulfate, filtered, and concentrated under reduced pressure. The concentrate was purified and concentrated by column chromatography (SiO₂, 4 g cartridge; ethyl acetate/hexane=0% to 60%), and then the obtained product was purified and concentrated again by chromatography (SiO₂, 4 g cartridge; methanol/dichloromethane=0% to 10%) to obtain the title compound (0.37 g, 99.20%, white solid).

### [Step 5] Synthesis of 3-[[5-[5-(difluoromethyl)-1,3,4-oxadiazol-2-yl]-2-pyridyl]methyl]-5-[4-(piperazin-1-ylmethyl)phenyl]-1,3,4-oxadiazol-2-one

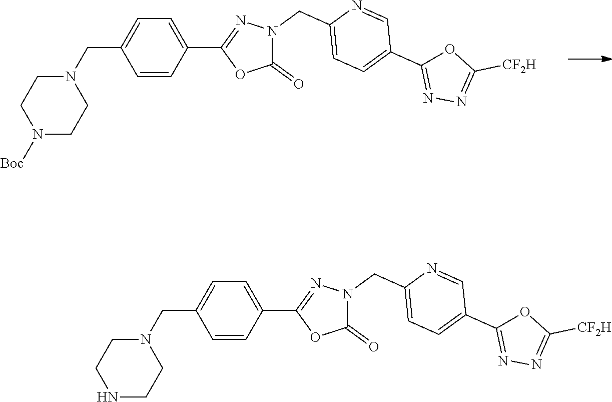 [SMILES: CC(C)(C)OC(=O)N1CCN(Cc2ccc(-c3nn(Cc4ccc(-c5nnc(C(F)F)o5)cn4)c(=O)o3)cc2)CC1 ; O=c1oc(-c2ccc(CN3CCNCC3)cc2)nn1Cc1ccc(-c2nnc(C(F)F)o2)cn1]

A solution in which tert-butyl 4-[[4-[4-[[5-[5-(difluoromethyl)-1,3,4-oxadiazol-2-yl]-2-pyridyl]methyl]-5-oxo-1,3,4-oxadiazol-2-yl]phenyl]methyl]piperazin-1-carboxylate (100.00%, 0.370 g, 0.650 mmol) synthesized in Step 4 and trifluoroacetic acid (100.00% solution, 0.497 mL, 6.490 mmol) were dissolved in dichloromethane (3 mL) at room temperature was stirred overnight at the same temperature. A saturated aqueous solution of sodium hydrogen carbonate was poured into the reaction mixture, followed by extraction with dichloromethane. The organic layer was washed with a saturated aqueous sodium chloride solution, dried over anhydrous magnesium sulfate, filtered, and concentrated under reduced pressure. The obtained product was used without further purification (0.3 g, 98.39%, yellow oil).

### [Step 6] Synthesis of Compound 83

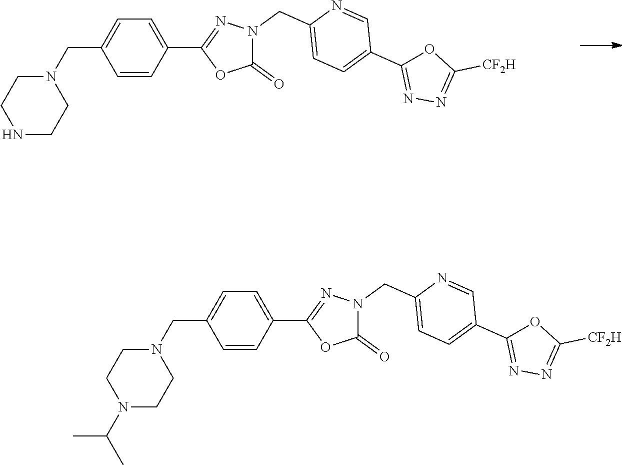 [SMILES: O=c1oc(-c2ccc(CN3CCNCC3)cc2)nn1Cc1ccc(-c2nnc(C(F)F)o2)cn1 ; CC(C)N1CCN(Cc2ccc(-c3nn(Cc4ccc(-c5nnc(C(F)F)o5)cn4)c(=O)o3)cc2)CC1]

A solution in which 3-[[5-[5-(difluoromethyl)-1,3,4-oxadiazol-2-yl]-2-pyridyl]methyl]-5-[4-(piperazin-1-yl methyl)phenyl]-1,3,4-oxadiazol-2-one (100.00%, 0.075 g, 0.160 mmol) synthesized in Step 5, acetone (100.00% solution, 0.019 mL, 0.257 mmol) and N-ethyl-N-isopropyl-propan-2-amine (100.00% solution, 0.056 mL, 0.322 mmol) were dissolved in dichloromethane (1 mL) was stirred at room temperature for 30 minutes. Then, sodium triacetoxyborohydride (100.00%, 0.101 g, 0.479 mmol) was added and further stirred at the same temperature for 18 hours. A saturated aqueous solution of sodium hydrogen carbonate was poured into the reaction mixture, followed by extraction with dichloromethane. The organic layer was washed with a saturated aqueous sodium chloride solution, dried over anhydrous magnesium sulfate, filtered, and concentrated under reduced pressure. The concentrate was purified and concentrated by column chromatography (SiO₂, 4 g cartridge; dichloromethane/methanol=0% to 10%) to obtain the title compound (0.014 g, 17.13%) as a pale yellow solid.

¹H NMR (400 MHz, CD₃OD) δ 9.25 (d, J=1.6 Hz, 1H), 8.53 (dd, J=8.2, 2.2 Hz, 1H), 7.85 (d, J=8.4 Hz, 2H), 7.72 (d, J=8.0 Hz, 1H), 7.53 (d, J=8.4 Hz, 2H), 7.26 (t, J=51.6 Hz, 1H), 5.26 (s, 2H), 3.63 (s, 2H), 2.74-2.59 (m, 9H), 1.15 (d, J=6.8 Hz, 6H); LRMS (ES) m/z 512.8 (M⁺+1).

### Example 66: Synthesis of Compound 66, 3-[[5-[5-(difluoromethyl)-1,3,4-oxadiazol-2-yl]-2-pyridyl]methyl]-5-[4-(4-isopropyl piperazin-1-yl)phenyl]-1,3,4-thiadiazol-2-one

### [Step 1] Synthesis of tert-butyl 4-[[4-(aminocarbonothioyl)phenyl]methyl]piperazin-1-carboxylate

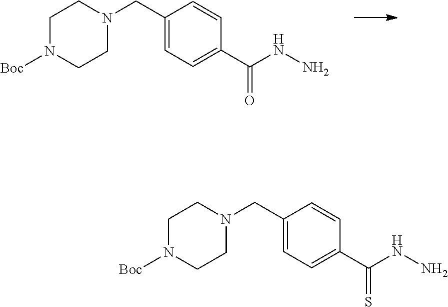 [SMILES: CC(C)(C)OC(=O)N1CCN(Cc2ccc(C(=O)NN)cc2)CC1 ; CC(C)(C)OC(=O)N1CCN(Cc2ccc(C(=S)NN)cc2)CC1]

A solution in which tert-butyl 4-[[4-(hydrazinecarbonyl)phenyl]methyl]piperazine-1-carboxylate (100.00%, 1.000 g, 2.990 mmol) synthesized in Step 2 of Example 86 and Lawesson's reagent (100.00%, 1.451 g, 3.587 mmol) were dissolved in toluene (40 mL) at 80° C. was stirred overnight at the same temperature. Then, the reaction was terminated by lowering the temperature to room temperature. Water was poured into the reaction mixture, and the mixture was extracted with ethyl acetate. The organic layer was washed with a saturated aqueous sodium chloride solution, dried over anhydrous magnesium sulfate, filtered, and concentrated under reduced pressure. The concentrate was purified and concentrated by column chromatography (SiO₂, 24 g cartridge; ethyl acetate/hexane=0% to 60%) to obtain the title compound (0.61 g, 58.20%, white solid).

### [Step 2] Synthesis of tert-butyl 4-[[4-(2-oxo-3H-1,3,4-thiadiazol-5-yl)phenyl]methyl]piperazin-1-carboxylate

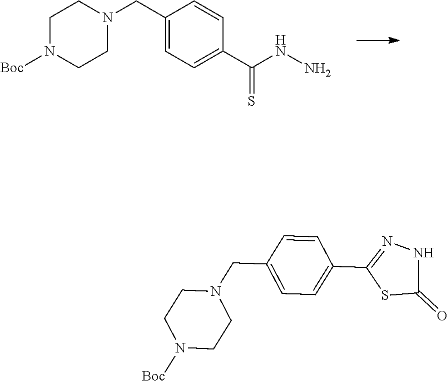 [SMILES: CC(C)(C)OC(=O)N1CCN(Cc2ccc(C(=S)NN)cc2)CC1 ; CC(C)(C)OC(=O)N1CCN(Cc2ccc(-c3n[nH]c(=O)s3)cc2)CC1]

A solution in which tert-butyl 4-[[4-(aminocarbonothioyl)phenyl]methyl]piperazin-1-carboxylate (100.00%, 0.500 g, 1.427 mmol) synthesized in Step 1 and 1,1′-carbonylbis-1H-imidazole (100.00%, 0.278 g, 1.715 mmol) were dissolved in dichloromethane (30 mL) at 50° C. was stirred overnight at the same temperature. Then, the reaction was terminated by lowering the temperature to room temperature. Water was poured into the reaction mixture, and the mixture was extracted with dichloromethane. The organic layer was washed with a saturated aqueous ammonium chloride solution, dried over anhydrous magnesium sulfate, filtered, and concentrated under reduced pressure. The concentrate was purified and concentrated by column chromatography (SiO₂, 12 g cartridge; ethyl acetate/hexane=0% to 40%) to obtain the title compound (0.25 g, 46.55%, white solid).

### [Step 3] Synthesis of tert-butyl 4-[[4-[4-[[5-[5-(difluoromethyl)-1,3,4-oxadiazol-2-yl]-2-pyridyl]methyl]-5-oxo-1,3,4-thiadiazol-2-yl]phenyl]methyl]piperazin-1-carboxylate

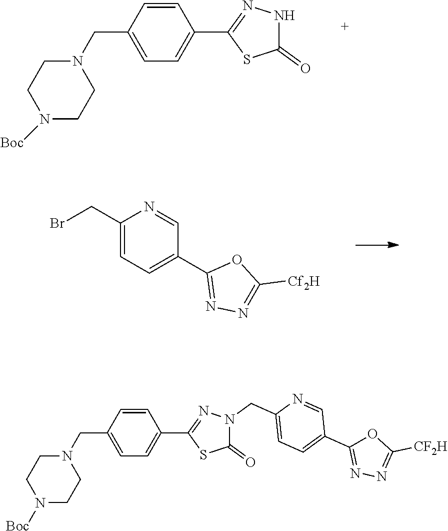

A solution in which tert-butyl 4-[[4-(2-oxo-3H-1,3,4-thiadiazol-5-yl)phenyl]methyl]piperazin-1-carboxylate (100.00%, 0.100 g, 0.266 mmol) synthesized in Step 2 and potassium carbonate (100.00%, 0.055 g, 0.398 mmol) were dissolved in N,N-dimethylformamide (10 mL) was stirred at room temperature for 30 minutes. Then, 2-[6-(bromomethyl)-3-pyridyl]-5-(difluoromethyl)-1,3,4-oxadiazole (100.00%, 0.081 g, 0.279 mmol) and potassium iodide (100.00%, 0.022 g, 0.133 mmol) were added and further stirred overnight at the same temperature. Water was poured into the reaction mixture, and the mixture was extracted with ethyl acetate. The organic layer was washed with a saturated aqueous ammonium chloride solution, dried over anhydrous magnesium sulfate, filtered, and concentrated under reduced pressure. The concentrate was purified and concentrated by column chromatography (SiO₂, 12 g cartridge; ethyl acetate/hexane=0% to 40%) to obtain the title compound (0.095 g, 61.08%, white solid).

### [Step 4] Synthesis of 3-[[5-[5-(difluoromethyl)-1,3,4-oxadiazol-2-yl]-2-pyridyl]methyl]-5-[4-(piperazin-1-ylmethyl)phenyl]-1,3,4-thiadiazol-2-one 2,2,2-trifluoroacetic acid

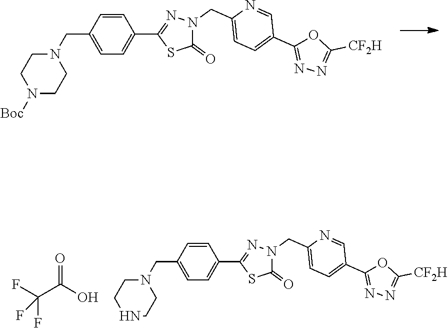

A solution in which tert-butyl 4-[[4-[4-[[5-[5-(difluoromethyl)-1,3,4-oxadiazol-2-yl]-2-pyridyl]methyl]-5-oxo-1,3,4-thiadiazol-2-yl]phenyl]methyl]piperazine-1-carboxylate (100.00%, 0.090 g, 0.154 mmol) synthesized in Step 3 and trifluoroacetic acid (100.00%, 0.053 g, 0.465 mmol) were dissolved in dichloromethane (10 mL) at room temperature was stirred overnight at the same temperature. After removing the solvent from the reaction mixture under reduced pressure, the obtained product was used without further purification (0.088 g, 95.51%, yellow oil).

### [Step 5] Synthesis of Compound 66

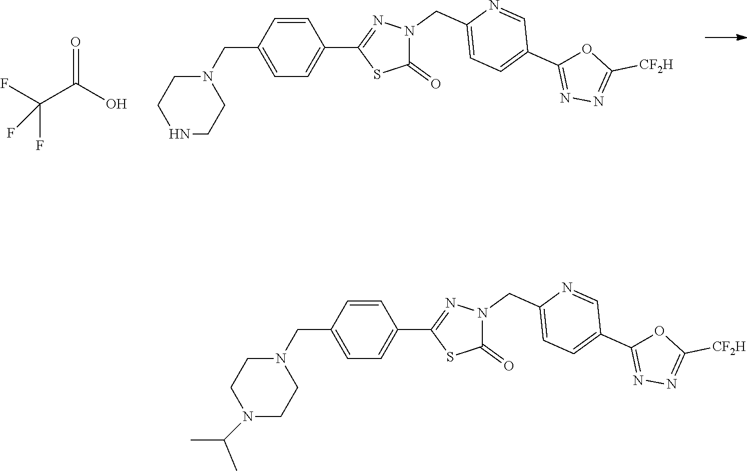

A solution in which 3-[[5-[5-(difluoromethyl)-1,3,4-oxadiazol-2-yl]-2-pyridyl]methyl]-5-[4-(piperazin-1-yl methyl)phenyl]-1,3,4-thiadiazol-2-one 2,2,2-trifluoroacetic acid (100.00%, 0.030 g, 0.051 mmol) synthesized in Step 4, acetone (100.00%, 0.006 g, 0.103 mmol), N-ethyldiisopropylamine (100.00% solution, 0.018 mL, 0.103 mmol) and sodium triacetoxyborohydride (100.00%, 0.033 g, 0.156 mmol) were dissolved in dichloromethane (5 mL) was stirred at room temperature for 30 minutes and further stirred overnight at the same temperature. Water was poured into the reaction mixture, and the mixture was extracted with dichloromethane. The organic layer was washed with a saturated aqueous solution of sodium hydrogen carbonate, dried over anhydrous magnesium sulfate, filtered, and concentrated under reduced pressure. The concentrate was purified and concentrated by column chromatography (SiO₂, 4 g cartridge; methanol/dichloromethane=0% to 5%) to obtain the title compound (0.011 g, 41.80%, white solid).

¹H NMR (400 MHz, CDCl₃) δ 9.33 (d, J=1.6 Hz, 1H), 8.41 (dd, J=8.2, 2.2 Hz, 1H), 7.64 (d, J=8.2 Hz, 2H), 7.47 (d, J=8.2 Hz, 1H), 7.41 (d, J=8.2 Hz, 2H), 7.08 (s, 0.2H), 6.95 (s, 0.5H), 6.82 (s, 0.2H), 5.42 (s, 2H), 3.56 (s, 2H), 2.78-2.77 (m, 1H), 2.64-2.48 (m, 8H), 1.11 (d, J=6.5 Hz, 6H); LRMS (ES) m/z 529.35 (M⁺+1).

Compounds in Table 8 below were synthesized according to substantially the same method as in Example 66.

TABLE 8

| | Com- | |
|---|---|---|
| Exam- | pound | |
| ple | No. | Compound Name, ¹H-NMR, MS (ESI) |
| 63 | 63 | 3-[[5-[5-(difluoromethyl)-1,3,4-oxadiazol-2-yl]- |
| | | 2-pyridyl]methyl]-5-[3-[(4-isopropylpiperazin-1- |
| | | yl)methyl]phenyl]-1,3,4-thiadiazol-2-one |
| | | ¹H NMR (400 MHz, CDCl₃) δ 9.33 (d, J = 1.6 Hz, |
| | | 1H), 8.41 (dd, J = 8.2, 2.2 Hz, 1H), 7.64 (s, |
| | | 1H), 7.58-7.56 (m, 1H), 7.49-7.37 (m, 3H), 7.08 |
| | | (s, 0.2H), 6.95 (s, 0.5H), 6.82 (s, 0.2H), 5.42 |
| | | (s, 2H), 3.56 (s, 2H), 2.82-2.75 (m, 1H), 2.65- |
| | | 2.47 (m, 8H), 1.11 (d, J = 6.5 Hz, 6H); LRMS (ES) |
| | | m/z 528.78 (M⁺ + 1). |
| 64 | 64 | 5-[3-[(4-cyclobutylpiperazin-1- |
| | | yl)methyl]phenyl]-3-[[5-[5-(difluoromethyl)- |
| | | 1,3,4-oxadiazol-2-yl]-2-pyridyl]methyl]-1,3,4- |
| | | thiadiazol-2-one |
| | | ¹H NMR (400 MHz, CDCl₃) δ 9.33 (d, J = 1.7 Hz, |
| | | 1H), 8.41 (dd, J = 8.2, 2.2 Hz, 1H), 7.63 (s, |
| | | 1H), 7.58-7.56 (m, 1H), 7.48-7.36 (m, 3H), 7.08 |
| | | (s, 0.2H), 6.95 (s, 0.5H), 6.82 (s, 0.2H), 5.42 |
| | | (s, 2H), 3.55 (s, 2H), 2.78-2.74 (m, 1H), 2.51- |
| | | 2.29 (m, 6H), 2.06-2.00 (m, 6H), 1.88-1.66 (m, |
| | | 2H); LRMS (ES) m/z 540.88 (M⁺ + 1). |
| 65 | 65 | 3-[[5-[5-(difluoromethyl)-1,3,4-oxadiazol-2-yl]- |
| | | 2-pyridyl]methyl]-5-[3-[[4-(1,1-dioxothietan-3- |
| | | yl)piperazin-1-yl]methyl]phenyl]-1,3,4- |
| | | thiadiazol-2-one |
| | | ¹H NMR (400 MHz, CDCl₃) δ 9.33 (d, J = 2.1 Hz, |
| | | 1H), 8.41 (dd, J = 8.2, 2.2 Hz, 1H), 7.63 (s, |
| | | 1H), 7.60-7.57 (m, 1H), 7.49-7.38 (m, 3H), 7.08 |
| | | (s, 0.2H), 6.95 (s, 0.5H), 6.82 (s, 0.2H), 5.42 |
| | | (s, 2H), 4.15-4.03 (m, 4H), 3.55 (s, 2H), 3.26- |
| | | 3.18 (m, 1H), 2.71-2.21 (m, 8H); LRMS (ES) m/z |
| | | 590.68 (M⁺ + 1). |
| 67 | 67 | 5-[4-(4-cyclobutylpiperazin-1-yl)phenyl]-3-[[5- |
| | | [5-(difluoromethyl)-1,3,4-oxadiazol-2-yl]-2- |
| | | pyridyl]methyl]-1,3,4-thiadiazol-2-one |
| | | ¹H NMR (400 MHz, CDCl₃) δ 9.33 (d, J = 1.6 Hz, |
| | | 1H), 8.41 (dd, J = 8.2, 2.2 Hz, 1H), 7.64 (d, J = |
| | | 8.3 Hz, 2H), 7.47 (d, J = 8.1 Hz, 1H), 7.41 (d, |
| | | J = 8.3 Hz, 2H), 7.08 (s, 0.2H), 6.95 (s, 0.5H), |
| | | 6.82 (s, 0.2H), 5.42 (s, 2H), 3.56 (s, 2H), 2.80- |
| | | 2.73 (m, 1H), 2.61-2.25 (m, 6H), 2.06-1.88 (m, |
| | | 6H), 1.76-1.66 (m, 2H); LRMS (ES) m/z 540.50 |
| | | (M⁺ + 1). |
| 68 | 68 | 3-[[5-[5-(difluoromethyl)-1,3,4-oxadiazol-2-yl]- |
| | | 2-pyridyl]methyl]-5-[4-[4-(1,1-dioxothietan-3- |
| | | yl)piperazin-1-yl]phenyl]-1,3,4-thiadiazol-2-one |
| | | ¹H NMR (400 MHz, CDCl₃) δ 9.32 (d, J = 2.1 Hz, |
| | | 1H), 8.41 (dd, J = 8.2, 2.2 Hz, 1H), 7.65 (d, J = |
| | | 8.2 Hz, 2H), 7.47 (d, J = 8.2 Hz, 1H), 7.40 (d, |
| | | J = 8.2 Hz, 2H), 7.08 (s, 0.2H), 6.95 (s, 0.5H), |
| | | 6.82 (s, 0.2H), 5.42 (s, 2H), 4.15-4.03 (m, 4H), |
| | | 3.56 (s, 2H), 3.24-3.20 (m, 1H), 2.70-2.24 (m, |
| | | 8H); LRMS (ES) m/z 590.68 (M⁺ + 1). |

### Example 77: Synthesis of Compound 77, 3-[[5-[5-(difluoromethyl)-1,3,4-oxadiazol-2-yl]-2-pyridyl]methyl]-5-[4-[(4-isopropylpiperazin-1-yl)methyl]phenyl]-1,3,4-oxadiazol-2-thione

### [Step 1] Synthesis of tert-butyl 4-(4-(5-thioxo-4,5-dihydro-1,3,4-oxadiazol-2-yl)benzyl)piperazin-1-carboxylate

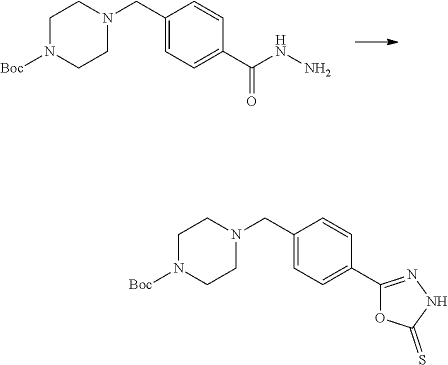 [SMILES: CC(C)(C)OC(=O)N1CCN(Cc2ccc(C(=O)NN)cc2)CC1 ; CC(C)(C)OC(=O)N1CCN(Cc2ccc(-c3n[nH]c(=S)o3)cc2)CC1]

A mixture in which tert-butyl 4-[[4-(hydrazinecarbonyl)phenyl]methyl]piperazin-1-carboxylate (100.00%, 1.000 g, 2.990 mmol) prepared in Step 2 of Example 86 and potassium ethylxanthate (100.00%, 0.575 g, 3.587 mmol) were mixed in ethanol (80 mL) at room temperature was heated to reflux overnight. The mixture was cooled to room temperature, and water was poured into the reaction mixture, followed by extraction with dichloromethane. The organic layer was washed with a saturated aqueous sodium chloride solution, dried over anhydrous magnesium sulfate, filtered, and concentrated under reduced pressure. The concentrate was purified and concentrated by column chromatography (SiO₂, 24 g cartridge; ethyl acetate/hexane=0% to 40%) to obtain the title compound (0.85 g, 75.50%, white solid).

### [Step 2] Synthesis of tert-butyl 4-[[4-[4-[[5-[5-(difluoromethyl)-1,3,4-oxadiazol-2-yl]-2-pyridyl]methyl]-5-thioxo-1,3,4-oxadiazol-2-yl]phenyl]methyl]piperazin-1-carboxylate

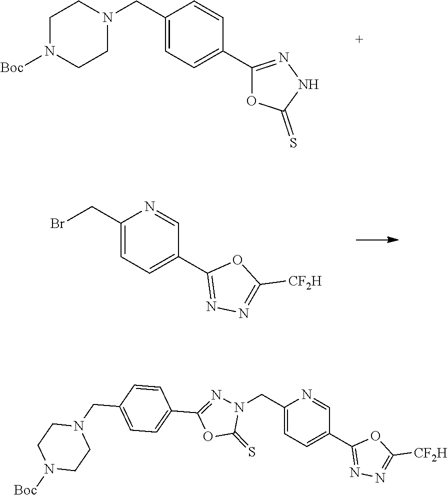 [SMILES: CC(C)(C)OC(=O)N1CCN(Cc2ccc(-c3n[nH]c(=S)o3)cc2)CC1 ; FC(F)c1nnc(-c2ccc(CBr)nc2)o1 ; CC(C)(C)OC(=O)N1CCN(Cc2ccc(-c3nn(Cc4ccc(-c5nnc(C(F)F)o5)cn4)c(=S)o3)cc2)CC1]

A solution in which tert-butyl 4-[[4-(2-thioxo-3H-1,3,4-oxadiazol-5-yl)phenyl]methyl]piperazin-1-carboxylate (100.00%, 0.500 g, 1.328 mmol) synthesized in Step 1 and potassium carbonate (100.00%, 0.197 g, 1.988 mmol) were dissolved in N,N-dimethylformamide (30 mL) was stirred at room temperature for 30 minutes. Then, 2-[6-(bromomethyl)-3-pyridyl]-5-(difluoromethyl)-1,3,4-oxadiazole (100.00%, 0.404 g, 1.393 mmol) and potassium iodide (100.00%, 0.110 g, 0.663 mmol) were added and further stirred overnight at the same temperature. Water was poured into the reaction mixture, and the mixture was extracted with ethyl acetate. The organic layer was washed with a saturated aqueous ammonium chloride solution, dried over anhydrous magnesium sulfate, filtered, and concentrated under reduced pressure. The concentrate was purified and concentrated by column chromatography (SiO₂, 24 g cartridge; ethyl acetate/hexane=0% to 40%) to obtain the title compound (0.65 g, 83.58%, white solid).

### [Step 3] Synthesis of 3-[[5-[5-(difluoromethyl)-1,3,4-oxadiazol-2-yl]-2-pyridyl]methyl]-5-[4-(piperazin-1-ylmethyl)phenyl]-1,3,4-oxadiazol-2-thione 2,2,2-trifluoroacetic acid

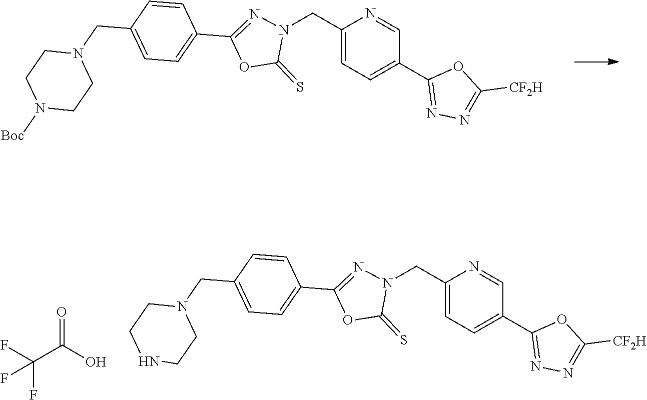 [SMILES: CC(C)(C)OC(=O)N1CCN(Cc2ccc(-c3nn(Cc4ccc(-c5nnc(C(F)F)o5)cn4)c(=S)o3)cc2)CC1 ; O=C(O)C(F)(F)F ; FC(F)c1nnc(-c2ccc(Cn3nc(-c4ccc(CN5CCNCC5)cc4)oc3=S)nc2)o1]

A solution in which tert-butyl 4-[[4-[4-[[5-[5-(difluoromethyl)-1,3,4-oxadiazol-2-yl]-2-pyridyl]methyl]-5-thioxo-1,3, 4-oxadiazol-2-yl]phenyl]methyl]piperazin-1-carboxylate (100.00%, 0.300 g, 0.512 mmol) synthesized in Step 2 and trifluoroacetic acid (100.00%, 0.175 g, 1.535 mmol) were dissolved in dichloromethane (20 mL) at room temperature was stirred for 3 hours at the same temperature. After removing the solvent from the reaction mixture under reduced pressure, the obtained product was used without further purification (0.28 g, 91.17%, yellow oil).

### [Step 4] Synthesis of Compound 77

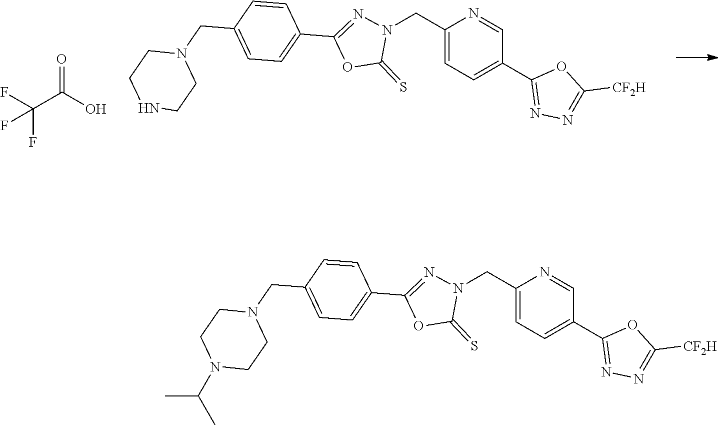 [SMILES: O=C(O)C(F)(F)F ; FC(F)c1nnc(-c2ccc(Cn3nc(-c4ccc(CN5CCNCC5)cc4)oc3=S)nc2)o1 ; CC(C)N1CCN(Cc2ccc(-c3nn(Cc4ccc(-c5nnc(C(F)F)o5)cn4)c(=S)o3)cc2)CC1]

A solution in which 3-[[5-[5-(difluoromethyl)-1,3,4-oxadiazol-2-yl]-2-pyridyl]methyl]-5-[4-(piperazin-1-yl methyl)phenyl]-1,3,4-oxadiazol-2-thione 2,2,2-trifluoroacetic acid (100.00%, 0.050 g, 0.083 mmol) synthesized in Step 3, acetone (100.00%, 0.010 g, 0.172 mmol) and N-ethyldiisopropylamine (100.00% solution, 0.029 mL, 0.167 mmol) were dissolved in dichloromethane (5 mL) was stirred at room temperature for 30 minutes. Then, sodium triacetoxyborohydride (100.00%, 0.053 g, 0.250 mmol) was added and further stirred overnight at the same temperature. Water was poured into the reaction mixture, and the mixture was extracted with dichloromethane. The organic layer was washed with a saturated aqueous solution of sodium hydrogen carbonate, dried over anhydrous magnesium sulfate, filtered, and concentrated under reduced pressure. The concentrate was purified and concentrated by column chromatography (SiO₂, 4 g cartridge; methanol/dichloromethane=0% to 5%) to obtain the title compound (0.022 g, 50.00%, white solid).

¹H NMR (400 MHz, CDCl₃) δ 9.31 (d, J=2.2 Hz, 1H), 8.39 (dd, J=8.2, 2.2 Hz, 1H), 7.95 (d, J=8.3 Hz, 2H), 7.82 (d, J=8.2 Hz, 1H), 7.46 (d, J=8.3 Hz, 2H), 7.08 (s, 0.2H), 6.95 (s, 0.5H), 6.82 (s, 0.2H), 4.73 (s, 2H), 3.60 (s, 2H), 3.16-2.92 (m, 1H), 2.81-2.76 (m, 8H), 1.19 (d, J=6.5 Hz, 6H); LRMS (ES) m/z 528.87 (M⁺+1).

Compounds in Table 9 below were synthesized according to substantially the same method as in Example 77.

TABLE 9

| | Com- | |
|---|---|---|
| Exam- | pound | |
| ple | No. | Compound Name, ¹H-NMR, MS (ESI) |
| 78 | 78 | 5-[4-[(4-cyclobutylpiperazin-1- |
| | | yl)methyl]phenyl]-3-[[5-[5-(difluoromethyl)- |
| | | 1,3,4-oxadiazol-2-yl]-2-pyridyl]methyl]-1,3,4- |
| | | oxadiazol-2-thione |
| | | ¹H NMR (400 MHz, CDCl₃) δ 9.31 (d, J = 1.7 Hz, |
| | | 1H), 8.38 (dd, J = 8.2, 2.2 Hz, 1H), 7.93 (d, J = |
| | | 8.3 Hz, 2H), 7.82 (d, J = 8.2 Hz, 1H), 7.46 (d, |
| | | J = 8.3 Hz, 2H), 7.08 (s, 0.2H), 6.95 (s, 0.5H), |
| | | 6.82 (s, 0.2H), 4.73 (s, 2H), 3.57 (s, 2H), 2.81- |
| | | 2.62 (m, 1H), 2.52-2.18 (m, 8H), 2.07-2.03 (m, |
| | | 2H), 2.00-1.86 (m, 2H), 1.76-1.72 (m, 2H); LRMS |
| | | (ES) m/z 540.88 (M⁺ + 1). |
| 79 | 79 | 5-[4-[(4-cyclopentylpiperazin-1- |
| | | yl)methyl]phenyl]-3-[[5-[5-(difluoromethyl)- |
| | | 1,3,4-oxadiazol-2-yl]-2-pyridyl]methyl]-1,3,4- |
| | | oxadiazol-2-thione |
| | | ¹H NMR (400 MHz, CDCl₃) δ 9.32 (d, J = 1.6 Hz, |
| | | 1H), 8.39 (dd, J = 8.2, 2.2 Hz, 1H), 7.94 (d, J = |
| | | 8.3 Hz, 2H), 7.83 (d, J = 8.2 Hz, 1H), 7.47 (d, |
| | | J = 8.3 Hz, 2H), 7.08 (s, 0.2H), 6.95 (s, 0.5H), |
| | | 6.82 (s, 0.2H), 4.73 (s, 2H), 3.58 (s, 2H), 2.81- |
| | | 2.39 (m, 9H), 1.90-1.86 (m, 2H), 1.72-1.61 (m, |
| | | 2H), 1.58-1.55 (m, 4H); LRMS (ES) m/z 554.86 |
| | | (M⁺ + 1). |

### Example 26: Synthesis of Compound 26, 3-[[5-[5-(difluoromethyl)-1,3,4-oxadiazol-2-yl]-2-pyridyl]methyl]-5-[2-fluoro-3-[1-(oxetan-3-yl)-4-piperidyl]phenyl]-1,3,4-oxadiazol-2-one

### [Step 1] Synthesis of tert-butyl 4-(2-fluoro-3-methoxycarbonyl-phenyl)-3,6-dihydro-2H-pyridin-1-carboxylate

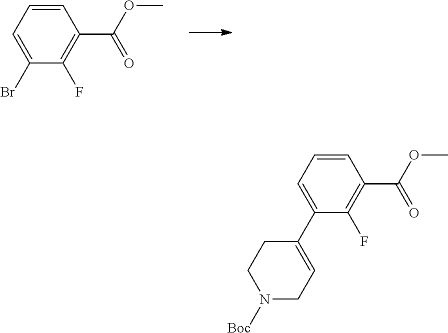 [SMILES: COC(=O)c1cccc(Br)c1F ; COC(=O)c1cccc(C2=CCN(C(=O)OC(C)(C)C)CC2)c1F]

A solution in which methyl 3-bromo-2-fluoro-benzoate (100.00%, 1.000 g, 4.291 mmol), tert-butyl 4-(4,4,5,5-tetramethyl-1,3,2-dioxaborolan-2-yl)-3,6-dihydro-2H-pyridine-1-carboxylate (100.00%, 1.460 g, 4.722 mmol), bis(triphenylphosphine)palladium(II) dichloride (100.00%, 0.301 g, 0.429 mmol) and sodium carbonate (100.00%, 1.365 g, 12.879 mmol) were dissolved in N,N-dimethylformamide (20 mL)/water (5 mL) at room temperature was stirred overnight at the same temperature. Water was poured into the reaction mixture, and the mixture was extracted with ethyl acetate. The organic layer was washed with a saturated aqueous ammonium chloride solution, dried over anhydrous magnesium sulfate, filtered, and concentrated under reduced pressure. The concentrate was purified and concentrated by column chromatography (SiO₂, 24 g cartridge; ethyl acetate/hexane=0% to 30%) to obtain the title compound (1.100 g, 76.43%, white solid).

### [Step 2] Synthesis of tert-butyl 4-(2-fluoro-3-methoxycarbonyl-phenyl)piperidine-1-carboxylate

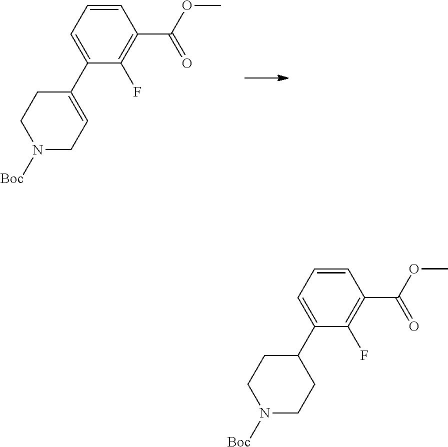 [SMILES: COC(=O)c1cccc(C2=CCN(C(=O)OC(C)(C)C)CC2)c1F ; COC(=O)c1cccc(C2CCN(C(=O)OC(C)(C)C)CC2)c1F]

A solution in which tert-butyl 4-(2-fluoro-3-methoxycarbonyl-phenyl)-3,6-dihydro-2H-pyridin-1-carboxylate (100.00%, 0.900 g, 2.683 mmol) synthesized in Step 1 and Pd/C (100.00%, 0.286 g, 2.688 mmol) were dissolved in methanol (20 mL) was stirred overnight at the same temperature. The reaction mixture was filtered through a celite pad to remove solids, and the solvent was removed from the filtrate under reduced pressure. The concentrate was purified and concentrated by column chromatography (SiO₂, 12 g cartridge; ethyl acetate/hexane=0% to 40%) to obtain the title compound (0.74 g, 81.73%, yellow oil).

### [Step 3] Synthesis of tert-butyl 4-[2-fluoro-3-(hydrazinecarbonyl)phenyl]piperidine-1-carboxylate

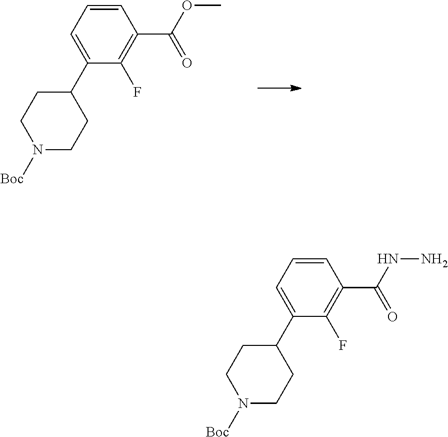 [SMILES: COC(=O)c1cccc(C2CCN(C(=O)OC(C)(C)C)CC2)c1F ; CC(C)(C)OC(=O)N1CCC(c2cccc(C(=O)NN)c2F)CC1]

A solution in which tert-butyl 4-(2-fluoro-3-methoxycarbonyl-phenyl)piperidine-1-carboxylate (100.00%, 0.700 g, 2.075 mmol) synthesized in Step 2 and hydrazine monohydrate (100.00%, 1.039 g, 20.755 mmol) were dissolved in ethanol (30 mL) at room temperature was stirred overnight at the same temperature. Water was poured into the reaction mixture, and the mixture was extracted with dichloromethane. The organic layer was washed with a saturated aqueous solution of sodium hydrogen carbonate, dried over anhydrous magnesium sulfate, filtered, and concentrated under reduced pressure. The obtained product was used without further purification (0.65 g, 92.86%, white solid).

### [Step 4] Synthesis of tert-butyl 4-[2-fluoro-3-(2-oxo-3H-1,3,4-oxadiazol-5-yl)phenyl]piperidin-1-carboxylate

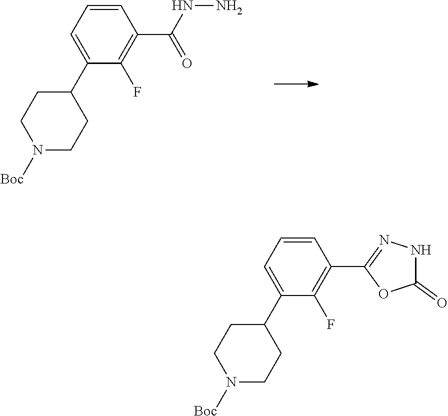 [SMILES: CC(C)(C)OC(=O)N1CCC(c2cccc(C(=O)NN)c2F)CC1 ; CC(C)(C)OC(=O)N1CCC(c2cccc(-c3n[nH]c(=O)o3)c2F)CC1]

A solution in which tert-butyl 4-[2-fluoro-3-(hydrazinecarbonyl)phenyl]piperidine-1-carboxylate (100.00%, 0.700 g, 2.075 mmol) synthesized in Step 3 and 1,1′-carbonylbis-1H-imidazole (100.00%, 0.404 g, 2.492 mmol) were dissolved in dichloromethane (30 mL) at room temperature was stirred overnight at the same temperature. Water was poured into the reaction mixture, and the mixture was extracted with dichloromethane. The organic layer was washed with a saturated aqueous ammonium chloride solution, dried over anhydrous magnesium sulfate, filtered, and concentrated under reduced pressure. The concentrate was purified and concentrated by column chromatography (SiO₂, 12 g cartridge; ethyl acetate/hexane=0% to 40%) to obtain the title compound (0.55 g, 72.95%, white solid).

### [Step 5] Synthesis of tert-butyl 4-[3-[4-[[5-[5-(difluoromethyl)-1,3,4-oxadiazol-2-yl]-2-pyridyl]methyl]-5-oxo-1,3,4-oxadiazol-2-yl]-2-fluoro-phenyl]piperidin-1-carboxylate

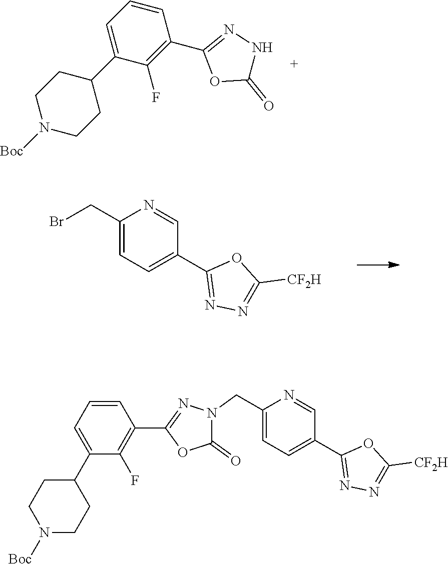 [SMILES: CC(C)(C)OC(=O)N1CCC(c2cccc(-c3n[nH]c(=O)o3)c2F)CC1 ; FC(F)c1nnc(-c2ccc(CBr)nc2)o1 ; CC(C)(C)OC(=O)N1CCC(c2cccc(-c3nn(Cc4ccc(-c5nnc(C(F)F)o5)cn4)c(=O)o3)c2F)CC1]

To a solution in which tert-butyl 4-[2-fluoro-3-(2-oxo-3H-1,3,4-oxadiazol-5-yl)phenyl]piperidin-1-carboxylate (100.00%, 0.400 g, 1.101 mmol) synthesized in Step 4 and potassium carbonate (100.00%, 0.164 g, 1.655 mmol) were dissolved in N,N-dimethylformamide (20 mL) at room temperature, 2-[6-(bromomethyl)-3-pyridyl]-5-(difluoromethyl)-1,3,4-oxadiazole (100.00%, 0.335 g, 1.155 mmol) and potassium iodide (100.00%, 0.091 g, 0.548 mmol) were added and stirred at the same temperature. Water was poured into the reaction mixture, and the mixture was extracted with ethyl acetate. The organic layer was washed with a saturated aqueous ammonium chloride solution, dried over anhydrous magnesium sulfate, filtered, and concentrated under reduced pressure. The concentrate was purified and concentrated by column chromatography (SiO₂, 12 g cartridge; ethyl acetate/hexane=0% to 40%) to obtain the title compound (0.54 g, 85.69%, white solid).

### [Step 6] Synthesis of 3-[[5-[5-(difluoromethyl)-1,3,4-oxadiazol-2-yl]-2-pyridyl]methyl]-5-[2-fluoro-3-(4-piperidyl)phenyl]-1,3,4-oxadiazol-2-one 2,2,2-trifluoroacetic acid

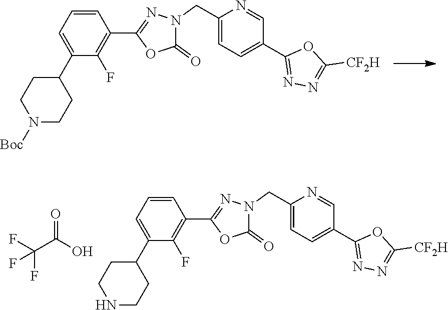 [SMILES: CC(C)(C)OC(=O)N1CCC(c2cccc(-c3nn(Cc4ccc(-c5nnc(C(F)F)o5)cn4)c(=O)o3)c2F)CC1 ; O=C(O)C(F)(F)F ; O=c1oc(-c2cccc(C3CCNCC3)c2F)nn1Cc1ccc(-c2nnc(C(F)F)o2)cn1]

A solution in which tert-butyl 4-[3-[4-[[5-[5-(difluoromethyl)-1,3,4-oxadiazol-2-yl]-2-pyridyl]methyl]-5-oxo-1,3,4-oxadiazol-2-yl]-2-fluoro-phenyl]piperidin-1-carboxylate (100.00%, 0.300 g, 0.524 mmol) synthesized in Step 5 and 2,2,2-trifluoroacetic acid (100.00%, 0.179 g, 1.570 mmol) were dissolved in dichloromethane (20 mL) at room temperature was stirred for 3 hours at the same temperature. After removing the solvent from the reaction mixture under reduced pressure, the obtained product was used without further purification (0.3 g, 97.63%, yellow oil).

### [Step 7] Synthesis of Compound 26

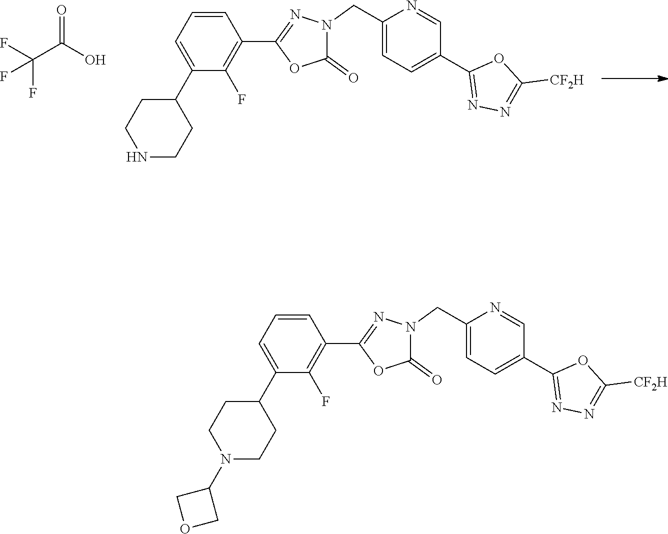 [SMILES: O=C(O)C(F)(F)F ; O=c1oc(-c2cccc(C3CCNCC3)c2F)nn1Cc1ccc(-c2nnc(C(F)F)o2)cn1 ; O=c1oc(-c2cccc(C3CCN(C4COC4)CC3)c2F)nn1Cc1ccc(-c2nnc(C(F)F)o2)cn1]

A solution in which 3-[[5-[5-(difluoromethyl)-1,3,4-oxadiazol-2-yl]-2-pyridyl]methyl]-5-[2-fluoro-3-(4-piperidyl)phenyl]-1,3,4-oxadiazol-2-one 2,2,2-trifluoroacetic acid (100.00%, 0.050 g, 0.085 mmol) synthesized in Step 6, N-ethyldiisopropylamine (100.00% solution, 0.03 mL, 0.172 mmol), oxetan-3-one (100.00%, 0.012 g, 0.167 mmol), and sodium triacetoxyborohydride (100.00%, 0.054 g, 0.255 mmol) were dissolved in dichloromethane (5 mL) at room temperature was stirred overnight at the same temperature. Water was poured into the reaction mixture, and the mixture was extracted with dichloromethane. The organic layer was washed with a saturated aqueous solution of sodium hydrogen carbonate, dried over anhydrous magnesium sulfate, filtered, and concentrated under reduced pressure. The concentrate was purified and concentrated by column chromatography (SiO₂, 4 g cartridge; methanol/dichloromethane=0% to 5%) to obtain the title compound (0.031 g, 68.79%, white solid).

¹H NMR (400 MHz, CDCl₃) δ 9.32 (d, J=1.6 Hz, 1H), 8.43 (dd, J=8.2, 2.2 Hz, 1H), 7.70-7.66 (m, 1H), 7.53 (d. J=8.2 Hz, 1H), 7.47-7.43 (m, 1H), 7.26-7.23 (m, 1H), 7.08 (s, 0.2H), 6.95 (s, 0.5H), 6.82 (s, 0.2H) 5.26 (s, 2H), 4.71-4.64 (m, 4H), 3.56-3.50 (m, 1H), 3.00-2.94 (m, 1H), 2.91-2.88 (m, 2H), 2.02-1.96 (m, 2H), 1.87-1.81 (m, 4H); LRMS (ES) m/z 529.82 (M⁺+1).

Compounds in Table 10 below were synthesized according to substantially the same method as in Example 26.

TABLE 10

| | Com- | |
|---|---|---|
| Exam- | pound | |
| ple | No. | Compound Name, ¹H-NMR, MS (ESI) |
| 8 | 8 | 3-[[5-[5-(difluoromethyl)-1,3,4-oxadiazol-2-yl]- |
| | | 2-pyridyl]methyl]-5-[3-(1-methyl-4- |
| | | piperidyl)phenyl]-1,3,4-oxadiazol-2-one |
| | | ¹H NMR (400 MHz, CDCl₃) δ 9.31 (d, J = 2.1 Hz, |
| | | 1H), 8.44 (dd, J = 8.2, 2.2 Hz, 1H), 7.74 (s, |
| | | 1H), 7.72-7.70 (m, 1H), 7.54 (d, J = 8.2 Hz, 1H), |
| | | 7.43-7.39 (m, 2H), 7.08 (s, 0.2H), 6.95 (s, 0.5H), |
| | | 6.82 (s, 0.2H), 5.23 (s, 2H), 3.17-3.14 (m, 2H), |
| | | 2.65-2.60 (m, 1H), 2.46 (s, 3H), 2.26-2.19 (m, |
| | | 2H), 2.02-1.89 (m, 4H); LRMS (ES) m/z 469.62 |
| | | (M⁺ + 1). |
| 9 | 9 | 3-[[5-[5-(difluoromethyl)-1,3,4-oxadiazol-2-yl]- |
| | | 2-pyridyl]methyl]-5-[3-[1-(oxetane-3-yl)-4- |
| | | piperidyl]phenyl]-1,3,4-oxadiazol-2-one |
| | | ¹H NMR (400 MHz, CDCl₃) δ 9.34 (d, J = 2.1 Hz, |
| | | 1H), 8.44 (dd, J = 8.2, 2.2 Hz, 1H), 7.77 (s, |
| | | 1H), 7.72-7.69 (m, 1H), 7.54 (d, J = 8.2 Hz, 1H), |
| | | 7.43-7.38 (m, 2H), 7.08 (s, 0.2H), 6.95 (0.5H), |
| | | 6.82 (s, 0.2H), 5.23 (s, 2H), 4.71-4.64 (m, 4H), |
| | | 3.56-3.49 (m, 1H), 2.91-2.88 (m, 2H), 2.63-2.55 |
| | | (m, 1H), 1.99-1.80 (m, 6H); LRMS (ES) m/z 511.67 |
| | | (M⁺ + 1). |
| 10 | 10 | 3-[[5-[5-(difluoromethyl)-1,3,4-oxadiazol-2-yl]- |
| | | 2-pyridyl]methyl]-5-[3-[1-(3-methylcyclobutyl)- |
| | | 4-piperidyl]phenyl]-1,3,4-oxadiazol-2-one |
| | | ¹H NMR (400 MHz, CDCl₃) δ 9.34 (d, J = 2.1 Hz, |
| | | 1H), 8.44 (dd, J = 8.2, 2.2 Hz, 1H), 7.77 (s, |
| | | 1H), 7.71-7.67 (m, 1H), 7.53 (d, J = 7.7 Hz, 1H), |
| | | 7.43-7.40 (m, 2H), 7.08 (s, 0.2H), 6.95 (s, 0.5H), |
| | | 6.82 (s, 0.2H), 5.23 (s, 2H), 3.03 (brs, 2H), |
| | | 2.55-2.54 (m, 2H), 2.29-2.23 (m, 2H), 2.07-2.04 |
| | | (m, 1H), 1.99-1.84 (m, 7H), 1.56-1.43 (m, 1H), |
| | | 1.23-1.18 (m, 1H), 1.15-1.08 (m, 2H); LRMS (ES) |
| | | m/z 523.68 (M⁺ + 1). |
| 29 | 29 | 3-[[5-[5-(difluoromethyl)-1,3,4-oxadiazol-2-yl]- |
| | | 2-pyridyl]methyl]-5-[4-(1-methyl-4- |
| | | piperidyl)phenyl]-1,3,4-oxadiazol-2-one |
| | | ¹H NMR (400 MHz, CDCl₃) δ 9.32 (d, J = 1.6 Hz, |
| | | 1H), 8.43 (dd, J = 8.2, 2.2 Hz, 1H), 7.81 (d, J = |
| | | 8.4 Hz, 2H), 7.52 (d, J = 8.2 Hz, 1H), 7.35 (d, |
| | | J = 8.3 Hz, 2H), 7.08 (s, 0.2H), 6.95 (s, 0.5H), |
| | | 6.82 (s, 0.2H), 5.23 (s, 2H), 3.16-3.13 (m, 2H), |
| | | 2.64-2.56 (m, 1H), 2.45 (s, 3H), 2.27-2.22 (m, |
| | | 2H), 2.05-1.88 (m, 4H); LRMS (ES) m/z 469.5 |
| | | (M⁺ + 1). |
| 30 | 30 | 3-[[5-[5-(difluoromethyl)-1,3,4-oxadiazol-2-yl]- |
| | | 2-pyridyl]methyl]-5-[4-(1-isopropyl-4- |
| | | piperidyl)phenyl]-1,3,4-oxadiazol-2-one |
| | | ¹H NMR (400 MHz, CDCl₃) δ 9.32 (d, J = 1.6 Hz, |
| | | 1H), 8.43 (dd, J = 8.2, 2.2 Hz, 1H), 7.80 (d, J = |
| | | 8.4 Hz, 2H), 7.52 (d, J = 8.2 Hz, 1H), 7.36 (d, |
| | | J = 8.3 Hz, 2H), 7.08 (s, 0.2H), 6.95 (s, 0.5H), |
| | | 6.82 (s, 0.2H), 5.23 (s, 2H), 3.32-3.10 (m, 2H), |
| | | 3.02 (brs, 1H), 2.71-2.59 (m, 1H), 2.57-2.35 (m, |
| | | 2H), 2.06-1.91 (m, 4H), 1.32-1.20 (m, 6H); LRMS |
| | | (ES) m/z 497.8 (M⁺ + 1). |

### Example 27: Synthesis of Compound 27, 3-[[5-[5-(difluoromethyl)-1,3,4-oxadiazol-2-yl]-2-pyridyl]methyl]-5-[2-fluoro-3-[1-(1-methylazetidin-3-yl)-4-piperidyl]phenyl]-1,3,4-oxadiazol-2-one

### [Step 1] Synthesis of tert-butyl 3-[4-[3-[4-[[5-[5-(difluoromethyl)-1,3,4-oxadiazol-2-yl]-2-pyridyl]methyl]-5-oxo-1,3,4-oxadiazol-2-yl]-2-fluoro-phenyl]-1-piperidyl]azetidin-1-carboxylate

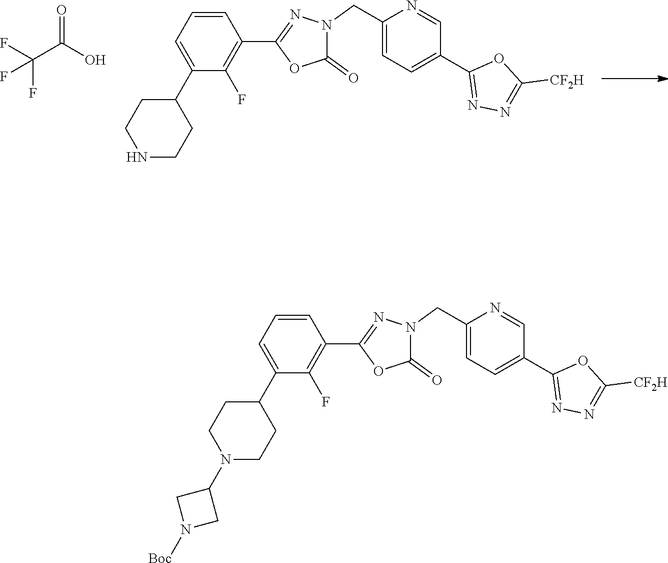 [SMILES: O=C(O)C(F)(F)F ; O=c1oc(-c2cccc(C3CCNCC3)c2F)nn1Cc1ccc(-c2nnc(C(F)F)o2)cn1 ; CC(C)(C)OC(=O)N1CC(N2CCC(c3cccc(-c4nn(Cc5ccc(-c6nnc(C(F)F)o6)cn5)c(=O)o4)c3F)CC2)C1]

A solution in which 3-[[5-[5-(difluoromethyl)-1,3,4-oxadiazol-2-yl]-2-pyridyl]methyl]-5-[2-fluoro-3-(4-piperidyl)phenyl]-1,3,4-oxadiazol-2-one 2,2,2-trifluoroacetic acid (100.00%, 0.150 g, 0.256 mmol) synthesized in Step 6 of Example 26, N-ethyldiisopropylamine (100.00% solution, 0.089 mL, 0.511 mmol), tert-butyl 3-oxoazetidin-1l-carboxylate (100.00%, 0.088 g, 0.514 mmol), and sodium triacetoxyborohydride (100.00%, 0.163 g, 0.769 mmol) were dissolved in dichloromethane (5 mL) at room temperature was stirred overnight at the same temperature. Water was poured into the reaction mixture, and the mixture was extracted with dichloromethane. The organic layer was washed with a saturated aqueous solution of sodium hydrogen carbonate, dried over anhydrous magnesium sulfate, filtered, and concentrated under reduced pressure. The concentrate was purified and concentrated by column chromatography (SiO₂, 4 g cartridge; methanol/dichloromethane=0% to 5%) to obtain the title compound (0.11 g, 68.52%, white solid).

### [Step 2] Synthesis of 5-[3-[1-(azetidin-3-yl)-4-piperidyl]-2-fluoro-phenyl]-3-[[5-[5-(difluoromethyl)-1,3,4-oxadiazol-2-yl]-2-pyridyl]methyl]-1,3,4-oxadiazol-2-one 2,2,2-trifluoroacetic acid

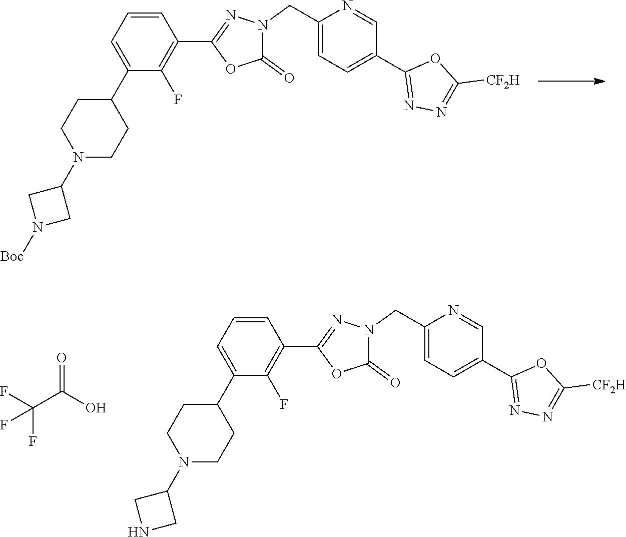 [SMILES: CC(C)(C)OC(=O)N1CC(N2CCC(c3cccc(-c4nn(Cc5ccc(-c6nnc(C(F)F)o6)cn5)c(=O)o4)c3F)CC2)C1 ; O=C(O)C(F)(F)F ; O=c1oc(-c2cccc(C3CCN(C4CNC4)CC3)c2F)nn1Cc1ccc(-c2nnc(C(F)F)o2)cn1]

A solution in which tert-butyl 3-[4-[3-[4-[[5-[5-(difluoromethyl)-1,3,4-oxadiazol-2-yl]-2-pyridyl]methyl]-5-oxo-1,3,4-oxadiazol-2-yl]-2-fluoro-phenyl]-1-piperidyl]azetidin-1-carboxylate (100.00%, 0.110 g, 0.175 mmol) synthesized in Step 1 and 2,2,2-trifluoroacetic acid (100.00%, 0.060 g, 0.526 mmol) were dissolved in dichloromethane (5 mL) at room temperature was stirred overnight at the same temperature. After removing the solvent from the reaction mixture under reduced pressure, the obtained product was used without further purification (0.100 g, 88.94%, yellow oil).

### [Step 3] Synthesis of Compound 27

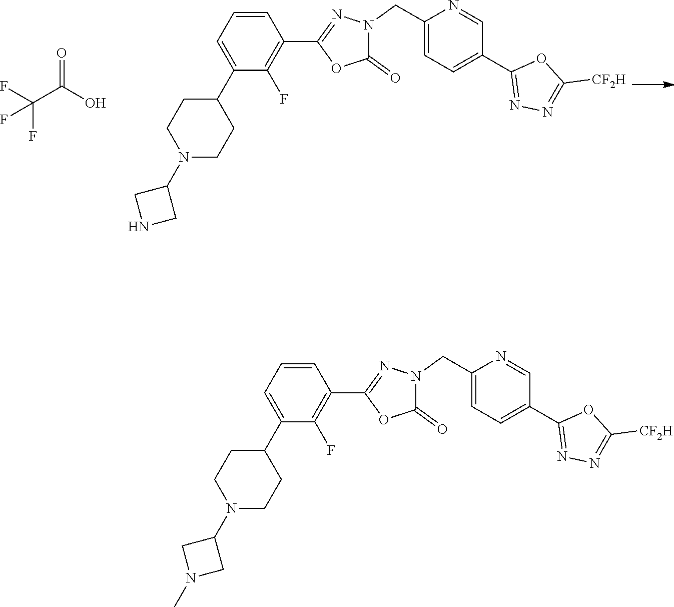 [SMILES: O=C(O)C(F)(F)F ; O=c1oc(-c2cccc(C3CCN(C4CNC4)CC3)c2F)nn1Cc1ccc(-c2nnc(C(F)F)o2)cn1 ; CN1CC(N2CCC(c3cccc(-c4nn(Cc5ccc(-c6nnc(C(F)F)o6)cn5)c(=O)o4)c3F)CC2)C1]

A solution in which 5-[3-[1-(azetidin-3-yl)-4-piperidyl]-2-fluoro-phenyl]-3-[[5-[5-(difluoromethyl)-1,3,4-oxadiazol-2-yl]-2-pyridyl]methyl]-1,3,4-oxadiazol-2-one 2,2,2-trifluoroacetic acid (100.00%, 0.050 g, 0.078 mmol) synthesized in Step 2, formaldehyde (100.00%, 0.005 g, 0.167 mmol), N-ethyldiisopropylamine (100.00% solution, 0.027 mL, 0.155 mmol) and sodium triacetoxyborohydride (100.00%, 0.050 g, 0.236 mmol) were dissolved in dichloromethane (5 mL) at room temperature was stirred overnight at the same temperature. Water was poured into the reaction mixture, and the mixture was extracted with dichloromethane. The organic layer was washed with a saturated aqueous solution of sodium hydrogen carbonate, dried over anhydrous magnesium sulfate, filtered, and concentrated under reduced pressure. The concentrate was purified and concentrated by column chromatography (SiO₂, 4 g cartridge; methanol/dichloromethane=0% to 5%) to obtain the title compound (0.029 g, 68.71%, white solid).

¹H NMR (400 MHz, CDCl₃) δ 9.31 (d, J=1.6 Hz, 1H), 8.43 (dd, J=8.2, 2.2 Hz, 1H), 7.69-7.65 (m, 1H), 7.53 (d. J=8.2 Hz, 1H), 7.45-7.41 (m, 1H), 7.25-7.21 (m, 1H), 7.08 (s, 0.2H), 6.95 (s, 0.5H), 6.82 (s, 0.2H) 5.26 (s, 2H), 3.76 (brs, 2H), 3.09 (brs, 3H), 2.99-2.90 (m, 3H), 2.50 (s, 3H), 2.09-1.94 (m, 2H), 1.87-1.80 (m, 4H); LRMS (ES) m/z 542.86 (M⁺+1).

Compounds in Table 11 below were synthesized according to substantially the same method as in Example 27.

TABLE 11

| | Com- | |
|---|---|---|
| Exam- | pound | |
| ple | No. | Compound Name, ¹H-NMR, MS (ESI) |
| 28 | 28 | 5-[3-[1-(1-acetylazetidine-3-yl)-4-piperidyl]-2- |
| | | fluoro-phenyl]-3-[[5-[5-(difluoromethyl)-1,3,4- |
| | | oxadiazol-2-yl]-2-pyridyl]methyl]-1,3,4- |
| | | oxadiazol-2-one |
| | | ¹H NMR (400 MHz, CDCl₃) δ 9.31 (d, J = 1.6 Hz, |
| | | 1H), 8.43 (dd, J = 8.2, 2.2 Hz, 1H), 7.70-7.66 |
| | | (m, 1H), 7.53 (d, J = 8.2 Hz, 1H), 7.46-7.42 (m, |
| | | 1H), 7.26-7.22 (m, 1H), 7.08 (s, 0.2H), 6.95 |
| | | (0.5H), 6.82 (s, 0.2H), 5.26 (s, 2H), 4.18-4.14 |
| | | (m, 1H), 4.08-4.02 (m, 2H), 3.92-3.88 (m, 1H), |
| | | 3.20-3.17 (m, 1H), 3.00-2.94 (m, 3H), 2.07-2.00 |
| | | (m, 2H), 1.91-1.76 (m, 7H); LRMS (ES) m/z 570.55 |
| | | (M⁺ + 1). |

### Example 81: Synthesis of Compound 81, 5-[3-(1-cyclobutyl-4-piperidyl)-2-fluoro-phenyl]-3-[[5-[5-(difluoromethyl)-1,3,4-oxadiazol-2-yl]-2-pyridyl]methyl]-1,3,4-oxadiazol-2-thione

### [Step 1] Synthesis of tert-butyl 4-[2-fluoro-3-(2-thioxo-3H-1,3,4-oxadiazol-5-yl)phenyl]piperidin-1-carboxylate

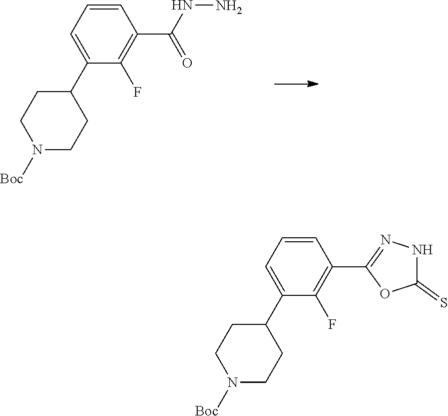 [SMILES: CC(C)(C)OC(=O)N1CCC(c2cccc(C(=O)NN)c2F)CC1 ; CC(C)(C)OC(=O)N1CCC(c2cccc(-c3n[nH]c(=S)o3)c2F)CC1]

A mixture in which tert-butyl 4-[2-fluoro-3-(hydrazinecarbonyl)phenyl]piperidine-1-carboxylate (100.00%, 1.100 g, 3.260 mmol) prepared in Step 3 of Example 26 and potassium ethylxanthate (100.00%, 0.627 g, 3.911 mmol) were mixed in ethanol (80 mL) at room temperature was heated to reflux overnight. The mixture was cooled to room temperature, and water was poured into the reaction mixture, followed by extraction with dichloromethane. The organic layer was washed with a saturated aqueous ammonium chloride solution, dried over anhydrous magnesium sulfate, filtered, and concentrated under reduced pressure. The concentrate was purified and concentrated by column chromatography (SiO₂, 24 g cartridge; ethyl acetate/hexane=0% to 40%) to obtain the title compound (0.9 g, 72.76%, white solid).

### [Step 2] Synthesis of tert-butyl 4-[3-[4-[[5-[5-(difluoromethyl)-1,3,4-oxadiazol-2-yl]-2-pyridyl]methyl]-5-thioxo-1, 3,4-oxadiazol-2-yl]-2-fluoro-phenyl]piperidine-1-carboxylate

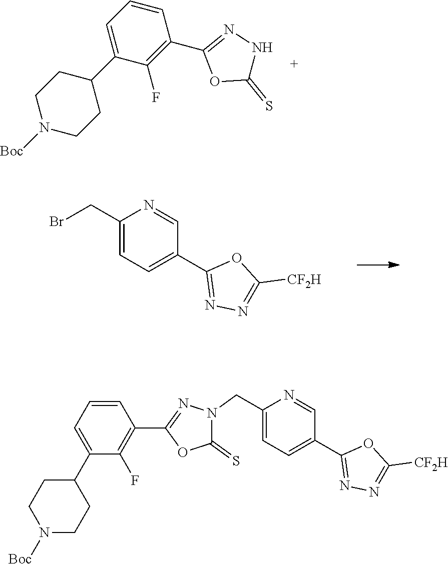 [SMILES: CC(C)(C)OC(=O)N1CCC(c2cccc(-c3n[nH]c(=S)o3)c2F)CC1 ; FC(F)c1nnc(-c2ccc(CBr)nc2)o1 ; CC(C)(C)OC(=O)N1CCC(c2cccc(-c3nn(Cc4ccc(-c5nnc(C(F)F)o5)cn4)c(=S)o3)c2F)CC1]

A solution in which tert-butyl 4-[2-fluoro-3-(2-thioxo-3H-1,3,4-oxadiazol-5-yl)phenyl]piperidine-1-carboxylate (100.00%, 0.500 g, 1.318 mmol) synthesized in Step 1 and potassium carbonate (100.00%, 0.196 g, 1.978 mmol) were dissolved in N,N-dimethylformamide (20 mL) was stirred at room temperature for 30 minutes. Then, 2-[6-(bromomethyl)-3-pyridyl]-5-(difluoromethyl)-1,3,4-oxadiazole (100.00%, 0.401 g, 1.383 mmol) and potassium iodide (100.00%, 0.109 g, 0.657 mmol) were added and further stirred overnight at the same temperature. Water was poured into the reaction mixture, and the mixture was extracted with ethyl acetate. The organic layer was washed with a saturated aqueous ammonium chloride solution, dried over anhydrous magnesium sulfate, filtered, and concentrated under reduced pressure. The concentrate was purified and concentrated by column chromatography (SiO₂, 24 g cartridge; ethyl acetate/hexane=0% to 30%) to obtain the title compound (0.61 g, 78.64%, white solid).

### [Step 3] Synthesis of 3-[[5-[5-(difluoromethyl)-1,3,4-oxadiazol-2-yl]-2-pyridyl]methyl]-5-[2-fluoro-3-(4-piperidyl)phenyl]-1,3,4-oxadiazol-2-thione 2,2,2-trifluoroacetic acid

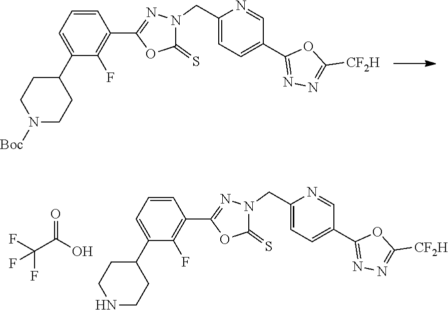 [SMILES: CC(C)(C)OC(=O)N1CCC(c2cccc(-c3nn(Cc4ccc(-c5nnc(C(F)F)o5)cn4)c(=S)o3)c2F)CC1 ; O=C(O)C(F)(F)F ; Fc1c(-c2nn(Cc3ccc(-c4nnc(C(F)F)o4)cn3)c(=S)o2)cccc1C1CCNCC1]

A solution in which tert-butyl 4-[3-[4-[[5-[5-(difluoromethyl)-1,3,4-oxadiazol-2-yl]-2-pyridyl]methyl]-5-thioxo-1,3,4-oxadiazol-2-yl]-2-fluoro-phenyl]piperidine-1-carboxylate (100.00%, 0.400 g, 0.680 mmol) synthesized in Step 2 and trifluoroacetic acid (100.00%, 0.232 g, 2.035 mmol) were dissolved in dichloromethane (20 mL) at room temperature was stirred for 3 hours at the same temperature. After removing the solvent from the reaction mixture under reduced pressure, the obtained product was used without further purification (0.37 g, 90.37%, yellow oil).

### [Step 4] Synthesis of Compound 81

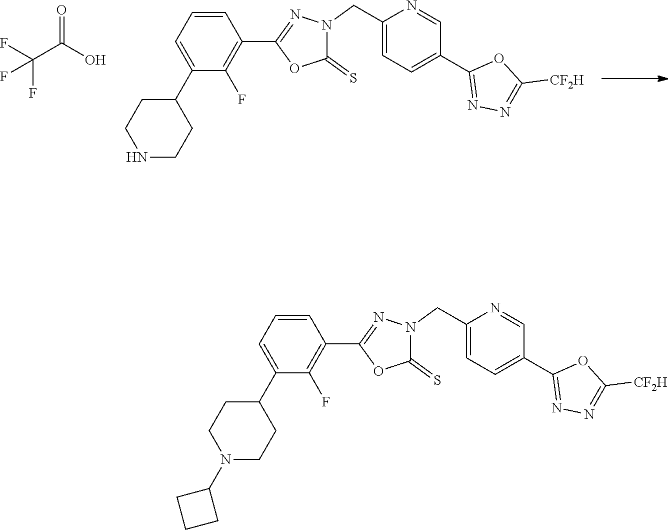 [SMILES: O=C(O)C(F)(F)F ; Fc1c(-c2nn(Cc3ccc(-c4nnc(C(F)F)o4)cn3)c(=S)o2)cccc1C1CCNCC1 ; Fc1c(-c2nn(Cc3ccc(-c4nnc(C(F)F)o4)cn3)c(=S)o2)cccc1C1CCN(C2CCC2)CC1]

A solution in which 3-[[5-[5-(difluoromethyl)-1,3,4-oxadiazol-2-yl]-2-pyridyl]methyl]-5-[2-fluoro-3-(4-piperidyl)phenyl]-1,3,4-oxadiazol-2-thione 2,2,2-trifluoroacetic acid (100.00%, 0.050 g, 0.083 mmol) synthesized in Step 3, cyclobutanone (100.00%, 0.012 g, 0.171 mmol), and N-ethyldiisopropylamine (100.00% solution, 0.029 mL, 0.167 mmol) were dissolved in dichloromethane (5 mL) was stirred at room temperature for 30 minutes. Then, sodium triacetoxyborohydride (100.00%, 0.053 g, 0.250 mmol) was added and further stirred overnight at the same temperature. Water was poured into the reaction mixture, and the mixture was extracted with dichloromethane. The organic layer was washed with a saturated aqueous solution of sodium hydrogen carbonate, dried over anhydrous magnesium sulfate, filtered, and concentrated under reduced pressure. The concentrate was purified and concentrated by column chromatography (SiO₂, 4 g cartridge; methanol/dichloromethane=0% to 5%) to obtain the title compound (0.029 g, 64.40%, white solid).

¹H NMR (400 MHz, CDCl₃) δ 9.31 (d, J=2.1 Hz, 1H), 8.38 (dd, J=8.2, 2.3 Hz, 1H), 7.83-7.79 (m, 2H), 7.49-7.45 (m, 1H), 7.26-7.22 (m, 1H), 7.08 (s, 0.2H), 6.95 (s, 0.5H), 6.82 (s, 0.2H), 4.73 (s, 2H), 3.15-3.12 (m, 2H), 3.03-3.00 (m, 1H), 2.88-2.84 (m, 1H), 2.12-2.02 (m, 6H), 1.98-1.88 (m, 4H), 1.79-1.68 (m, 2H); LRMS (ES) m/z 543.90 (M⁺+1).

Compounds in Table 12 below were synthesized according to substantially the same method as in Example 81.

TABLE 12

| | Com- | |
|---|---|---|
| Exam- | pound | |
| ple | No. | Compound Name, ¹H-NMR, MS (ESI) |
| 80 | 80 | 3-[[5-[5-(difluoromethyl)-1,3,4-oxadiazol-2-yl]- |
| | | 2-pyridyl]methyl]-5-[2-fluoro-3-(1-isopropyl-4- |
| | | piperidyl)phenyl]-1,3,4-oxadiazol-2-thione |
| | | ¹H NMR (400 MHz, CDCl₃) δ 9.31 (d, J = 2.1 Hz, |
| | | 1H), 8.39 (dd, J = 8.2, 2.2 Hz, 1H), 7.87-7.81 |
| | | (m, 2H), 7.54-7.50 (m, 1H), 7.32-7.29 (m, 1 H), |
| | | 7.08 (s, 0.2H), 6.95 (s, 0.5H), 6.82 (s, 0.2H), |
| | | 4.74 (s, 2H), 3.57-3.48 (m, 3H), 3.24-3.17 (m, |
| | | 1H), 2.89-2.83 (m, 2H), 2.43-2.34 (m, 2H), 2.07- |
| | | 2.04 (m, 2H), 1.39 (d, J = 6.7 Hz, 6H); LRMS (ES) |
| | | m/z 531.80 (M⁺ + 1). |
| 82 | 82 | 5-[3-(1-cyclopentyl-4-piperidyl)-2-fluoro- |
| | | phenyl]-3-[[5-[5-(difluoromethyl)-1,3,4- |
| | | oxadiazol-2-yl]-2-pyridyl]methyl]-1,3,4- |
| | | oxadiazol-2-thione |
| | | ¹H NMR (400 MHz, CDCl₃) δ 9.31 (d, J = 1.6 Hz, |
| | | 1H), 8.38 (dd, J = 8.2, 2.2 Hz, 1H), 7.85-7.81 |
| | | (m, 2H), 7.50-7.46 (m, 1H), 7.29-7.25 (m, 1 H), |
| | | 7.08 (s, 0.2H), 6.95 (s, 0.5H), 6.82 (s, 0.2H), |
| | | 4.73 (s, 2H), 3.56-3.54 (m, 2H), 3.16-3.11 (m, |
| | | 1H), 3.10-3.03 (m, 1H), 2.57-2.51 (m, 2H), 2.22- |
| | | 2.13 (m, 2H), 2.05-1.95 (m, 4H), 1.81 (brs, 4H), |
| | | 1.62 (brs, 2H); LRMS (ES) m/z 557.89 (M⁺ + 1). |
| 115 | 115 | 3-[[5-[5-(difluoromethyl)-1,3,4-oxadiazol-2-yl]- |
| | | 2-pyridyl]methyl]-5-[4-fluoro-3-(1-methyl-4- |
| | | piperidyl)phenyl]-1,3,4-oxadiazol-2-thione |
| | | ¹H NMR (400 MHz, CDCl₃) δ 9.31 (d, J = 2.8 Hz, |
| | | 1H), 8.40 (dd, J = 8.2, 2.2 Hz, 1H), 7.92-7.86 |
| | | (m, 2H), 7.82 (d, J = 8.4 Hz, 1H), 7.18-7.14 (m, |
| | | 1H), 6.96 (t, J = 51.6 Hz, 1H), 4.74 (s, 2H), |
| | | 3.22-3.20 (m, 2H), 3.00-2.94 (m, 1H), 2.50-2.33 |
| | | (m, 5H), 2.06-1.91 (m, 5H); LRMS (ES) m/z 503.0 |
| | | (M⁺ + 1). |
| 116 | 116 | 3-[[5-[5-(difluoromethyl)-1,3,4-oxadiazol-2-yl]- |
| | | 2-pyridyl]methyl]-5-[4-fluoro-3-(1-isopropyl-4- |
| | | piperidyl)phenyl]-1,3,4-oxadiazol-2-thione |
| | | ¹H NMR (400 MHz, CDCl₃) δ 9.323-9.316 (m, 1H), |
| | | 8.39 (dd, J = 8.2, 2.2 Hz, 1H), 7.96-7.94 (m, |
| | | 1H), 7.88-7.81 (m, 2H), 7.14 (dd, J = 9.8, 8.6 |
| | | Hz, 1H), 6.95 (t, J = 51.6 Hz, 1H), 4.74 (s, 2H), |
| | | 3.10 (s, 2H), 2.99-2.88 (m, 2H), 2.38 (s, 2H), |
| | | 1.91 (s, 4H), 1.14 (d, J = 6.0 Hz, 6H); LRMS (ES) |
| | | m/z 529.8 (M⁻ − 1). |

### Example 100: Synthesis of Compound 100, 3-[[5-[5-(difluoromethyl)-1,3,4-oxadiazol-2-yl]-2-pyridyl]methyl]-5-[6-(4-ethylpiperazin-1-yl)-2-pyridyl]-1,3,4-oxadiazol-2-thione

### [Step 1] Synthesis of tert-butyl

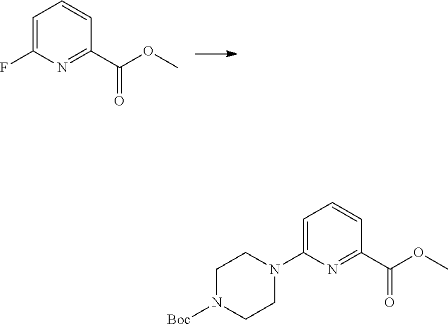 [SMILES: COC(=O)c1cccc(F)n1 ; COC(=O)c1cccc(N2CCN(C(=O)OC(C)(C)C)CC2)n1]

A solution in which methyl 6-fluoropyridin-2-carboxylate (100.00%, 0.500 g, 3.223 mmol), tert-butyl piperazin-1-carboxylate (100.00%, 1.200 g, 6.443 mmol) and N,N-diisopropylethylamine (100.00% solution, 0.844 mL, 4.800 mmol) were dissolved in dimethylsulfoxide (15 mL) at 130° C. was stirred overnight at the same temperature, and the reaction was terminated by lowering the temperature to room temperature. Water was poured into the reaction mixture, and the mixture was extracted with ethyl acetate. The organic layer was washed with a saturated aqueous sodium chloride solution, dried over anhydrous magnesium sulfate, filtered, and concentrated under reduced pressure. The concentrate was purified and concentrated by column chromatography (SiO₂, 24 g cartridge; ethyl acetate/hexane=0% to 60%) to obtain the title compound (0.736 g, 71.05%, yellow solid).

### [Step 2] Synthesis of tert-butyl 4-[6-(hydrazinecarbonyl)-2-pyridyl]piperazin-1-carboxylate

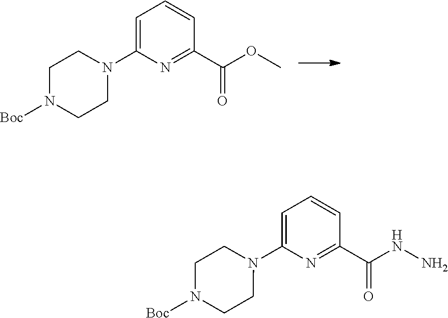 [SMILES: COC(=O)c1cccc(N2CCN(C(=O)OC(C)(C)C)CC2)n1 ; CC(C)(C)OC(=O)N1CCN(c2cccc(C(=O)NN)n2)CC1]

To a solution in which tert-butyl 4-(6-methoxycarbonyl-2-pyridyl)piperazin-1-carboxylate (100.00%, 0.736 g, 2.290 mmol) synthesized in Step 1 was dissolved in ethanol (10 mL) at room temperature, hydrazine monohydrate (100.00% solution, 1.113 mL, 22.900 mmol) was added and stirred at 60° C. for 18 hours. Then, the reaction was terminated by lowering the temperature to room temperature. A saturated aqueous ammonium chloride solution was poured into the reaction mixture, followed by extraction with dichloromethane. The organic layer was washed with a saturated aqueous sodium chloride solution, dried over anhydrous magnesium sulfate, filtered, and concentrated under reduced pressure. The concentrate was purified and concentrated by column chromatography (SiO₂, 4 g cartridge; methanol/dichloromethane=0% to 10%) to obtain the title compound (0.73 g, 99.18%, yellow solid).

### [Step 3] Synthesis of tert-butyl 4-[6-(2-thioxo-3H-1,3,4-oxadiazol-5-yl)-2-pyridyl]piperazin-1-carboxylate

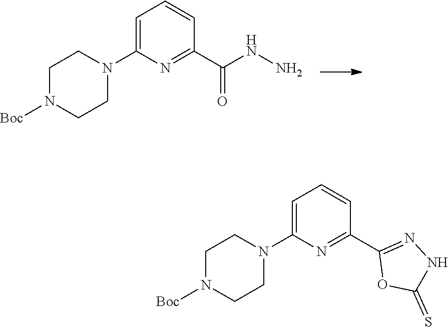 [SMILES: CC(C)(C)OC(=O)N1CCN(c2cccc(C(=O)NN)n2)CC1 ; CC(C)(C)OC(=O)N1CCN(c2cccc(-c3n[nH]c(=S)o3)n2)CC1]

A solution in which tert-butyl 4-[6-(hydrazinecarbonyl)-2-pyridyl]piperazin-1-carboxylate (100.00%, 0.730 g, 2.271 mmol) synthesized in Step 2 and potassium ethylxanthate (100.00%, 0.364 g, 2.271 mmol) were dissolved in ethanol (10 mL) at room temperature was stirred at 90° C. overnight. Then, the reaction was terminated by lowering the temperature to room temperature. A saturated aqueous ammonium chloride solution was poured into the reaction mixture, followed by extraction with dichloromethane. The organic layer was washed with a saturated aqueous sodium chloride solution, dried over anhydrous magnesium sulfate, filtered, and concentrated under reduced pressure. The concentrate was purified and concentrated by column chromatography (SiO₂, 40 g cartridge; dichloromethane/methanol=0% to 10%) to obtain the title compound (0.604 g, 73.18%, yellow solid).

### [Step 4] Synthesis of tert-butyl 4-[6-[4-[[5-[5-(difluoromethyl)-1,3,4-oxadiazol-2-yl]-2-pyridyl]methyl]-5-thioxo-1,3,4-oxadiazol-2-yl]-2-pyridyl]piperazin-1-carboxylate

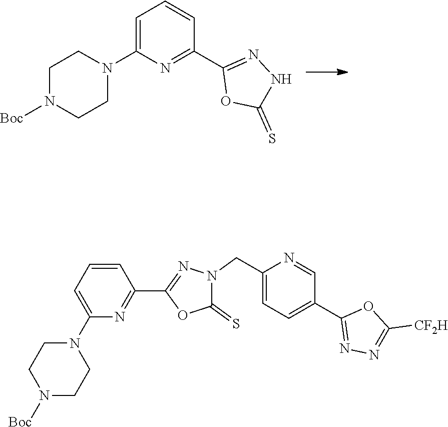 [SMILES: CC(C)(C)OC(=O)N1CCN(c2cccc(-c3n[nH]c(=S)o3)n2)CC1 ; CC(C)(C)OC(=O)N1CCN(c2cccc(-c3nn(Cc4ccc(-c5nnc(C(F)F)o5)cn4)c(=S)o3)n2)CC1]

A solution in which tert-butyl 4-[3-(2-thioxo-3H-1,3,4-oxadiazol-5-yl)phenyl]piperazin-1-carboxylate (100.00%, 0.700 g, 1.932 mmol) synthesized in Step 3, 2-[6-(bromomethyl)-3-pyridyl]-5-(difluoromethyl)-1,3,4-oxadiazole (100.00%, 1.000 equiv., 1.932 mmol), potassium carbonate (100.00%, 2.000 equiv., 3.863 mmol) and potassium iodide (100.00%, 2.000 equiv., 3.863 mmol) were dissolved in N,N-dimethylformamide (5 mL) at room temperature was stirred overnight at the same temperature. Water was poured into the reaction mixture, and the mixture was extracted with ethyl acetate. The organic layer was washed with a saturated aqueous sodium chloride solution, dried over anhydrous magnesium sulfate, filtered, and concentrated under reduced pressure. The concentrate was purified and concentrated by column chromatography (SiO₂, 12 g cartridge; ethyl acetate/hexane=0% to 50%) to obtain the title compound (0.14 g, 12.68%, yellow solid).

### [Step 5] Synthesis of 3-[[5-[5-(difluoromethyl)-1,3,4-oxadiazol-2-yl]-2-pyridyl]methyl]-5-(6-piperazin-1-yl-2-pyridyl)-1,3,4-oxadiazol-2-thione

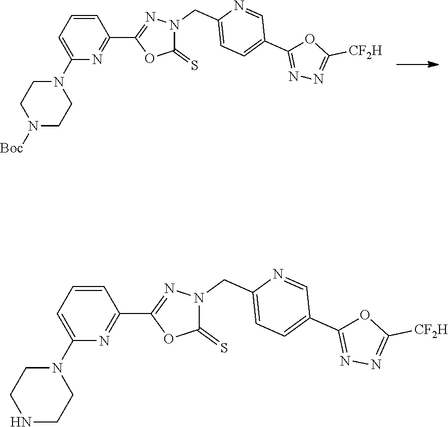 [SMILES: CC(C)(C)OC(=O)N1CCN(c2cccc(-c3nn(Cc4ccc(-c5nnc(C(F)F)o5)cn4)c(=S)o3)n2)CC1 ; FC(F)c1nnc(-c2ccc(Cn3nc(-c4cccc(N5CCNCC5)n4)oc3=S)nc2)o1]

A solution in which tert-butyl 4-[6-[4-[[5-[5-(difluoromethyl)-1,3,4-oxadiazol-2-yl]-2-pyridyl]methyl]-5-thioxo-1,3,4-oxadiazol-2-yl]-2-pyridyl]piperazin-1-carboxylate (100.00%, 0.384 g, 0.671 mmol) synthesized in Step 4 and trifluoroacetic acid (100.00% solution, 0.513 mL, 6.700 mmol) were dissolved in dichloromethane (3 mL) at room temperature was stirred overnight at the same temperature. A saturated aqueous solution of sodium hydrogen carbonate was poured into the reaction mixture, followed by extraction with dichloromethane. The organic layer was washed with a saturated aqueous sodium chloride solution, dried over anhydrous magnesium sulfate, filtered, and concentrated under reduced pressure. The obtained product was used without further purification (0.31 g, 97.83%, yellow oil).

### [Step 6] Synthesis of Compound 100

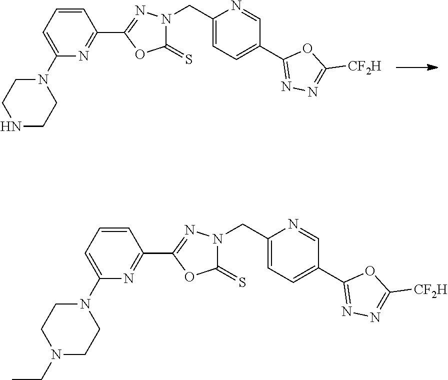 [SMILES: FC(F)c1nnc(-c2ccc(Cn3nc(-c4cccc(N5CCNCC5)n4)oc3=S)nc2)o1 ; CCN1CCN(c2cccc(-c3nn(Cc4ccc(-c5nnc(C(F)F)o5)cn4)c(=S)o3)n2)CC1]

A solution in which 3-[[5-[5-(difluoromethyl)-1,3,4-oxadiazol-2-yl]-2-pyridyl]methyl]-5-(6-piperazin-1-yl-2-pyridyl)-1,3,4-oxadiazol-2-thione (100.00%, 0.100 g, 0.212 mmol) synthesized in Step 5, acetaldehyde (100.00% solution, 0.024 mL, 0.429 mmol) and N-ethyl-N-isopropyl-propan-2-amine (100.00% solution, 0.074 mL, 0.425 mmol) were dissolved in dichloromethane (1 mL) was stirred at room temperature for 30 minutes, and sodium triacetoxyborohydride (100.00%, 0.134 g, 0.635 mmol) was added and further stirred at the same temperature for 18 hours. A saturated aqueous solution of sodium hydrogen carbonate was poured into the reaction mixture, followed by extraction with dichloromethane. The organic layer was washed with a saturated aqueous solution of sodium hydrogen carbonate, dried over anhydrous magnesium sulfate, filtered, and concentrated under reduced pressure. The concentrate was purified and concentrated by column chromatography (SiO₂, 4 g cartridge; dichloromethane/methanol=0% to 10%) to obtain the title compound (0.026 g, 24.55%, yellow solid).

¹H NMR (400 MHz, CDCl₃) δ 9.32 (d, J=1.6 Hz, 1H), 8.37 (dd, J=8.2, 2.2 Hz, 1H), 7.84 (d, J=8.4 Hz, 1H), 7.63-7.59 (m, 1H), 7.43 (d, J=7.2 Hz, 1H), 6.96 (t, J=51.6 Hz, 1H), 6.78 (d, J=8.8 Hz, 1H), 4.75 (s, 2H), 3.69 (t, J=5.0 Hz, 4H), 2.59 (t, J=5.0 Hz, 4H), 2.50 (q, J=7.2 Hz, 2H), 1.16 (t, J=7.2 Hz, 3H); LRMS (ES) m/z 501.5 (M⁺+1).

Compounds in Table 13 below were synthesized according to substantially the same method as in Example 100.

TABLE 13

| | Com- | |
|---|---|---|
| Exam- | pound | |
| ple | No. | Compound Name, ¹H-NMR, MS (ESI) |
| 75 | 75 | 3-[[5-[5-(difluoromethyl)-1,3,4-oxadiazol-2-yl]- |
| | | 2-pyridyl]methyl]-5-[3-(4-methylpiperazin-1- |
| | | yl)phenyl]-1,3,4-oxadiazol-2-thione |
| | | ¹H NMR (400 MHz, CDCl₃) δ 9.28 (s, 1H), 8.36 (d, |
| | | J = 10.0 Hz, 1H), 7.80 (d, J = 8.0 Hz, 1H), 7.51 |
| | | (s, 1H), 7.42 (d, J = 7.6 Hz, 1H), 7.35 (t, J = |
| | | 7.8 Hz, 1H), 7.06-6.80 (m, 2H), 4.71 (s, 2H), |
| | | 3.31 (t, J = 4.8 Hz, 4H), 2.69 (t, J = 4.8 Hz, |
| | | 4H), 2.40 (s, 3H); LRMS (ES) m/z 486.7 (M⁺ + 1). |
| 76 | 76 | 3-[[5-[5-(difluoromethyl)-1,3,4-oxadiazol-2-yl]- |
| | | 2-pyridyl]methyl]-5-[3-(4-isopropylpiperazin-1- |
| | | yl)phenyl]-1,3,4-oxadiazol-2-thione |
| | | ¹H NMR (400 MHz, CDCl₃) δ 9.27 (s, 1H), 8.35 (d, |
| | | J = 10.4 Hz, 1H), 7.79 (d, J = 8.4 Hz, 1H), 7.50 |
| | | (s, 1H), 7.42 (d, J = 7.6 Hz, 1H), 7.36-7.32 (m, |
| | | 1H), 7.06-6.80 (m, 2H), 4.70 (s, 2H), 3.37-3.32 |
| | | (m, 4H), 2.99-2.95 (m, 1H), 2.88-2.85 (m, 4H), |
| | | 1.17 (d, J = 9.6 Hz, 6H); LRMS (ES) m/z 514.7 |
| | | (M⁺ + 1). |
| 101 | 101 | 3-[[5-[5-(difluoromethyl)-1,3,4-oxadiazol-2-yl]- |
| | | 2-pyridyl]methyl]-5-[6-(4-isopropylpiperazin-1- |
| | | yl)-2-pyridyl]-1,3,4-oxadiazol-2-thione |
| | | ¹H NMR (400 MHz, CDCl₃) δ 9.32 (d, J = 2.0 Hz, |
| | | 1H), 8.37 (dd, J = 8.2, 2.2 Hz, 1H), 7.84 (d, J = |
| | | 8.0 Hz, 1H), 7.62-7.58 (m, 1H), 7.43 (d, J = |
| | | 7.2 Hz, 1H), 6.96 (t, J = 51.6 Hz, 1H), 6.78 (d, |
| | | J = 8.4 Hz, 1H), 4.75 (s, 2H), 3.68-3.67 (m, 4H), |
| | | 2.79-2.67 (m, 5H), 1.12 (d, J = 6.4 Hz, 6H); LRMS |
| | | (ES) m/z 514.6 (M⁺ + 1). |
| 102 | 102 | 5-[6-(4-cyclopentylpiperazin-1-yl)-2-pyridyl]-3- |
| | | [[5-[5-(difluoromethyl)-1,3,4-oxadiazol-2-yl]-2- |
| | | pyridyl]methyl]-1,3,4-oxadiazol-2-thione |
| | | ¹H NMR (400 MHz, CDCl₃) δ 9.31 (d, J = 1.6 Hz, |
| | | 6H), 8.37 (dd, J = 8.2, 2.2 Hz, 1H), 7.84 (d, J = |
| | | 8.4 Hz, 1H), 7.62-7.58 (m, 1H), 7.43 (d, J = |
| | | 7.2 Hz, 6H), 6.95 (t, J = 51.6 Hz, 1H), 6.77 (d, |
| | | J = 8.4 Hz, 1H), 4.75 (s, 2H), 3.68 (t, J = 5.0 |
| | | Hz, 4H), 2.66-2.51 (m, 5H), 1.96-1.89 (m, 2H), |
| | | 1.78-1.70 (m, 2H), 1.62-1.45 (m, 4H); LRMS (ES) |
| | | m/z 541.8 (M⁺ + 1). |
| 103 | 103 | 3-[[5-[5-(difluoromethyl)-1,3,4-oxadiazol-2-yl]- |
| | | 3-fluoro-2-pyridyl]methyl]-5-[6-(4- |
| | | ethylpiperazin-1-yl)-2-pyridyl]-1,3,4-oxadiazol- |
| | | 2-thione |
| | | ¹H NMR (400 MHz, CDCl₃) δ 9.12 (s, 1H), 8.15 (dd, |
| | | J = 9.2, 1.6 Hz, 1H), 7.63-7.59 (m, 1H), 7.45 (d, |
| | | J = 7.2 Hz, 1H), 6.96 (t, J = 51.6 Hz, 1H), 6.79 |
| | | (d, J = 8.4 Hz, 1H), 4.83 (d, J = 1.6 Hz, 2H), |
| | | 3.69 (t, J = 5.2 Hz, 4H), 2.59 (t, J = 5.0 Hz, |
| | | 4H), 2.51 (q, J = 7.2 Hz, 2H), 1.17 (t, J = 7.2 |
| | | Hz, 3H); LRMS (ES) m/z 519.8 (M⁺ + 1). |
| 104 | 104 | 3-[[5-[5-(difluoromethyl)-1,3,4-oxadiazol-2-yl]- |
| | | 3-fluoro-2-pyridyl]methyl]-5-[6-(4- |
| | | isopropylpiperazin-1-yl)-2-pyridyl]-1,3,4- |
| | | oxadiazol-2-thione |
| | | ¹H NMR (400 MHz, CDCl₃) δ 9.12 (s, 1H), 8.15 (dd, |
| | | J = 9.0, 1.8 Hz, 1H), 7.63-7.59 (m, 1H), 7.45 (d, |
| | | J = 7.2 Hz, 1H), 6.96 (t, J = 51.6 Hz, 1H), 6.78 |
| | | (d, J = 8.8 Hz, 1H), 4.83 (d, J = 1.6 Hz, 2H), |
| | | 3.68 (t, J = 4.8 Hz, 4H), 2.82-2.66 (m, 5H), 1.12 |
| | | (d, J = 6.4 Hz, 6H); LRMS (ES) m/z 533.8 (M⁺ + 1). |
| 105 | 105 | 3-[[5-[5-(difluoromethyl)-1,3,4-oxadiazol-2-yl]- |
| | | 2-pyridyl]methyl]-5-[2-(4-isopropylpiperazin-1- |
| | | yl)-4-pyridyl]-1,3,4-oxadiazol-2-thione |
| | | ¹H NMR (400 MHz, CDCl₃) δ 9.32 (d, J = 1.6 Hz, |
| | | 1H), 8.40 (dd, J = 8.0, 2.4 Hz, 1H), 8.33 (d, J = |
| | | 4.8 Hz, 1H), 7.82 (d, J = 8.0 Hz, 1H), 7.20 (s, |
| | | 1H), 7.11 (dd, J = 5.2, 1.2 Hz, 1H), 6.96 (t, J = |
| | | 51.6 Hz, 1H), 4.76 (s, 2H), 3.67-3.65 (m, 4H), |
| | | 2.78-2.75 (m, 1H), 2.68-2.65 (m, 4H), 1.11 (d, J = |
| | | 6.4 Hz, 6H); LRMS (ES) m/z 515.2 (M⁺ + 1). |
| 106 | 106 | 5-[2-(4-cyclopentylpiperazin-1-yl)-4-pyridyl]-3- |
| | | [[5-[5-(difluoromethyl)-1,3,4-oxadiazol-2-yl]-2- |
| | | pyridyl]methyl]-1,3,4-oxadiazol-2-thione |
| | | ¹H NMR (400 MHz, CDCl₃) δ 9.32 (d, J = 2.0 Hz, |
| | | 1H), 8.40 (dd, J = 8.2, 2.2 Hz, 1H), 8.33 (d, J = |
| | | 5.2 Hz, 1H), 7.82 (d, J = 8.0 Hz, 1H), 7.19 (s, |
| | | 1H), 7.13-7.12 (m, 1H), 6.96 (t, J = 51.6 Hz, |
| | | 1H), 4.76 (s, 2H), 3.68-3.65 (m, 4H), 2.66-2.54 |
| | | (m, 5H), 1.94-1.92 (m, 2H), 1.77-1.71 (m, 2H), |
| | | 1.59-1.54 (m, 2H), 1.52-1.46 (m, 2H); LRMS (ES) |
| | | m/z 541.8 (M⁺ + 1). |
| 126 | 126 | 3-[[5-[5-(difluoromethyl)-1,3,4-oxadiazol-2-yl]- |
| | | 2-pyridyl]methyl]-5-[2-(4-methylpiperazin-1- |
| | | yl)pyrimidin-4-yl]-1,3,4-oxadiazol-2-thione |
| | | ¹H NMR (400 MHz, CDCl₃) δ 9.32 (d, J = 1.6 Hz, |
| | | 1H), 8.50 (d, J = 4.8 Hz, 1H), 8.39 (dd, J = 8.2, |
| | | 2.2 Hz, 1H), 7.82 (d, J = 8.4 Hz, 1H), 7.21 (d, |
| | | J = 4.8 Hz, 1H), 6.96 (t, J = 51.6 Hz, 1H), 4.77 |
| | | (s, 2H), 3.94 (t, J = 4.8 Hz, 4H), 2.51 (t, J = |
| | | 5.2 Hz, 4H), 2.37 (s, 3H); LRMS (ES) m/z 488.3 |
| | | (M⁺ + 1). |
| 127 | 127 | 3-[[5-[5-(difluoromethyl)-1,3,4-oxadiazol-2-yl]- |
| | | 2-pyridyl]methyl]-5-[2-(4-isopropylpiperazin-1- |
| | | yl)pyrimidin-4-yl]-1,3,4-oxadiazol-2-thione |
| | | ¹H NMR (400 MHz, CDCl₃) δ 9.32 (d, J = 1.6 Hz, |
| | | 1H), 8.49 (d, J = 4.8 Hz, 1H), 8.39 (dd, J = 8.2, |
| | | 2.2 Hz, 1H), 7.82 (d, J = 8.0 Hz, 1H), 7.20 (d, |
| | | J = 4.8 Hz, 1H), 6.96 (t, J = 51.6 Hz, 1H), 4.77 |
| | | (s, 2H), 3.93 (t, J = 5.0 Hz, 4H), 2.80-2.73 (m, |
| | | 1H), 2.61 (t, J = 5.0 Hz, 4H), 1.10 (d, J = 6.4 |
| | | Hz, 6H); LRMS (ES) m/z 516.3 (M⁺ + 1). |

### Example 41: Synthesis of Compound 41, 3-[[5-[5-(difluoromethyl)-1,3,4-oxadiazol-2-yl]-2-pyridyl]methyl]-5-[2-fluoro-3-(1-methyl-4-piperidyl)phenyl]-1,3,4-thiadiazol-2-one

### [Step 1] Synthesis of tert-butyl 4-[3-(aminocarbonothioyl)-2-fluoro-phenyl]piperidin-1-carboxylate

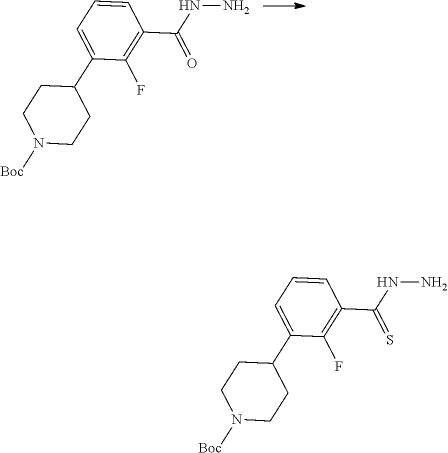 [SMILES: CC(C)(C)OC(=O)N1CCC(c2cccc(C(=O)NN)c2F)CC1 ; CC(C)(C)OC(=O)N1CCC(c2cccc(C(=S)NN)c2F)CC1]

A solution in which tert-butyl 4-[2-fluoro-3-(hydrazinecarbonyl)phenyl]piperidin-1-carboxylate (100.00%, 0.200 g, 0.593 mmol) prepared in Step 3 of Example 26 and Lawesson's reagent (100.00%, 0.288 g, 0.712 mmol) were dissolved in toluene (20 mL) at room temperature was stirred overnight at the same temperature. Water was poured into the reaction mixture, and the mixture was extracted with dichloromethane. The organic layer was washed with a saturated aqueous ammonium chloride solution, dried over anhydrous magnesium sulfate, filtered, and concentrated under reduced pressure. The obtained product was used without further purification (0.11 g, 52.51%, yellow oil).

### [Step 2] Synthesis of tert-butyl 4-[2-fluoro-3-(2-oxo-3H-1,3,4-thiadiazol-5-yl)phenyl]piperidin-1-carboxylate

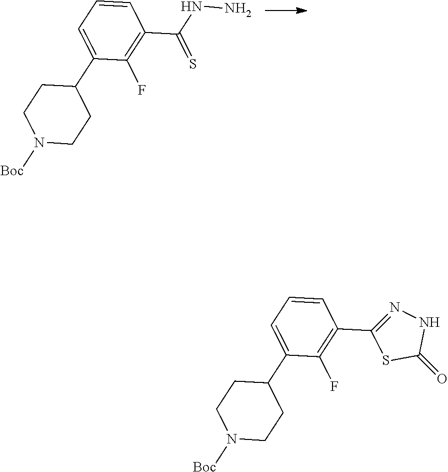 [SMILES: CC(C)(C)OC(=O)N1CCC(c2cccc(C(=S)NN)c2F)CC1 ; CC(C)(C)OC(=O)N1CCC(c2cccc(-c3n[nH]c(=O)s3)c2F)CC1]

A solution in which tert-butyl 4-[3-(aminocarbonothioyl)-2-fluorophenyl]piperidin-1-carboxylate (100.00%, 0.200 g, 0.566 mmol) synthesized in Step 1 and 1,1′-carbonylbis-1H-imidazole (100.00%, 0.110 g, 0.678 mmol) were dissolved in dichloromethane (20 mL) at room temperature was stirred overnight at the same temperature. Water was poured into the reaction mixture, and the mixture was extracted with dichloromethane. The organic layer was washed with a saturated aqueous ammonium chloride solution, dried over anhydrous magnesium sulfate, filtered, and concentrated under reduced pressure. The concentrate was purified and concentrated by column chromatography (SiO₂, 12 g cartridge; ethyl acetate/hexane=0% to 40%) to obtain the title compound (0.15 g, 69.86%, white solid).

### [Step 3] Synthesis of tert-butyl 4-[3-[4-[[5-[5-(difluoromethyl)-1,3,4-oxadiazol-2-yl]-2-pyridyl]methyl]-5-oxo-1,3,4-thiadiazol-2-yl]-2-fluoro-phenyl]piperidin-1-carboxylate

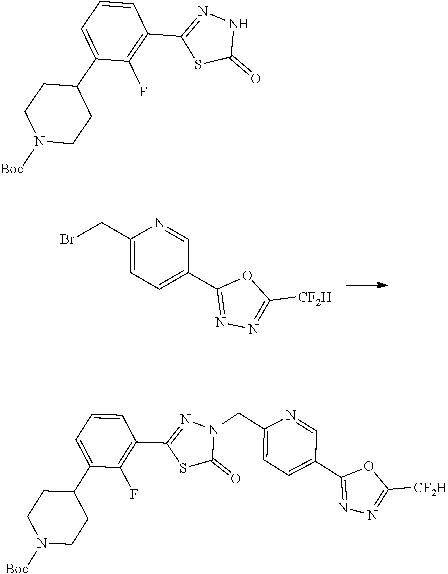 [SMILES: CC(C)(C)OC(=O)N1CCC(c2cccc(-c3n[nH]c(=O)s3)c2F)CC1 ; FC(F)c1nnc(-c2ccc(CBr)nc2)o1 ; CC(C)(C)OC(=O)N1CCC(c2cccc(-c3nn(Cc4ccc(-c5nnc(C(F)F)o5)cn4)c(=O)s3)c2F)CC1]

To a solution in which tert-butyl 4-[2-fluoro-3-(2-oxo-3H-1,3,4-thiadiazol-5-yl)phenyl]piperidin-1-carboxylate (100.00%, 0.073 g, 0.192 mmol) synthesized in Step 2 and potassium carbonate (100.00%, 0.029 g, 0.293 mmol) were dissolved in N,N-dimethylformamide (20 mL) at room temperature, 2-[6-(bromomethyl)-3-pyridyl]-5-(difluoromethyl)-1,3,4-oxadiazole (100.00%, 0.059 g, 0.203 mmol) and potassium iodide (100.00%, 0.016 g, 0.096 mmol) were added and stirred at the same temperature for 30 minutes. Water was poured into the reaction mixture, and the mixture was extracted with ethyl acetate. The organic layer was washed with a saturated aqueous ammonium chloride solution, dried over anhydrous magnesium sulfate, filtered, and concentrated under reduced pressure. The concentrate was purified and concentrated by column chromatography (SiO₂, 12 g cartridge; ethyl acetate/hexane=0% to 30%) to obtain the title compound (0.05 g, 44.15%, white solid).

### [Step 4] Synthesis of 3-[[5-[5-(difluoromethyl)-1,3,4-oxadiazol-2-yl]-2-pyridyl]methyl]-5-[2-fluoro-3-(4-piperidyl)phenyl]-1,3,4-thiadiazol-2-one 2,2,2-trifluoroacetic acid

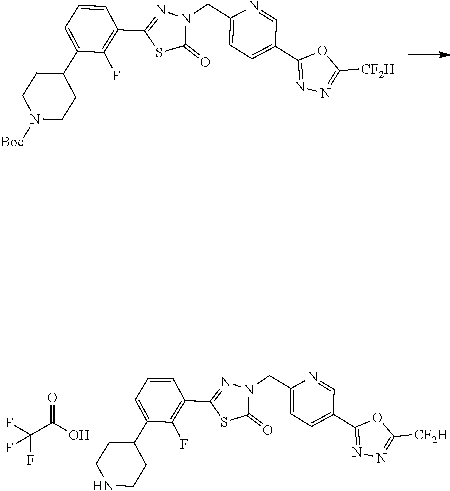 [SMILES: CC(C)(C)OC(=O)N1CCC(c2cccc(-c3nn(Cc4ccc(-c5nnc(C(F)F)o5)cn4)c(=O)s3)c2F)CC1 ; O=C(O)C(F)(F)F ; O=c1sc(-c2cccc(C3CCNCC3)c2F)nn1Cc1ccc(-c2nnc(C(F)F)o2)cn1]

A solution in which tert-butyl 4-[3-[4-[[5-[5-(difluoromethyl)-1,3,4-oxadiazol-2-yl]-2-pyridyl]methyl]-5-oxo-1,3,4-thiadiazol-2-yl]-2-fluoro-phenyl]piperidin-1-carboxylate (100.00%, 0.058 g, 0.099 mmol) synthesized in Step 3 and trifluoroacetic acid (100.00%, 0.034 g, 0.298 mmol) were dissolved in dichloromethane (10 mL) at room temperature was stirred for 3 hours at the same temperature. After removing the solvent from the reaction mixture under reduced pressure, the obtained product was used without further purification (0.055 g, 92.64%, yellow oil).

### [Step 5] Synthesis of Compound 41

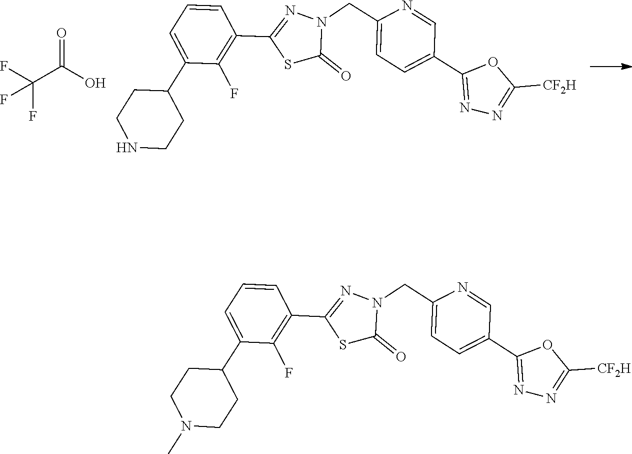

A solution in which 3-[[5-[5-(difluoromethyl)-1,3,4-oxadiazol-2-yl]-2-pyridyl]methyl]-5-[2-fluoro-3-(4-piperidyl)phenyl]-1,3,4-thiadiazol-2-one 2,2,2-trifluoroacetic acid (100.00%, 0.055 g, 0.091 mmol) synthesized in Step 4, N-ethyldiisopropylamine (100.00% solution, 0.032 mL, 0.184 mmol), formaldehyde (100.00%, 0.005 g, 0.167 mmol) and sodium triacetoxyborohydride (100.00%, 0.039 g, 0.184 mmol) were dissolved in dichloromethane (5 mL) at room temperature was stirred overnight at the same temperature. Water was poured into the reaction mixture, and the mixture was extracted with dichloromethane. The organic layer was washed with a saturated aqueous solution of sodium hydrogen carbonate, dried over anhydrous magnesium sulfate, filtered, and concentrated under reduced pressure. The concentrate was purified and concentrated by column chromatography (SiO₂, 4 g cartridge; methanol/dichloromethane=0% to 5%) to obtain the title compound (0.031 g, 67.58%, white solid).

¹H NMR (400 MHz, CDCl₃) δ 9.32 (d, J=1.6 Hz, 1H), 8.41 (dd, J=8.2, 2.2 Hz, 1H), 7.77-7.73 (m, 1H), 7.46 (d, J=8.2 Hz, 1H), 7.40-7.36 (m, 1H), 7.28-7.17 (m, 1H), 5.44 (s, 2H), 3.13-3.10 (m, 2H), 2.99-2.92 (m, 1H), 2.43 (s, 3H), 2.26-2.20 (m, 2H), 1.98-1.86 (m, 4H); LRMS (ES) m/z 503.7 (M⁺+1).

Compounds in Table 14 below were synthesized according to substantially the same method as in Example 41.

TABLE 14

| | Com- | |
|---|---|---|
| Exam- | pound | |
| ple | No. | Compound Name, ¹H-NMR, MS (ESI) |
| 55 | 55 | 3-[[5-[5-(difluoromethyl)-1,3,4-oxadiazol-2-yl]- |
| | | 2-pyridyl]methyl]-5-[4-(1-isopropyl-4- |
| | | piperidyl)phenyl]-1,3,4-thiadiazol-2-one |
| | | ¹H NMR (400 MHz, CDCl₃) δ 9.32 (d, J = 1.6 Hz, |
| | | 1H), 8.40 (dd, J = 8.2, 2.2 Hz, 1H), 7.63 (d, J = |
| | | 8.3 Hz, 2H), 7.46 (d, J = 8.2 Hz, 1H), 7.33 (d, |
| | | J = 8.3 Hz, 2H), 7.08 (s, 0.2H), 6.95 (s, 0.5H), |
| | | 6.82 (s, 0.2H), 5.41 (s, 2H), 3.19-3.16 (m, 2H), |
| | | 3.01-2.97 (m, 1H), 2.63-2.56 (m, 1H), 2.46-2.40 |
| | | (m, 2H), 2.06-2.02 (m, 2H), 1.99-1.89 (m, 2H), |
| | | 1.19 (d, J = 6.6 Hz, 6H); LRMS (ES) m/z 513.85 |
| | | (M⁺ + 1). |
| 56 | 56 | 3-[[5-[5-(difluoromethyl)-1,3,4-oxadiazol-2-yl]- |
| | | 2-pyridyl]methyl]-5-[4-[1-(oxetane-3-yl)-4- |
| | | piperidyl]phenyl]-1,3,4-thiadiazol-2-one |
| | | ¹H NMR (400 MHz, CDCl₃) δ 9.32 (d, J = 1.6 Hz, |
| | | 1H), 8.40 (dd, J = 8.2, 2.2 Hz, 1H), 7.64 (d, J = |
| | | 8.3 Hz, 2H), 7.46 (d, J = 8.2 Hz, 1H), 7.31 (d, |
| | | J = 8.3 Hz, 2H), 7.08 (s, 0.2H), 6.95 (s, 0.5H), |
| | | 6.82 (s, 0.2H), 5.41 (s, 2H), 4.71-4.64 (m, 4H), |
| | | 3.55-3.49 (m, 1H), 2.91-2.88 (m, 2H), 2.62-2.54 |
| | | (m, 1H), 1.98-1.92 (m, 2H), 1.89-1.80 (m, 4H); |
| | | LRMS (ES) m/z 527.84 (M⁺ + 1). |
| 57 | 57 | 3-[[5-[5-(difluoromethyl)-1,3,4-oxadiazol-2-yl]- |
| | | 2-pyridyl]methyl]-5-[2-fluoro-3-(1-isopropyl-4- |
| | | piperidyl)phenyl]-1,3,4-thiadiazol-2-one |
| | | ¹H NMR (400 MHz, CDCl₃) δ 9.32 (d, J = 1.6 Hz, |
| | | 1H), 8.41 (dd, J = 8.2, 2.2 Hz, 1H), 7.77-7.73 |
| | | (m, 1H), 7.47-7.41 (m, 2H), 7.21-7.17 (m, 1H), |
| | | 7.08 (s, 0.2H), 6.95 (s, 0.5H), 6.82 (s, 0.2H), |
| | | 5.44 (s, 2H), 3.19-3.16 (m, 2H), 3.03-2.97 (m, |
| | | 2H), 2.55-2.44 (m, 2H), 2.06-2.02 (m, 2H), 1.93- |
| | | 1.90 (m, 2H), 1.20 (d, J = 6.4 Hz, 6H); LRMS (ES) |
| | | m/z 531.9 (M⁺ + 1). |
| 58 | 58 | 3-[[5-[5-(difluoromethyl)-1,3,4-oxadiazol-2-yl]- |
| | | 2-pyridyl]methyl]-5-[2-fluoro-3-[1-(oxetane-3- |
| | | yl)-4-piperidyl]phenyl]-1,3,4-thiadiazol-2-one |
| | | ¹H NMR (400 MHz, CDCl₃) δ 9.32 (d, J = 1.6 Hz, |
| | | 1H), 8.41 (dd, J = 8.2, 2.2 Hz, 1H), 7.78-7.73 |
| | | (m, 1H), 7.46 (d, J = 8.2 Hz, 1H), 7.40-7.28 (m, |
| | | 1H), 7.25-7.18 (m, 1H), 7.08 (s, 0.2H), 6.95 (s, |
| | | 0.5H), 6.82 (s, 0.2H), 5.44 (s, 2H), 4.72-4.65 |
| | | (m, 4H), 3.57-3.51 (m, 1H), 3.00-2.89 (m, 3H), |
| | | 2.03-1.96 (m, 2H), 1.88-1.80 (m, 4H); LRMS (ES) |
| | | m/z 545.9 (M⁺ + 1). |

### Example 20: Synthesis of Compound 20, 3-[[5-[5-(difluoromethyl)-1,3,4-oxadiazol-2-yl]-2-pyridyl]methyl]-5-(4-piperazin-1-ylphenyl)-1,3,4-oxadiazol-2-one

### [Step 1] Synthesis of tert-butyl 4-(4-methoxycarbonylphenyl)piperazin-1-carboxylate

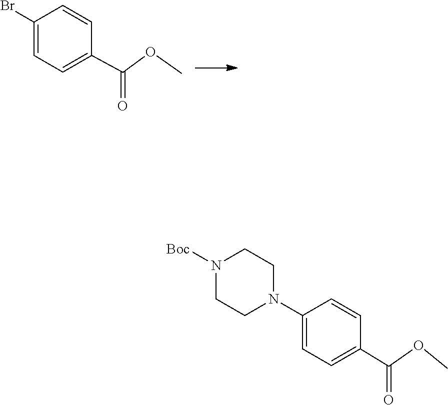 [SMILES: COC(=O)c1ccc(Br)cc1 ; COC(=O)c1ccc(N2CCN(C(=O)OC(C)(C)C)CC2)cc1]

A solution in which methyl 4-bromobenzoate (100.00%, 0.500 g, 2.325 mmol), tert-butyl piperazin-1-carboxylate (100.00%, 1.200 equiv., 2.790 mmol), tris(dibenzylideneacetone)dipalladium (100.00%, 5.000 mol %, 0.116 mmol), Xphos (100.00%, 5.000 mol %, 0.116 mmol) and cesium carbonate (100.00%, 2.000 equiv., 4.650 mmol) were dissolved in toluene (50 mL) at 110° C. was stirred overnight at the same temperature. Then, the reaction was terminated by lowering the temperature to room temperature. Water was poured into the reaction mixture, and the mixture was extracted with dichloromethane. The organic layer was washed with a saturated aqueous sodium chloride solution, dried over anhydrous magnesium sulfate, filtered, and concentrated under reduced pressure. The concentrate was purified and concentrated by column chromatography (SiO₂, 12 g cartridge; ethyl acetate/hexane=0% to 30%) to obtain the title compound (0.73 g, 97.99%, brown solid).

### [Step 2] Synthesis of tert-butyl 4-[4-(hydrazinecarbonyl)phenyl]piperazin-1-carboxylate

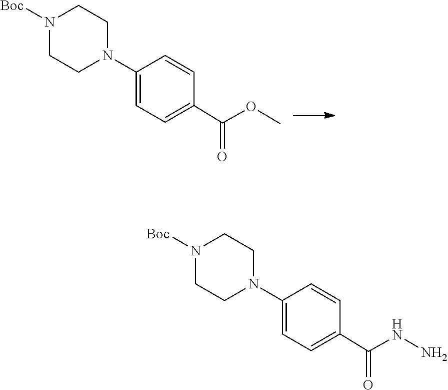 [SMILES: COC(=O)c1ccc(N2CCN(C(=O)OC(C)(C)C)CC2)cc1 ; CC(C)(C)OC(=O)N1CCN(c2ccc(C(=O)NN)cc2)CC1]

A solution in which tert-butyl 4-(4-methoxycarbonylphenyl)piperazin-1-carboxylate (100.00%, 0.770 g, 2.403 mmol) synthesized in Step 1 and hydrazine monohydrate (100.00%, 10.000 equiv., 24.030 mmol) were dissolved in dichloromethane (5 mL) at room temperature was stirred at the same temperature. Water was poured into the reaction mixture, and the mixture was extracted with dichloromethane. The organic layer was washed with a saturated aqueous sodium chloride solution, dried over anhydrous magnesium sulfate, filtered, and concentrated under reduced pressure. The concentrate was purified and concentrated by column chromatography (SiO₂, 12 g cartridge; ethyl acetate/hexane=0% to 30%) to obtain the title compound (0.77 g, 100.0%, yellow solid).

### [Step 3] Synthesis of tert-butyl 4-[4-(2-oxo-3H-1,3,4-oxadiazol-5-yl)phenyl]piperazin-1-carboxylate

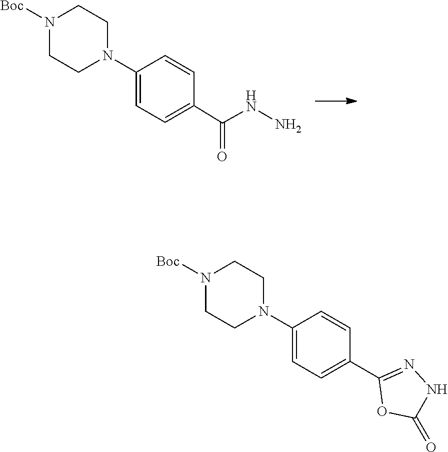 [SMILES: CC(C)(C)OC(=O)N1CCN(c2ccc(C(=O)NN)cc2)CC1 ; CC(C)(C)OC(=O)N1CCN(c2ccc(-c3n[nH]c(=O)o3)cc2)CC1]

A solution in which tert-butyl 4-[4-(hydrazinecarbonyl)phenyl]piperazin-1-carboxylate (100.00%, 0.770 g, 2.403 mmol) synthesized in Step 2 and 1,1′-carbonylbis-1H-imidazole (100.00%, 1.500 equiv., 3.605 mmol) were dissolved in dichloromethane (5 mL) at room temperature was heated under reflux overnight. Then, the reaction was terminated by lowering the temperature to room temperature. Water was poured into the reaction mixture, and the mixture was extracted with dichloromethane. The organic layer was washed with a saturated aqueous sodium chloride solution, dried over anhydrous magnesium sulfate, filtered, and concentrated under reduced pressure. The concentrate was purified and concentrated by column chromatography (SiO₂, 12 g cartridge; ethyl acetate/hexane=0% to 100%) to obtain the title compound (0.5 g, 60.06%, yellow solid).

### [Step 4] Synthesis of tert-butyl 4-[4-[4-[[5-[5-(difluoromethyl)-1,3,4-oxadiazol-2-yl]-2-pyridyl]methyl]-5-oxo-1,3,4-oxadiazol-2-yl]phenyl]piperazin-1-carboxylate

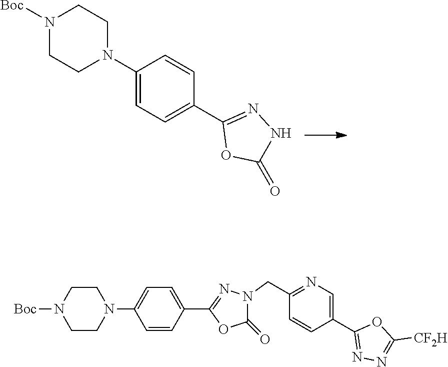 [SMILES: CC(C)(C)OC(=O)N1CCN(c2ccc(-c3n[nH]c(=O)o3)cc2)CC1 ; CC(C)(C)OC(=O)N1CCN(c2ccc(-c3nn(Cc4ccc(-c5nnc(C(F)F)o5)cn4)c(=O)o3)cc2)CC1]

A solution in which tert-butyl 4-[4-(2-oxo-3H-1,3,4-oxadiazol-5-yl)phenyl]piperazin-1-carboxylate (100.00%, 0.500 g, 1.443 mmol) synthesized in Step 3 and 2-[6-(bromomethyl)-3-pyridyl]-5-(difluoromethyl)-1,3,4-oxadiazole (100.00%, 1.200 equiv., 1.732 mmol), potassium carbonate (100.00%, 2.000 equiv., 2.887 mmol) and potassium iodide (100.00%, 1.100 equiv., 1.588 mmol) were dissolved in N,N-dimethylformamide (25 mL) at room temperature was stirred at the same temperature. Water was poured into the reaction mixture, and the mixture was extracted with ethyl acetate. The organic layer was washed with a saturated aqueous sodium chloride solution, dried over anhydrous magnesium sulfate, filtered, and concentrated under reduced pressure. The concentrate was purified and concentrated by column chromatography (SiO₂, 12 g cartridge; ethyl acetate/hexane=0% to 50%) to obtain the title compound (0.9 g, 112.2%, yellow solid).

### [Step 5] Synthesis of Compound 20, 3-[[5-[5-(difluoromethyl)-1,3,4-oxadiazol-2-yl]-2-pyridyl]methyl]-5-(4-piperazin-1-ylphenyl)-1,3,4-oxadiazol-2-one

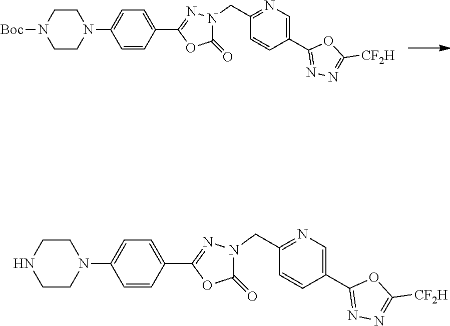 [SMILES: CC(C)(C)OC(=O)N1CCN(c2ccc(-c3nn(Cc4ccc(-c5nnc(C(F)F)o5)cn4)c(=O)o3)cc2)CC1 ; O=c1oc(-c2ccc(N3CCNCC3)cc2)nn1Cc1ccc(-c2nnc(C(F)F)o2)cn1]

A solution in which tert-butyl 4-[4-[4-[[5-[5-(difluoromethyl)-1,3,4-oxadiazol-2-yl]-2-pyridyl]methyl]-5-oxo-1,3,4-oxadiazol-2-yl]phenyl]piperazin-1-carboxylate (100.00%, 0.900 g, 1.620 mmol) synthesized in Step 4 and 2,2,2-trifluoroacetic acid (100.00%, 10.000 equiv., 16.200 mmol) were dissolved in dichloromethane (50 mL) at room temperature was stirred at the same temperature. An aqueous solution of N-sodium hydrogen carbonate was poured into the reaction mixture, followed by extraction with dichloromethane. The organic layer was washed with a saturated aqueous sodium chloride solution, dried over anhydrous magnesium sulfate, filtered, and concentrated under reduced pressure. The concentrate was purified and concentrated by column chromatography (SiO₂, 4 g cartridge; dichloromethane/methanol=0% to 10%) to obtain the title compound (0.12 g, 16.26%, white solid).

¹H NMR (400 MHz, CDCl₃) δ 9.29 (s, 1H), 8.39 (d, J=10.0 Hz, 1H), 7.70 (d, J=8.8 Hz, 2H), 7.49 (d, J=8.0 Hz, 1H), 7.06-6.80 (m, 3H), 5.18 (s, 2H), 3.27 (s, 4H), 3.02 (s, 4H); LRMS (ES) m/z 455.5 (M⁺+1).

Compounds in Table 15 below were synthesized according to substantially the same method as in Example 20.

TABLE 15

| | Com- | |
|---|---|---|
| Exam- | pound | |
| ple | No. | Compound Name, ¹H-NMR, MS (ESI) |
| 23 | 23 | 3-[[5-[5-(difluoromethyl)-1,3,4-oxadiazol-2-yl]- |
| | | 2-pyridyl]methyl]-5-(2-fluoro-4-piperazin-1-yl- |
| | | phenyl)-1,3,4-oxadiazol-2-one |
| | | ¹H NMR (400 MHz, CDCl₃) δ 9.29 (s, 1H), 8.40 (d, |
| | | J = 10.4 Hz, 1H), 7.64 (t, J = 8.8 Hz, 1H), 7.50 |
| | | (d, J = 8.4 Hz, 1H), 6.93 (t, J = 51.6 Hz, 1H), |
| | | 6.74 (d, J = 49.2 Hz, 1H), 6.60 (d, J = 16.8 Hz, |
| | | 1H), 5.22 (s, 2H), 3.34-3.31 (m, 4H), 3.07-3.05 |
| | | (m, 4H); LRMS (ES) m/z 474.6 (M⁺ + 1). |
| 36 | 36 | 3-[[5-[5-(difluoromethyl)-1,3,4-oxadiazol-2-yl]- |
| | | 2-pyridyl]methyl]-5-(3-piperazin-1-ylphenyl)- |
| | | 1,3,4-oxadiazol-2-one |
| | | ¹H NMR (400 MHz, CDCl₃) δ 9.29 (s, 1H), 8.40 (d, |
| | | J = 10.4 Hz, 1H), 7.50 (d, J = 8.0 Hz, 1H), 7.35- |
| | | 7.30 (m, 3H), 7.06-6.80 (m, 2H), 5.21 (s, 2H), |
| | | 3.23-3.20 (m, 4H), 3.07-3.05 (m, 4H); LRMS (ES) |
| | | m/z 456.5. (M⁺ + 1). |

### Example 21: Synthesis of Compound 21, 3-[[5-[5-(difluoromethyl)-1,3,4-oxadiazol-2-yl]-2-pyridyl]methyl]-5-[4-(4-methylpiperazin-1-yl)phenyl]-1,3,4-oxadiazol-2-one

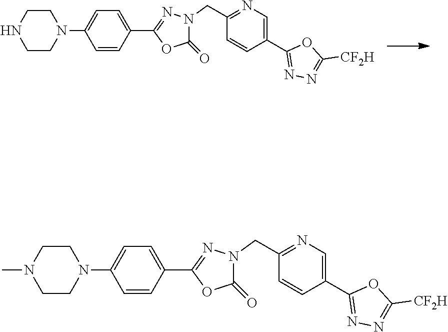 [SMILES: O=c1oc(-c2ccc(N3CCNCC3)cc2)nn1Cc1ccc(-c2nnc(C(F)F)o2)cn1 ; CN1CCN(c2ccc(-c3nn(Cc4ccc(-c5nnc(C(F)F)o5)cn4)c(=O)o3)cc2)CC1]

A solution in which 3-[[5-[5-(difluoromethyl)-1,3,4-oxadiazol-2-yl]-2-pyridyl]methyl]-5-(4-piperazin-1-ylphenyl)-1,3,4-oxadiazol-2-one (100.00%, 0.060 g, 0.132 mmol) synthesized in Step 5 of Example 20, sodium triacetoxyborohydride (100.00%, 2.000 equiv., 0.264 mmol) and formaldehyde (37.00%, 2.000 equiv., 0.264 mmol) were dissolved in dichloromethane (5 mL) at room temperature was stirred at the same temperature. Water was poured into the reaction mixture, and the mixture was extracted with dichloromethane. The organic layer was washed with a saturated aqueous sodium chloride solution, dried over anhydrous magnesium sulfate, filtered, and concentrated under reduced pressure. The concentrate was purified and concentrated by column chromatography (SiO₂, 4 g cartridge; dichloromethane/methanol=0% to 10%) to obtain the title compound (0.025 g, 40.42%, yellow solid).

¹H NMR (400 MHz, CDCl₃) δ 9.29 (s, 1H), 8.39 (d, J=10.4 Hz, 1H), 7.70 (d, J=8.8 Hz, 2H), 7.50 (d, J=8.0 Hz, 1H), 6.93-6.80 (m, 3H), 5.18 (s, 2H), 3.34 (s, 4H), 2.59 (s, 4H), 2.36 (s, 3H); LRMS (ES) m/z 470.6 (M⁺+1).

Compounds in Table 16 below were synthesized according to substantially the same method as in Example 21.

TABLE 16

| | Com- | |
|---|---|---|
| Exam- | pound | |
| ple | No. | Compound Name, ¹H-NMR, MS (ESI) |
| 22 | 22 | 3-[[5-[5-(difluoromethyl)-1,3,4-oxadiazol-2-yl]- |
| | | 2-pyridyl]methyl]-5-[4-(4-isopropylpiperazin-1- |
| | | yl)phenyl]-1,3,4-oxadiazol-2-one |
| | | ¹H NMR (400 MHz, CDCl₃) δ 9.28 (s, 1H), 8.38 (d, |
| | | J = 10.4 Hz, 1H), 7.69 (d, J = 9.2 Hz, 2H), 7.49 |
| | | (d, J = 8.0 Hz, 1H), 7.06-6.80 (m, 3H), 5.17 (s, |
| | | 2H), 3.38 (s, 4H), 2.91-2.84 (m, 1H), 2.77 (s, |
| | | 4H), 1.12 (d, J = 6.8 Hz, 6H); LRMS (ES) m/z 498.6 |
| | | (M⁺ + 1). |
| 24 | 24 | 3-[[5-[5-(difluoromethyl)-1,3,4-oxadiazol-2-yl]- |
| | | 2-pyridyl]methyl]-5-[2-fluoro-4-(4- |
| | | methylpiperazin-1-yl)phenyl]-1,3,4-oxadiazol-2- |
| | | one |
| | | ¹H NMR (400 MHz, CDCl₃) δ 9.30 (s, 1H), 8.40 (d, |
| | | J = 10.0 Hz, 1H), 7.63 (t, J = 8.8 Hz, 1H), 7.50 |
| | | (d, J = 8.4 Hz, 1H), 6.93 (t, J = 51.8 Hz, 1H), |
| | | 6.69 (d, J = 11.2 Hz, 1H), 6.60 (d, J = 16.4 Hz, |
| | | 1H), 5.22 (s, 2H), 3.38 (s, 4H), 2.64 (s, 4H), |
| | | 2.39 (s, 3H); LRMS (ES) m/z 488.6 (M⁺ + 1). |
| 25 | 25 | 3-[[5-[5-(difluoromethyl)-1,3,4-oxadiazol-2-yl]- |
| | | 2-pyridyl]methyl]-5-[2-fluoro-4-(4- |
| | | isopropylpiperazin-1-yl)phenyl]-1,3,4-oxadiazol- |
| | | 2-one |
| | | ¹H NMR (400 MHz, CDCl₃) δ 9.27 (s, 1H), 8.38 (d, |
| | | J = 10.4 Hz, 1H), 7.61 (d, J = 8.8 Hz, 1H), 7.48 |
| | | (d, J = 8.4 Hz, 1H), 6.93 (t, J = 51.6 Hz, 1H), |
| | | 6.67 (d, J = 11.2 Hz, 1H), 6.57 (d, J = 16.4 Hz, |
| | | 1H), 5.20 (s, 2H), 3.33 (s, 4H), 2.77-2.72 (m, |
| | | 1H), 2.66 (s, 4H), 1.07 (d, J = 6.4 Hz, 6H); LRMS |
| | | (ES) m/z 516.5 (M⁺ + 1). |
| 37 | 37 | 3-[[5-[5-(difluoromethyl)-1,3,4-oxadiazol-2-yl]- |
| | | 2-pyridyl]methyl]-5-[3-(4-methylpiperazin-1- |
| | | yl)phenyl]-1,3,4-oxadiazol-2-one |
| | | ¹H NMR (400 MHz, CDCl₃) δ 9.28 (s, 1H), 8.40 (d, |
| | | J = 10.0 Hz, 1H), 7.50 (d, J = 8.4 Hz, 1H), 7.34- |
| | | 7.33 (m, 3H), 7.06-6.80 (m, 2H), 5.20 (s, 2H), |
| | | 3.40 (t, J = 4.8 Hz, 4H), 2.84 (t, J = 4.8 Hz, |
| | | 4H), 2.51 (s, 3H); LRMS (ES) m/z 470.5 (M⁺ + 1). |
| 38 | 38 | 3-[[5-[5-(difluoromethyl)-1,3,4-oxadiazol-2-yl]- |
| | | 2-pyridyl]methyl]-5-[3-(4-isopropylpiperazin-1- |
| | | yl)phenyl]-1,3,4-oxadiazol-2-one |
| | | ¹H NMR (400 MHz, CDCl₃) δ 9.29 (s, 1H), 7.50 (d, |
| | | J = 8.4 Hz, 1H), 7.34-7.30 (m, 3H), 7.06-6.80 (m, |
| | | 2H), 5.21 (s, 2H), 3.31 (t, J = 4.8 Hz, 4H), 2.89- |
| | | 2.87 (m, 1H), 2.79 (t, J = 4.8 Hz, 4H), 1.14 (d, |
| | | J = 6.8 Hz, 6H); LRMS (ES) m/z 498.6 (M⁺ + 1). |
| 39 | 39 | 5-[3-(4-cyclobutylpiperazin-1-yl)phenyl]-3-[[5- |
| | | [5-(difluoromethyl)-1,3,4-oxadiazol-2-yl]-2- |
| | | pyridyl]methyl]-1,3,4-oxadiazol-2-one |
| | | ¹H NMR (400 MHz, CDCl₃) δ 9.29 (s, 1H), 8.40 (d, |
| | | J = 10.0 Hz, 1H), 7.50 (d, J = 8.0 Hz, 1H), 7.33- |
| | | 7.29 (m, 3H), 5.20 (s, 2H), 3.28 (t, J = 4.6 Hz, |
| | | 4H), 2.87-2.83 (m, 1H), 2.57 (t, J = 4.6 Hz, 4H), |
| | | 2.05-2.03 (m, 4H), 1.75-1.71 (m, 2H); LRMS (ES) |
| | | m/z 510.5 (M⁺ + 1). |
| 40 | 40 | 3-[[5-[5-(difluoromethyl)-1,3,4-oxadiazol-2-yl]- |
| | | 2-pyridyl]methyl]-5-[3-[4-(oxetane-3- |
| | | yl)piperazin-1-yl]phenyl]-1,3,4-oxadiazol-2-one |
| | | ¹H NMR (400 MHz, CDCl₃) δ 9.28 (s, 1H), 8.40 (d, |
| | | J = 10.4 Hz, 1H), 7.35-7.32 (m, 3H), 7.06-6.80 |
| | | (m, 2H), 5.20 (s, 2H), 4.71-4.63 (m, 4H), 3.56- |
| | | 3.53 (m, 1H), 3.27 (t, J = 4.8 Hz, 4H), 2.49 (t, |
| | | J = 4.8 Hz, 4H); LRMS (ES) m/z 512.5 (M⁺ + 1). |

### Example 6: Synthesis of Compound 6, 3-[[5-[5-(difluoromethyl)-1,3,4-oxadiazol-2-yl]-2-pyridyl]methyl]-5-[6-(4-isopropyl piperazin-1-yl)-2-pyridyl]-1,3,4-oxadiazol-2-one

### [Step 1] Synthesis of 6-fluoropyridin-2-carbohydrazide

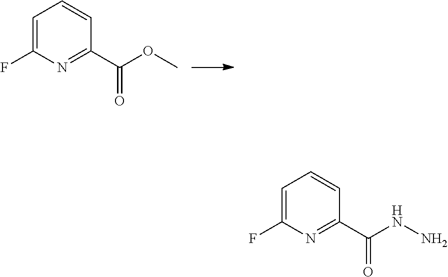

To a solution in which methyl 6-fluoropyridin-2-carboxylate (100.00%, 1.000 g, 6.446 mmol) was dissolved in ethanol (30 mL) at room temperature, hydrazine monohydrate (100.00% solution, 6.266 mL, 129.000 mmol) was added and stirred overnight at the same temperature. To the concentrate obtained by removing the solvent from the reaction mixture under reduced pressure, a saturated aqueous solution of sodium hydrogen carbonate was poured, and the resulting mixture was extracted with dichloromethane. The organic layer was washed with a saturated aqueous sodium chloride solution, dried over anhydrous magnesium sulfate, filtered, and concentrated under reduced pressure. The obtained product was used without further purification (1 g, 100.00%, pale yellow solid).

### [Step 2] Synthesis of 5-(6-fluoro-2-pyridyl)-3H-1,3,4-oxadiazol-2-one

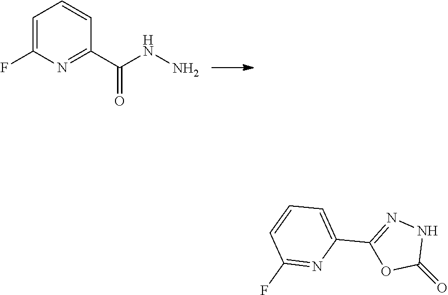 [SMILES: NNC(=O)c1cccc(F)n1 ; O=c1[nH]nc(-c2cccc(F)n2)o1]

To a solution in which 6-fluoropyridin-2-carbohydrazide (100.00%, 1.000 g, 6.446 mmol) synthesized in Step 1 and 1,1′-carbonylbis-1H-imidazole (100.00%, 1.254 g, 7.734 mmol) were dissolved in tetrahydrofuran (30 mL) at room temperature, triethylamine (100.00% solution, 1.251 mL, 9.000 mmol) was added and stirred overnight at the same temperature. Water was poured into the reaction mixture, and the mixture was extracted with dichloromethane. The organic layer was washed with a saturated aqueous sodium chloride solution, dried over anhydrous magnesium sulfate, filtered, and concentrated under reduced pressure. The concentrate was purified and concentrated by column chromatography (SiO₂, 4 g cartridge; methanol/dichloromethane=0% to 10%) to obtain the title compound (0.21 g, 17.987%, yellow solid).

### [Step 3] Synthesis of 3-[[5-[5-(difluoromethyl)-1,3,4-oxadiazol-2-yl]-2-pyridyl]methyl]-5-(6-fluoro-2-pyridyl)-1,3,4-oxadiazol-2-one

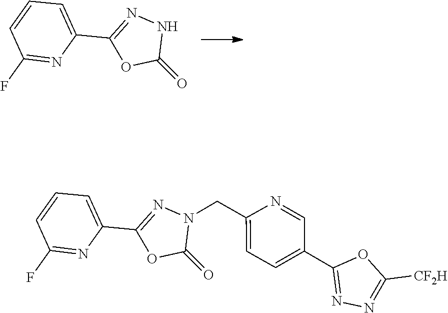 [SMILES: O=c1[nH]nc(-c2cccc(F)n2)o1 ; O=c1oc(-c2cccc(F)n2)nn1Cc1ccc(-c2nnc(C(F)F)o2)cn1]

To a solution in which 5-(6-fluoro-2-pyridyl)-3H-1,3,4-oxadiazol-2-one (100.00%, 0.210 g, 1.160 mmol) synthesized in Step 2 and potassium carbonate (100.00%, 0.244 g, 1.765 mmol) were dissolved in N,N-dimethylformamide (5 mL) at room temperature, 2-[6-(bromomethyl)-3-pyridyl]-5-(difluoromethyl)-1,3,4-oxadiazole (100.00%, 0.404 g, 1.393 mmol) was added and stirred overnight at the same temperature. Water was poured into the reaction mixture, and the mixture was extracted with ethyl acetate. The organic layer was washed with a saturated aqueous sodium chloride solution, dried over anhydrous magnesium sulfate, filtered, and concentrated under reduced pressure. The concentrate was purified and concentrated by column chromatography (SiO₂, 4 g cartridge; methanol/dichloromethane=0% to 10%) to obtain the title compound (0.185 g, 40.88%, pale yellow solid).

### [Step 4] Synthesis of Compound 6

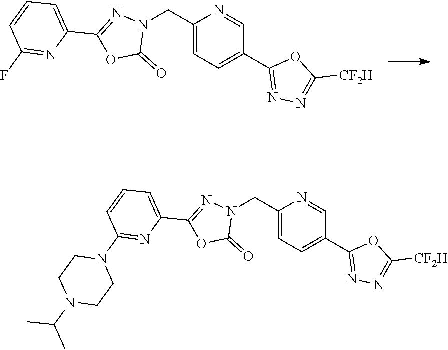 [SMILES: O=c1oc(-c2cccc(F)n2)nn1Cc1ccc(-c2nnc(C(F)F)o2)cn1 ; CC(C)N1CCN(c2cccc(-c3nn(Cc4ccc(-c5nnc(C(F)F)o5)cn4)c(=O)o3)n2)CC1]

A solution in which 3-[[5-[5-(difluoromethyl)-1,3,4-oxadiazol-2-yl]-2-pyridyl]methyl]-5-(6-fluoro-2-pyrid yl)-1,3,4-oxadiazol-2-one (100.00%, 0.040 g, 0.103 mmol) synthesized in Step 3, 1-isopropylpiperazine (100.00% solution, 0.029 mL, 0.203 mmol) and N,N-diisopropylethylamine (100.00% solution, 0.027 mL, 0.150 mmol) were dissolved in dimethylsulfoxide (1 mL) at 130° C. was stirred at the same temperature overnight, and then the reaction was terminated by lowering the temperature to room temperature. Water was poured into the reaction mixture, and the mixture was extracted with ethyl acetate. The organic layer was washed with a saturated aqueous sodium chloride solution, dried over anhydrous magnesium sulfate, filtered, and concentrated under reduced pressure. The concentrate was purified and concentrated by column chromatography (SiO₂, 4 g cartridge; methanol/dichloromethane=0% to 10%) to obtain the title compound (0.02 g, 39.15%) in the form of an orange solid.

¹H NMR (400 MHz, CDCl₃) δ 9.32 (d, J=1.6 Hz, 1H), 8.43 (dd, J=8.2, 2.2 Hz, 1H), 7.61-7.57 (m, 1H), 7.52 (d, J=8.0 Hz, 1H), 7.23 (d, J=7.6 Hz, 1H), 6.96 (t, J=51.6 Hz, 1H), 6.78 (d, J=8.8 Hz, 1H), 5.27 (s, 2H), 3.76 (s, 4H), 2.89-2.75 (m, 5H), 1.18 (d, J=5.6 Hz, 6H); LRMS (ES) m/z 499.8 (M⁺+1).

Compounds in Table 17 below were synthesized according to substantially the same method as in Example 6.

TABLE 17

| | Com- | |
|---|---|---|
| Exam- | pound | |
| ple | No. | Compound Name, ¹H-NMR, MS (ESI) |
| 7 | 7 | 3-[[5-[5-(difluoromethyl)-1,3,4-oxadiazol-2-yl]- |
| | | 2-pyridyl]methyl]-5-[6-[4-(oxetane-3- |
| | | yl)piperazin-1-yl]-2-pyridyl]-1,3,4-oxadiazol-2- |
| | | one |
| | | ¹H NMR (400 MHz, CDCl₃) δ 9.32 (d, J = 1.2 Hz, |
| | | 1H), 8.43 (dd, J = 8.2, 2.2 Hz, 1H), 7.63-7.59 |
| | | (m, 1H), 7.52 (d, J = 8.4 Hz, 1H), 7.26 (d, J = |
| | | 7.6 Hz, 1H), 6.96 (t, J = 51.6 Hz, 1H), 6.79 (d, |
| | | J = 8.4 Hz, 1H), 5.27 (s, 2H), 4.75-4.72 (m, 4H), |
| | | 3.76-3.64 (m, 5H), 2.55 (s, 4H); LRMS (ES) m/z |
| | | 513.8 (M⁺ + 1). |

### Example 19: Synthesis of Compound 1, 3(5-(5-(difluoromethyl)-1,3,4-oxadiazol-2-yl)pyridin-2-yl)methyl)-5-(1-methyl-1H-indole-5-yl)-1,3,4-oxadiazol-2(3H)-thion

### [Step 1] Synthesis of methyl 1-methylindole-5-carboxylate

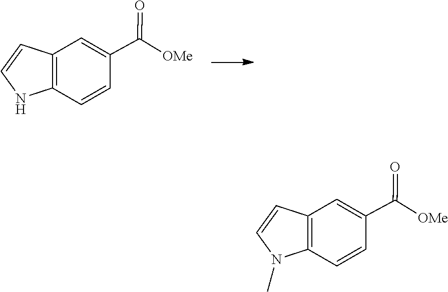

To a solution in which methyl 1H-indole-5-carboxylate (100.00%, 0.500 g, 2.854 mmol) was dissolved in N,N-dimethylformamide (7 mL) at 0° C., sodium hydride (60.00%, 140.000 mg, 3.500 mmol) was added and stirred at the same temperature for 0.2 hours. Iodomethane (100.00% solution, 0.27 ML, 4.340 mmol) was added to the reaction mixture and further stirred at room temperature for 4 hours. Water was poured into the reaction mixture, and the mixture was extracted with ethyl acetate. The organic layer was washed with a saturated aqueous sodium chloride solution, dried over anhydrous magnesium sulfate, filtered, and concentrated under reduced pressure. The concentrate was purified and concentrated by column chromatography (SiO₂, 12 g cartridge; ethyl acetate/hexane=0% to 30%) to obtain the title compound (0.27 g, 49.996%, white solid).

### [Step 2] Synthesis of 1-methylindole-5-carbohydrazide

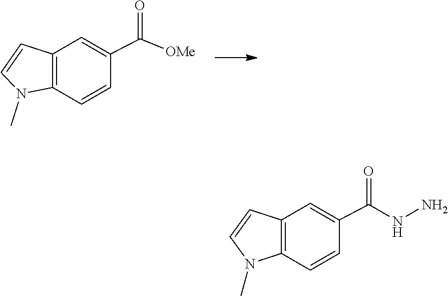

Methyl 1-methylindole-5-carboxylate (100.00%, 160.000 mg, 0.846 mmol) synthesized in Step 1 was dissolved in ethanol (2.5 mL), and hydrazine monohydrate (100.00% solution, 0.8 mL, 16.490 mmol) was added at room temperature and stirred at 60° C. for 18 hours. Then, the reaction was terminated by lowering the temperature to room temperature. After removing the solvent from the reaction mixture under reduced pressure, the obtained product was used without further purification (160 mg, 100.00%, pale yellow solid).

### [Step 3] Synthesis of 5-(1-methylindole-5-yl)-3H-1,3,4-oxadiazol-2-one

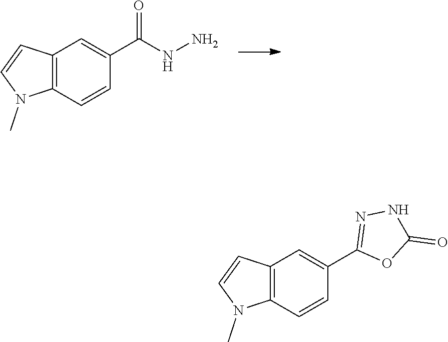 [SMILES: Cn1ccc2cc(C(=O)NN)ccc21 ; Cn1ccc2cc(-c3n[nH]c(=O)o3)ccc21]

To a solution in which 1-methylindole-5-carbohydrazide (100.00%, 160.000 mg, 0.846 mmol) synthesized in Step 2 and N,N-diisopropylethylamine (DIPEA, 100.00% solution, 0.3 mL, 1.700 mmol) were dissolved in dichloromethane (3 mL) at 0° C., triphosgene (100.00%, 100.000 mg, 0.337 mmol) was added and stirred at the same temperature. After removing the solvent from the reaction mixture under reduced pressure, the concentrate was purified and concentrated by column chromatography (SiO₂, 12 g cartridge; ethyl acetate/hexane=10% to 50%) to obtain the title compound (270 mg, 148.36%, white solid).

### [Step 4] Synthesis of Compound 19

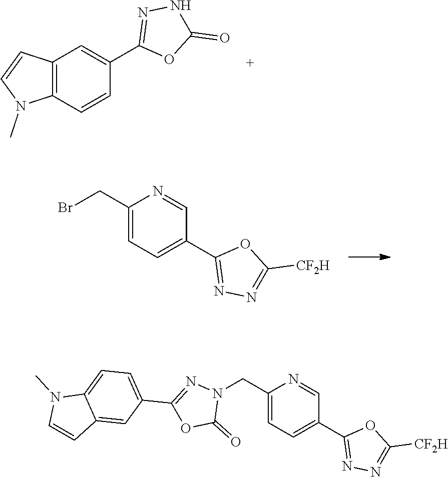 [SMILES: Cn1ccc2cc(-c3n[nH]c(=O)o3)ccc21 ; FC(F)c1nnc(-c2ccc(CBr)nc2)o1 ; Cn1ccc2cc(-c3nn(Cc4ccc(-c5nnc(C(F)F)o5)cn4)c(=O)o3)ccc21]

To a solution in which 5-(1-methylindole-5-yl)-3H-1,3,4-oxadiazol-2-one (100.00%, 50.000 mg, 0.232 mmol) synthesized in Step 3 was dissolved in N,N-dimethylformamide (2 mL) at room temperature, potassium carbonate (100.00%, 35,000 mg, 0.353 mmol) was added and stirred at the same temperature for 0.3 hours. To the reaction mixture, 2-[6-(bromomethyl)-3-pyridyl]-5-(difluoromethyl)-1,3,4-oxadiazole (100.00%, 80,000 mg, 0.276 mmol) was added and further stirred at 35° C. for 18 hours. A saturated aqueous sodium chloride solution was poured into the reaction mixture and extracted with ethyl acetate. The organic layer was washed with a saturated aqueous sodium chloride solution, dried over anhydrous magnesium sulfate, filtered, and concentrated under reduced pressure. The concentrate was purified and concentrated by column chromatography (SiO₂, 4 g cartridge; hexane/ethyl acetate=100% to 50%) to obtain the title compound (43 mg, 43.61%, white solid).

¹H NMR (400 MHz, CD₃OD) δ 9.27 (d, J=1.2 Hz, 1H), 8.52 (dd, J=8.4, 2.4 Hz, 1H), 8.10 (d, J=0.8 Hz, 1H), 7.80 (s, 1H), 7.70 (t, J=8.8 Hz, 1H), 7.48 (d, J=8.8 Hz, 1H), 7.26 (d, J=3.2 Hz, 1H), 7.21 (t, J=51.6 Hz, 1H), 6.57 (dd, J=3.2, 0.4 Hz, 1H), 5.25 (s, 2H), 3.86 (s, 3H); LRMS (ES) m/z 425.7 (M⁺+1).

Compounds in Table 18 below were synthesized according to substantially the same method as in Example 19.

TABLE 18

| | Com- | |
|---|---|---|
| Exam- | pound | |
| ple | No. | Compound Name, ¹H-NMR, MS (ESI) |
| 31 | 31 | 3-[[5-[5-(difluoromethyl)-1,3,4-oxadiazol-2-yl]- |
| | | 2-pyridyl]methyl]-5-(1-methylindol-6-yl)-1,3,4- |
| | | oxadiazol-2-one |
| | | ¹H NMR (400 MHz, CDCl₃) δ 9.35 (d, J = 1.6 Hz, |
| | | 1H), 8.45 (dd, J = 8.2, 2.2 Hz, 1H), 7.88 (s, |
| | | 1H), 7.70 (d, J = 8.4 Hz, 1H), 7.63 (dd, J = 8.4, |
| | | 1.2 Hz, 1H), 7.57 (d, J = 8.4 Hz, 1H), 7.22 (d, |
| | | J = 3.2 Hz, 1H), 6.96 (t, J = 51.6 Hz, 1H), 6.56 |
| | | (d, J = 3.2 Hz, 1H), 5.27 (s, 2H), 3.88 (s, 3H); |
| | | LRMS (ES) m/z 425.9 (M⁺ + 1). |
| 32 | 32 | 3-[[5-[5-(difluoromethyl)-1,3,4-oxadiazol-2-yl]- |
| | | 2-pyridyl]methyl]-5-(1-methylpyrrolo[2,3- |
| | | b]pyridin-5-yl)-1,3,4-oxadiazol-2-one |
| | | ¹H NMR (400 MHz, CDCl₃) δ 9.35 (d, J = 1.6 Hz, |
| | | 1H), 8.85 (d, J = 2.0 Hz, 1H), 8.46 (dd, J = 8.2, |
| | | 2.2 Hz, 1H), 8.39 (d, J = 2.0 Hz, 1H), 7.57 (d, |
| | | J = 8.0 Hz, 1H), 7.31 (d, J = 3.2 Hz, 1H), 6.96 |
| | | (t, J = 51.6 Hz, 1H), 6.58 (d, J = 3.6 Hz, 1H), |
| | | 5.26 (s, 2H), 3.96 (s, 3H); LRMS (ES) m/z 426.9 |
| | | (M⁺ + 1). |

### Example 33: Synthesis of Compound 33, 3-[[5-[5-(difluoromethyl)-1,3,4-oxadiazol-2-yl]-2-pyridyl]methyl]-5-(1H-indole-6-yl)-1,3,4-oxadiazol-2-one

### [Step 1] Synthesis of 1-tert-butoxycarbonylindole-6-carboxylic acid

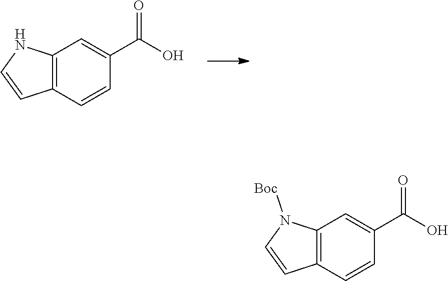 [SMILES: O=C(O)c1ccc2cc[nH]c2c1 ; CC(C)(C)OC(=O)n1ccc2ccc(C(=O)O)cc21]

To a solution in which 1H-indole-6-carboxylic acid (100.00%, 1.000 g, 6.205 mmol) and di-tert-butyl dicarbonate (100.00%, 2.000 g, 9.164 mmol) were dissolved in tetrahydrofuran (12 mL) at room temperature, 4-(dimethylamino)pyridine (100.00%, 0.150 g, 1.228 mmol) was added and stirred at the same temperature for 18 hours. Water was poured into the reaction mixture, and the mixture was extracted with ethyl acetate. The organic layer was washed with a saturated aqueous sodium chloride solution, dried over anhydrous magnesium sulfate, filtered, and concentrated under reduced pressure. The concentrate was purified and concentrated by column chromatography (SiO₂, 24 g cartridge; ethyl acetate/hexane=0% to 30%) to obtain the title compound (0.8 g, 49.34%, white solid).

### [Step 2] Synthesis of tert-butyl 6-(hydrazinecarbonyl)-1H-indole-1-carboxylate

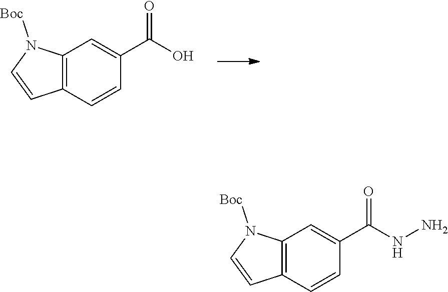 [SMILES: CC(C)(C)OC(=O)n1ccc2ccc(C(=O)O)cc21 ; CC(C)(C)OC(=O)n1ccc2ccc(C(=O)NN)cc21]

To a solution in which 1-tert-butoxycarbonylindole-6-carboxylic acid (100.00%, 250 mg, 0.9568 mmol) synthesized in Step 1 was dissolved in tetrahydrofuran (5 mL) at room temperature, 1,1′-carbonylbis-1H-imidazole (100.00%, 200.000 mg, 1.233 mmol) was added and stirred at the same temperature for 3 hours. Hydrazine monohydrate (100.00%, 145.000 mg, 2.897 mmol) was added to the reaction mixture and further stirred at the same temperature for 18 hours. After removing the solvent from the reaction mixture under reduced pressure, the obtained product was used without further purification (mixture, pale yellow solid).

### [Step 3] Synthesis of tert-butyl 6-(2-oxo-3H-1,3,4-oxadiazol-5-yl)indole-1-carboxylate

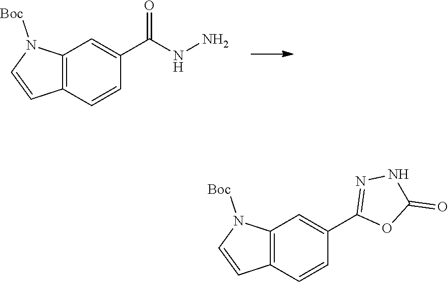 [SMILES: CC(C)(C)OC(=O)n1ccc2ccc(C(=O)NN)cc21 ; CC(C)(C)OC(=O)n1ccc2ccc(-c3n[nH]c(=O)o3)cc21]

Tert-butyl 6-(hydrazinecarbonyl)indole-1-carboxylate (100.00%, 450.000 mg, 1.635 mmol) synthesized in Step 2 and N,N-diisopropylethylamine (100.00% solution, 0.56 mL, 3.200 mmol) were dissolved in dichloromethane (5 mL), and triphosgene (100.00%, 190.000 mg, 0.640 mmol) was added at 0° C. and stirred at room temperature for 18 hours. After removing the solvent from the reaction mixture under reduced pressure, the concentrate was purified and concentrated by column chromatography (SiO₂, 12 g cartridge; ethyl acetate/hexane=10% to 50%) to obtain the title compound (270 mg, 54.82%, white solid).

### [Step 4] Synthesis of tert-butyl 6-[4-[[5-[5-(difluoromethyl)-1,3,4-oxadiazol-2-yl]-2-pyridyl]methyl]-5-oxo-1,3,4-oxadiazol-2-yl]indole-1-carboxylate

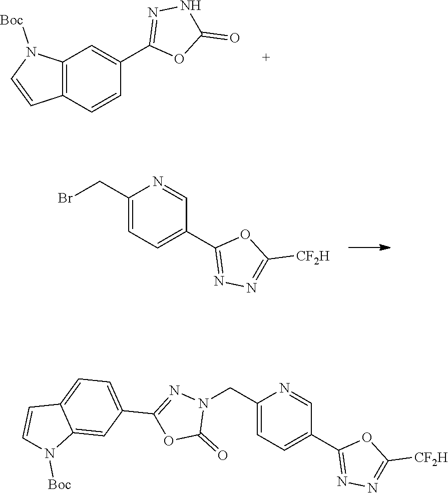 [SMILES: CC(C)(C)OC(=O)n1ccc2ccc(-c3n[nH]c(=O)o3)cc21 ; FC(F)c1nnc(-c2ccc(CBr)nc2)o1 ; CC(C)(C)OC(=O)n1ccc2ccc(-c3nn(Cc4ccc(-c5nnc(C(F)F)o5)cn4)c(=O)o3)cc21]

To a solution in which tert-butyl 6-(2-oxo-3H-1,3,4-oxadiazol-5-yl)indole-1-carboxylate (100.00%, 55.000 mg, 0.183 mmol) synthesized in Step 3 was dissolved in N,N-dimethylformamide (2 mL) at room temperature, potassium carbonate (100.00%, 25.000 mg, 0.252 mmol) was added and stirred at the same temperature for 0.3 hours. To the reaction mixture, 2-[6-(bromomethyl)-3-pyridyl]-5-(difluoromethyl)-1,3,4-oxadiazole (100.00%, 64,000 mg, 0.221 mmol) was added and further stirred at 35° C. for 18 hours. Water was poured into the reaction mixture, and the mixture was extracted with ethyl acetate. The organic layer was washed with a saturated aqueous sodium chloride solution, dried over anhydrous magnesium sulfate, filtered, and concentrated under reduced pressure. The concentrate was purified and concentrated by column chromatography (SiO₂, 4 g cartridge; hexane/ethyl acetate=100% to 50%) to obtain the title compound (58 mg, 62.25%, white solid).

### [Step 5] Synthesis of Compound 33

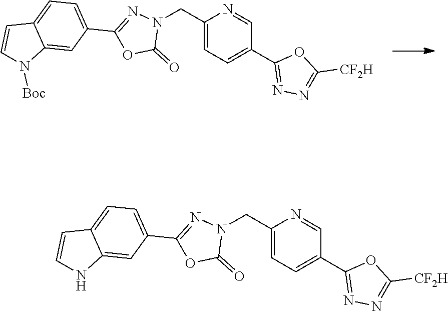 [SMILES: CC(C)(C)OC(=O)n1ccc2ccc(-c3nn(Cc4ccc(-c5nnc(C(F)F)o5)cn4)c(=O)o3)cc21 ; O=c1oc(-c2ccc3cc[nH]c3c2)nn1Cc1ccc(-c2nnc(C(F)F)o2)cn1]

To a solution in which tert-butyl 6-[4-[[5-[5-(difluoromethyl)-1,3,4-oxadiazol-2-yl]-2-pyridyl]methyl]-5-oxo-1,3,4-oxadiazol-2-yl]indole-1-carboxylate (100.00%, 50.000 mg, 0.098 mmol) synthesized in Step 4 was dissolved in dichloromethane (1 mL) at room temperature, trifluoroacetic acid (100.00% solution, 0.3 mL, 3.918 mmol) was added and stirred at the same temperature for 1 hour. To the concentrate obtained by removing the solvent from the reaction mixture under reduced pressure, a saturated aqueous solution of sodium hydrogen carbonate was poured, and the resulting mixture was extracted with ethyl acetate. The organic layer was washed with a saturated aqueous sodium chloride solution, dried over anhydrous magnesium sulfate, filtered, and concentrated under reduced pressure. The concentrate was purified and concentrated by column chromatography (SiO₂, 4 g cartridge; dichloromethane/methanol=100% to 80%) to obtain the title compound (34 mg, 84.59%, pale yellow solid).

¹H NMR (400 MHz, CD₃OD) δ 9.27 (d, J=1.6 Hz, 1H), 8.54 (dd, J=8.2, 2.2 Hz, 1H), 7.93 (d, J=0.8 Hz, 1H), 7.73 (d, J=8.4 Hz, 1H), 7.67 (d, J=8.4 Hz, 1H), 7.53 (dd, J=8.4, 1.6 Hz, 1H), 7.44 (d, J=1.2 Hz, 1H), 7.26 (t, J=51.6 Hz, 1H), 5.26 (s, 2H); LRMS (ES) m/z 412.8 (M⁺+1).

Compounds in Table 19 below were synthesized according to substantially the same method as in Example 33.

TABLE 19

| | Compound | |
|---|---|---|
| Example | No. | Compound Name, ¹H-NMR, MS (ESI) |
| 3 | 3 | 3-[[5-[5-(difluoromethyl)-1,3,4-oxadiazol-2-yl]- |
| | | 2-pyridyl]methyl]-5-(1H-indol-5-yl)-1,3,4- |
| | | oxadiazol-2-one |
| | | ¹H NMR (400 MHz, CD₃OD) δ 9.32 (d, J = 2.0 Hz, |
| | | 1H), 8.58 (dd, J = 8.2, 2.2 Hz, 1H), 8.18 (d, J = |
| | | 1.6 Hz, 1H), 7.81 (d, J = 8.4 Hz, 1H), 7.69 |
| | | (dd, J = 8.8, 1.6 Hz, 1H), 7.61 (d, J = 8.4 Hz, |
| | | 1H), 7.50 (d, J = 3.2 Hz, 1H), 7.44 (t, J = 51.6 |
| | | Hz, 1H), 6.68 (d, J = 3.2 Hz, 1H), 5.32 (s, 2H); |
| | | LRMS (ES) m/z 411.6 (M⁺ + 1). |
| 34 | 34 | 3-[[5-[5-(difluoromethyl)-1,3,4-oxadiazol-2-yl]- |
| | | 2-pyridyl]methyl]-5-(1H-indol-4-yl)-1,3,4- |
| | | oxadiazol-2-one |
| | | ¹H NMR (400 MHz, CD₃OD) δ 9.28 (d, J = 1.6 Hz, |
| | | 1H), 8.54 (dd, J = 8.2, 2.2 Hz, 1H), 7.76 (d, J = |
| | | 8.4 Hz, 1H), 7.62 (d, J = 8.0 Hz, 1H), 7.40 (t, |
| | | J = 1.6 Hz, 1H), 7.25 (t, J = 51.6 Hz, 1H), 7.25 |
| | | (t, J = 7.8 Hz, 1H), 6.97 (d, J = 3.2 Hz, 1H), |
| | | 5.32 (s, 2H); LRMS (ES) m/z 411.9 (M⁺ + 1). |
| 35 | 35 | 3-[[5-[5-(difluoromethyl)-1,3,4-oxadiazol-2-yl]- |
| | | 3-fluoro-2-pyridyl]methyl]-5-(1H-indol-4-yl)- |
| | | 1,3,4-oxadiazol-2-one |
| | | ¹H NMR (400 MHz, CD₃OD) δ 9.11 (s, 1H), 8.37 (dd, |
| | | J = 9.8, 1.8 Hz, 1H), 7.62-7.59 (m, 2H), 7.40 (t, |
| | | J = 1.4 Hz, 1H), 7.25 (t, J = 51.4 Hz, 1H), 7.24 |
| | | (t, J = 7.8 Hz, 1H), 6.94 (t, J = 1.6 Hz, 1H), |
| | | 5.41 (d, J = 1.6 Hz, 2H); LRMS (ES) m/z 429.8 |
| | | (M⁺ + 1). |

### Example 11: Synthesis of Compound 11, 3-[[5-[5-(difluoromethyl)-1,3,4-oxadiazol-2-yl]-2-pyridyl]methyl]-5-(1,2,3,4-tetrahydroisoquinolin-6-yl)-1,3,4-oxadiazol-2-one

### [Step 1] Synthesis of tert-butyl 7-(hydrazinecarbonyl)-3,4-dihydro-1H-isoquinolin-2-carboxylate

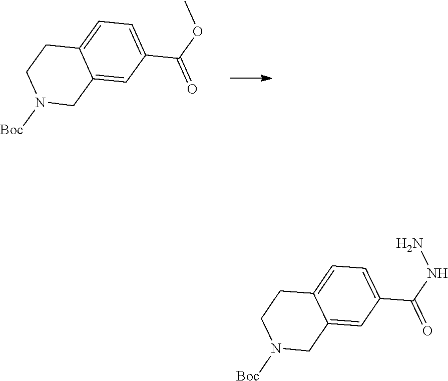 [SMILES: COC(=O)c1ccc2c(c1)CN(C(=O)OC(C)(C)C)CC2 ; CC(C)(C)OC(=O)N1CCc2ccc(C(=O)NN)cc2C1]

A solution in which 2-tert-butyl, 7-methyl 3,4-dihydro-1H-isoquinolin-2,7-dicarboxylate (100.00%, 0.603 g, 2.070 mmol) and hydrazine monohydrate (100.00%, 10.000 equiv., 20.700 mmol) were dissolved in ethanol (20 mL) at 70° C. was stirred overnight at the same temperature, and then the reaction was terminated by lowering the temperature to room temperature. After removing the solvent from the reaction mixture under reduced pressure, the obtained product was used without further purification (0.603 g, 100.0%, white solid).

### [Step 2] Synthesis of tert-butyl 7-(2-oxo-3H-1,3,4-oxadiazol-5-yl)-3,4-dihydro-1H-isoquinolin-2-carboxylate

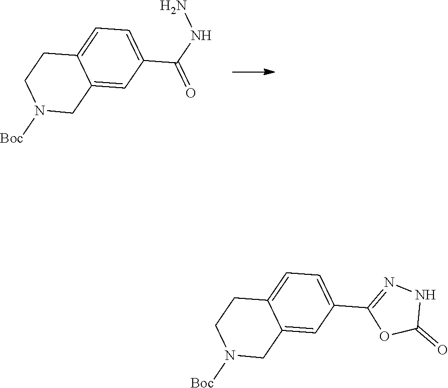 [SMILES: CC(C)(C)OC(=O)N1CCc2ccc(C(=O)NN)cc2C1 ; CC(C)(C)OC(=O)N1CCc2ccc(-c3n[nH]c(=O)o3)cc2C1]

A solution in which tert-butyl 7-(hydrazinecarbonyl)-3,4-dihydro-1H-isoquinolin-2-carboxylate (100.00%, 0.640 g, 2.197 mmol) synthesized in Step 1 and 1,1′-carbonylbis-1H-imidazole (100.00%, 1.500 equiv., 3.296 mmol) were dissolved in dichloromethane (5 mL) at 50° C. was stirred overnight at the same temperature, and then the reaction was terminated by lowering the temperature to room temperature. Water was poured into the reaction mixture, and the mixture was extracted with dichloromethane. The organic layer was washed with a saturated aqueous sodium chloride solution, dried over anhydrous magnesium sulfate, filtered, and concentrated under reduced pressure. The concentrate was purified and concentrated by column chromatography (SiO₂, 4 g cartridge; ethyl acetate/hexane=0% to 100%) to obtain the title compound (0.42 g, 60.25%, white solid).

### [Step 3] Synthesis of tert-butyl 7-[4-[[5-[5-(difluoromethyl)-1,3,4-oxadiazol-2-yl]-2-pyridyl]methyl]-5-oxo-1,3,4-oxadiazol-2-yl]-3,4-dihydro-1H-isoquinolin-2-carboxylate

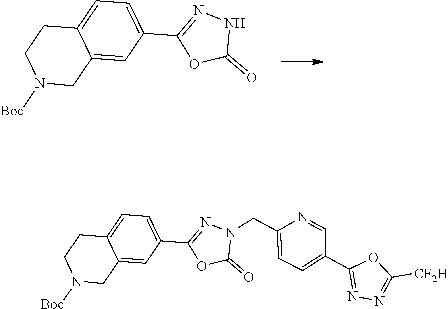 [SMILES: CC(C)(C)OC(=O)N1CCc2ccc(-c3n[nH]c(=O)o3)cc2C1 ; CC(C)(C)OC(=O)N1CCc2ccc(-c3nn(Cc4ccc(-c5nnc(C(F)F)o5)cn4)c(=O)o3)cc2C1]

A solution in which tert-butyl 7-(2-oxo-3H-1,3,4-oxadiazol-5-yl)-3,4-dihydro-1H-isoquinolin-2-carboxylate (100.00%, 0.420 g, 1.324 mmol) synthesized in Step 2, 2-[6-(bromomethyl)-3-pyridyl]-5-(difluoromethyl)-1,3,4-oxadiazole (100.00%, 1.200 equiv., 1.588 mmol), potassium carbonate (100.00%, 2.000 equiv., 2.647 mmol) and potassium iodide (100.00%, 1.100 equiv., 1.456 mmol) were dissolved in N,N-dimethylformamide (25 mL) at room temperature was stirred overnight at the same temperature. Water was poured into the reaction mixture, and the mixture was extracted with dichloromethane. The organic layer was washed with a saturated aqueous sodium chloride solution, dried over anhydrous magnesium sulfate, filtered, and concentrated under reduced pressure. The concentrate was purified and concentrated by column chromatography (SiO₂, 12 g cartridge; ethyl acetate/hexane=0% to 100%) to obtain the title compound (0.55 g, 78.92%, yellow solid).

### [Step 4] Synthesis of Compound 11

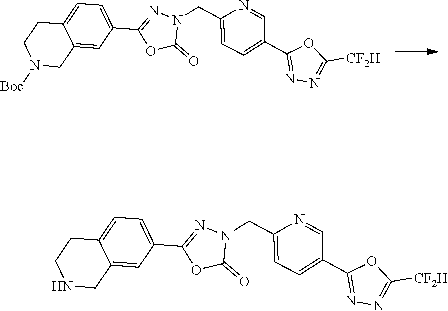 [SMILES: CC(C)(C)OC(=O)N1CCc2ccc(-c3nn(Cc4ccc(-c5nnc(C(F)F)o5)cn4)c(=O)o3)cc2C1 ; O=c1oc(-c2ccc3c(c2)CNCC3)nn1Cc1ccc(-c2nnc(C(F)F)o2)cn1]

A solution in which tert-butyl 7-[4-[[5-[5-(difluoromethyl)-1,3,4-oxadiazol-2-yl]-2-pyridyl]methyl]-5-oxo-1,3,4-oxadiazol-2-yl]-3,4-dihydro-1H-isoquinolin-2-carboxylate (100.00%, 0.550 g, 1.045 mmol) synthesized in Step 3 and 2,2,2-trifluoroacetic acid (100.00%, 10.000 equiv., 10.450 mmol) were dissolved in dichloromethane (20 mL) at room temperature was stirred overnight at the same temperature. An aqueous solution of N-sodium hydrogen carbonate was poured into the reaction mixture, followed by extraction with dichloromethane. The organic layer was washed with a saturated aqueous sodium chloride solution, dried over anhydrous magnesium sulfate, filtered, and concentrated under reduced pressure. The concentrate was purified and concentrated by column chromatography (SiO₂, 4 g cartridge; dichloromethane/methanol=0% to 10%) to obtain the title compound (0.42 g, 94.29%, yellow solid).

¹H NMR (400 MHz, CDCl₃) δ 9.31 (s, 1H), 8.42 (d, J=8.4 Hz, 1H), 7.60 (s, 2H), 7.51 (d, J=7.6 Hz, 1H), 7.12 (d, J=8.4 Hz, 1H), 6.93 (t, J=51.6 Hz, 1H), 5.21 (s, 2H), 4.09 (s, 2H), 3.19 (s, 2H), 2.88 (s, 2H); LRMS (ES) m/z 427.4 (M⁺+1).

### Example 12: Synthesis of Compound 12, 3-[[5-[5-(difluoromethyl)-1,3,4-oxadiazol-2-yl]-2-pyridyl]methyl]-5-(2-methyl-3,4-dihydro-1H-isoquinolin-7-yl)-1,3,4-oxadiazol-2-one

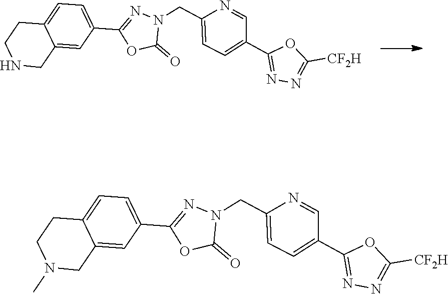 [SMILES: O=c1oc(-c2ccc3c(c2)CNCC3)nn1Cc1ccc(-c2nnc(C(F)F)o2)cn1 ; CN1CCc2ccc(-c3nn(Cc4ccc(-c5nnc(C(F)F)o5)cn4)c(=O)o3)cc2C1]

A solution in which 3-[[5-[5-(difluoromethyl)-1,3,4-oxadiazol-2-yl]-2-pyridyl]methyl]-5-(1,2,3,4-tetrahydroisoquinolin-7-yl)-1,3,4-oxadiazol-2-one (100.00%, 0.080 g, 0.188 mmol) synthesized in Step 4 of Example 11, formaldehyde (100.00%, 1.500 equiv., 0.281 mmol) and sodium triacetoxyborohydride (100.00%, 2.000 equiv., 0.375 mmol) were dissolved in dichloromethane (5 mL) at room temperature was stirred at the same temperature. Water was poured into the reaction mixture, and the mixture was extracted with dichloromethane. The organic layer was washed with a saturated aqueous sodium chloride solution, dried over anhydrous magnesium sulfate, filtered, and concentrated under reduced pressure. The concentrate was purified and concentrated by column chromatography (SiO₂, 4 g cartridge; dichloromethane/methanol=0% to 10%) to obtain the title compound (0.02 g, 24.21%, yellow solid).

¹H NMR (400 MHz, CDCl₃) δ 9.27 (s, 1H), 8.38 (d, J=10.4 Hz, 1H), 7.57 (d, J=8.0 Hz, 1H), 7.52 (d, J=25.6 Hz, 2H), 7.17 (d, J=8.0 Hz, 1H), 6.93 (t, J=51.6 Hz, 1H), 5.18 (s, 2H), 3.60 (s, 2H), 2.95 (t, J=5.6 Hz, 2H), 2.72 (t, J=6.0 Hz, 2H), 2.45 (s, 3H); LRMS (ES) m/z 441.4 (M⁺+1).

Compounds in Table 20 below were synthesized according to substantially the same method as in Example 12.

TABLE 20

| | Com- | |
|---|---|---|
| Exam- | pound | |
| ple | No. | Compound Name, ¹H-NMR, MS (ESI) |
| 13 | 13 | 3-[[5-[5-(difluoromethyl)-1,3,4-oxadiazol-2-yl]- |
| | | 2-pyridyl]methyl]-5-(2-isopropyl-3,4-dihydro-1H- |
| | | isoquinolin-7-yl)-1,3,4-oxadiazol-2-one |
| | | ¹H NMR (400 MHz, CDCl₃) δ 9.28 (s, 1H), 8.39 (d, |
| | | J = 10.4 Hz, 1H), 7.57 (d, J = 8.0 Hz, 1H), 7.52- |
| | | 7.49 (m, 2H), 7.17 (d, J = 8.0 Hz, 1H), 6.93 (t, |
| | | J = 51.8 Hz, 1H), 5.18 (s, 2H), 3.79 (s, 2H), |
| | | 3.03-2.84 (m, 5H), 1.15 (d, J = 6.4 Hz, 6H); LRMS |
| | | (ES) m/z 469.7 (M⁺ + 1). |
| 14 | 14 | 5-(2-cyclobutyl-3,4-dihydro-1H-isoquinolin-7- |
| | | yl)-3-[[5-[5-(difluoromethyl)-1,3,4-oxadiazol-2- |
| | | yl]-2-pyridyl]methyl]-1,3,4-oxadiazol-2-one |
| | | ¹H NMR (400 MHz, CDCl₃) δ 9.25 (s, 1H), 8.38 (d, |
| | | J = 10.4 Hz, 1H), 7.58-7.48 (m, 3H), 7.17 (d, J = |
| | | 8.0 Hz, 1H), 6.92 (t, J = 51.8 Hz, 1H), 5.17 |
| | | (s, 2H), 3.65 (s, 2H), 3.05-2.97 (m, 3H), 2.77 |
| | | (t, J = 6.0 Hz, 2H), 2.12-1.93 (m, 4H), 1.78-1.67 |
| | | (m, 2H); LRMS (ES) m/z 481.6 (M⁺ + 1). |

### Example 135: Synthesis of Compound 135, 3-[[5-[5-(difluoromethyl)-1,3,4-oxadiazol-2-yl]-3-fluoro-2-pyridyl]methyl]-5-(2-methyl-3,4-dihydro-1H-isoquinolin-6-yl)-1,3,4-oxadiazol-2-thione

### [Step 1] Synthesis of tert-butyl 6-(hydrazinecarbonyl)-3,4-dihydro-1H-isoquinolin-2-carboxylate

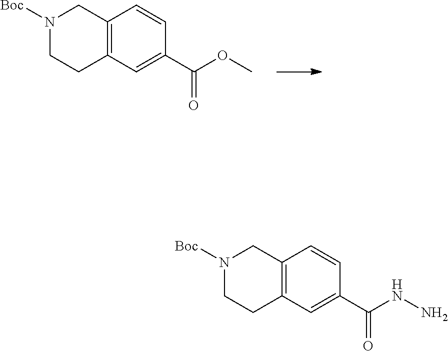 [SMILES: COC(=O)c1ccc2c(c1)CCN(C(=O)OC(C)(C)C)C2 ; CC(C)(C)OC(=O)N1CCc2cc(C(=O)NN)ccc2C1]

To a solution in which 2-tert-butyl, 6-methyl 3,4-dihydro-1H-isoquinolin-2,6-dicarboxylate (100.00%, 1.000 g, 3.433 mmol) was dissolved in ethanol (20 mL) at room temperature, hydrazine monohydrate (100.00% solution, 1.668 mL, 34.320 mmol) was added and stirred overnight at 60° C. Then, the reaction was terminated by lowering the temperature to room temperature. After removing the solvent from the reaction mixture under reduced pressure, the concentrate was purified and concentrated by column chromatography (SiO₂, 24 g cartridge; methanol/dichloromethane=0% to 10%) to obtain the title compound (1 g, 100.0%, white solid).

### [Step 2] Synthesis of tert-butyl 6-(2-thioxo-3H-1,3,4-oxadiazol-5-yl)-3,4-dihydro-H-isoquinolin-2-carboxylate

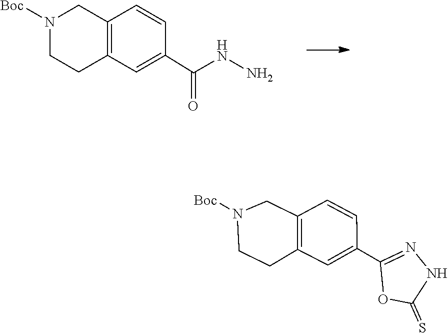 [SMILES: CC(C)(C)OC(=O)N1CCc2cc(C(=O)NN)ccc2C1 ; CC(C)(C)OC(=O)N1CCc2cc(-c3n[nH]c(=S)o3)ccc2C1]

A solution in which tert-butyl 6-(hydrazinecarbonyl)-3,4-dihydro-1H-isoquinolin-2-carboxylate (100.00%, 0.642 g, 2.204 mmol) synthesized in Step 1 and potassium ethylxanthate (100.00%, 0.353 g, 2.202 mmol) were dissolved in ethanol (10 mL) at room temperature was stirred at 90° C. overnight. Then, the reaction was terminated by lowering the temperature to room temperature. A saturated aqueous ammonium chloride solution was poured into the reaction mixture, followed by extraction with dichloromethane. The organic layer was washed with a saturated aqueous sodium chloride solution, dried over anhydrous magnesium sulfate, filtered, and concentrated under reduced pressure. The concentrate was purified and concentrated by column chromatography (SiO₂, 4 g cartridge; dichloromethane/methanol=0% to 10%) to obtain the title compound (0.03 g, 4.083%, colorless oil).

[Step 3] Synthesis of tert-butyl 6-[4-[[5-[5-(difluoromethyl)-1,3,4-oxadiazol-2-yl]-3-fluoro-2-pyridyl]methyl]-5-oxo-1,3,4-oxadiazol-2-yl]-3,4-dihydro-1H-isoquinolin-2-carboxylate

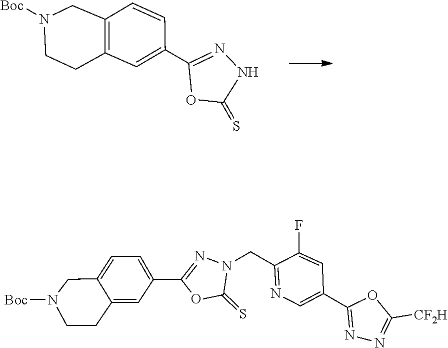 [SMILES: CC(C)(C)OC(=O)N1CCc2cc(-c3n[nH]c(=S)o3)ccc2C1 ; CC(C)(C)OC(=O)N1CCc2cc(-c3nn(Cc4ncc(-c5nnc(C(F)F)o5)cc4F)c(=S)o3)ccc2C1]

To a solution in which ter-butyl 6-(2-thioxo-3H-1,3,4-oxadiazol-5-yl)-3,4-dihydro-1H-isoquinolin-2-carboxylate (100.00%, 0.550 g, 1.650 mmol) synthesized in Step 2 and potassium carbonate (100.00%, 0.229 g, 2.311 mmol) were dissolved in N,N-dimethylformamide (10 mL) at room temperature, 2-[6-(bromomethyl)-5-fluoro-3-pyridyl]-5-(difluoromethyl)-1,3,4-oxadiazole (100.00%, 0.610 g, 1.980 mmol) was added and stirred overnight at the same temperature. Water was poured into the reaction mixture, and the mixture was extracted with ethyl acetate. The organic layer was washed with a saturated aqueous sodium chloride solution, dried over anhydrous magnesium sulfate, filtered, and concentrated under reduced pressure. The concentrate was purified and concentrated by column chromatography (SiO₂, 4 g cartridge; ethyl acetate/hexane=0% to 60%) to obtain the title compound (0.395 g, 42.71%, yellow solid).

### [Step 4] Synthesis of 3-[[5-[5-(difluoromethyl)-1,3,4-oxadiazol-2-yl]-3-fluoro-2-pyridyl]methyl]-5-(1,2,3, 4-tetrahydroisoquinolin-6-yl)-1,3,4-oxadiazol-2-thione

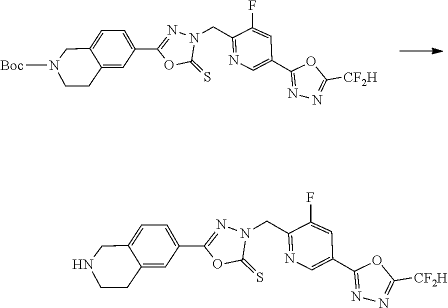 [SMILES: CC(C)(C)OC(=O)N1CCc2cc(-c3nn(Cc4ncc(-c5nnc(C(F)F)o5)cc4F)c(=S)o3)ccc2C1 ; Fc1cc(-c2nnc(C(F)F)o2)cnc1Cn1nc(-c2ccc3c(c2)CCNC3)oc1=S]

A solution in which tert-butyl 6-[4-[[5-[5-(difluoromethyl)-1,3,4-oxadiazol-2-yl]-3-fluoro-2-pyridyl]methyl]-5-thioxo-1,3,4-oxadiazol-2-yl]-3,4-dihydro-1H-isoquinolin-2-carboxylate (100.00%, 0.395 g, 0.705 mmol) synthesized in Step 3 and trifluoroacetic acid (100.00% solution, 0.539 mL, 7.040 mmol) were dissolved in dichloromethane (5 mL) at room temperature was stirred overnight at the same temperature. An aqueous solution of N-sodium hydrogen carbonate was poured into the reaction mixture, followed by extraction with dichloromethane. The organic layer was washed with a saturated aqueous sodium chloride solution, dried over anhydrous magnesium sulfate, filtered, and concentrated under reduced pressure. The obtained product was used without further purification (0.32 g, 98.64%, yellow oil).

### [Step 5] Synthesis of Compound 135

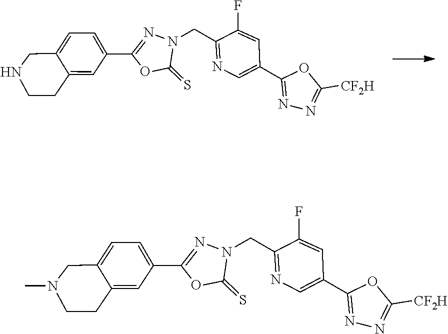 [SMILES: Fc1cc(-c2nnc(C(F)F)o2)cnc1Cn1nc(-c2ccc3c(c2)CCNC3)oc1=S ; CN1CCc2cc(-c3nn(Cc4ncc(-c5nnc(C(F)F)o5)cc4F)c(=S)o3)ccc2C1]

A solution in which 3-[[5-[5-(difluoromethyl)-1,3,4-oxadiazol-2-yl]-3-fluoro-2-pyridyl]methyl]-5-(1,2,3,4-tetrahydroisoquinolin-6-yl)-1,3,4-oxadiazol-2-thione (100.00%, 0.060 g, 0.130 mmol) synthesized in Step 4, formaldehyde (37.00% solution, 0.026 mL, 0.261 mmol) and N-ethyl-N-isopropyl-propan-2-amine (100.00% solution, 0.045 mL, 0.258 mmol) were dissolved in dichloromethane (1 mL) was stirred at room temperature for 30 minutes, and sodium triacetoxyborohydride (100.00%, 0.082 g, 0.389 mmol) was added and further stirred at the same temperature for 18 hours. Water was poured into the reaction mixture, and the mixture was extracted with dichloromethane. The organic layer was washed with a saturated aqueous sodium chloride solution, dried over anhydrous magnesium sulfate, filtered, and concentrated under reduced pressure. The concentrate was purified and concentrated by column chromatography (SiO₂, 4 g cartridge; dichloromethane/methanol=0% to 10%) to obtain the title compound (0.021 g, 33.96%, pale yellow solid).

¹H NMR (400 MHz, CDCl₃) δ 9.12 (s, 1H), 8.15 (dd, J=9.0, 1.8 Hz, 1H), 7.79-7.76 (m, 2H), 7.17 (d, J=8.0 Hz, 1H), 6.96 (t, J=52.2 Hz, 1H), 4.80 (d, J=1.6 Hz, 2H), 3.65 (s, 2H), 3.01 (t, J=5.8 Hz, 2H), 2.76-2.73 (m, 2H), 2.50 (s, 3H); LRMS (ES) m/z 475.9 (M⁺+1).

Compounds in Table 21 below were synthesized according to substantially the same method as in Example 135.

TABLE 21

| | Com- | |
|---|---|---|
| Exam- | pound | |
| ple | No. | Compound Name, ¹H-NMR, MS (ESI) |
| 136 | 136 | 3-[[5-[5-(difluoromethyl)-1,3,4-oxadiazol-2-yl]- |
| | | 3-fluoro-2-pyridyl]methyl]-5-(2-isopropyl-3,4- |
| | | dihydro-1H-isoquinolin-6-yl)-1,3,4-oxadiazol-2- |
| | | thione |
| | | ¹H NMR (400 MHz, CDCl₃) δ 9.12 (d, J = 0.8 Hz, |
| | | 2H), 8.15 (dd, J = 8.8, 1.6 Hz, 1H), 7.77-7.75 |
| | | (m, 2H), 7.18 (d, J = 8.0 Hz, 1H), 6.96 (t, J = |
| | | 51.6 Hz, 1H), 4.80 (d, J = 2.0 Hz, 2H), 3.80 (s, |
| | | 2H), 2.99-2.93 (m, 3H), 2.84-2.81 (m, 2H), 1.18 |
| | | (d, J = 6.4 Hz, 6H); LRMS (ES) m/z 503.9 (M⁺ + 1). |
| 137 | 137 | 5-(2-cyclobutyl-3,4-dihydro-1H-isoquinolin-6- |
| | | yl)-3-[[5-[5-(difluoromethyl)-1,3,4-oxadiazol-2- |
| | | yl]-3-fluoro-2-pyridyl]methyl]-1,3,4-oxadiazol- |
| | | 2-thione |
| | | ¹H NMR (400 MHz, CDCl₃) δ 9.12 (s, 1H), 8.15 (d, |
| | | J = 8.8 Hz, 1H), 7.76 (d, J = 8.8 Hz, 2H), 7.17 |
| | | (d, J = 8.0 Hz, 1H), 6.96 (t, J = 51.6 Hz, 1H), |
| | | 4.80 (s, 2H), 3.57 (s, 2H), 2.99-2.92 (m, 3H), |
| | | 2.67-2.64 (m, 2H), 2.18-2.16 (m, 2H), 2.05-1.99 |
| | | (m, 2H), 1.84-1.73 (m, 2H); LRMS (ES) m/z 515.9 |
| | | (M⁺ + 1). |
| 138 | 138 | 5-(2-cyclohexyl-3,4-dihydro-1H-isoquinolin-6- |
| | | yl)-3-[[5-[5-(difluoromethyl)-1,3,4-oxadiazol-2- |
| | | yl]-3-fluoro-2-pyridyl]methyl]-1,3,4-oxadiazol- |
| | | 2-thione |
| | | ¹H NMR (400 MHz, CDCl₃) δ 9.12 (s, 1H), 8.15 (d, |
| | | J = 9.2 Hz, 1H), 7.76-7.74 (m, 2H), 7.16 (d, J = |
| | | 8.0 Hz, 1H), 6.96 (t, J = 51.6 Hz, 1H), 4.80 (s, |
| | | 2H), 3.86 (s, 2H), 2.97-2.88 (m, 4H), 2.52 (s, |
| | | 1H), 1.98-1.85 (m, 4H), 1.71-1.68 (m, 1H), 1.41- |
| | | 1.30 (m, 5H); LRMS (ES) m/z 544.0 (M⁺ + 1). |
| 139 | 139 | 3-[[5-[5-(difluoromethyl)-1,3,4-oxadiazol-2-yl]- |
| | | 2-pyridyl]methyl]-5-(6-methyl-5,7-dihydro-4H- |
| | | thieno[2,3-c]pyridin-2-yl)-1,3,4-oxadiazol-2- |
| | | thione |
| | | ¹H NMR (400 MHz, CDCl₃) δ 9.32 (s, 1H), 8.40-8.37 |
| | | (m, 1H), 7.81 (d, J = 8.4 Hz, 1H), 7.40 (s, 1H), |
| | | 6.96 (t, J = 51.6 Hz, 1H), 4.71 (s, 2H), 3.68 (s, |
| | | 2H), 2.80-2.76 (m, 4H), 2.52 (s, 3H); LRMS (ES) |
| | | m/z 463.8 (M⁺ + 1). |
| 140 | 140 | 3-[[5-[5-(difluoromethyl)-1,3,4-oxadiazol-2-yl]- |
| | | 2-pyridyl]methyl]-5-(6-ethyl-5,7-dihydro-4H- |
| | | thieno[2,3-c]pyridin-2-yl)-1,3,4-oxadiazol-2- |
| | | thione |
| | | ¹H NMR (400 MHz, CDCl₃) δ 9.31 (s, 1H), 8.38 (d, |
| | | J = 8.4 Hz, 1H), 7.81 (d, J = 8.4 Hz, 1H), 7.39 |
| | | (s, 1H), 6.96 (t, J = 51.4 Hz, 1H), 4.71 (s, 2H), |
| | | 3.74 (s, 2H), 2.81 (s, 4H), 2.70-2.66 (m, 2H), |
| | | 1.23-1.20 (m, 3H); LRMS (ES) m/z 477.8 (M⁺ + 1). |
| 141 | 141 | 3-[[5-[5-(difluoromethyl)-1,3,4-oxadiazol-2-yl]- |
| | | 2-pyridyl]methyl]-5-(6-isopropyl-5,7-dihydro-4H- |
| | | thieno[2,3-c]pyridin-2-yl)-1,3,4-oxadiazol-2- |
| | | thione |
| | | ¹H NMR (400 MHz, CDCl₃) δ 9.31 (d, J = 1.6 Hz, |
| | | 1H), 8.38 (dd, J = 8.0, 2.0 Hz, 1H), 7.81 (d, J = |
| | | 8.4 Hz, 1H), 6.96 (t, J = 51.6 Hz, 1H), 4.71 |
| | | (s, 2H), 3.82 (s, 2H), 3.02-2.96 (m, 1H), 2.85- |
| | | 2.77 (m, 4H), 1.17 (d, J = 6.8 Hz, 6H); LRMS (ES) |
| | | m/z 491.8 (M⁺ + 1). |

### Example 69: Synthesis of Compound 69, 3-[[5-[5-(difluoromethyl)-1,3,4-oxadiazol-2-yl]-2-pyridyl]methyl]-5-(2-thienyl)-6H-1,3,4-oxadiazin-2-one

### [Step 1] Synthesis of [2-oxo-2-(2-thienyl)ethyl]acetate

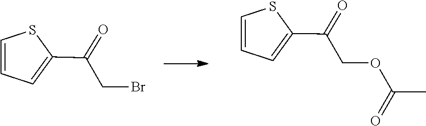

To a solution in which 2-bromo-1-(2-thienyl)ethanone (100.00%, 0.500 g, 2.438 mmol) was dissolved in N,N-dimethylformamide (10 mL) at room temperature, potassium acetate (100.00%, 0.718 g, 7.316 mmol) and potassium iodide (100.00%, 0.405 g, 2.440 mmol) were added and stirred at the same temperature for 18 hours. Water was poured into the reaction mixture, and the mixture was extracted with ethyl acetate. The organic layer was washed with a saturated aqueous sodium chloride solution, dried over anhydrous magnesium sulfate, filtered, and concentrated under reduced pressure. The obtained product was used without further purification (0.445 g, 99.078%, yellow oil).

### [Step 2] Synthesis of [(2E)-2-(methoxycarbonylhydrazono)-2-(2-thienyl)ethyl]acetate

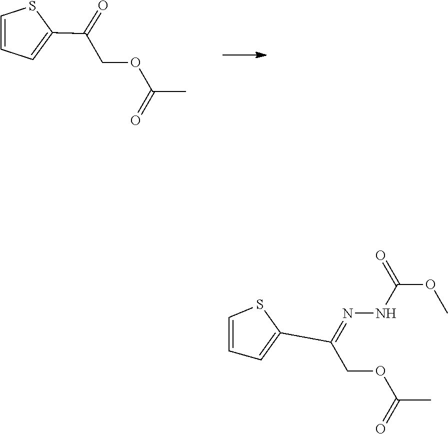 [SMILES: CC(=O)OCC(=O)c1cccs1 ; COC(=O)N/N=C(\COC(C)=O)c1cccs1]

To a solution in which [2-oxo-2-(2-thienyl)ethyl]acetate (100.00%, 0.445 g, 2.416 mmol) synthesized in Step 1 and hydrogen chloride (1.00 M solution, 0.048 mL, 0.048 mmol) were dissolved in methanol (10 mL) at room temperature, methyl hydrazinocarboxylate (100.00%, 0.239 g, 2.653 mmol) was added and stirred at the same temperature for 18 hours. Water was poured into the reaction mixture, and the mixture was extracted with dichloromethane. The organic layer was washed with a saturated aqueous sodium chloride solution, dried over anhydrous magnesium sulfate, filtered, and concentrated under reduced pressure. The concentrate was purified and concentrated by column chromatography (SiO₂, 12 g cartridge; ethyl acetate/hexane=0% to 50%) to obtain the title compound (0.425 g, 68.648%, yellow solid).

### [Step 3] Synthesis of 5-(2-thienyl)-3,6-dihydro-1,3,4-oxadiazin-2-one

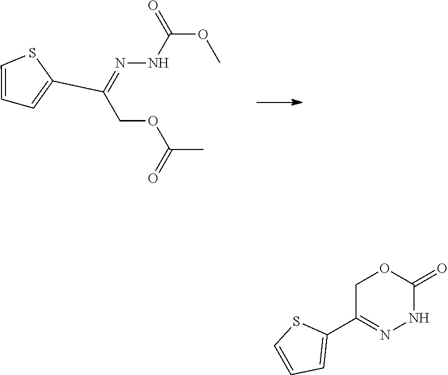 [SMILES: COC(=O)N/N=C(\COC(C)=O)c1cccs1 ; O=C1NN=C(c2cccs2)CO1]

A solution in which [(2E)-2-(methoxycarbonylhydrazono)-2-(2-thienyl)ethyl]acetate (100.00%, 0.425 g, 1.658 mmol) synthesized in Step 2 and sodium ethoxide (ca. 20% in ethanol, 20.00% solution, 0.962 mL, 2.500 mmol) were dissolved in ethanol (10 mL) at room temperature was stirred for 30 minutes at the same temperature. A saturated aqueous ammonium chloride solution was poured into the reaction mixture, followed by extraction with dichloromethane. The organic layer was washed with a saturated aqueous sodium chloride solution, dried over anhydrous magnesium sulfate, filtered, and concentrated under reduced pressure. The concentrate was purified and concentrated by column chromatography (SiO₂, 12 g cartridge; ethyl acetate/hexane=0% to 30%) to obtain the title compound (0.2 g, 66.192%, yellow solid).

### [Step 4] Synthesis of Compound 69

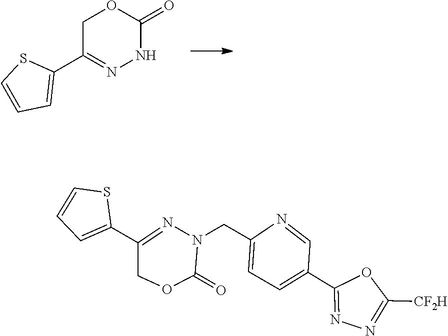 [SMILES: O=C1NN=C(c2cccs2)CO1 ; O=C1OCC(c2cccs2)=NN1Cc1ccc(-c2nnc(C(F)F)o2)cn1]

A solution in which 5-(2-thienyl)-3,6-dihydro-1,3,4-oxadiazin-2-one (100.00%, 0.045 g, 0.247 mmol) synthesized in Step 3, 2-[6-(bromomethyl)-3-pyridyl]-5-(difluoromethyl)-1,3,4-oxadiazole (100.00%, 0.075 g, 0.259 mmol), potassium carbonate (100.00%, 0.037 g, 0.373 mmol) and potassium iodide (100.00%, 0.020 g, 0.120 mmol) were dissolved in N,N-dimethylformamide (1 mL) at room temperature was stirred overnight at the same temperature. Water was poured into the reaction mixture, and the mixture was extracted with ethyl acetate. The organic layer was washed with a saturated aqueous sodium chloride solution, dried over anhydrous magnesium sulfate, filtered, and concentrated under reduced pressure. The concentrate was purified and concentrated by column chromatography (SiO₂, 4 g cartridge; ethyl acetate/hexane=0% to 60%) to obtain the title compound (0.042 g, 43.45%, pale yellow solid).

¹H NMR (400 MHz, CDCl₃) δ 9.32 (d, J=1.6 Hz, 1H), 8.40 (dd, J=8.2, 2.2 Hz, 1H), 7.53 (d, J=8.4 Hz, 1H), 7.47 (dd, J=5.2, 1.2 Hz, 1H), 7.25-7.24 (m, 1H), 7.11-7.09 (m, 1H), 6.96 (t, J=51.6 Hz, 1H), 5.27 (s, 2H), 5.25 (s, 2H); LRMS (ES) m/z 392.3 (M⁺+1).

Compounds in Table 22 below were synthesized according to substantially the same method as in Example 69.

TABLE 22

| | Com- | |
|---|---|---|
| Exam- | pound | |
| ple | No. | Compound Name, ¹H-NMR, MS (ESI) |
| 70 | 70 | 3-[[5-[5-(difluoromethyl)-1,3,4-oxadiazol-2-yl]- |
| | | 3-fluoro-2-pyridyl]methyl]-5-(2-thienyl)-6H- |
| | | 1,3,4-oxadiazin-2-one |
| | | ¹H NMR (400 MHz, CDCl₃) δ 9.11 (s, 1H), 8.13-8.11 |
| | | (m, 1H), 7.45 (dd, J = 5.0, 1.0 Hz, 1H), 7.23 |
| | | (dd, J = 3.8, 1.0 Hz, 1H), 7.10-7.08 (m, 1H), |
| | | 6.96 (t, J = 51.6 Hz, 1H), 5.32 (d, J = 1.6 Hz, |
| | | 2H), 5.27 (s, 2H); LRMS (ES) m/z 410.4 (M⁺ + 1). |
| 71 | 71 | 3-[[5-[5-(difluoromethyl)-1,3,4-oxadiazol-2-yl]- |
| | | 3-fluoro-2-pyridyl]methyl]-5-phenyl-6H-1,3,4- |
| | | oxadiazin-2-one |
| | | ¹H NMR (400 MHz, CDCl₃) δ 9.11 (s, 1H), 8.13-8.10 |
| | | (m, 1H), 7.62-7.60 (m, 2H), 7.46-7.40 (m, 3H), |
| | | 7.08 (s, 0.3H), 6.95 (s, 0.5H), 6.82 (s, 0.2H), |
| | | 5.35 (s, 2H), 5.32 (s, 2H); LRMS (ES) m/z 404.6 |
| | | (M⁺ + 1). |
| 72 | 72 | 3-[[5-[5-(difluoromethyl)-1,3,4-oxadiazol-2-yl]- |
| | | 2-pyridyl]methyl]-5-(2-pyridyl)-6H-1,3,4- |
| | | oxadiazin-2-one |
| | | ¹H NMR (400 MHz, CDCl₃) δ 9.32 (s, 1H), 8.59-8.58 |
| | | (m, 1H), 8.40 (dd, J = 8.2, 2.2 Hz, 1H), 7.99- |
| | | 7.96 (m, 1H), 7.76-7.72 (m, 1H), 7.56-7.54 (m, |
| | | 1H), 7.35-7.32 (m, 1H), 7.08 (s, 0.3H), 6.95 (s, |
| | | 0.5H), 6.82 (s, 0.2H), 5.56 (s, 2H), 5.29 (s, |
| | | 2H); LRMS (ES) m/z 387.7 (M⁺ + 1). |
| 73 | 73 | 3-[[5-[5-(difluoromethyl)-1,3,4-oxadiazol-2-yl]- |
| | | 3-fluoro-2-pyridyl]methyl]-5-(2-pyridyl)-6H- |
| | | 1,3,4-oxadiazin-2-one |
| | | ¹H NMR (400 MHz, CDCl₃) δ 9.12 (s, 1H), 8.59-8.58 |
| | | (m, 1H), 8.13 (dd, J = 9.3, 1.7 Hz, 1H), 7.95- |
| | | 7.92 (m, 1H), 7.75-7.70 (m, 1H), 7.34-7.31 (m, |
| | | 1H), 7.08 (s, 0.3H), 6.95 (s, 0.5H), 6.82 (s, |
| | | 0.2H), 5.57 (s, 2H), 5.36 (s, 2H); LRMS (ES) m/z |
| | | 405.7 (M⁺ + 1). |
| 74 | 74 | 3-[[5-[5-(difluoromethyl)-1,3,4-oxadiazol-2-yl]- |
| | | 2-pyridyl]methyl]-5-[5-(2-pyridyl)-2-thienyl]- |
| | | 6H-1,3,4-oxadiazin-2-one |
| | | ¹H NMR (400 MHz, CDCl₃) δ 9.32 (d, J = 2.0 Hz, |
| | | 1H), 8.59 (d, J = 4.8 Hz, 1H), 8.40 (dd, J = 8.4, |
| | | 2.0 Hz, 1H), 7.76-7.67 (m, 2H), 7.55-7.54 (m, |
| | | 2H), 7.25-7.21 (m, 2H), 6.96 (t, J = 51.6 Hz, |
| | | 1H), 5.28 (s, 2H), 5.25 (s, 2H); LRMS (ES) m/z |
| | | 469.7 (M⁺ + 1). |

### Example 142: Synthesis of Compound 142, 3-((5-(5-(difluoromethyl)-1,3,4-oxadiazol-2-yl)pyridin-2-yl)methyl)-5-phenyloxazol-2(3H)-one

### [Step 1] Synthesis of methyl 6-(((tert-butoxycarbonyl)amino)methyl)nicotinate

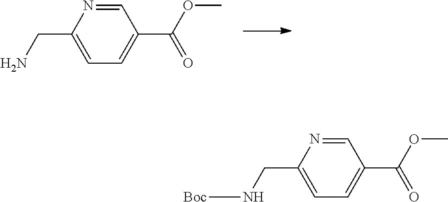 [SMILES: COC(=O)c1ccc(CN)nc1 ; COC(=O)c1ccc(CNC(=O)OC(C)(C)C)nc1]

To a solution in which methyl 6-(aminomethyl)nicotinate (2.000 g, 12.035 mmol) was dissolved in dichloromethane (20 mL) at room temperature, di-tert-butyl dicarbonate (3.152 g, 14.442 mmol) and triethylamine (2.516 mL, 18.053 mmol) were added and stirred at the same temperature for 18 hours. Water was poured into the reaction mixture, and the mixture was extracted with dichloromethane. The organic layer was washed with a saturated aqueous sodium chloride solution, dried over anhydrous sodium sulfate, filtered, and concentrated under reduced pressure. The concentrate was purified and concentrated by column chromatography (SiO₂, 24 g cartridge; ethyl acetate/hexane=0% to 40%) to obtain the title compound (1.700 g, 53.0%, white solid).

### [Step 2] Synthesis of methyl 6-(((tert-butoxycarbonyl)(phenylethynyl)amino)methyl)nicotinate

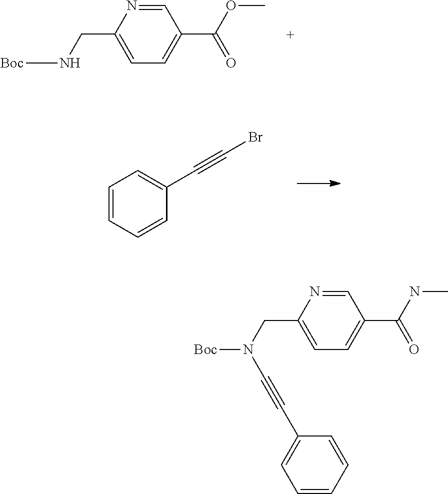 [SMILES: COC(=O)c1ccc(CNC(=O)OC(C)(C)C)nc1 ; BrC#Cc1ccccc1 ; CNC(=O)c1ccc(CN(C#Cc2ccccc2)C(=O)OC(C)(C)C)nc1]

(Bromoethynyl)benzene (0.200 g, 1.105 mmol) was dissolved in toluene (5 mL), and methyl 6-(((tert-butoxycarbonyl)amino)methyl)nicotinate (0.353 g, 1.326 mmol) synthesized in Step 1 at room temperature, Cooper (II) sulfate pentahydrate (0.055 g, 0.221 mmol), potassium phosphate (0.563 g, 2.651 mmol) and 1,10-phenanthroline (0.080 g, 0.442 mmol) were added and stirred at 80° C. for 36 hours. Then, the reaction was terminated by lowering the temperature to room temperature. The reaction mixture was filtered through a celite pad to remove solids, and the solvent was removed from the filtrate under reduced pressure. Then, the concentrate was purified and concentrated by column chromatography (SiO₂, 12 g cartridge; ethyl acetate/hexane=0% to 70%) to obtain the title compound (0.110 g, 27.2%, pale yellow solid).

### [Step 3] Synthesis of methyl 6-((2-oxo-5-phenyloxazol-3(2H)-yl)methyl)nicotinate

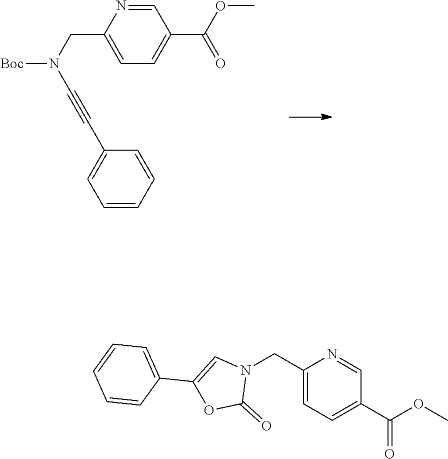 [SMILES: COC(=O)c1ccc(CN(C#Cc2ccccc2)C(=O)OC(C)(C)C)nc1 ; COC(=O)c1ccc(Cn2cc(-c3ccccc3)oc2=O)nc1]

To a solution in which methyl 6-(((tert-butoxycarbonyl)(phenylethynyl)amino)methyl)nicotinate (0.050 g, 0.136 mmol) synthesized in Step 2 was dissolved in dichloromethane (0.5 mL) at room temperature, silver bis(trifluoromethanesulfonyl)imide (0.003 g, 0.007 mmol) was added and stirred at the same temperature for 1 hour. A saturated aqueous ammonium chloride solution was poured into the reaction mixture and extracted with ethyl acetate. The organic layer was washed with a saturated aqueous sodium chloride solution, dried over anhydrous sodium sulfate, filtered, and concentrated under reduced pressure. The concentrate was purified and concentrated by column chromatography (SiO₂, 4 g cartridge; ethyl acetate/hexane=0% to 30%) to obtain the title compound (0.030 g, 70.8%, yellow solid).

### [Step 4] Synthesis of 6-((2-oxo-5-phenyloxazol-3(2H)-yl)methyl)nicotinohydrazide

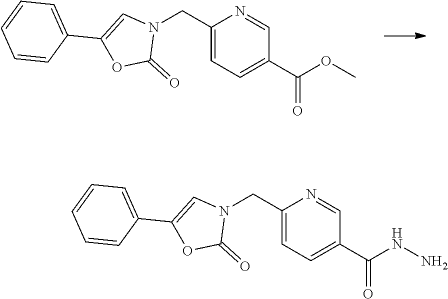 [SMILES: COC(=O)c1ccc(Cn2cc(-c3ccccc3)oc2=O)nc1 ; NNC(=O)c1ccc(Cn2cc(-c3ccccc3)oc2=O)nc1]

A solution in which methyl 6-((2-oxo-5-phenyloxazol-3(2H)-yl)methyl)nicotinate (0.016 g, 0.052 mmol) synthesized in Step 3 and hydrazine monohydrate (0.050 mL, 1.031 mmol) were dissolved in ethanol (1 mL) at 80° C. was stirred for 18 hours at the same temperature. Then, the reaction was terminated by lowering the temperature to room temperature. After removing the solvent from the reaction mixture under reduced pressure, the obtained product was used without further purification (0.016 g, 100.0%, white solid).

### [Step 5] Synthesis of Compound 142

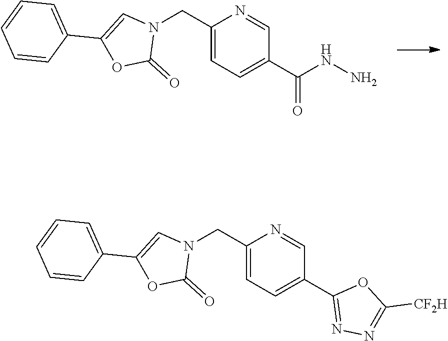 [SMILES: NNC(=O)c1ccc(Cn2cc(-c3ccccc3)oc2=O)nc1 ; O=c1oc(-c2ccccc2)cn1Cc1ccc(-c2nnc(C(F)F)o2)cn1]

To a solution in which 6-((2-oxo-5-phenyloxazol-3(2H)-yl)methyl)nicotinohydrazide (0.015 g, 0.048 mmol) synthesized in Step 4 and triethylamine (0.040 mL, 0.290 mmol) were dissolved in tetrahydrofuran (0.7 mL) at room temperature, 2,2-difluoroacetic anhydride (0.024 mL, 0.193 mmol) was added, and stirred at 80° C. for 6 hours. Then, the reaction was terminated by lowering the temperature to room temperature. A saturated aqueous solution of sodium hydrogen carbonate was poured into the reaction mixture, followed by extraction with ethyl acetate. The organic layer was washed with a saturated aqueous sodium chloride solution, dried over anhydrous sodium sulfate, filtered, and concentrated under reduced pressure. The concentrate was purified and concentrated by column chromatography (SiO₂, 4 g cartridge; hexane/ethyl acetate=100% to 50%) to obtain the title compound (0.008 g, 44.7%, white solid).

¹H NMR (400 MHz, CD₃OD) δ 9.28 (s, 1H), 8.53 (dd, J=7.8, 2.2 Hz, 1H), 7.67 (d, J=8.4 Hz, 1H), 7.56 (d, J=7.6 Hz, 2H), 7.44-7.40 (i, 3H), 7.33 (t, J=7.4 Hz, 1H), 7.26 (t, J=51.4 Hz, 1H), 7.26 (t, J=51.4 Hz, 1H), 5.09 (s, 2H); LRMS (ES) m/z 371.2 (Mm+1).

Compounds in Table 23 below were synthesized according to substantially the same method as in Example 142.

TABLE 23

| | Com- | |
|---|---|---|
| Exam- | pound | |
| ple | No. | Compound Name, ¹H-NMR, MS (ESI) |
| 143 | 143 | 3-((5-(5-(difluoromethyl)-1,3,4-oxadiazol-2- |
| | | yl)pyridin-2-yl)methyl)-5-(3- |
| | | fluorophenyl)oxazol-2(3H)-one |
| | | ¹H NMR (400 MHz, CD₃OD) δ 9.28 (d, J = 2.0 Hz, |
| | | 1H), 8.53 (dd, J = 8.2, 2.2 Hz, 1H), 7.68 (d, J = |
| | | 8.0 Hz, 1H), 7.54 (s, 1H), 7.47-7.38 (m, 2H), |
| | | 7.30 (d, J = 10.0 Hz, 1H), 7.27 (t, J = 51.4 Hz, |
| | | 1H), 7.08 (d, J = 8.8 Hz, 1H), 5.10 (s, 2H); LRMS |
| | | (ES) m/z 389.1 (M⁺ + 1). |

### Example 144: Synthesis of Compound 144, 1-((5-(5-(difluoromethyl)-1,3,4-oxadiazol-2-yl)pyridin-2-yl)methyl)-3-methyl-4-phenyl-1,3-dihydro-2H-imidazol-2-one

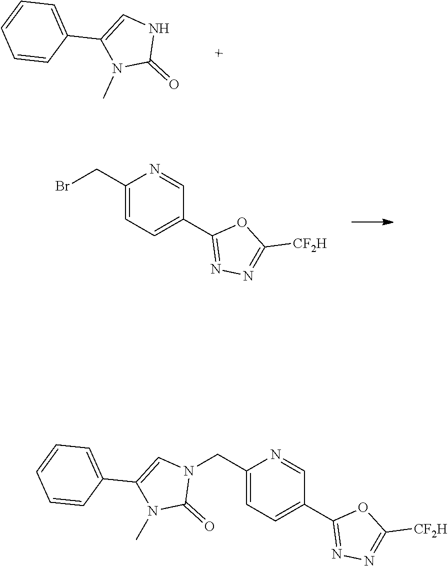 [SMILES: Cn1c(-c2ccccc2)c[nH]c1=O ; FC(F)c1nnc(-c2ccc(CBr)nc2)o1 ; Cn1c(-c2ccccc2)cn(Cc2ccc(-c3nnc(C(F)F)o3)cn2)c1=O]

To a solution in which 1-methyl-5-phenyl-1,3-dihydro-2H-imidazol-2-one (0.050 g, 0.287 mmol) and 2-(6-(bromomethyl)pyridin-3-yl)-5-(difluoromethyl)-1,3,4-oxadiazole (0.100 g, 0.345 mmol) were dissolved in N,N-dimethylformamide (1 mL) at room temperature, potassium carbonate (0.060 g, 0.431 mmol) and potassium iodide (0.010 g, 0.057 mmol) were added and stirred at 80° C. for 18 hours. Then, the reaction was terminated by lowering the temperature to room temperature. Water was poured into the reaction mixture, and the mixture was extracted with ethyl acetate. The organic layer was washed with a saturated aqueous sodium chloride solution, dried over anhydrous sodium sulfate, filtered, and concentrated under reduced pressure. The concentrate was purified and concentrated by column chromatography (SiO₂, 4 g cartridge; dichloromethane/methanol=100% to 80%) to obtain the title compound (0.012 g, 10.9%, white solid).

¹H NMR (400 MHz, CDCl₃) δ 8.18 (d, J=8.4 Hz, 2H), 8.15 (dd, J=4.2, 1.8 Hz, 1H), 7.71 (d, J=8.0 Hz, 2H), 7.62-7.57 (m, 2H), 7.51-7.37 (m, 3H), 6.95 (t, J=51.8 Hz, 1H), 6.79 (s, 1H), 5.66 (s, 2H), 3.51 (s, 3H); LRMS (ES) m/z 384.2 (M⁺+1).

### Activity Measurement and Analysis Protocol of Compounds According to Present Invention

### <Experimental Example 1> Confirmation of Inhibition of HDAC Enzyme Activity (In Vitro)

The following experiment was conducted to confirm the HDAC6 selectivity of the compound represented by Chemical Formula I of the present invention through the HDAC1 and HDAC6 enzyme activity inhibition test.

HDAC enzyme activity was measured using HDAC Fluorometric Drug Discovery Kit (BML-AK511, 516) manufactured by Enzo Life Science. For the HDAC1 enzyme activity test, human recombinant HDAC1 (BML-SE456) was used as an enzyme source and Fluor de Lys-®-″SIRT1 (BNL-KI177) was used as a substrate. The 5-fold diluted compound was dispensed in a 96-well plate, 0.3 μg of enzyme and 10 μM substrate were added to each well and reacted at 30° C. for 60 minutes, and Fluor de Lys® Developer II(BML-KI176) was added and reacted for 30 minutes to complete the reaction. Then, the fluorescence value (Ex 360, Em 460) was measured using a multiplate reader (Flexstation 3, Molecular Device). The HDAC6 enzyme was tested using the same protocol as the HDAC1 enzyme activity test method employing human recombinant HDAC6 (382180) from Calbiochem. For the final result, each IC₅ value was calculated using the GraphPad Prism 4.0 program. Results thereof are shown in Table 24 below.

TABLE 24

| | | | HDAC6 |
|---|---|---|---|
| | HDAC1 | HDAC6 | selectivity |
| Example | (μM) | (μM) | (fold) |
| 1 | >50 | 120.2 | 416 |
| 2 | >50 | 98.8 | 506 |
| 3 | >50 | 14.3 | 3497 |
| 4 | >50 | 84.1 | 595 |
| 5 | >50 | 84.9 | 589 |
| 6 | >50 | 46.4 | 1078 |
| 7 | >50 | 83.3 | 600 |
| 8 | >50 | 91.6 | 546 |
| 9 | >50 | 85.5 | 585 |
| 10 | >50 | 75.1 | 666 |
| 11 | >50 | 108.3 | 462 |
| 12 | >50 | 82.4 | 607 |
| 13 | >50 | 105.3 | 475 |
| 14 | >50 | 104.7 | 478 |
| 15 | >50 | 95.5 | 524 |
| 16 | >50 | 90.8 | 551 |
| 17 | >50 | 45.9 | 1089 |
| 18 | >50 | 65.1 | 768 |
| 19 | >50 | 33.1 | 1511 |
| 20 | >50 | 76.6 | 653 |
| 21 | >50 | 112.0 | 446 |
| 22 | >50 | 104.3 | 479 |
| 23 | >50 | 53.1 | 942 |
| 24 | >50 | 102.7 | 487 |
| 25 | >50 | 77.9 | 642 |
| 26 | >50 | 99.4 | 503 |
| 27 | >50 | 118.9 | 420 |
| 28 | >50 | 79.9 | 626 |
| 29 | >50 | 67.1 | 745 |
| 30 | >50 | 96.5 | 518 |
| 31 | >50 | 77.8 | 643 |
| 32 | >50 | 64.6 | 774 |
| 33 | >50 | 33.2 | 1506 |
| 34 | >50 | 29.5 | 1695 |
| 35 | >50 | 77.8 | 643 |
| 36 | >50 | 78.1 | 640 |
| 37 | >50 | 97.7 | 512 |
| 38 | >50 | 50.6 | 988 |
| 39 | >50 | 82.7 | 605 |
| 40 | >50 | 76.7 | 652 |
| 41 | >50 | 55.0 | 909 |
| 42 | >50 | 51.3 | 975 |
| 43 | >50 | 139.3 | 359 |
| 44 | >50 | 71.0 | 704 |
| 45 | >50 | 92.0 | 543 |
| 46 | >50 | 42.7 | 1171 |
| 47 | >50 | 32.3 | 1548 |
| 48 | >50 | 114.5 | 437 |
| 49 | >50 | 76.2 | 656 |
| 50 | >50 | 85.1 | 588 |
| 51 | >50 | 83.2 | 601 |
| 52 | >50 | 105.8 | 473 |
| 53 | >50 | 89.3 | 560 |
| 54 | >50 | 112.6 | 444 |
| 55 | >50 | 78.9 | 634 |
| 56 | >50 | 118.4 | 422 |
| 57 | >50 | 52.4 | 954 |
| 58 | >50 | 77.8 | 643 |
| 59 | >50 | 74.7 | 669 |
| 60 | >50 | 81.4 | 614 |
| 61 | >50 | 92.6 | 540 |
| 62 | >50 | 100.0 | 500 |
| 63 | >50 | 80.7 | 620 |
| 64 | >50 | 119.5 | 418 |
| 65 | >50 | 93.1 | 537 |
| 66 | >50 | 33.4 | 1497 |
| 67 | >50 | 56.2 | 890 |
| 68 | >50 | 105.7 | 473 |
| 69 | >50 | 115.1 | 434 |
| 70 | >50 | 108.7 | 460 |
| 71 | >50 | 98.0 | 510 |
| 72 | >50 | 119.1 | 420 |
| 73 | >50 | 113.2 | 442 |
| 74 | >50 | 60.4 | 828 |
| 75 | >50 | 43.5 | 1149 |
| 76 | >50 | 52.9 | 945 |
| 77 | >50 | 74.7 | 669 |
| 78 | >50 | 100.4 | 498 |
| 79 | >50 | 108.5 | 461 |
| 80 | >50 | 77.1 | 649 |
| 81 | >50 | 86.8 | 576 |
| 82 | >50 | 110.3 | 453 |
| 83 | >50 | 90.3 | 554 |
| 84 | >50 | 100.6 | 497 |
| 85 | >50 | 111.8 | 447 |
| 86 | >50 | 65.8 | 760 |
| 87 | >50 | 89.9 | 556 |
| 88 | >50 | 111.4 | 449 |
| 89 | >50 | 48.7 | 1027 |
| 90 | >50 | 59.9 | 835 |
| 91 | >50 | 27.3 | 1832 |
| 92 | >50 | 15.7 | 3185 |
| 93 | >50 | 17.3 | 2890 |
| 94 | >50 | 29.9 | 1672 |
| 95 | >50 | 87.1 | 574 |
| 96 | >50 | 41.4 | 1208 |
| 97 | >50 | 58.1 | 862 |
| 98 | >50 | 90.6 | 552 |
| 99 | >50 | 97.8 | 511 |
| 100 | >50 | 24.4 | 2049 |
| 101 | >50 | 19.7 | 2538 |
| 102 | >50 | 42.1 | 1188 |
| 103 | >50 | 38.4 | 1302 |
| 104 | >50 | 43.0 | 1163 |
| 105 | >50 | 68.1 | 734 |
| 106 | >50 | 78.2 | 639 |
| 107 | >50 | 43.0 | 1163 |
| 108 | >50 | 33.9 | 1475 |
| 109 | >50 | 49.0 | 1020 |
| 110 | >50 | 54.4 | 919 |
| 111 | >50 | 91.0 | 549 |
| 112 | >50 | 51.0 | 980 |
| 113 | >50 | 64.3 | 778 |
| 114 | >50 | 60.7 | 824 |
| 115 | >50 | 103.5 | 483 |
| 116 | >50 | 116.5 | 429 |
| 117 | >50 | 74.1 | 675 |
| 118 | >50 | 67.4 | 742 |
| 119 | >50 | 84.8 | 590 |
| 120 | >50 | 79.5 | 630 |
| 121 | >50 | 78.2 | 639 |
| 122 | >50 | 110.3 | 453 |
| 123 | >50 | 45.1 | 1109 |
| 124 | >50 | 101.8 | 491 |
| 125 | >50 | 88.8 | 563 |
| 126 | >50 | 51.2 | 977 |
| 127 | >50 | 56.9 | 879 |
| 128 | >50 | 85.7 | 583 |
| 129 | >50 | 109.3 | 457 |
| 130 | >50 | 66.7 | 750 |
| 131 | >50 | 81.7 | 612 |
| 132 | >50 | 103.3 | 484 |
| 133 | >50 | 113.0 | 442 |
| 134 | >50 | 73.5 | 680 |
| 135 | >50 | 67.5 | 741 |
| 136 | >50 | 59.5 | 840 |
| 137 | >50 | 46.5 | 1075 |
| 138 | >50 | 48.7 | 1042 |
| 139 | >50 | 90.6 | 552 |
| 140 | >50 | 44.9 | 1114 |
| 141 | >50 | 56.0 | 893 |
| 142 | >50 | 103.3 | 484 |
| 143 | >50 | 133.7 | 374 |
| 144 | >50 | 271.0 | 185 |

As described in Table 24 above, it was confirmed from the results of the HDAC 1 and HDAC6 activity inhibition test that the 1,3,4-oxadiazole derivative compound of the present invention, stereoisomers thereof, or pharmaceutically acceptable salts thereof exhibited about 185 to about 3497 times excellent selective HDAC6 inhibitory activity.

1. A 1,3,4-oxadiazole derivative compound represented by the following Chemical Formula I, a stereoisomer thereof or a pharmaceutically acceptable salt thereof: 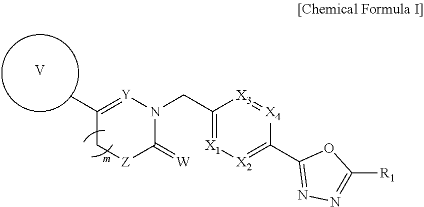
in Chemical Formula I above,
R₁ is —C₁₋₄haloalkyl;
X₁ to X₄ are each independently CR_{X} or N;
Rₓ is —H, —C₁₋₄alkyl, —C₁₋₄haloalkyl, or -halo;
Y is CR_{Y} or N;
R_{Y} is —H or —C₁₋₄alkyl;
Z is NR_{Z}, O, or S;
R_{z} is —H or —C₁₋₄alkyl;
W is O or S;
m is 0 or 1;
a ring V is an aryl, heteroaryl, or hydroheteroaryl, wherein at least one H of the aryl, heteroaryl, or hydroheteroaryl ring may be substituted with —C₁₋₄alkyl, —C₁₋₄aminoalkyl, —C₁₋₄hydroxyalkyl, —C₁₋₄haloalkyl, -halo, —(CH₂)n-cycloalkyl, —(CH₂)n-heterocycloalkyl, or —(CH₂)n-heteroaryl, wherein at least one H of the —(CH₂)n-cycloalkyl, —(CH₂)n-heterocycloalkyl, or —(CH₂)n-heteroaryl ring may be substituted with —C₁₋₄alkyl, —C₁₋₄haloalkyl, -halo, cycloalkyl, or heterocycloalkyl (wherein at least one H of the cycloalkyl or heterocycloalkyl ring may be substituted with —C₁₋₄alkyl); and
n is 0, 1, or 2.

2. The 1,3,4-oxadiazole derivative compound, the stereoisomer thereof or the pharmaceutically acceptable salt thereof according to claim 1,
wherein
R₁ is —C₁₋₄haloalkyl;
X₁ is N;
X₂ to X₄ are each independently CR_{X};
R_{X} is —H or -halo;
Y is CR_{Y} or N;
R_{Y} is —H;
Z is NR_{Z}, O, or S;
R_{z} is —C₁₋₄alkyl;
W is O or S;
m is 0 or 1;
the ring V is an aryl, heteroaryl, or hydroheteroaryl, wherein at least one H of the aryl, heteroaryl, or hydroheteroaryl ring may be substituted with —C₁₋₄alkyl, —C₁₋₄aminoalkyl, -halo, —(CH₂)n-cycloalkyl, —(CH₂)n-heterocycloalkyl, or —(CH₂)n-heteroaryl, wherein at least one H of the —(CH₂)n-cycloalkyl, —(CH₂)n-heterocycloalkyl, or —(CH₂)n-heteroaryl ring may be substituted with —C₁₋₄alkyl, -halo, cycloalkyl, or heterocycloalkyl (wherein at least one H of the cycloalkyl or heterocycloalkyl ring may be substituted with —C₁₋₄alkyl); and
n is 0 or 1.

3. The 1,3,4-oxadiazole derivative compound, the stereoisomer thereof or the pharmaceutically acceptable salt thereof according to claim 1, wherein
R₁ is —CF₂H or —CF₃.

4. The 1,3,4-oxadiazole derivative compound, the stereoisomer thereof or the pharmaceutically acceptable salt thereof according to claim 1, wherein
X₁ is N; and
X₂ to X₄ are each independently CH or CF.

5. The 1,3,4-oxadiazole derivative compound, the stereoisomer thereof or the pharmaceutically acceptable salt thereof according to claim 1, wherein
Y is CH or N;
Z is N—C₁₋₄alkyl, O, or S;
W is O or S; and
m is 0 or 1.

6. The 1,3,4-oxadiazole derivative compound, the stereoisomer thereof or the pharmaceutically acceptable salt thereof according to claim 1, wherein
the ring V is phenyl, 5-10 membered heteroaryl, or 9-10 membered hydroheteroaryl, wherein at least one H of the phenyl, 5-10 membered heteroaryl, or 9-10 membered hydroheteroaryl ring may be substituted with —C₁₋₄alkyl, —C₁₋₄aminoalkyl, -halo, —(CH₂)n-cycloalkyl, —(CH₂)n-heterocycloalkyl, or —(CH₂)n-heteroaryl, wherein at least one H of the —(CH₂)n-cycloalkyl, —(CH₂)n-heterocycloalkyl, or —(CH₂)n-heteroaryl ring may be substituted with —C₁₋₄alkyl, -halo, 4-6 membered cycloalkyl, or 4-6 membered heterocycloalkyl (wherein at least one H of the 4-6 membered cycloalkyl or 4-6 membered heterocycloalkyl ring may be substituted with —C₁₋₄alkyl); and
n is 0 or 1.

7. The 1,3,4-oxadiazole derivative compound, the stereoisomer thereof or the pharmaceutically acceptable salt thereof according to claim 1, wherein
the compound represented by Chemical Formula I is any one selected from the group consisting of the following compounds:

| Example | Compound | Structural Formula |
|---|---|---|
| 1 | 1 | 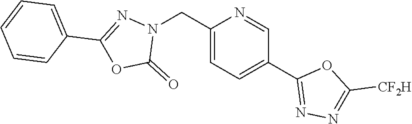 [SMILES: O=c1oc(-c2ccccc2)nn1Cc1ccc(-c2nnc(C(F)F)o2)cn1] |
| 2 | 2 | 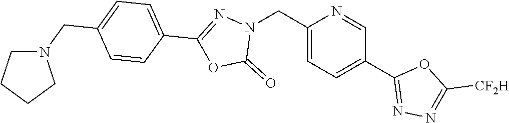 [SMILES: O=c1oc(-c2ccc(CN3CCCC3)cc2)nn1Cc1ccc(-c2nnc(C(F)F)o2)cn1] |
| 3 | 3 | 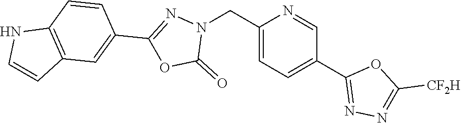 [SMILES: O=c1oc(-c2ccc3[nH]ccc3c2)nn1Cc1ccc(-c2nnc(C(F)F)o2)cn1] |
| 4 | 4 | 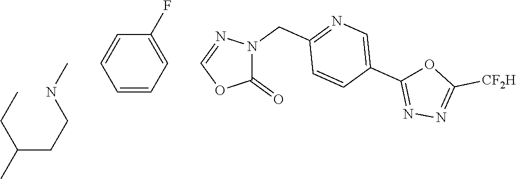 |
| 5 | 5 | 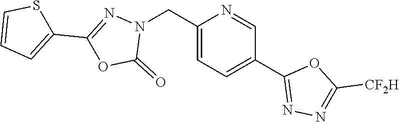 [SMILES: O=c1oc(-c2cccs2)nn1Cc1ccc(-c2nnc(C(F)F)o2)cn1] |
| 6 | 6 | 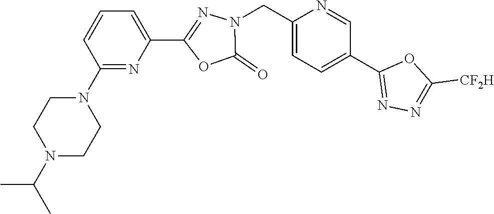 [SMILES: CC(C)N1CCN(c2cccc(-c3nn(Cc4ccc(-c5nnc(C(F)F)o5)cn4)c(=O)o3)n2)CC1] |
| 7 | 7 | 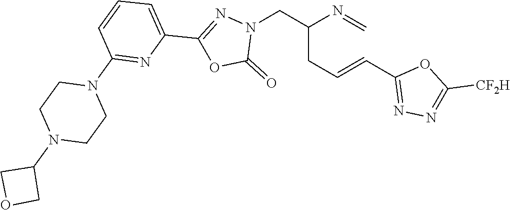 [SMILES: C=NC(C/C=C/c1nnc(C(F)F)o1)Cn1nc(-c2cccc(N3CCN(C4COC4)CC3)n2)oc1=O] |
| 8 | 8 | 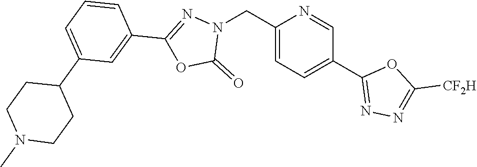 [SMILES: CN1CCC(c2cccc(-c3nn(Cc4ccc(-c5nnc(C(F)F)o5)cn4)c(=O)o3)c2)CC1] |
| 9 | 9 | 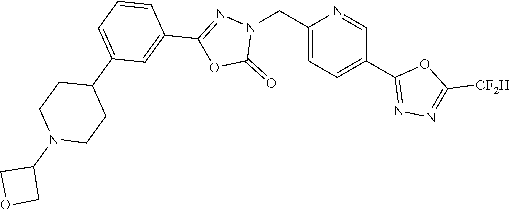 [SMILES: O=c1oc(-c2cccc(C3CCN(C4COC4)CC3)c2)nn1Cc1ccc(-c2nnc(C(F)F)o2)cn1] |
| 10 | 10 | 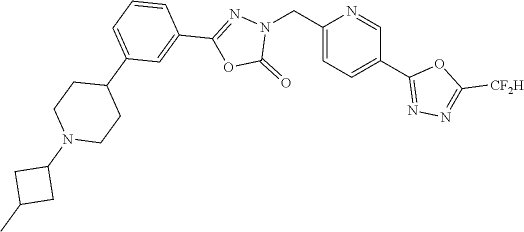 [SMILES: CC1CC(N2CCC(c3cccc(-c4nn(Cc5ccc(-c6nnc(C(F)F)o6)cn5)c(=O)o4)c3)CC2)C1] |
| 11 | 11 | 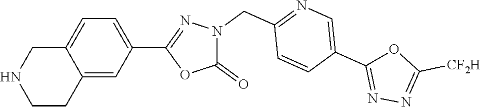 [SMILES: O=c1oc(-c2ccc3c(c2)CCNC3)nn1Cc1ccc(-c2nnc(C(F)F)o2)cn1] |
| 12 | 12 | 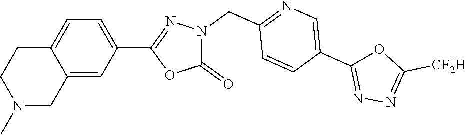 [SMILES: CN1CCc2ccc(-c3nn(Cc4ccc(-c5nnc(C(F)F)o5)cn4)c(=O)o3)cc2C1] |
| 13 | 13 | 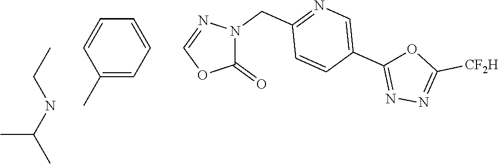 |
| 14 | 14 | 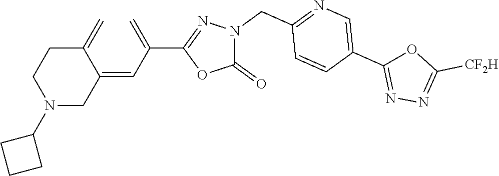 [SMILES: C=C1CCN(C2CCC2)C/C1=C/C(=C)c1nn(Cc2ccc(-c3nnc(C(F)F)o3)cn2)c(=O)o1] |
| 15 | 15 | 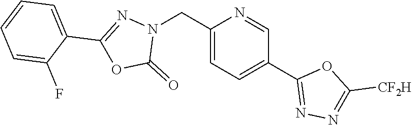 [SMILES: O=c1oc(-c2ccccc2F)nn1Cc1ccc(-c2nnc(C(F)F)o2)cn1] |
| 16 | 16 | 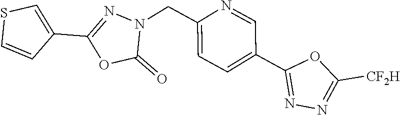 [SMILES: O=c1oc(-c2ccsc2)nn1Cc1ccc(-c2nnc(C(F)F)o2)cn1] |
| 17 | 17 | 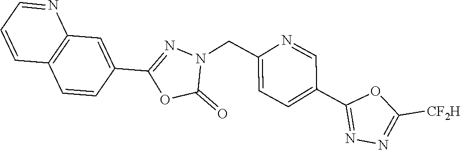 [SMILES: O=c1oc(-c2ccc3cccnc3c2)nn1Cc1ccc(-c2nnc(C(F)F)o2)cn1] |
| 18 | 18 | 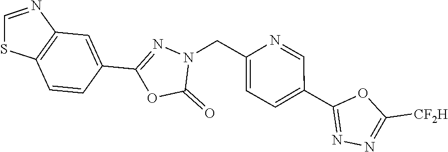 [SMILES: O=c1oc(-c2ccc3scnc3c2)nn1Cc1ccc(-c2nnc(C(F)F)o2)cn1] |
| 19 | 19 | 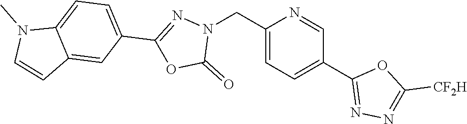 [SMILES: Cn1ccc2cc(-c3nn(Cc4ccc(-c5nnc(C(F)F)o5)cn4)c(=O)o3)ccc21] |
| 20 | 20 | 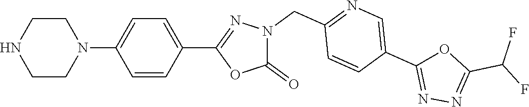 [SMILES: O=c1oc(-c2ccc(N3CCNCC3)cc2)nn1Cc1ccc(-c2nnc(C(F)F)o2)cn1] |
| 21 | 21 | 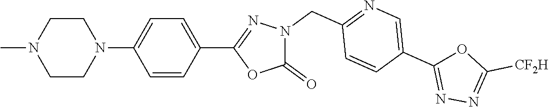 [SMILES: CN1CCN(c2ccc(-c3nn(Cc4ccc(-c5nnc(C(F)F)o5)cn4)c(=O)o3)cc2)CC1] |
| 22 | 22 | 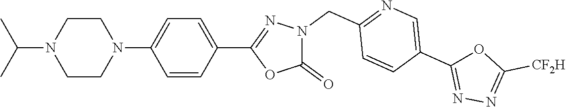 [SMILES: CC(C)N1CCN(c2ccc(-c3nn(Cc4ccc(-c5nnc(C(F)F)o5)cn4)c(=O)o3)cc2)CC1] |
| 23 | 23 | 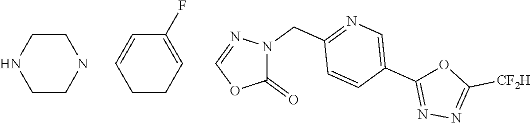 |
| 24 | 24 | 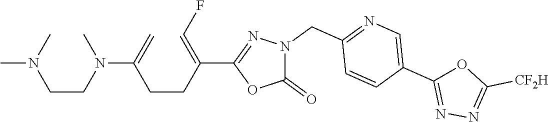 [SMILES: C=C(CC/C(=C/F)c1nn(Cc2ccc(-c3nnc(C(F)F)o3)cn2)c(=O)o1)N(C)CCN(C)C] |
| 25 | 25 | 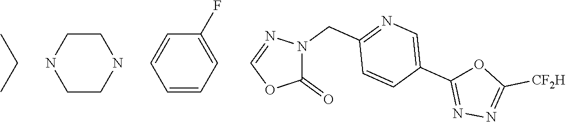 |
| 26 | 26 | 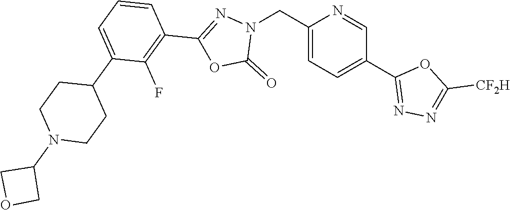 [SMILES: O=c1oc(-c2cccc(C3CCN(C4COC4)CC3)c2F)nn1Cc1ccc(-c2nnc(C(F)F)o2)cn1] |
| 27 | 27 | 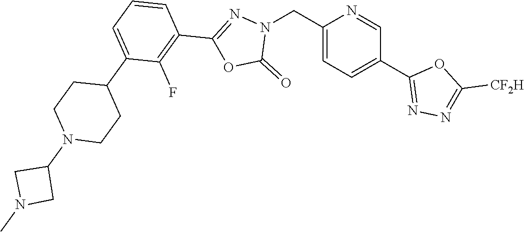 [SMILES: CN1CC(N2CCC(c3cccc(-c4nn(Cc5ccc(-c6nnc(C(F)F)o6)cn5)c(=O)o4)c3F)CC2)C1] |
| 28 | 28 | 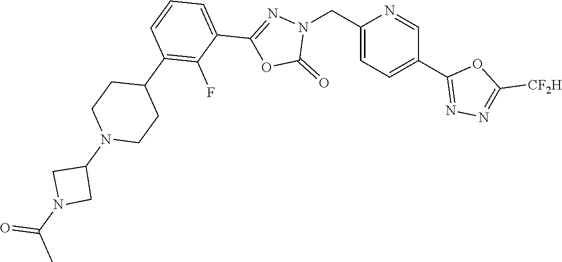 [SMILES: CC(=O)N1CC(N2CCC(c3cccc(-c4nn(Cc5ccc(-c6nnc(C(F)F)o6)cn5)c(=O)o4)c3F)CC2)C1] |
| 29 | 29 | 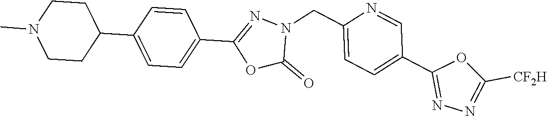 [SMILES: CN1CCC(c2ccc(-c3nn(Cc4ccc(-c5nnc(C(F)F)o5)cn4)c(=O)o3)cc2)CC1] |
| 30 | 30 | 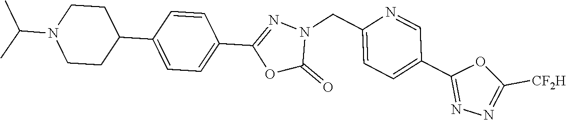 [SMILES: CC(C)N1CCC(c2ccc(-c3nn(Cc4ccc(-c5nnc(C(F)F)o5)cn4)c(=O)o3)cc2)CC1] |
| 31 | 31 | 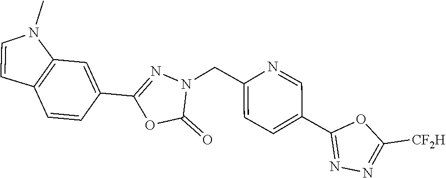 [SMILES: Cn1ccc2ccc(-c3nn(Cc4ccc(-c5nnc(C(F)F)o5)cn4)c(=O)o3)cc21] |
| 32 | 32 | 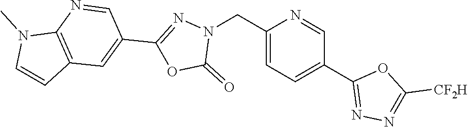 [SMILES: Cn1ccc2cc(-c3nn(Cc4ccc(-c5nnc(C(F)F)o5)cn4)c(=O)o3)cnc21] |
| 33 | 33 | 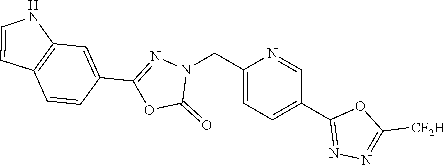 [SMILES: O=c1oc(-c2ccc3cc[nH]c3c2)nn1Cc1ccc(-c2nnc(C(F)F)o2)cn1] |
| 34 | 34 | 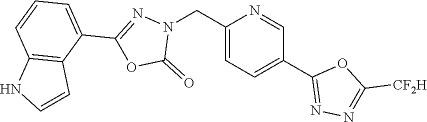 [SMILES: O=c1oc(-c2cccc3[nH]ccc23)nn1Cc1ccc(-c2nnc(C(F)F)o2)cn1] |
| 35 | 35 | 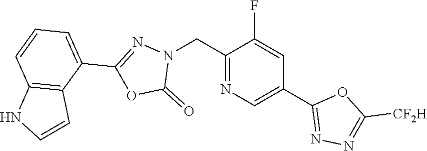 [SMILES: O=c1oc(-c2cccc3[nH]ccc23)nn1Cc1ncc(-c2nnc(C(F)F)o2)cc1F] |
| 36 | 36 | 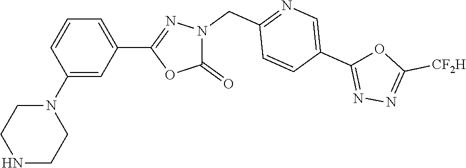 [SMILES: O=c1oc(-c2cccc(N3CCNCC3)c2)nn1Cc1ccc(-c2nnc(C(F)F)o2)cn1] |
| 37 | 37 | 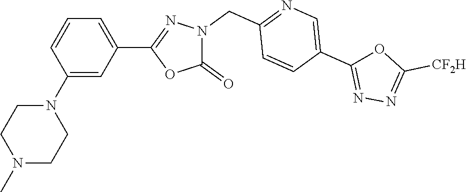 [SMILES: CN1CCN(c2cccc(-c3nn(Cc4ccc(-c5nnc(C(F)F)o5)cn4)c(=O)o3)c2)CC1] |
| 38 | 38 | 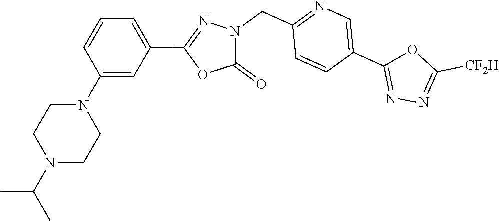 [SMILES: CC(C)N1CCN(c2cccc(-c3nn(Cc4ccc(-c5nnc(C(F)F)o5)cn4)c(=O)o3)c2)CC1] |
| 39 | 39 | 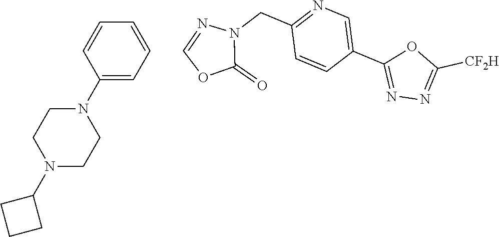 |
| 40 | 40 | 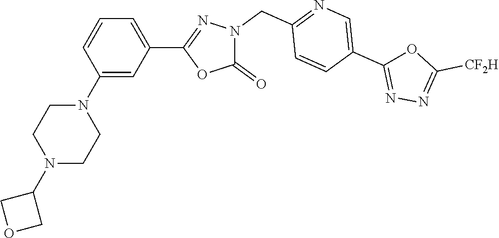 [SMILES: O=c1oc(-c2cccc(N3CCN(C4COC4)CC3)c2)nn1Cc1ccc(-c2nnc(C(F)F)o2)cn1] |
| 41 | 41 | 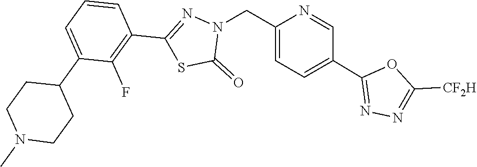 [SMILES: CN1CCC(c2cccc(-c3nn(Cc4ccc(-c5nnc(C(F)F)o5)cn4)c(=O)s3)c2F)CC1] |
| 42 | 42 | 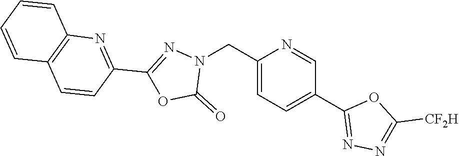 [SMILES: O=c1oc(-c2ccc3ccccc3n2)nn1Cc1ccc(-c2nnc(C(F)F)o2)cn1] |
| 43 | 43 | 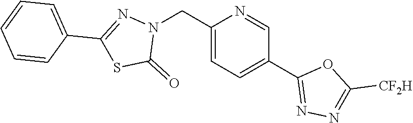 [SMILES: O=c1sc(-c2ccccc2)nn1Cc1ccc(-c2nnc(C(F)F)o2)cn1] |
| 44 | 44 | 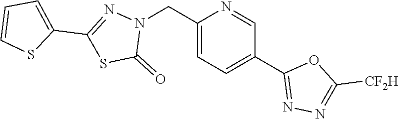 [SMILES: O=c1sc(-c2cccs2)nn1Cc1ccc(-c2nnc(C(F)F)o2)cn1] |
| 45 | 45 | 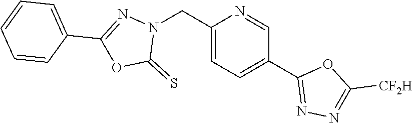 [SMILES: FC(F)c1nnc(-c2ccc(Cn3nc(-c4ccccc4)oc3=S)nc2)o1] |
| 46 | 46 | 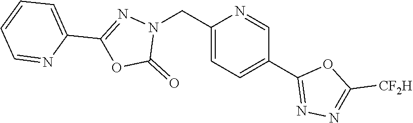 [SMILES: O=c1oc(-c2ccccn2)nn1Cc1ccc(-c2nnc(C(F)F)o2)cn1] |
| 47 | 47 | 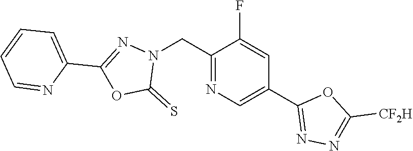 [SMILES: Fc1cc(-c2nnc(C(F)F)o2)cnc1Cn1nc(-c2ccccn2)oc1=S] |
| 48 | 48 | 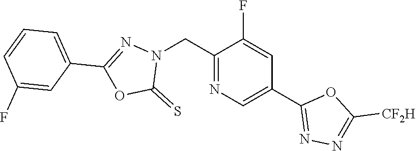 [SMILES: Fc1cccc(-c2nn(Cc3ncc(-c4nnc(C(F)F)o4)cc3F)c(=S)o2)c1] |
| 49 | 49 | 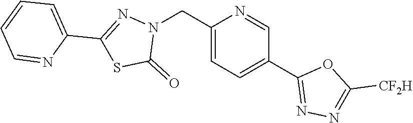 [SMILES: O=c1sc(-c2ccccn2)nn1Cc1ccc(-c2nnc(C(F)F)o2)cn1] |
| 50 | 50 | 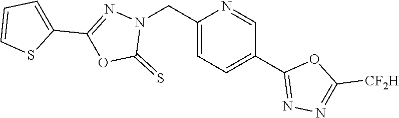 [SMILES: FC(F)c1nnc(-c2ccc(Cn3nc(-c4cccs4)oc3=S)nc2)o1] |
| 51 | 51 | 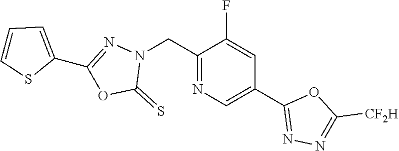 [SMILES: Fc1cc(-c2nnc(C(F)F)o2)cnc1Cn1nc(-c2cccs2)oc1=S] |
| 52 | 52 | 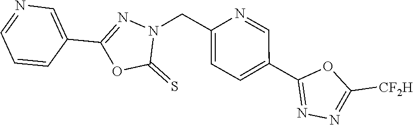 [SMILES: FC(F)c1nnc(-c2ccc(Cn3nc(-c4cccnc4)oc3=S)nc2)o1] |
| 53 | 53 | 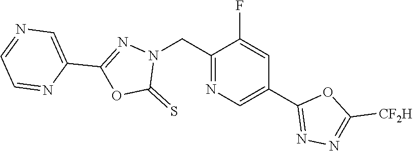 [SMILES: Fc1cc(-c2nnc(C(F)F)o2)cnc1Cn1nc(-c2cnccn2)oc1=S] |
| 54 | 54 | 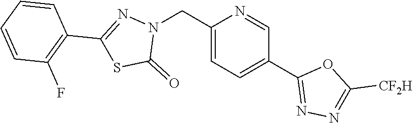 [SMILES: O=c1sc(-c2ccccc2F)nn1Cc1ccc(-c2nnc(C(F)F)o2)cn1] |
| 55 | 55 | 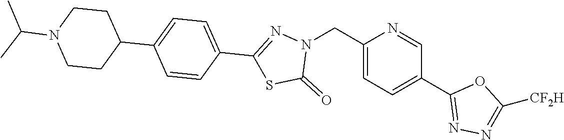 [SMILES: CC(C)N1CCC(c2ccc(-c3nn(Cc4ccc(-c5nnc(C(F)F)o5)cn4)c(=O)s3)cc2)CC1] |
| 56 | 56 | 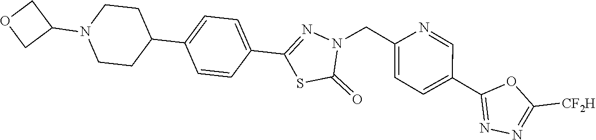 [SMILES: O=c1sc(-c2ccc(C3CCN(C4COC4)CC3)cc2)nn1Cc1ccc(-c2nnc(C(F)F)o2)cn1] |
| 57 | 57 | 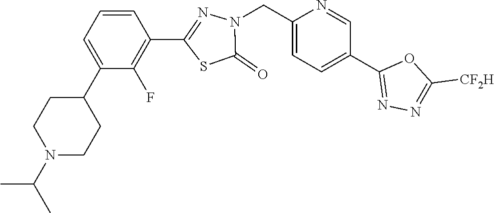 [SMILES: CC(C)N1CCC(c2cccc(-c3nn(Cc4ccc(-c5nnc(C(F)F)o5)cn4)c(=O)s3)c2F)CC1] |
| 58 | 58 | 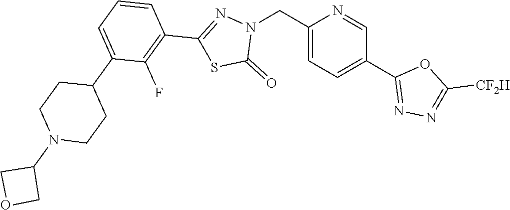 [SMILES: O=c1sc(-c2cccc(C3CCN(C4COC4)CC3)c2F)nn1Cc1ccc(-c2nnc(C(F)F)o2)cn1] |
| 59 | 59 | 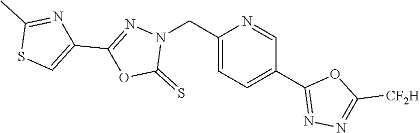 [SMILES: Cc1nc(-c2nn(Cc3ccc(-c4nnc(C(F)F)o4)cn3)c(=S)o2)cs1] |
| 60 | 60 | 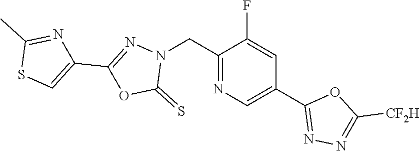 [SMILES: Cc1nc(-c2nn(Cc3ncc(-c4nnc(C(F)F)o4)cc3F)c(=S)o2)cs1] |
| 61 | 61 | 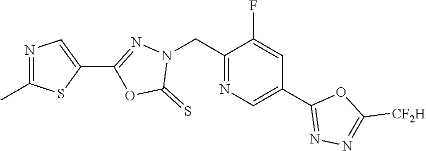 [SMILES: Cc1ncc(-c2nn(Cc3ncc(-c4nnc(C(F)F)o4)cc3F)c(=S)o2)s1] |
| 62 | 62 | 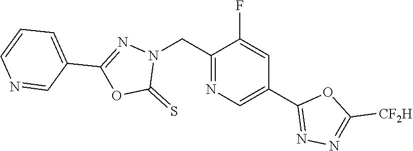 [SMILES: Fc1cc(-c2nnc(C(F)F)o2)cnc1Cn1nc(-c2cccnc2)oc1=S] |
| 63 | 63 | 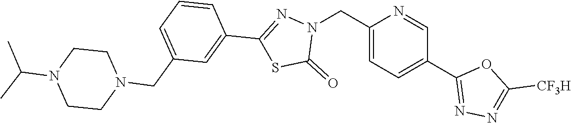 |
| 64 | 64 | 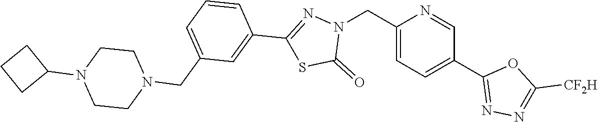 [SMILES: O=c1sc(-c2cccc(CN3CCN(C4CCC4)CC3)c2)nn1Cc1ccc(-c2nnc(C(F)F)o2)cn1] |
| 65 | 65 | 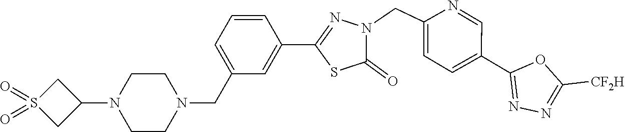 [SMILES: O=c1sc(-c2cccc(CN3CCN(C4CS(=O)(=O)C4)CC3)c2)nn1Cc1ccc(-c2nnc(C(F)F)o2)cn1] |
| 66 | 66 | 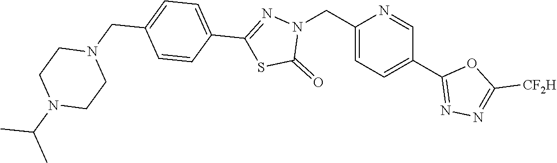 [SMILES: CC(C)N1CCN(Cc2ccc(-c3nn(Cc4ccc(-c5nnc(C(F)F)o5)cn4)c(=O)s3)cc2)CC1] |
| 67 | 67 | 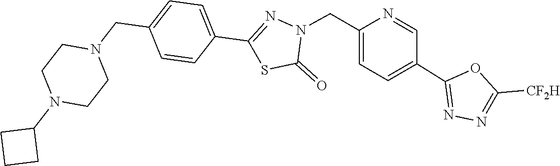 [SMILES: O=c1sc(-c2ccc(CN3CCN(C4CCC4)CC3)cc2)nn1Cc1ccc(-c2nnc(C(F)F)o2)cn1] |
| 68 | 68 | 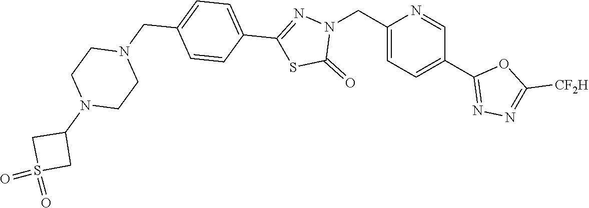 [SMILES: O=c1sc(-c2ccc(CN3CCN(C4CS(=O)(=O)C4)CC3)cc2)nn1Cc1ccc(-c2nnc(C(F)F)o2)cn1] |
| 69 | 69 | 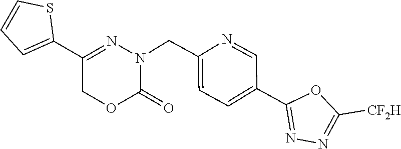 [SMILES: O=C1OCC(c2cccs2)=NN1Cc1ccc(-c2nnc(C(F)F)o2)cn1] |
| 70 | 70 | 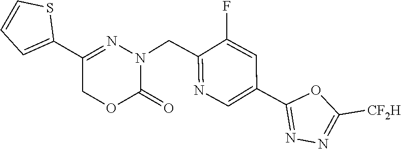 [SMILES: O=C1OCC(c2cccs2)=NN1Cc1ncc(-c2nnc(C(F)F)o2)cc1F] |
| 71 | 71 | 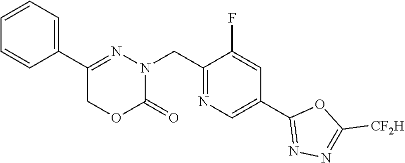 [SMILES: O=C1OCC(c2ccccc2)=NN1Cc1ncc(-c2nnc(C(F)F)o2)cc1F] |
| 72 | 72 | 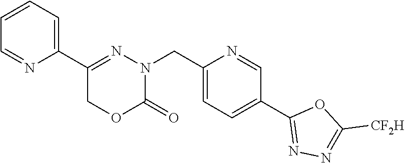 [SMILES: O=C1OCC(c2ccccn2)=NN1Cc1ccc(-c2nnc(C(F)F)o2)cn1] |
| 73 | 73 | 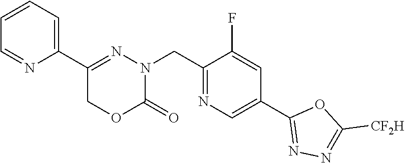 [SMILES: O=C1OCC(c2ccccn2)=NN1Cc1ncc(-c2nnc(C(F)F)o2)cc1F] |
| 74 | 74 | 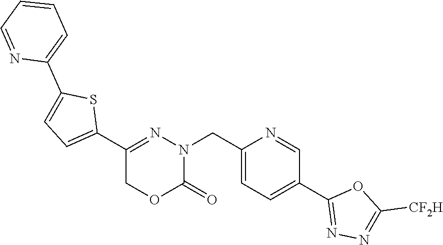 [SMILES: O=C1OCC(c2ccc(-c3ccccn3)s2)=NN1Cc1ccc(-c2nnc(C(F)F)o2)cn1] |
| 75 | 75 | 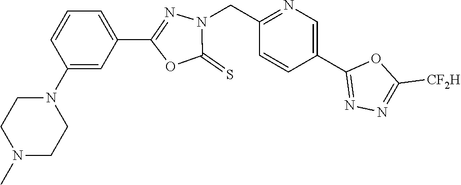 [SMILES: CN1CCN(c2cccc(-c3nn(Cc4ccc(-c5nnc(C(F)F)o5)cn4)c(=S)o3)c2)CC1] |
| 76 | 76 | 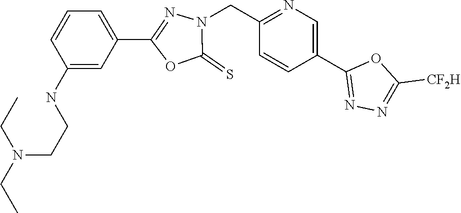 [SMILES: CCN(CC)CCNc1cccc(-c2nn(Cc3ccc(-c4nnc(C(F)F)o4)cn3)c(=S)o2)c1] |
| 77 | 77 | 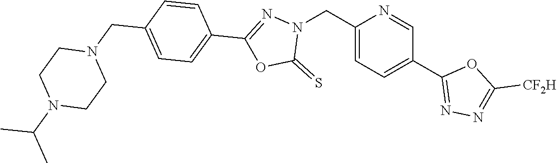 [SMILES: CC(C)N1CCN(Cc2ccc(-c3nn(Cc4ccc(-c5nnc(C(F)F)o5)cn4)c(=S)o3)cc2)CC1] |
| 78 | 78 | 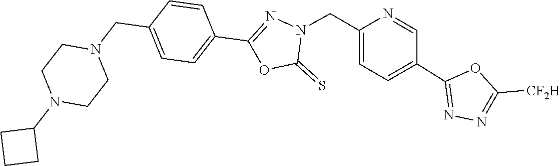 [SMILES: FC(F)c1nnc(-c2ccc(Cn3nc(-c4ccc(CN5CCN(C6CCC6)CC5)cc4)oc3=S)nc2)o1] |
| 79 | 79 | 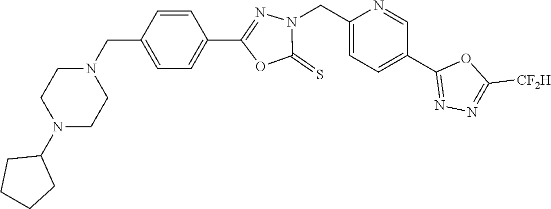 [SMILES: FC(F)c1nnc(-c2ccc(Cn3nc(-c4ccc(CN5CCN(C6CCCC6)CC5)cc4)oc3=S)nc2)o1] |
| 80 | 80 | 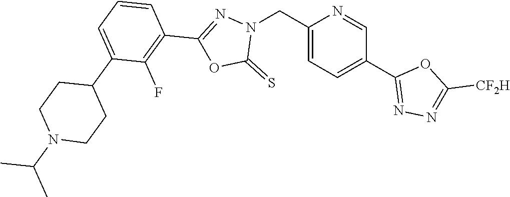 [SMILES: CC(C)N1CCC(c2cccc(-c3nn(Cc4ccc(-c5nnc(C(F)F)o5)cn4)c(=S)o3)c2F)CC1] |
| 81 | 81 | 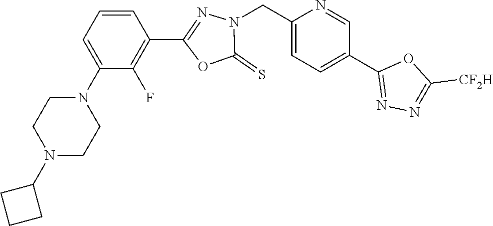 [SMILES: Fc1c(-c2nn(Cc3ccc(-c4nnc(C(F)F)o4)cn3)c(=S)o2)cccc1N1CCN(C2CCC2)CC1] |
| 82 | 82 | 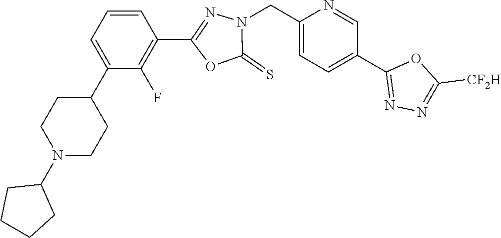 [SMILES: Fc1c(-c2nn(Cc3ccc(-c4nnc(C(F)F)o4)cn3)c(=S)o2)cccc1C1CCN(C2CCCC2)CC1] |
| 83 | 83 | 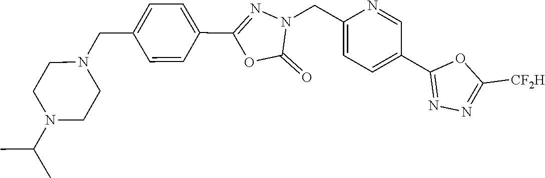 [SMILES: CC(C)N1CCN(Cc2ccc(-c3nn(Cc4ccc(-c5nnc(C(F)F)o5)cn4)c(=O)o3)cc2)CC1] |
| 84 | 84 | 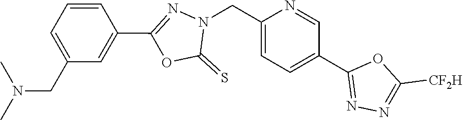 [SMILES: CN(C)Cc1cccc(-c2nn(Cc3ccc(-c4nnc(C(F)F)o4)cn3)c(=S)o2)c1] |
| 85 | 85 | 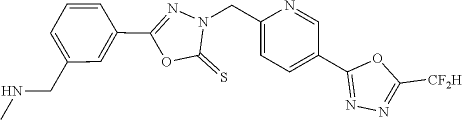 [SMILES: CNCc1cccc(-c2nn(Cc3ccc(-c4nnc(C(F)F)o4)cn3)c(=S)o2)c1] |
| 86 | 86 | 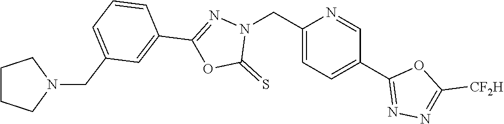 [SMILES: FC(F)c1nnc(-c2ccc(Cn3nc(-c4cccc(CN5CCCC5)c4)oc3=S)nc2)o1] |
| 87 | 87 | 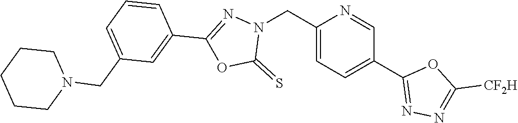 [SMILES: FC(F)c1nnc(-c2ccc(Cn3nc(-c4cccc(CN5CCCCC5)c4)oc3=S)nc2)o1] |
| 88 | 88 | 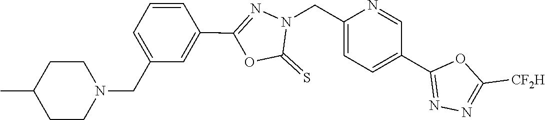 [SMILES: CC1CCN(Cc2cccc(-c3nn(Cc4ccc(-c5nnc(C(F)F)o5)cn4)c(=S)o3)c2)CC1] |
| 89 | 89 | 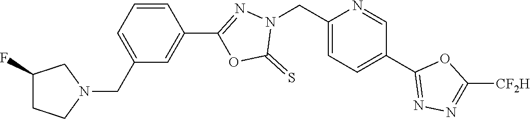 [SMILES: FC(F)c1nnc(-c2ccc(Cn3nc(-c4cccc(CN5CC[C@@H](F)C5)c4)oc3=S)nc2)o1] |
| 90 | 90 | 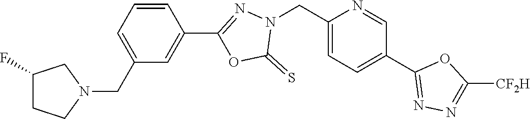 [SMILES: FC(F)c1nnc(-c2ccc(Cn3nc(-c4cccc(CN5CC[C@H](F)C5)c4)oc3=S)nc2)o1] |
| 91 | 91 | 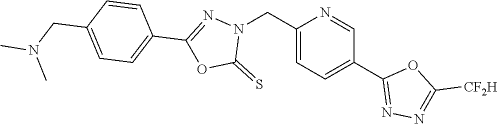 [SMILES: CN(C)Cc1ccc(-c2nn(Cc3ccc(-c4nnc(C(F)F)o4)cn3)c(=S)o2)cc1] |
| 92 | 92 | 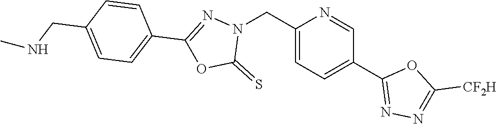 [SMILES: CNCc1ccc(-c2nn(Cc3ccc(-c4nnc(C(F)F)o4)cn3)c(=S)o2)cc1] |
| 93 | 93 | 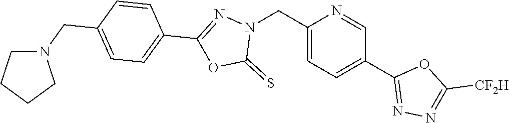 [SMILES: FC(F)c1nnc(-c2ccc(Cn3nc(-c4ccc(CN5CCCC5)cc4)oc3=S)nc2)o1] |
| 94 | 94 | 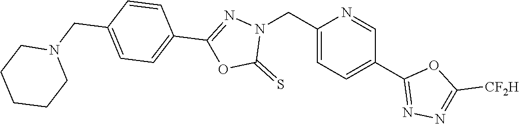 [SMILES: FC(F)c1nnc(-c2ccc(Cn3nc(-c4ccc(CN5CCCCC5)cc4)oc3=S)nc2)o1] |
| 95 | 95 | 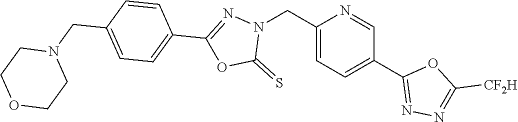 [SMILES: FC(F)c1nnc(-c2ccc(Cn3nc(-c4ccc(CN5CCOCC5)cc4)oc3=S)nc2)o1] |
| 96 | 96 | 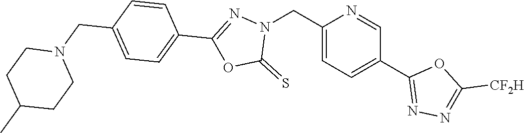 [SMILES: CC1CCN(Cc2ccc(-c3nn(Cc4ccc(-c5nnc(C(F)F)o5)cn4)c(=S)o3)cc2)CC1] |
| 97 | 97 | 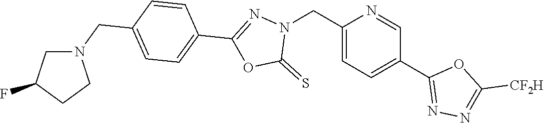 [SMILES: FC(F)c1nnc(-c2ccc(Cn3nc(-c4ccc(CN5CC[C@@H](F)C5)cc4)oc3=S)nc2)o1] |
| 98 | 98 | 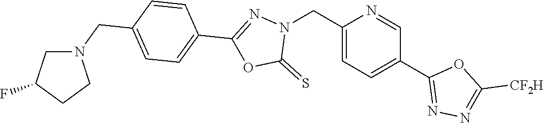 [SMILES: FC(F)c1nnc(-c2ccc(Cn3nc(-c4ccc(CN5CC[C@H](F)C5)cc4)oc3=S)nc2)o1] |
| 99 | 99 | 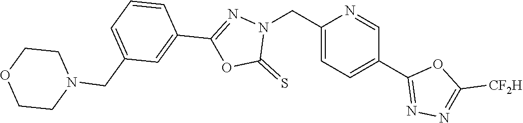 [SMILES: FC(F)c1nnc(-c2ccc(Cn3nc(-c4cccc(CN5CCOCC5)c4)oc3=S)nc2)o1] |
| 100 | 100 | 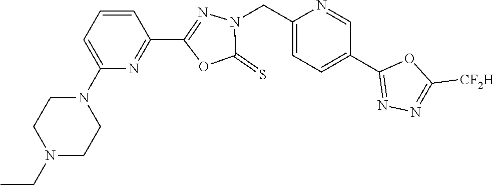 [SMILES: CCN1CCN(c2cccc(-c3nn(Cc4ccc(-c5nnc(C(F)F)o5)cn4)c(=S)o3)n2)CC1] |
| 101 | 101 | 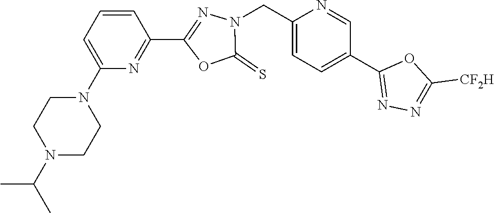 [SMILES: CC(C)N1CCN(c2cccc(-c3nn(Cc4ccc(-c5nnc(C(F)F)o5)cn4)c(=S)o3)n2)CC1] |
| 102 | 102 | 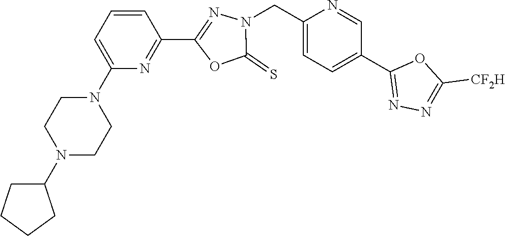 [SMILES: FC(F)c1nnc(-c2ccc(Cn3nc(-c4cccc(N5CCN(C6CCCC6)CC5)n4)oc3=S)nc2)o1] |
| 103 | 103 | 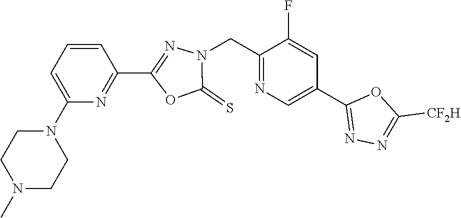 [SMILES: CN1CCN(c2cccc(-c3nn(Cc4ncc(-c5nnc(C(F)F)o5)cc4F)c(=S)o3)n2)CC1] |
| 104 | 104 | 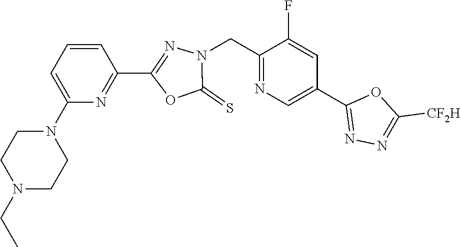 [SMILES: CCN1CCN(c2cccc(-c3nn(Cc4ncc(-c5nnc(C(F)F)o5)cc4F)c(=S)o3)n2)CC1] |
| 105 | 105 | 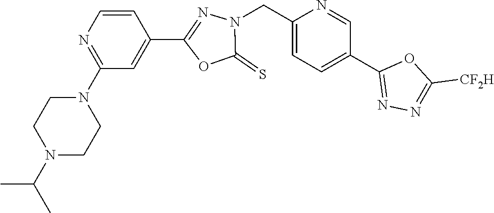 [SMILES: CC(C)N1CCN(c2cc(-c3nn(Cc4ccc(-c5nnc(C(F)F)o5)cn4)c(=S)o3)ccn2)CC1] |
| 106 | 106 | 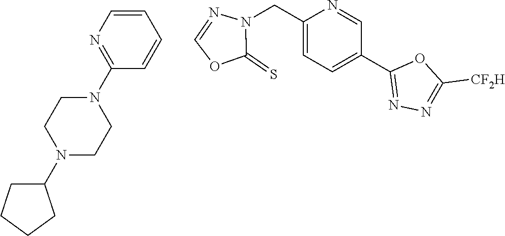 |
| 107 | 107 | 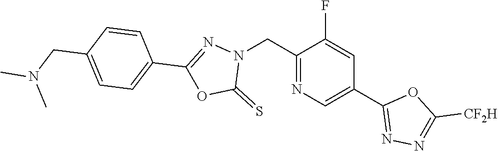 [SMILES: CN(C)Cc1ccc(-c2nn(Cc3ncc(-c4nnc(C(F)F)o4)cc3F)c(=S)o2)cc1] |
| 108 | 108 | 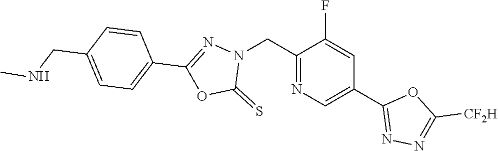 [SMILES: CNCc1ccc(-c2nn(Cc3ncc(-c4nnc(C(F)F)o4)cc3F)c(=S)o2)cc1] |
| 109 | 109 | 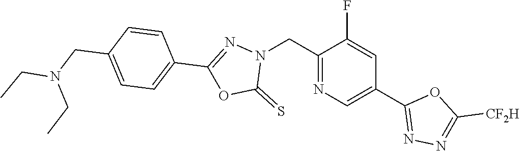 [SMILES: CCN(CC)Cc1ccc(-c2nn(Cc3ncc(-c4nnc(C(F)F)o4)cc3F)c(=S)o2)cc1] |
| 110 | 110 | 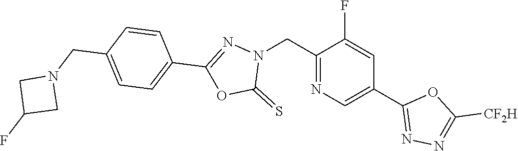 [SMILES: Fc1cc(-c2nnc(C(F)F)o2)cnc1Cn1nc(-c2ccc(CN3CC(F)C3)cc2)oc1=S] |
| 111 | 111 | 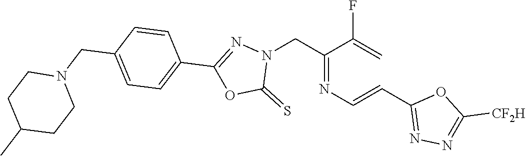 [SMILES: C=C(F)/C(Cn1nc(-c2ccc(CN3CCC(C)CC3)cc2)oc1=S)=N\C=C\c1nnc(C(F)F)o1] |
| 112 | 112 | 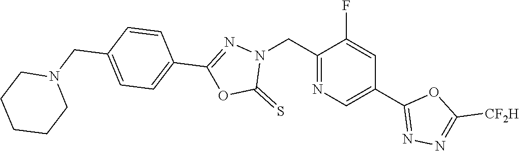 [SMILES: Fc1cc(-c2nnc(C(F)F)o2)cnc1Cn1nc(-c2ccc(CN3CCCCC3)cc2)oc1=S] |
| 113 | 113 | 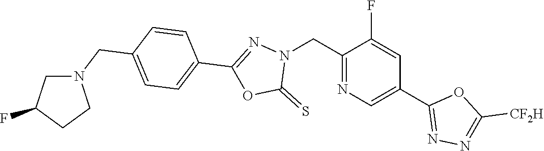 [SMILES: Fc1cc(-c2nnc(C(F)F)o2)cnc1Cn1nc(-c2ccc(CN3CC[C@@H](F)C3)cc2)oc1=S] |
| 114 | 114 | 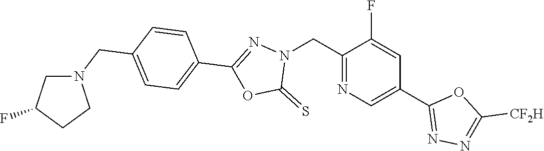 [SMILES: Fc1cc(-c2nnc(C(F)F)o2)cnc1Cn1nc(-c2ccc(CN3CC[C@H](F)C3)cc2)oc1=S] |
| 115 | 115 | 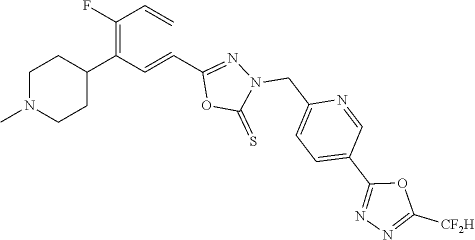 [SMILES: C=C/C(F)=C(\C=C\c1nn(Cc2ccc(-c3nnc(C(F)F)o3)cn2)c(=S)o1)C1CCN(C)CC1] |
| 116 | 116 | 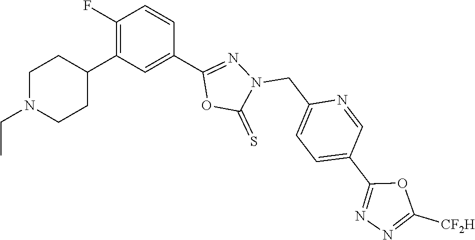 [SMILES: CCN1CCC(c2cc(-c3nn(Cc4ccc(-c5nnc(C(F)F)o5)cn4)c(=S)o3)ccc2F)CC1] |
| 117 | 117 | 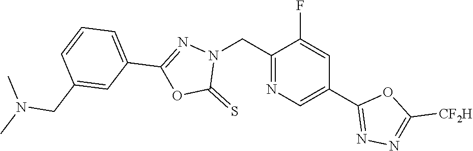 [SMILES: CN(C)Cc1cccc(-c2nn(Cc3ncc(-c4nnc(C(F)F)o4)cc3F)c(=S)o2)c1] |
| 118 | 118 | 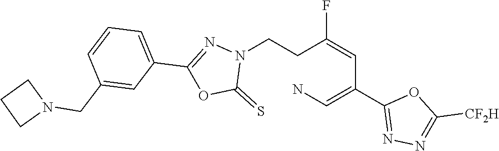 [SMILES: N/C=C(\C=C(\F)CCn1nc(-c2cccc(CN3CCC3)c2)oc1=S)c1nnc(C(F)F)o1] |
| 119 | 119 | 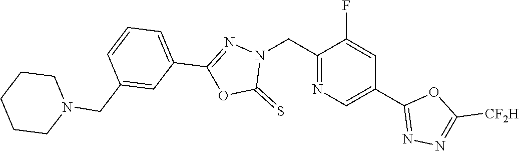 [SMILES: Fc1cc(-c2nnc(C(F)F)o2)cnc1Cn1nc(-c2cccc(CN3CCCCC3)c2)oc1=S] |
| 120 | 120 | 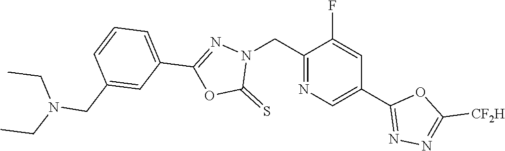 [SMILES: CCN(CC)Cc1cccc(-c2nn(Cc3ncc(-c4nnc(C(F)F)o4)cc3F)c(=S)o2)c1] |
| 121 | 121 | 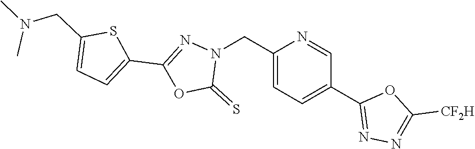 [SMILES: CN(C)Cc1ccc(-c2nn(Cc3ccc(-c4nnc(C(F)F)o4)cn3)c(=S)o2)s1] |
| 122 | 122 | 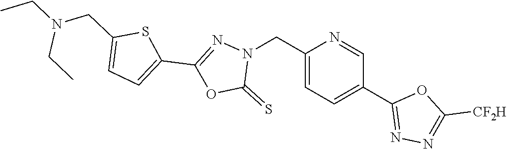 [SMILES: CCN(CC)Cc1ccc(-c2nn(Cc3ccc(-c4nnc(C(F)F)o4)cn3)c(=S)o2)s1] |
| 123 | 123 | 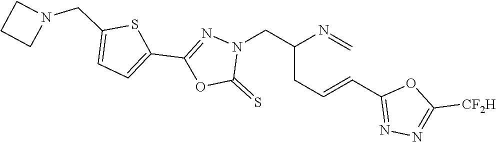 [SMILES: C=NC(C/C=C/c1nnc(C(F)F)o1)Cn1nc(-c2ccc(CN3CCC3)s2)oc1=S] |
| 124 | 124 | 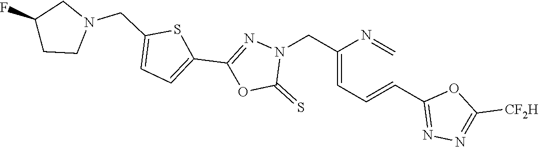 [SMILES: C=N/C(=C\C=C\c1nnc(C(F)F)o1)Cn1nc(-c2ccc(CN3CC[C@@H](F)C3)s2)oc1=S] |
| 125 | 125 | 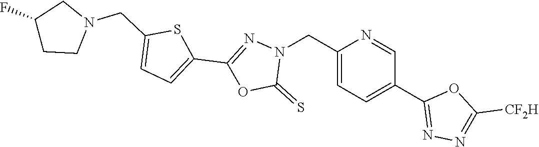 [SMILES: FC(F)c1nnc(-c2ccc(Cn3nc(-c4ccc(CN5CC[C@H](F)C5)s4)oc3=S)nc2)o1] |
| 126 | 126 | 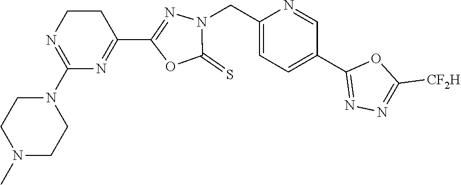 [SMILES: CN1CCN(C2=NCCC(c3nn(Cc4ccc(-c5nnc(C(F)F)o5)cn4)c(=S)o3)=N2)CC1] |
| 127 | 127 | 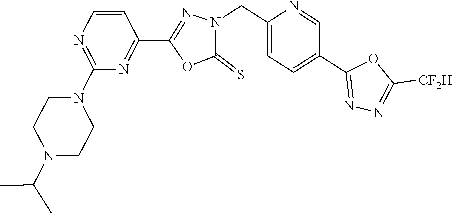 [SMILES: CC(C)N1CCN(c2nccc(-c3nn(Cc4ccc(-c5nnc(C(F)F)o5)cn4)c(=S)o3)n2)CC1] |
| 128 | 128 | 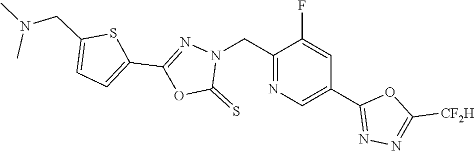 [SMILES: CN(C)Cc1ccc(-c2nn(Cc3ncc(-c4nnc(C(F)F)o4)cc3F)c(=S)o2)s1] |
| 129 | 129 | 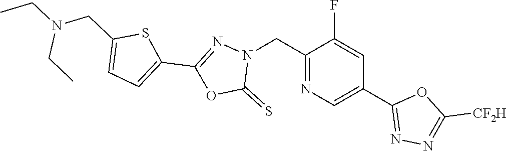 [SMILES: CCN(CC)Cc1ccc(-c2nn(Cc3ncc(-c4nnc(C(F)F)o4)cc3F)c(=S)o2)s1] |
| 130 | 130 | 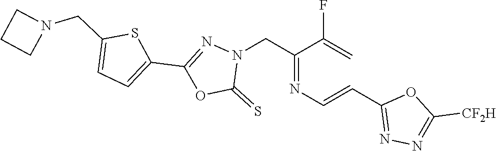 [SMILES: C=C(F)/C(Cn1nc(-c2ccc(CN3CCC3)s2)oc1=S)=N\C=C\c1nnc(C(F)F)o1] |
| 131 | 131 | 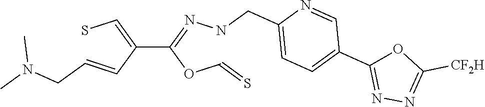 [SMILES: CN(C)C/C=C/C(=C\S)C(=N/NCc1ccc(-c2nnc(C(F)F)o2)cn1)/OC=S] |
| 132 | 132 | 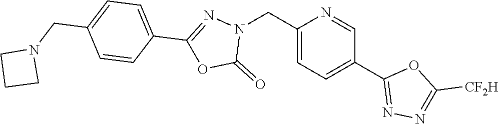 [SMILES: O=c1oc(-c2ccc(CN3CCC3)cc2)nn1Cc1ccc(-c2nnc(C(F)F)o2)cn1] |
| 133 | 133 | 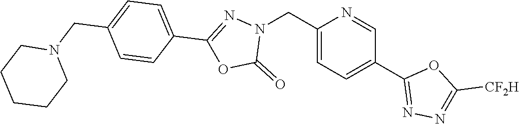 [SMILES: O=c1oc(-c2ccc(CN3CCCCC3)cc2)nn1Cc1ccc(-c2nnc(C(F)F)o2)cn1] |
| 134 | 134 | 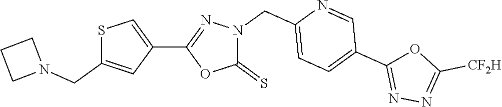 [SMILES: FC(F)c1nnc(-c2ccc(Cn3nc(-c4csc(CN5CCC5)c4)oc3=S)nc2)o1] |
| 135 | 135 | 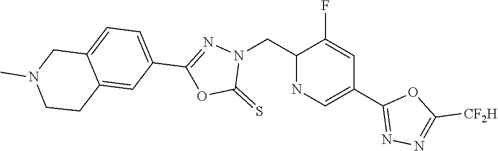 [SMILES: CN1CCc2cc(-c3nn(CC4NC=C(c5nnc(C(F)F)o5)C=C4F)c(=S)o3)ccc2C1] |
| 136 | 136 | 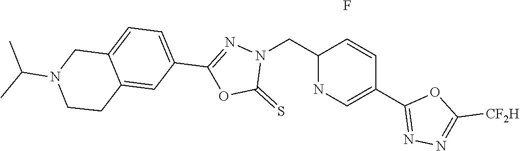 |
| 137 | 137 | 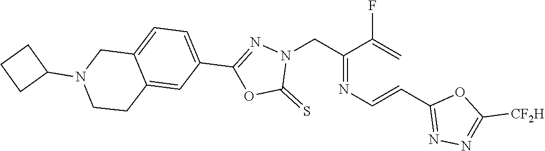 [SMILES: C=C(F)/C(Cn1nc(-c2ccc3c(c2)CCN(C2CCC2)C3)oc1=S)=N\C=C\c1nnc(C(F)F)o1] |
| 138 | 138 | 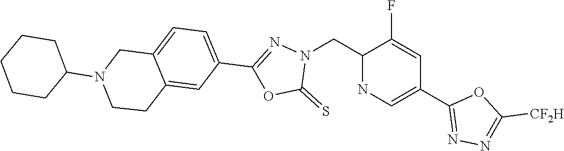 [SMILES: FC1=CC(c2nnc(C(F)F)o2)=CNC1Cn1nc(-c2ccc3c(c2)CCN(C2CCCCC2)C3)oc1=S] |
| 139 | 139 | 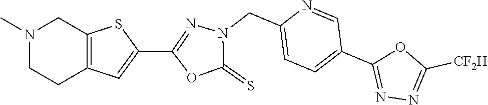 [SMILES: CN1CCc2cc(-c3nn(Cc4ccc(-c5nnc(C(F)F)o5)cn4)c(=S)o3)sc2C1] |
| 140 | 140 | 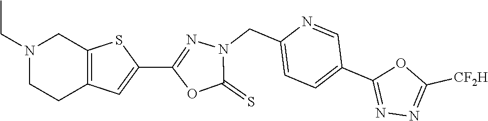 [SMILES: CCN1CCc2cc(-c3nn(Cc4ccc(-c5nnc(C(F)F)o5)cn4)c(=S)o3)sc2C1] |
| 141 | 141 | 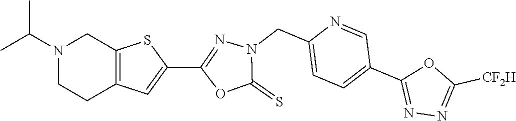 [SMILES: CC(C)N1CCc2cc(-c3nn(Cc4ccc(-c5nnc(C(F)F)o5)cn4)c(=S)o3)sc2C1] |
| 142 | 142 | 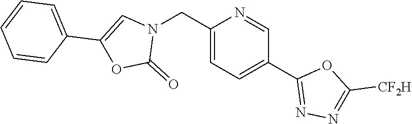 [SMILES: O=c1oc(-c2ccccc2)cn1Cc1ccc(-c2nnc(C(F)F)o2)cn1] |
| 143 | 143 | 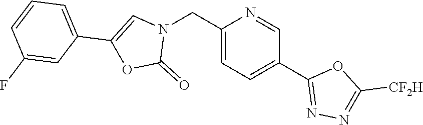 [SMILES: O=c1oc(-c2cccc(F)c2)cn1Cc1ccc(-c2nnc(C(F)F)o2)cn1] |
| 144 | 144 | 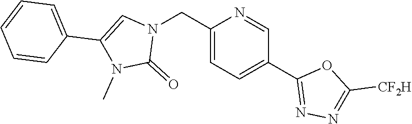 [SMILES: Cn1c(-c2ccccc2)cn(Cc2ccc(-c3nnc(C(F)F)o3)cn2)c1=O] |

8. A pharmaceutical composition for preventing or treating histone deacetylase 6-mediated diseases, comprising:
the compound represented by Chemical Formula I according to any one of claims 1 to 7, the stereoisomer thereof, or the pharmaceutically acceptable salt thereof as an active ingredient.

9. The pharmaceutical composition for preventing or treating the histone deacetylase 6-mediated diseases according to claim 8, wherein
the histone deacetylase 6-mediated diseases are infectious diseases; neoplasm; endocrine, nutritional, and metabolic diseases; mental and behavioral disorders; neurological diseases; eye and ocular adnexal diseases; circulatory diseases; respiratory diseases; digestive diseases; skin and subcutaneous tissue diseases; musculoskeletal and connective tissue diseases; or congenital malformations, alterations, and chromosomal abnormalities.

10. The pharmaceutical composition for preventing or treating the histone deacetylase 6-mediated diseases according to claim 9, wherein the endocrine, nutritional and metabolic disease is Wilson's disease, amyloidosis or diabetes,
the mental and behavioral disorder is depression or Rett syndrome, the neurological disease is central nervous system atrophy, neurodegenerative disease, movement disorder, neuropathy, motor neuron disease, and central nervous system demyelinating disease,
the eye and ocular adnexal disease is uveitis,
the skin and subcutaneous tissue disease is psoriasis,
the musculoskeletal and connective tissue disease is rheumatoid arthritis, osteoarthritis or systemic lupus erythematosus (SLE),
the congenital malformation, alteration and chromosomal abnormality is autosomal dominant polycystic kidney disease,
the infectious disease is prion disease,
the neoplasm is a benign tumor or a malignant tumor,
the circulatory disease is atrial fibrillation or stroke,
the respiratory disease is asthma, and
the digestive disease is alcoholic liver disease, inflammatory bowel disease, Crohn's disease, or ulcerative bowel disease.

11. A method for preventing or treating the histone deacetylase 6-mediated diseases comprising administering a therapeutically effective amount of the compound represented by Chemical Formula I, the stereoisomer thereof, or the pharmaceutically acceptable salt thereof according to any one of claims 1 to 7 as an active ingredient.

12. Use of the compound represented by Chemical Formula I, the stereoisomer thereof or the pharmaceutically acceptable salt thereof according to any one of claims 1 to 7 for preparing a medicament for preventing or treating the histone deacetylase 6-mediated diseases.

13. Use of the compound represented by Chemical Formula I, the stereoisomer thereof or the pharmaceutically acceptable salt thereof according to any one of claims 1 to 7 for preventing or treating the histone deacetylase 6-mediated diseases.
